(19) United States
(12) Patent Application Publication
(10) Pub. No.: US 2014/0065154 A1
(43) Pub. Date: Mar. 6, 2014

(54) TLR3 BINDING AGENTS

(71) Applicants: Laurent Gauthier, Marseille (FR); Catherine Massacrier, Tassin la demi lune (FR); Yannis Morel, Marseille (FR); Carine Paturel, Marcy I'E-toile (FR); Cecile Bonnafous, Marseille (FR); Mette Dahl Andersen, Vaerloese (DK); Benjamin Rossi, Marseille (FR)

(72) Inventors: Laurent Gauthier, Marseille (FR); Catherine Massacrier, Tassin la demi lune (FR); Yannis Morel, Marseille (FR); Carine Paturel, Marcy I'E-toile (FR); Cecile Bonnafous, Marseille (FR); Mette Dahl Andersen, Vaerloese (DK); Benjamin Rossi, Marseille (FR)

(73) Assignee: INNATE PHARMA, Marseille (FR)

(21) Appl. No.: 13/979,370

(22) PCT Filed: Jan. 10, 2012
(86) PCT No.: PCT/EP2012/050321

§ 371 Date: Nov. 19, 2013

Related U.S. Application Data
(60) Provisional application No. 61/431,967, filed on Jan. 12, 2011.

(51) Int. Cl. A61K 39/395; A61K 31/519; C07K 16/28

(52) U.S. Cl. CPC A61K 39/3955; C07K 16/28; A61K 31/519
USPC 424/139.1; 530/387.9; 530/391.7; 435/331; 506/9; 435/7.92; 536/23.53

(57) ABSTRACT

The invention provides anti-TLR3 antibodies as well as methods of making and using them. The antibodies are particularly adapted to the treatment of autoimmune or inflammatory diseases using anti-TLR3 antibodies.

### FIELD OF THE INVENTION

The present invention relates to antibodies (e.g. monoclonal antibodies), antibody fragments, and derivatives thereof that specifically bind TLR3, and that optionally further modulate, e.g. inhibit, signaling. The invention also relates to cells producing such antibodies; methods of making such antibodies; fragments, variants, and derivatives of the antibodies; pharmaceutical compositions comprising the same; methods of using the antibodies to diagnose, treat or prevent diseases, e.g. autoimmune diseases, inflammatory diseases and the like.

### BACKGROUND

Drosophila toll proteins control dorsal-ventral patterning and are thought to represent an ancient host defense mechanism. In humans, TLRs are believed to be an important component of innate immunity. Human and Drosophila Toll protein sequences show homology over the entire length of the protein chains. The family of human Toll-like receptors is comprised of ten highly conserved receptor proteins, TLR1-TLR10. Like Drosophila toll, human TLRs are type I transmembrane proteins with an extracellular domain consisting of a leucine-rich repeat (LRR) domain that recognizes pathogen-associated molecular patterns (PAMPs), and a cytoplasmic domain that is homologous to the cytoplasmic domain of the human interleukin-1 (IL-1) receptor. Similar to the signaling pathways for both Drosophila toll and the IL-1 receptor, human Toll-like receptors signal through the NF-κB pathway.

Although the different mammalian TLRs share many characteristics and signal transduction mechanisms, their biological functions are very different. This is due in part to the fact that four different adaptor molecules (MyD88, TIRAP, TRIF and TRAF) are associated in various combinations with the TLRs and mediate different signaling pathways. In addition, different ligands for one TLR may preferentially activate different signal transduction pathways. Furthermore, the TLRs are differentially expressed in various hematopoietic and non-hematopoietic cells. Accordingly, the response to a TLR ligand depends not only on the signal pathway activated by the TLR, but also on the nature of the cells in which the individual TLR is expressed.

Toll-like receptor 3 (TLR3) has received considerable attention as a therapeutic target as TLR3 signaling has been implicated in inflammatory and autoimmune conditions. Patent application WO98/50547 provides the nucleic acid and amino acid sequence of the hTLR3 protein. LeBouteiller et al. (2005) J. Biol. Chem. 280(46): 38133-38145) disclose use of an anti-TLR3 antibody to bind cell surface TLR3. Antibody C1130 is stated to be activatory toward TLR3 and has been described in WO 2007/051164. Polyclonal antibodies that inhibited TLR3 were described in Cavassani et al. (2008) J. Exp. Med. 205: 2609-2621. WO 03/106499 and Matsumoto et al. (2003) J. Immunol. 171:3154-3162 describes an antibody corresponding to antibody clone TLR3.7 (eBioScience Inc., San Diego) reported to bind and inhibit cell surface TLR3 but not cell compartment TLR3 or in myeloid-lineage DC. WO 06/060513 describes an antibody C1068 which is reported to inhibit cytokine production in epithelial cells, which are reported to express TLR3 on the cell surface. C1068 is stated to compete with antibody TLR3.7 for binding to TLR3 (see WO2010/051470). PCT patent application WO2010/051470 provides anti-TLR3 antibodies. Such antibodies are stated to block dsRNA and are proposed to prevent binding of dsRNA to TLR3. Other anti-TLR3 antibodies for research use include polyclonal anti-TLR3 antibodies from R&D Systems Corp., antibody 40C 1285 from Abcam and antibodies 619F7, 713E4, 716G10, IMG-5631 and -IMG-5348, all from Imgenex. Corp.

However, among currently available anti-TLR3 antibodies, they are not optimally suited for use as therapeutic agents, e.g. to modulate TLR3. There is therefore a need to provide improved antibodies directed to TLR3.

### SUMMARY OF THE INVENTION

While several anti-TLR3 antibodies have been generated to date, these antibodies have generally been intended for research only, or only demonstrated an effect in prevention of diseases, while not showing efficacy in curative treatments and/or have no demonstrated activity in the presence of TLR3 ligands. The present disclosure provides improved anti-TLR3 antibodies and demonstrates their use to ameliorate established autoimmune diseases.

In one aspect, the present invention provides methods for the treatment of an individual having an inflammatory or autoimmune disease, particularly an established inflammatory or autoimmune disease, or experiencing an attack, crisis, exacerbation or flare of an inflammatory or autoimmune disease, the method comprising administering to the individual an anti-TLR3 antibody.

The invention arises from the findings that anti-TLR3 antibodies can be obtained that are able not only to prevent a self-immunization caused by dsRNA ligands acting on TLR3, but also to inhibit TLR3 signaling once a natural TLR3 ligand such as dsRNA has induced TLR3 signaling. The antibodies function in the presence of dsRNA and furthermore are able to ameliorate ongoing and established inflammation. The inventors obtained anti-TLR3 antibodies bind human TLR3 under acidic conditions as well as surrogate antibodies that bind murine TLR3 under acidic conditions, in each case at high affinity. The acidic conditions are representative of those encountered in an acidified subcellular compartment of a cell (e.g. compartments of the endocytic, endosomic, lysosomal pathway), such as in a dendritic cell (DC). The antibodies in each case furthermore bind the respective TLR3 at neutral conditions as well, permitting binding to TLR3 polypeptides exposed at the cell surface. The antibodies can therefore bind efficiently to cell surface TLR3, are internalized upon binding, and maintain their efficacy when internalized into the endosomic pathway, as e.g. in dendritic cells.

The findings presented herein are important because they permit uses of anti-TLR3 antibodies for the treatment of patients having established disease. Consequently, TLR3 is not solely involved in the initial induction of a self-immunization by dsRNA (e.g. from infections, from autologous cells, etc.), but rather its blockade can decrease ongoing inflammation, even in chronic settings and acute inflammation.

The suitability of the anti-TLR3 antibodies for treatment of ongoing inflammation, even in chronic settings and acute inflammation, makes them suited for use in combination with a number of other agents used in inflammatory settings (a second therapeutic agent), in particular agents that decrease inflammation, e.g. such as disease modifying anti-rheumatic drugs (DMARDs, e.g. anti-TNFα and MTX) in the case of rheumatoid arthritis. Because mechanisms driving inflammation—particularly acute and chronic—are believed to often be redundant, the antibodies of the invention will be particularly useful for use in combination with agents that act on an inflammation mechanism other than TLR3, but have a similar biological objective, such as the reduction of pro-inflammatory cytokine production or action, notably the reduction or inhibition of TNFα. In one embodiment of the treatment methods of the invention, anti-TLR3 antibodies are administered before, concomitantly with or after a second therapeutic agent.

One aspect of the present invention is therefore the use of an anti-TLR3 antibody that inhibits TLR3 signalling to treat a patient experiencing an attack in the course of the inflammatory or autoimmune disease development or to treat a patient having an established disease, or disease characterized by ongoing inflammation. Still another aspect of the invention is the use of such an antibody to treat a patient having a chronic inflammatory or autoimmune disease. Still another aspect is the combined use of such antibody in combination with a second therapeutic agent, preferably a DMARD, used in the treatment of an inflammatory or autoimmune disease.

In one aspect, an anti-TLR3 antibody that inhibits TLR3 signalling is used to treat a patient having graft-versus-host disease.

In another embodiment, the finding that TLR3 inhibition using the anti-TLR3 antibodies according to the invention ameliorates ongoing disease in vivo suggests that the antibodies can be administered continuously to maintain a therapeutic effect by repeat dosing to maintain TLR3 inhibition. In one embodiment, the anti-TLR3 antibodies are administered to a patient at a frequency of from about twice per week to about once every 2 months, in an amount of from about 0.075 to about 3 mg/kg, or any other appropriate dosage as described herein. In one embodiment, provided is an article of manufacture comprising:
(a) a container comprising an anti-TLR3 antibody; and
(b) a package insert with instructions for treating cancer in a patient, wherein the instructions indicate that a dose of the anti-TLR3 antibody of about 0.075 to about 3 mg/kg is administered to the patient at a frequency of from about twice per week to about once every 2 months.

In one embodiment, provided is a method of treating an individual having an autoimmune or inflammatory disease, comprising administering to the individual an effective amount of an antibody according to the invention, wherein the effective amount is between about 0.05 and 20 mg/kg, administered to the individual at a frequency of from about once per week to about once every 2 months.

In one embodiment, provided is a method of treating an individual having an established or chronic autoimmune or inflammatory disease, comprising administering to the individual an antibody that binds a TLR3 polypeptide.

In one embodiment, provided is a method of treating an attack, crisis, flare or exacerbation in an individual having an established or chronic autoimmune or inflammatory disease, comprising administering to the individual an antibody that binds a TLR3 polypeptide.

In one embodiment, provided is a method for the treatment of an autoimmune or inflammatory disease in an individual, comprising:
(a) evaluating the presence, stage and/or evolution of disease in an individual;
(b) administering to said individual an effective dose of an antibody that binds a TLR3 polypeptide. Optionally, evaluating the presence, stage and/or evolution of disease in an individual comprises analysing levels of autoantibodies, CRP, or any proteolytic enzyme, inflammatory mediator or marker of ongoing inflammation. Optionally, evaluating the presence, stage and/or evolution of disease in an individual comprises conducting blood gas analysis, and if said individual is determined to be suitable for treatment with an antibody that binds a TLR3 polypeptide (e.g. the individual has COPD, an exacerbation, etc), administering to said individual an effective dose of an antibody that binds a TLR3 polypeptide.

In one embodiment, provided is a method for the treatment of an autoimmune or inflammatory disease in an individual, comprising:
(a) determining whether said individual has an established disease;
(b) if said individual has an established disease, administering to said patient an effective dose of an antibody that binds a TLR3 polypeptide.

In one embodiment, provided is a method for the treatment an autoimmune or inflammatory disease in an individual comprising:
(a) determining whether said individual is experiencing an attack, crisis, exacerbation or flare;
(b) if said individual experiences an attack, crisis, exacerbation or flare, administering to said individual an effective dose of an antibody that binds a TLR3 polypeptide.

In one embodiment, provided is a method for the treatment of an autoimmune or inflammatory disease in an individual, comprising:
(a) determining whether said individual has a disease characterized by the presence of dsRNA;
(b) if said individual has disease characterized by the presence of dsRNA, administering to said patient an effective dose of an antibody that binds a TLR3 polypeptide.

Optionally, in any methods of treatment, the methods further comprise administering to the individual a DMARD. In one embodiment, provided is a method of treating an individual having an autoimmune or inflammatory disease, comprising administering to the individual (a) an effective amount of an antibody that binds a TLR3 polypeptide, and (b) a DMARD.

In one embodiment, provided is a method for determining the suitability of treatment with an antibody that binds a TLR3 polypeptide for a patient, comprising determining whether said patient has an established autoimmune or inflammatory disease, whether said patient is experiencing an attack, crisis, exacerbation or flare, and/or whether said patient has a disease characterized by the presence of dsRNA.

In one embodiment, an anti-TLR3 antibody (e.g. an antibody that inhibits signaling by a TLR3 polypeptide) is delivered to nasal or pulmonary tissue. Absorption of various larger peptides and proteins such as full tetramer antibodies across the nasal mucosa is often insufficient to achieve sufficient amounts of these proteins in the bloodstream in order to provide an effective therapy. The first step in the absorption of peptides from the nasal cavity is passage through the mucus. However, smaller proteins such as antibody fragments and derivatives, e.g., Fv, Fab, Fab′, F (ab′)2, nanobodies, domain antibodies, a single domain antibodies or a “dAb” can be adapted to be more readily delivered to nasal or pulmonary tissue. It is demonstrated herein that F(ab)′2 fragments were as effective in mediating TLR3 inhibition in DC as full length antibodies, indicating that Fc regions are not required for internalization or activity. Consequently, anti-TLR3 antibodies fragments or derivatives can be administered to nasal or pulmonary tissue. Separately, antibodies administered to mice by i.p. were highly effective in a model of COPD, indicating that local delivery to pulmonary tissue may be yet furthermore effective in the treatment of COPD. Nanobodies, (single) domain antibodies or dAb's can, for example, be derived from the variable region of a tetramer antibody as well as from the variable region of a heavy chain antibody. For example, the amino acid sequence of (single) domain antibodies or dAb's may comprise four framework regions and three CDRs.

In one embodiment, an anti-TLR3 antibody-containing composition is formulated for administration to nasal or pulmonary tissue, preferably wherein the anti-TLR3 antibody is delivered by intranasal or inhalation administration. Preferably, the antibody is a Fv, Fab, Fab′, F (ab′)2 or a nanobody, domain antibody, single domain antibody or a “dAb”. In one embodiment, the antibodies are formulated such that they do not substantially enter the systemic circulation. In an alternative embodiment, the antibodies are formulated such that they do substantially enter the systemic circulation, e.g. the antibody is delivered to the bloodstream and/or other organ and/or tissue (e.g. the kidney, lung and/or brain). In one embodiment, the antibodies are delivered locally to nasal or pulmonary tissue, for the treatment of COPD or asthma.

The present invention further provides a delivery device, suitable for nasal administration of an anti-TLR3 antibody (e.g. an antibody that inhibits signaling by a TLR3 polypeptide). Accordingly, the present invention relates to a delivery device anti-TLR3 antibody and provided with a device capable of applying the anti-TLR3 antibody to the nasal mucosa. Delivery devices such as provided in the form of drops, a nasal spray, a nasal liquid or powder aerosol, a capsule or a nasal insert are known in the art. Such delivery devices are capable of (repeatedly) delivering a unit dose of the composition of the invention, and in particular of (repeatedly) delivering a unit dose of the invention that comprises a delivery volume that is suitable for nasal administration. Examples of nasal formulations are described in WO2008049897. The present invention further provides a delivery device, suitable for inhalation administration of an anti-TLR3 antibody. The antibody can be delivered using a nebulizer, inhaler, atomizer, aerosolizer, mister, dry powder inhaler, metered dose inhaler, metered dose sprayer, metered dose mister, metered dose atomizer, or other suitable delivery device.

The invention further provides a method for delivering an anti-TLR3 antibody to an individual, said method comprising the step of administering to said individual, by nasal or inhalation administration, a Fv, Fab, Fab′, F (ab′)2 or a nanobody, domain antibody, single domain antibody or a “dAb” that binds a TLR3 polypeptide. Preferably the antibody inhibits signaling by a TLR3 polypeptide.

Other embodiments of the invention include antibodies, antibody fragments, and derivatives that specifically bind human TLR3. The invention provides such antibody compositions, as well their use in any of the methods of the invention.

In one aspect, the antibodies bind human TLR3 under acidic conditions, and in particular under conditions representative of that encountered in an acidified subcellular compartment of a cell (e.g. compartments of the endocytic pathway endosomic, lysosomal). Such acidic conditions are generally characterized by a pH lower than about pH 6.5, or between about pH 4.5 to 6.5, or about pH 5.6. In one aspect of any of the embodiments herein, the antibodies modulate, optionally inhibit, TLR3 signaling in an acidified subcellular compartment of a cell (e.g. compartments of the endocytic pathway endosomic, lysosomal).

In one aspect of any of the embodiments herein, the antibodies modulate, optionally inhibit, TLR3 signaling in a dendritic cell (DC) (e.g. a myeloid DC (MdDC) or a monocyte derived DC).

In one aspect of any of the embodiments herein, the antibodies can optionally be characterized as not having substantially lower affinity for binding human TLR3 under acidic conditions than under neutral conditions e.g. where the K_{D} for binding to TLR3 decreases by no more than 0.2-, 0.3-, -0.4, 0.5-, 1.0-, or 1.5-log₁₀. Neutral conditions are generally characterized by a pH between 6.6 and 7.4, for example a slightly alkaline pH of 7.2 found in the cell cytosol. Optionally, the antibodies do not have substantially different (lower or higher) affinity for binding human TLR3 under acidic conditions than under neutral conditions e.g. where the K_{D} for binding to TLR3 under neutral and acidic conditional differs by no more than 0.2-, 0.3-, 0.4-, 0.5-, 1.0-, or 1.5-log₁₀.

In other aspects of any of the embodiments herein, the antibodies' bivalent binding affinity for TLR3 under acidic conditions can optionally be characterized by a mean K_{D} of no more than about (i.e. better affinity than) 100, 50, 10, 5, or 1 nanomolar, preferably sub-nanomolar or optionally no more than about 300, 200, 100 or 10 picomolar.

In one embodiment, the antibodies have binding affinity (K_{D}) for a human TLR3 polypeptide at an acidic pH, i.e. a pH of about 5.6, of less than 10⁻⁹ M, preferably less than 10⁻¹⁰M. In another embodiment, the antibodies have binding affinity (K_{D}) for a human TLR3 polypeptide at a neutral pH, i.e. a pH of about 7.2, of less than 10⁻⁹ M, preferably less than 10⁻¹⁰M. In another embodiment, the antibodies have an affinity of less than 10⁻⁹ M, preferably less than 10⁻¹⁰M at both an acidic pH and at a neutral pH.

In other aspects of any of the embodiments herein, the antibodies inhibit TLR3 signaling without blocking the binding of a TLR3 ligand to a TLR3 polypeptide, preferably without blocking binding of a dsRNA TLR3 ligand to the principal (C-terminal) dsRNA binding site on the TLR3 polypeptide. The TLR3 ligand will generally be a ligand other than an anti-TLR3 antibody and may be a naturally occurring or non-naturally occurring TLR3 ligand, optionally a dsRNA-based ligand such as polyAU (polyadenylic acid:polyuridylic acid) or polyIC (polyinosinic:polycytidylic acid). In particular, the inventors have established that the antibodies according to the invention are able to inhibit TLR3 signaling even when a TLR3 ligand such as dsRNA is already bound to the TLR3 polypeptide and/or when a TLR3-expression cell has been in contact with a TLR3 ligand. The antibodies according to the invention are also able to inhibit TLR3 signaling even in a pre-activated condition, e.g., in the presence of IFNα. The antibodies according to the invention are believed to be effective to treat a patient having an established autoimmune disease, e.g. naturally occurring TLR3 ligand such as dsRNA and/or the presence of, and in particular, high levels of, IFNα in the diseased cells. The antibodies will also have the advantage of binding TLR3 even if the C-terminal TLR3 ligand binding site is occupied by a dsRNA molecule thus potentially allowing broader overall binding.

The present disclosure shows that the antibodies that bind human TLR3 under acidic conditions have a strong ability to modulate, particularly inhibit, TLR3 signaling in cells (myeloid dendritic cells (MdDC); monocyte derived DC (MoDC)) which express TLR3 solely or primarily in their cytoplasmic compartments, and primarily in compartments of the endocytic pathway (e.g. endosomes). The antibodies bind to a region in TLR3 which is not involved in binding to dsRNA, and the antibodies do not prevent dsRNA from binding to TLR3, and in particular the C-terminal portion of TLR3 where the primary dsRNA binding site is found, under acidic conditions. The compositions and methods are useful for a multitude of applications, and are particularly well suited to modulating TLR3 signaling (e.g. in vivo) where cytosolic (e.g. endocytic pathway compartment-localized) TLR3 is targeted. Modulating cytosolic TLR3 signaling can be useful to treat or prevent a disease for which modulation of TLR3 signaling in DC or other cells that express TLR3 in acidic cytosolic compartments (e.g. in endosomes) is beneficial. For example, inhibiting TLR3 signaling in DC (e.g. as observed by inhibition of cytokine production by the DC) can be used the treatment or prevention of inflammatory or autoimmune disorders since DC have a well documented capacity to take up antigens from apoptotic or necrotic cells (Albert et al (2004) Nat. Rev. Immunol. 4: 223-231), including during tissue necrosis during acute inflammation (Cavassani et al. (2008). Optionally, the antibodies inhibit TLR3 signaling, e.g. inhibit cytokine production (e.g. IP10) induced by the stimulation of a TLR3 receptor by a TLR3 ligand.

Endosomes and lysosomes are membrane bound compartments inside cells, form part of the endocytic pathway and are usually acidic due to the action of a proton-pumping ATPase of the endosomal membrane. The earliest measurements of in situ lyosomal pH found a pH of 4.7-4.8 in macrophages; the pH of fibroblast endosomes involved in receptor-mediated endoxytosis was determined to be about 5.5. Early studies of TLR3 identified it as being expressed in the cytosol in monocyte-derived DCs and that it probably bound its ligand in subcellular compartments of the endocytic pathway (Matsumoto et al. (2003) J. Immunol. 171:3154-3162). TLR3 has since been reported to be expressed in cells' endosomic compartment in dendritic cells, astrocytes, macrophages, T cells, epithelial cells, fibroblasts and hepatocytes, although TLR3 has also been found on cell surface, particularly on epithelial cells, and in some cases of inflammation also on macrophages (Cavassani et al. 2008, supra). Endosomal acidification has been shown to have a role in TLR3 signaling since treatment with chloroquine, an inhibitor of endosomal acidification, inhibits TLR3 signaling in DC. The antibodies provided herein that bind TLR3 under acidic conditions corresponding to an acidified endosomic compartment (e.g. pH of about 5.6, or less than about 6.5) have the advantage of allowing efficient high affinity binding to, and optionally further modulation of, TLR3 in endosomic compartments compared to antibodies that lose their affinity under acidic conditions and thus may exert their effects more on cell surface TLR3. The antibodies exemplified have strong inhibitory activity on TLR3 in DC which are known for expressing TLR3 primarily in cytosomal compartments.

In one embodiment, the present invention provides monoclonal antibodies that specifically bind human TLR3 and inhibit TLR3 signaling, e.g. inhibit cytokine production induced by the stimulation of a TLR3 receptor by a TLR3 ligand, without blocking the binding of a ligand of TLR3 (e.g. a natural or synthetic ligand of TLR3, a nucleic acid based ligand, a dsRNA, viral dsRNA, polyIC, polyAU) to the C terminal dsRNA binding site of a TLR3 polypeptide. In one embodiment, the ligand is a high molecular weight dsRNA, optionally, a dsRNA (e.g., a polyAU or polyIC) having an Mₙ (also referred to as “number average molecular weight” or “mean molecular weight”) of at least 250 kDa, optionally 300, 500, 800, 1500, 2000, 3000 kDa, optionally an M_{w} greater than 250 kDa, optionally 300, 500, 800, 1500, 2000, 3000 kDa, wherein less than 5% of fragments have a molecular weight less than 100, 200, 500 or 1000 kDa, a PI of less than 2.0 (e.g. 1.4-1.6). In one embodiment, the dsRNA is a dsRNA that specifically activates TLR3, preferably that activates TLR3 without activating MDA-5 and/or RIG-I (e.g. a polyAU). In one embodiment, the dsRNA is any dsRNA described in WO2009/130616 (Innate Pharma), the disclosure of which is incorporated by reference herein. When TLR3 polypeptides are bound by such antibodies, dsRNA can still bind the TLR3 polypeptides, reducing dsRNA available to bind to remaining non antibody-bound TLR3 and/or other dsRNA receptors (i.e. RIG-I, MDA-5, TLR7, etc), thereby potentially reducing undesirable side effects such as increased toxicity, inappropriate signaling cascade activation and so on, and resulting conditions, e.g. chronic inflammation, that arise from dsRNA induced signaling. Such antibody compositions and methods are useful for a multitude of applications, particularly to treat or prevent a disease related to TLR3 signaling, and in view of their mechanism of action, the antibodies of the invention can be used for anergizing or inhibiting TLR3 polypeptides. Optionally, the antibody can be characterized as not detectably reducing the binding of a double-stranded RNA ligand of TLR3 to a TLR3 polypeptide. The antibody may or may not also be capable of binding with high affinity to human TLR3 under acidic conditions, e.g. under conditions representative of that encountered in an acidified endosomic compartment. In one embodiment, where an antibody is sought that can inhibit signaling by TLR3, it will be advantageous that an antibody that specifically binds TLR3 and inhibits TLR3 signaling without blocking the binding of a double-stranded RNA ligand of TLR3 to a TLR3 polypeptide can additionally be capable of binding and inhibiting human TLR3 under acidic conditions as described herein, and in particular under conditions representative of that encountered in an acidified endosomic compartment of a cell.

In one aspect of any of the embodiments of the invention, the antibody may have a heavy and/or light chain having one, two or three CDRs of an antibody selected from the group consisting of antibody 31C3, 28F11, 34A3, 29H3 and 23C8. In one aspect of any of the embodiments of the invention, the antibody is an antibody other than antibody 31C3, 28F11, 34A3, 29H3 and 23C8, optionally other than an antibody having a heavy and/or light chain having one, two or three CDRs of an antibody selected from the group consisting of antibody 31C3, 28F11, 34A3, 29H3 and 23C8.

In one aspect of any of the embodiments of the invention, the antibody competes for binding to a TLR3 polypeptide with any one or any combination of monoclonal antibodies 31C3, 29H3, 23C8, 28F11 or 34A3, optionally under acid and/or neutral conditions. In one embodiment, an antibody of the invention competes for binding to a TLR3 polypeptide, optionally under acid and/or neutral conditions, with an antibody selected from the group consisting of:
(a) an antibody having respectively a VH and VL region of SEQ ID NOS: 2 and 3 (31C3),
(b) an antibody having respectively a VH and VL region of SEQ ID NOS: 10 and 11 (29H3),
(c) an antibody having respectively a VH and VL region of SEQ ID NOS: 18 and 19 (28F11),
(d) an antibody having respectively a VH and VL region of SEQ ID NOS: 26 and 27 (23C8) and
(e) an antibody having respectively a VH and VL region of SEQ ID NOS: 34 and 35 (34A3).

In one aspect, the invention provides an antibody that specifically binds TLR3, wherein the antibody has one or more (including any combination thereof, or all of) of the following properties:
a. has a subnanomolar affinity for a TLR3 polypeptide at an acidic pH, e.g. a pH less than about 6.5, or between about 4.5 to 6.5 or about pH 5.6;
b inhibits TLR3 signaling in the presence of a TLR3 ligand;
c. inhibits TLR3 signaling in an inflammatory background, e.g. in the presence of inflammatory cytokines such as IFNα;
d. competes for binding to a TLR3 polypeptide with 31C3, 29H3, 28F11, 23C8 or 34A3;
e. does not compete with dsRNA for binding to a TLR3 polypeptide, preferably does not compete with dsRNA for binding to C-terminal portion the TLR3 polypeptide;
f. internalizes into a cell that expresses TLR3 on its surface; and/or
g. inhibits IP-10 secretion on DC (e.g. in human myeloid DC).

The inventors have further elucidated the mode by which certain antibodies exert their biological effect. The inventors have generated several antibodies against a single region of the TLR3 polypeptide (all antibodies compete for binding to TLR3 with each other), and each bind at both neutral and acid conditions to TLR3 and inhibit TLR3 signalling in dendritic cells where TLR3 signalling naturally occurs after internalization of the TLR3 polypeptide. The inventors have furthermore identified the epitopes on human TLR3 bound by the antibodies. The antibodies bind human TLR3 in a region on the N-terminal of the TLR3 protein, that is on the opposite terminal end of the principal dsRNA binding site (the C-terminal portion of the TLR3 polypeptide; see FIG. 20A-D). Thus, in one aspect, provided is an antibody that specifically binds a TLR3 polypeptide, wherein said antibody inhibits signaling by the TLR3 polypeptide without blocking binding of a dsRNA TLR3 ligand to the principal (e.g. C-terminal) dsRNA binding site on the TLR3 polypeptide. Optionally, the antibody and has a Kd of less than 10⁻⁹M for binding to a TLR3 polypeptide at acidic pH. Optionally, the antibody is further characterized by any other feature disclosed herein.

In one aspect, the invention provides a monoclonal antibody that specifically binds to at least one residue in the segment corresponding to residues 102-204 of the TLR3 polypeptide of SEQ ID NO: 1. Optionally, the antibody inhibits signaling by the TLR3 polypeptide. Optionally, the antibody binds to at least one residue in the segment corresponding to residues 174 to 191 of the TLR3 polypeptide of SEQ ID NO: 1. Optionally, the antibody does not bind residue 116, and/or residue 145 of the TLR3 polypeptide of SEQ ID NO: 1. Optionally, the antibody does not bind residue 171, and/or residue 196 of the TLR3 polypeptide of SEQ ID NO: 1. Optionally, the antibody binds amino acid residue 182 of the TLR3 polypeptide of SEQ ID NO:1. Optionally, binding of the antibody to a TLR3 polypeptide having a mutation at residues 116, 141, 196 and/or residue 171 of the TLR3 polypeptide of SEQ ID NO: 1 is not substantially reduced, in comparison to binding to a wild-type TLR3 polypeptide of SEQ ID NO: 1; preferably said mutation is a K145E, D116R, N196A and/or E171A mutation. Optionally, binding of the antibody to a TLR3 polypeptide having a mutation at residue 182 of the TLR3 polypeptide of SEQ ID NO: 1 is reduced, in comparison to binding to a wild-type TLR3 polypeptide of SEQ ID NO: 1; preferably said mutation is a K182E mutation. In another aspect, the antibody binds to at least one residue in the segment corresponding to residues 152 to 173 of the TLR3 polypeptide of SEQ ID NO: 1. In another aspect, the antibody binds to at least one residue in the segment corresponding to residues 102 to 151 of the TLR3 polypeptide of SEQ ID NO: 1. Such antibodies can further be characterized by having any properties described herein, e.g. subnanomolar affinity for a TLR3 polypeptide at an acidic pH, inhibits TLR3 signaling in the presence of a TLR3 ligand or in an inflammatory background (e.g. in the presence of inflammatory cytokines such as IFNα), competes for binding to a TLR3 polypeptide with 31C3, 29H3, 28F11, 23C8 or 34A3; does not compete with dsRNA for binding to a TLR3 polypeptide, preferably does not compete with dsRNA for binding to C-terminal portion the TLR3 polypeptide; or inhibits IP-10 secretion on DC (e.g. in human myeloid DC). Such antibodies can furthermore be used in any of the methods of the inventionln one embodiment, the invention provides an antibody that binds a TLR3 polypeptide, wherein the antibody is selected from the group consisting of:
(a) an antibody having (i) the heavy chain CDR 1, 2 and 3 (HCDR1, HCDR2, HCDR3) amino acid sequences as shown in SEQ ID NO: 20, 21 and 22, and (ii) the light chain CDR 1, 2 and 3 (LCDR1, LCDR2, LCDR3) amino acid sequences as shown in SEQ ID NO: 23, 24 and 25, respectively;
(b) an antibody having (i) the heavy chain CDR 1, 2 and 3 (HCDR1, HCDR2, HCDR3) amino acid sequences as shown in SEQ ID NO: 28, 29 and 30, and (ii) the light chain CDR 1, 2 and 3 (LCDR1, LCDR2, LCDR3) amino acid sequences as shown in SEQ ID NO: 31, 32 and 33, respectively; and
(c) an antibody having (i) the heavy chain CDR 1, 2 and 3 (HCDR1, HCDR2, HCDR3) amino acid sequences as shown in SEQ ID NO: 36, 37 and 38, and (ii) the light chain CDR 1, 2 and 3 (LCDR1, LCDR2, LCDR3) amino acid sequences as shown in SEQ ID NO: 39, 40 and 41, respectively;
   wherein one, two, three, four or more of the amino acids in any of said sequences may be substituted by a different amino acid.

In one aspect, an antibody of the invention that binds a TLR3 polypeptide inhibits signaling by the TLR3 polypeptide and binds to at least one residue in the segment corresponding to residues 102 to 204 of the mature TLR3 polypeptide of SEQ ID NO: 1. In one aspect, provided is a monoclonal antibody that specifically binds a TLR3 polypeptide, wherein said antibody inhibits signaling by the TLR3 polypeptide, has a Kd of less than 10⁻⁹M for binding to a TLR3 polypeptide at acidic pH, and binds to at least one residue in the segment corresponding to residues 102 to 204 of the mature TLR3 polypeptide of SEQ ID NO: 1. In one embodiment the antibody binds to at least one residue in the segment corresponding to residues 102 to 151, optionally further in combination with at least one residue in residues 152 to 173 of the mature TLR3 polypeptide of SEQ ID NO: 1. In one embodiment the antibody binds to at least one residue in the segment corresponding to residues 152 to 173, and/or at least one residue in the segment corresponding to residues 174-191, and/or residue 182, of the mature TLR3 polypeptide of SEQ ID NO: 1. Optionally, the antibody is further characterized by any other feature disclosed herein.

In another embodiment, the antibody of any of the embodiments herein is capable of being internalized by a cell that expresses TLR3 polypeptide on its surface.

In one embodiment, the antibody is chimeric, e.g. contains a non-murine, optionally a human, constant region. In one embodiment, the antibody is human or humanized. In another embodiment, the antibody is a mouse antibody. In another embodiment, the antibody does not substantially bind to other human TLRs (e.g. TLR4).

In one aspect of any of the embodiments of the invention, the isotype of the antibody is IgG, optionally IgG1 or IgG3. In one embodiment the antibody comprises an Fc domain or is of an isotype that is bound by FcγR.

In one aspect of any of the embodiments of the invention, the antibody is an antibody fragment selected from Fab, Fab′, Fab′-SH, F(ab′)2, Fv, diabodies, single-chain antibody fragment, or a multispecific antibody comprising multiple different antibody fragments. In one aspect of any of the embodiments of the invention, the antibody does not comprise an Fc domain or is of an isotype that is not substantially bound by FcγR. In one embodiment, the antibody is of an IgG4 or IgG2 isotype. As demonstrated in the Examples, F(ab′)2 fragments of the antibodies of the present invention retained their ability to modulate TLR3 signaling in DCs and were thus taken up by DC despite their lack of Fc domain. It has previously generally been thought that antibodies will enter the endosomal pathway in DC at least in part by Fc receptor-mediated uptake (human DC express several types of Fcγ receptors (FcγR), including type I (FcγRI, CD64) and type II (FcγRII, CD32)). The finding that isotypes and formats that do not bind FcγR can modulate TLR3 in DC enables antibodies to be developed that retain desired characteristics without a risk of inducing unwanted depletion (e.g. via FcγR-mediated antibody dependent cellular cytotoxicity) of TLR3-expressing cells. For example IgG4 isotypes or other IgG isotypes modified to reduce their FcγR binding can be used for their advantageous pharmacological properties such as serum half-life, while modulating TLR3 signaling, in e.g. a DC, without inducing the death of the cell. In one aspect of any of the embodiments of the invention, the anti-TLR3 antibody inhibits TLR3 signaling and comprises a constant region of IgG4 or IgG2 isotype. In one aspect, of any of the embodiments of the invention, the anti-TLR3 antibody inhibits TLR3 signaling and comprises a constant region (heavy chain constant region) that does not substantially bind FcγR.

In one preferred embodiment, the anti-TLR3 antibody comprises a heavy chain of human IgG4 isotype. In one embodiment, the anti-TLR3 antibody comprises an IgG4 heavy chain comprising a serine to proline mutation in residue 241, corresponding to position 228 according to the EU-index (Kabat et al., “Sequences of proteins of immunological interest”, 5^{th} ed., NIH, Bethesda, Md., 1991). Compositions comprising such antibodies can be characterized as having less than about 15%, such as less than about 10% (e.g., about 5% or less, about 4% or less, about 3% or less, or even about 1% or less) of IgG4 “half-antibodies” (comprising a single heavy chain/light chain pair). Such IgG4 “half-antibody” by-products form due to heterogeneity of inter-heavy chain disulphide bridges in the hinge region in a proportion of secreted human IgG4 (see Angal et al., Molecular Immunology, 30(1):105-108, 1993 for a description of IgG4 “half-antibodies”, S241P mutation, and related principles). This effect is typically only detectable under denaturing, non-reducing conditions.

In another embodiment, the antibody is conjugated or covalently bound to a detectable or toxic moiety.

In another embodiment, provided is a method of producing an antibody that specifically binds a TLR3 polypeptide in a mammalian subject, said method comprising the steps of: a) providing a plurality of antibodies, optionally immunizing a non-human mammal with an immunogen comprising a human TLR3 polypeptide; and b) selecting an antibody from said plurality, or optionally from said immunized animal, that:
(i) binds to the TLR3 polypeptide with high affinity under acidic and neutral conditions;
(ii) modulates TLR3 signaling; and
(iii) binds to at least one residue in the segment corresponding to residues 102 to 204 of the mature TLR3 polypeptide of SEQ ID NO: 1. Optionally, the antibody selected has no substantial loss of binding affinity for a TLR3 polypeptide at acidic conditions compared to neutral conditions.

Optionally, the antibody is selected to inhibit TLR3 signaling without blocking binding of a TLR3 ligand to the C-terminal dsRNA binding site of the TLR3 polypeptide.

In another embodiment, provided is a method of producing an antibody that specifically binds a TLR3 polypeptide in a mammalian subject, said method comprising the steps of: a) providing a plurality of antibodies, optionally, optionally immunizing a non-human mammal with an immunogen comprising a human TLR3 polypeptide; and b) selecting an antibody from said plurality of antibodies, optionally from said immunized animal, that inhibits TLR3 signaling without blocking binding of a TLR3 ligand to the C-terminal dsRNA binding site of the TLR3 polypeptide.

These and additional advantageous aspects and features of the invention may be further described elsewhere herein.

### BRIEF DESCRIPTION OF THE DRAWINGS

FIG. 1 shows dose dependant inhibition of TLR3 signaling using a 293T-human TLR3 luciferase assay with the human anti-TLR3 antibodies. FIG. 1A shows inhibition, with the commercial TLR3.7 antibody (black dots), 28F11 (open triangles), 23C8 (open squares) and 31C3 (black squares) antibodies, compared to a control (no Ab: open dots). FIG. 1B shows the same results in an assay comparing 31C3 (black squares), 23C8 (open squares) and 34A3 (black triangles) antibodies.

FIG. 2 shows dose dependant inhibition of TLR3 signaling using a 293T-mouse TLR3 luciferase assay with the mouse anti-TLR3 antibody 28G7 (full line, black squares) compared to a control antibody with no activity (control, dotted line, opened squares).

FIG. 3 shows results the kinetics assays obtained with the human anti-TLR3 antibodies, in vitro on human monoctye-derived dendritic cells (MdDC). FIG. 3A represents IP-10 secretion in pg/ml (depending on the polyAU doses) for antibody 31C3, FIG. 3B represents the results for antibody 23C8. The TLR3 mAb is added in the medium either 1 h30 before (black crosses), together with (black plus “+”), or 1 h30 after (open squares) the dsRNA, the dsRNA alone (black dots) is provided as a positive control).

FIG. 4 shows results of the kinetics assays obtained with the human and mouse anti-TLR3 antibodies using a 293T-TLR3 luciferase assay. FIG. 4 shows Ab dose-dependant inhibition of luciferase signal mediated by human TLR3, in the presence of a fixed dose of poly(A:U) dsRNA, for the 31C3 antibody (FIG. 4A), the 23C8 antibody (FIG. 4B) and the 34A4 antibody (FIG. 4C). The TLR3 mAb is added in the medium either 1 h before (black crosses), together with (black plus “+”), or 1 h after (open squares) the dsRNA, the dsRNA alone (black dots) is provided as a positive control.

FIG. 5 represents results for the anti-mouse TLR3 antibody 28G7. The TLR3 mAb is added in the medium either 30 min before (black crosses), together with (black plus “+”), or 30 min after (open squares) the dsRNA, the dsRNA alone (black dots) is provided as a positive control.

FIG. 6 shows in vitro dose effect of test antibodies on the expression of CD86 marker (marker of the activation of DCs) and on the inhibition of IP-10 secretion on myeloid DC for human anti-TLR3 antibodies. For antibody 31C3: CD86 (FIG. 6A), IP-10 (FIG. 6B). For antibody 29H3: CD86 (FIG. 6C), IP-10 (FIG. 6D). For antibodies 31C3, 28F11 and 23C8: CD86 (FIG. 6E), IP-10 (FIG. 6F, 31C3 and 23C8 are superposed). For antibody 34A3 and 31C3: IP-10 (FIG. 6G).

FIG. 7 shows in vivo dose effect of test anti-mouse antibodies on IL-6 secretion in sera of mice treated with poly(A:U) dsRNA. FIG. 7A shows the inhibition of IL-6 secretion (in pg/ml), 2 h post treatment with 20 μg of poly(A:U) dsRNA injected intravenously, for 100 μg dose of anti-mouse TLR3 antibody 28G7 (black triangles), in comparison with a control antibody (control—open lozenges) and a PBS treated group (black lozenges). FIG. 7B shows the inhibition of IL-6 secretion (in pg/ml), 2 h post treatment with 100 μg of poly(A:U), for 200 μg dose of anti-mouse TLR3 antibody 28G7 (black triangles) injected intravenously, in comparison with a control antibody (control—open lozenges) and a PBS treated group (black lozenges). Anti-mouse TLR3 antibodies were injected intra-peritoneally 3 h prior poly(A:U) treatment.

FIG. 8 shows binding of the antibodies encompassed by the invention on a human rec TLR3 chip measured in Biacore for antibodies 29H3 and 31C3 (FIG. 8A) compared to TLR3.7 and 40C1285 (bottom RU line), two commercially available antibodies, antibodies 29H3, 31C3, 28F11 and 23C8 (FIG. 8B), antibodies 34A3 and 31C3 (FIG. 8C).

FIG. 9 shows binding of the human anti-TLR3 antibodies encompassed by the invention on a TLR3 chip when the chip has been previously incubated with polyAU, a ligand for TLR3 receptors or not. FIG. 9A relates to antibodies 31C3 and 29H3, FIG. 9B refers to antibodies 28F11, 34A3, and FIG. 9C refers to antibody 23C8. The figures show that the binding of the antibodies is not impaired even in the presence of a dsRNA at the TLR3 dsRNA binding site (at least the C-terminal dsRNA binding site).

FIG. 10A shows binding of the antibodies according to the invention on a human TLR3 chip, when the chip has been previously incubated with polyAU, a TLR3 ligand. Buffer is represented in a thin full line, 31C3 is a bold full line, 28F11 in a thin dashed line, 23C3 in a bold dashed line and 29H3 in a dotted line. FIG. 10A underlines that in the presence of dsRNA, the antibodies are able to be bound efficiently. FIG. 10B shows the binding of the polyAU on a human TLR3 chip when the chip was previously incubated with antibodies of the invention. Buffer (followed by polyAU) is represented in the lower line, while 31C3 followed by polyAU is in the upper line. Incubation of TLR3 with anti-TLR3 antibody therefore does not prevent dsRNA from binding to TLR3. FIG. 10C shows the polyAU binding signals on a human TLR3 chip when the chip was first incubated with antibody 31C3; the upper line shows buffer followed by polyAU while the lower line shows 31C3 followed by polyAU.

FIG. 11 reports binding for antibodies 29H3 (full bold line) and 31C3 (dashed bold line), when human TLR3 chip has been previously saturated with 31C3 and then incubated with 29H3 (thin full line) or when human TLR3 chip has been previously saturated with 29H3 and then incubated with 31C3 (thin dashed line).

FIG. 12 represents binding of antibodies either alone (full line), after saturation with the 31C3 antibody and then binding of the test antibody (dotted line), and saturation of 31C3 after saturation with 31C3, as a control (dashed line). Results are provided for antibodies TLR3.7 (FIG. 12A), 23C8 (FIG. 12B) and 28F11 (FIG. 12C). This comparison of binding levels underlines that the antibodies according to the invention have an impaired binding to hTLR3 when the chip has previously been saturated with the 31C3 antibody, on the contrary, the commercial TLR3.7 antibody retains the same binding level in the presence or in the absence of 31C3 antibody.

FIG. 13 shows molecular surface maps of the extracellular domains of the human TLR3 protein, generated by computer modeling.

FIG. 14 shows FACS analysis of internalization assays as described in example 7. FIG. 14A represents the negative control, showing experimental standard fluorescence of the 293T-ISRE/TLR3 cells, in the absence of an antibody linking TLR3 proteins. FIGS. 14B and C shows fluorescence intensity when living unpermeabilized cells have been incubated with the 31C3 antibody at 37° C. after 24 h or 2 h incubation, respectively. Internalized anti-TLR3 Ab is further revealed by cell permeabilization followed by staining with GAM-APC, thus demonstrating the ability of anti-TLR3 antibody to be first internalized and to bind intracellular TLR3 proteins. FIG. 14D is the positive control, indicating the level of TLR3 expression in 293T-ISRE/TLR3 cell lines, representing experimental fluorescence of the 293T-ISRE/TLR3 cells, when permeabilized and incubated in the presence of 23E7 antibody linking TLR3 proteins. In FIGS. 14D and 14F, cells have been first incubated with the 31C3 antibody at 37° C. after 24 h or 2 h incubation, respectively, and level of TLR3 expression is further revealed with non competing 23E7 antibody staining, as in FIG. 14B. Both figures show a similar fluorescence than FIG. 14B demonstrating that the binding of TLR3 by antibody 31C3 does not down-modulate the expression of TLR3 on 293T-ISRE/TLR3 cell lines.

FIG. 15 shows results of a rheumatoid arthritis mouse models. FIG. 15A shows the results of a preventive rheumatoid arthritis mouse model. FIG. 15B shows the results of a curative rheumatoid arthritis mouse model. FIG. 15C shows the results of a curative rheumatoid arthritis mouse model when mice are treated with PBS, a control antibody, 28G7 and an anti-TNFα antibody (Humira™).

FIG. 16 shows results of the mouse colitis model. FIG. 16A shows the wall thickness measurements for the mice treated with saline (black dots), with TNBS only (black squares) with an anti-TNFα antibody and TNBS (black triangles), with 28G7 and TNBS (open dots), and with a control Ab and TNBS (open squares). FIG. 16B shows the macroscopic damage score for the mice treated with saline (black dots), with TNBS only (black squares) with an anti-TNFα antibody and TNBS (black triangles), with 28G7 and TNBS (open dots), and with a control Ab and TNBS (open squares). The anti-TLR3 antibody according to the invention ameliorating the development of the disease, under stringent conditions. (* p<0.05, ** p<0.01 vs saline).

FIG. 17 shows results of a COPD mouse model. FIG. 17A shows BAL differential cell counts for macrophages, eosinophils, neutrophils and lymphocytes. The anti-TLR3 antibodies strongly decreased the infiltration of neutrophils into the airways, while not substantially affecting macrophages eosinophils or lymphocytes. FIG. 17B shows venous blood saturated oxygen (in percent) for each of LPS/elastase alone and LPS/elastase in combination with anti-TLR3 antibodies or roflumilast. FIG. 17C shows IL17A in BAL fluid (BALF), where anti-TLR3 antibodies decreased IL17A (pg/ml) substantially, and as much as roflumilast. FIG. 17D shows IP-10 in BALF, where -TLR3 antibodies decreased IP-10 (pg/ml) substantially. FIG. 17E shows BAL differential cell counts for macrophages, neutrophils and lymphocytes for a second study comparing anti-TLR3 antibodies (28G7), roflumilast (Rofu) and the combination of roflumilast and anti-TLR3 antibodies (combo).

FIG. 18 shows results of a CLP (cecal ligation and puncture—sepsis) mouse model. In this acute model, mice experience an acute infection, mimicking septic shock.

FIG. 19 shows results of an H/D experiment described in example 16. FIG. 19A shows HX monitored by mass spectrometry identifies regions of TLR3 involved in mAb 34A2 binding (Left) Mass/charge spectra corresponding to the peptide fragment 102-111, KVLNLQHNEL (m/z=604.34, z=2). (Right) Mass/charge spectra corresponding to the peptide fragment 131-151, MSNSIQKIKNNPFVKQKNLIT (m/z=612.09, z=4). For all spectra the upper panels show the non-deuterated controls, middle and lower panels show the peptide after 100 sec in-exchange with D₂O in the absence or presence of 34A3, respectively. FIG. 19B shows hydrogen exchange time-plots of representative peptides of TLR3 in the presence or absence of mAb 34A3. Deuterium incorporation (Da) of TLR3 peptides is plotted against time on a logarithmic scale in the absence of mAb i.e. TLR3 (black lozenges) or in the presence of mAb, i.e. TLR3+23C8 (open squares), TLR3+31C3 (open triangles) or TLR3+34A3 (crosses). Peptide 27-42 represents a region of TLR3 that is unaffected by mAb binding. Peptides 102-111, 112-121 and 131-151 represent regions of TLR3 that are part of the binding epitope for mAb 34A3. FIG. 19C shows the sequence coverage of HX analyzed peptides of TLR3 (amino acids 1-711 of SEQ ID NO 1 are shown) in the presence and absence of mAb 34A3. The sequence (using mature numbering) is displayed above the HX analyzed peptides (shown as horizontal bars). Peptides showing similar exchange patterns both in the presence and absence of 34A3 are displayed in white whereas peptides showing reduced deuterium incorporation upon mAb 34A3 binding are coloured black. Potential N-glycosylation sites are double underlined. Peptides containing glycosylations cannot be analyzed due to the undefined and heterogeneous mass caused by the glycosylation. Peptides analyzed thus covers 70% the TLR3 sequence. The majority of the gaps in the sequence coverage of TLR3 are in vicinity of a N-glycosylation site. FIG. 19D shows the Hydrogen exchange time-plots of peptides in the 152-173 region of TLR3 in the presence or absence of mAb 23C8, 31C3 or 34A3. Deuterium incorporation (Da) of TLR3 peptides is plotted against time on a logarithmic scale in the absence of mAb i.e. TLR3 (black lozenges) or in the presence of mAb, i.e. TLR3+23C8 (open squares), TLR3+31C3.1 (open triangles) or TLR3+34A3 (crosses). FIG. 19E shows the sequence coverage of HX analyzed peptides of TLR3 (amino acids 1-399 of SEQ ID NO 1 are shown) in the presence and absence of mAbs 23C8, 31C3 or 34A3. Only residues 27-399 are shown. The figure is similar to FIG. 19C with a few modifications. Peptides showing similar exchange patterns both in the presence and absence of mAbs are displayed in white whereas peptides showing reduced deuterium incorporation upon mAb 34A3 binding are coloured black and peptides showing reduced exchange protection upon binding of 23C8, 31C3 or 34A3 are shown with black grid. The amino acid residues that are boxed represent the putative epitopes for 23C8 and 31C3. FIG. 19F shows the overview of the TLR3 structure (from pdb code 2A0Z). FIG. 19G shows the structural mapping of mAb 34A3 (19G-A and 19G-B) and 23C8 and 31C3 on TLR3. Regions depicted in black are 102-121 and 131-152 (19G-A), 102-121, 131-152 and 168-173 (19G-B) or 168-173 and 192-204 (19G-C). TLR3 structure from pdb 2A0Z was used.

FIG. 20 shows molecular surface maps of the extracellular domains of the human TLR3 protein, generated by computer modeling. FIG. 20A shows a TLR3 protein. The epitope region has been highlighted in grey and the dsRNA contact zones have been schematized by two circles. FIG. 20B shows the top view of the conformation of two TLR3 proteins in the presence of a dsRNA ligand. FIG. 20C shows the side view of the same construction. FIG. 20D shows the conformation of two TLR3 proteins in the presence of a dsRNA ligand, with the epitope identified in the present application in dark grey. FIG. 20E shows a view of the side of the N-terminal end of the TLR3 polypeptide, showing amino acid residues K145, D116, K182, N196 and E171. FIG. 20F shows a view of the non-glycosylated face of the TLR3 polypeptide, with the N-terminal end of the TLR3 polypeptide in the foreground, showing amino acid residues K145, D116, K182, N196 and E171.

FIG. 21 shows the phylogenetic trees of the CDRs of the antibodies according to the invention. FIG. 21A shows the phylogenetic tree for the light chains CDRs and FIG. 21B shows the phylogenetic tree for the heavy chains CDRs. The figures show that there is a high CDR homology between antibodies 28F11 (28.2), 31C3 (31) and 23C8 (23), and that 23H3 (29) and 34A3 (34) have more differences in amino acid sequences.

FIG. 22 shows reduction of IP-10 production in donor 2 in response to polyAU using anti-human TLR3 mAbs 31C3 or 34A3 in combination with methotrexate, dexamethasone or Humira®, showing that TLR3 mAbs reduce IP-10 compared with polyAU and polyAU in combination with methotrexate, dexamethasone or Humira®.

FIG. 23 shows reduction of IP-10 production in donor 1 in response to polyIC using anti-human TLR3 mAbs 31C3 or 34A3 in combination with methotrexate, dexamethasone or Humira®, showing that TLR3 mAbs reduce IP-10 compared with polyIC and polyIC in combination with methotrexate, dexamethasone or Humira®.

FIG. 24 shows reduction of IP-10 production in donor 2 in response to polyIC using anti-human TLR3 mAbs 31C3 or 34A3 in combination with methotrexate, dexamethasone or Humira®, showing that TLR3 mAbs reduce IP-10 compared with polyIC and polyIC in combination with methotrexate, dexamethasone or Humira®.

### DETAILED DESCRIPTION OF THE INVENTION

### Introduction

The present invention provides novel methods for treating an autoimmune or inflammatory disease in a subject in need thereof using an anti-TLR3 antibody which inhibits TLR3 signaling. The present invention also provides novel methods for treating relapses, attacks, or acute phases, occurring during the course of an inflammatory or autoimmune disease in a subject in need thereof using an anti-TLR3 antibody which inhibits TLR3 signaling. The present invention also provides novel methods for treating established inflammatory or autoimmune diseases in a subject in need thereof using an anti-TLR3 antibody which inhibits TLR3 signaling. The invention also provides treatment regimens and treatment combinations that can be used for the treatment of inflammatory or autoimmune disease in a subject in need thereof using an anti-TLR3 antibody which inhibits TLR3 signaling.

The present invention is based, at least in part, on the discovery of monoclonal antibodies that specifically and efficiently bind TLR3 under acidic conditions corresponding to that encountered in an acidified endosomic compartment. Among numerous antibodies assessed, certain antibodies emerged that retained binding to TLR3 at high affinities under acidic conditions, while other antibodies such as those available commercially and others selected for TLR3 binding or TLR3 modulation lost affinity despite initially displaying higher (e.g. 2-logᵢₙ higher) affinity for TLR3, and/or had low affinity even under neutral conditions. Acidic conditions used were pH 5.6 which is similar to that observed in an acidified endosomic compartment, corresponding to the conditions under which TLR3 signaling in inflammatory conditions is believed to take place.

Acidic conditions are generally known to affect the structure of proteins as well as to affect protein-protein interactions. It is known, for example, that MHC class II peptides that are not bound to other peptides are rapidly degraded in the acidic conditions of the endosome. However, in the present case the antibodies that lost their high binding affinity to immobilized TLR3 under acidic conditions of pH 5.6 had been previously purified under acidic conditions (pH 3). Without wishing to be bound by theory, this suggests that the loss of binding affinity arose not from inherent instability (degradation) of the antibody at acid conditions, but rather from modifications in the interaction between the antibodies and their target antigens.

Modifications in antibody-TLR3 interactions arising from changes in pH are believed to affect interactions of dsRNA with TLR3, since the TLR3 ligand poly(I-C) binds and activates TLR3 only at acidic pH. Studies have reported that poly(I-C) (and other dsRNA) bind TLR3 in a region of TLR3 of positive electrostatic potential at neutral pH that can undergo a change in electrostatic potential in acidic conditions (that is, acidic conditions in the range of pH 4.5 to 6.5, or around 5.6). The present antibodies, however, are believed to bind an epitope that does not undergo substantial change in electrostatic potential (or undergoes less change than e.g. a region of positive electrostatic potential) when conditions are acidified such that the binding affinity of the antibodies remains substantially unchanged. This can, in one aspect, manifest itself in terms of affinity of the antibodies for TLR3, since the antibodies do not have substantially different (lower and/or higher) affinity for binding human TLR3 under acidic conditions than under neutral conditions e.g. where the K_{D} for binding to TLR3 differs by no more than 0.2-, 0.3-, 0.4-, 0.5-, 1.0-, or 1.5-log₁₀. The K_{D} for binding to TLR3 under acid and neutral conditions differed by less than 0.5-log₁₀ for antibodies 31C3 and 29H7.

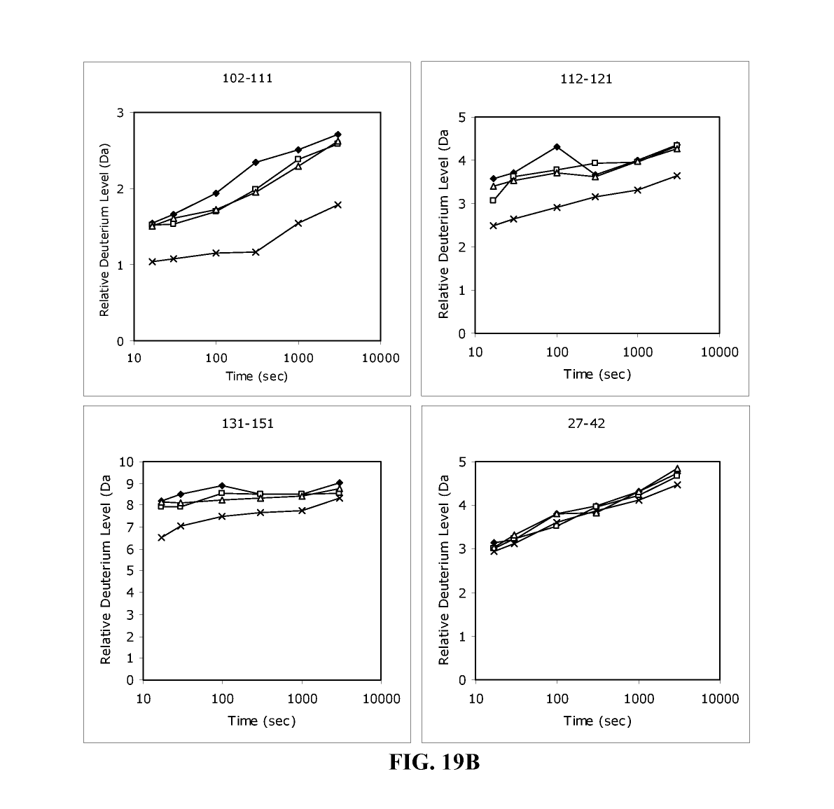

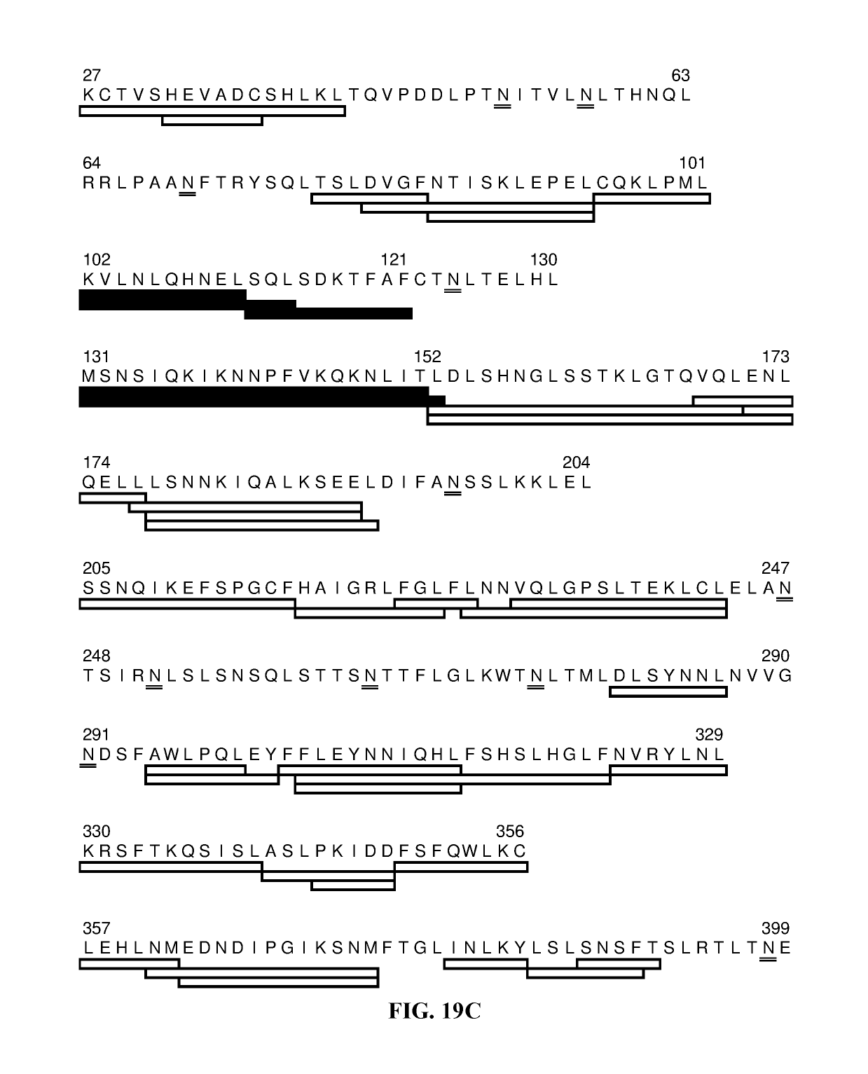

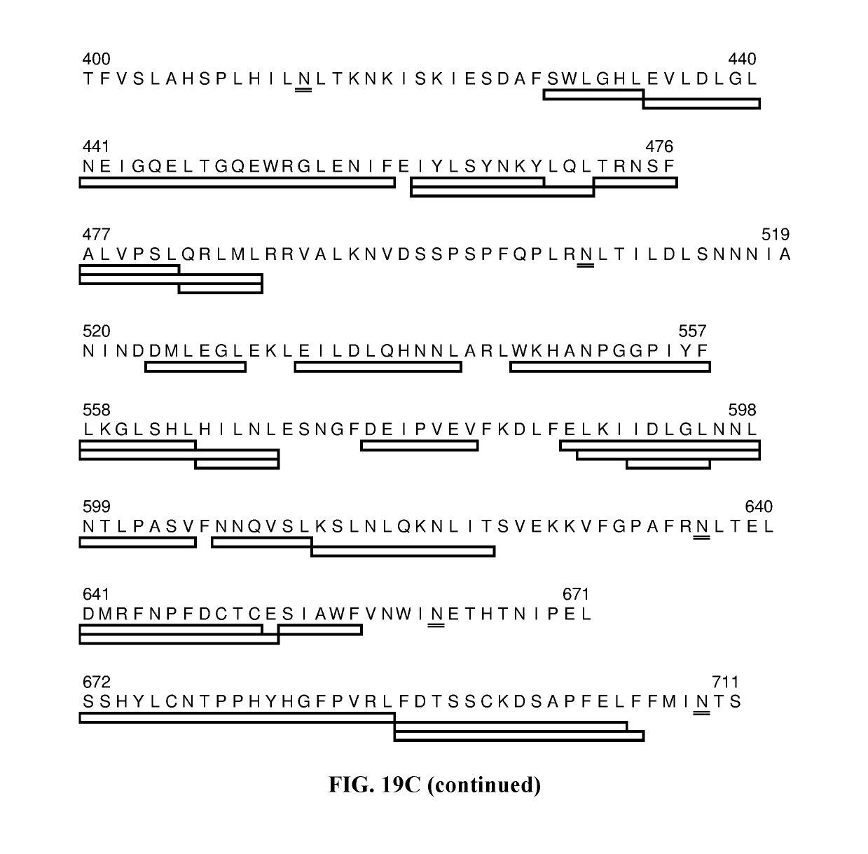

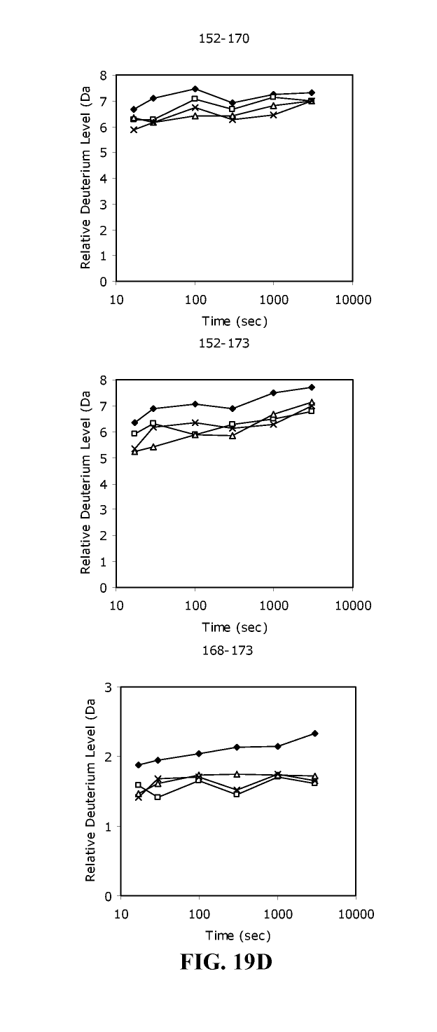

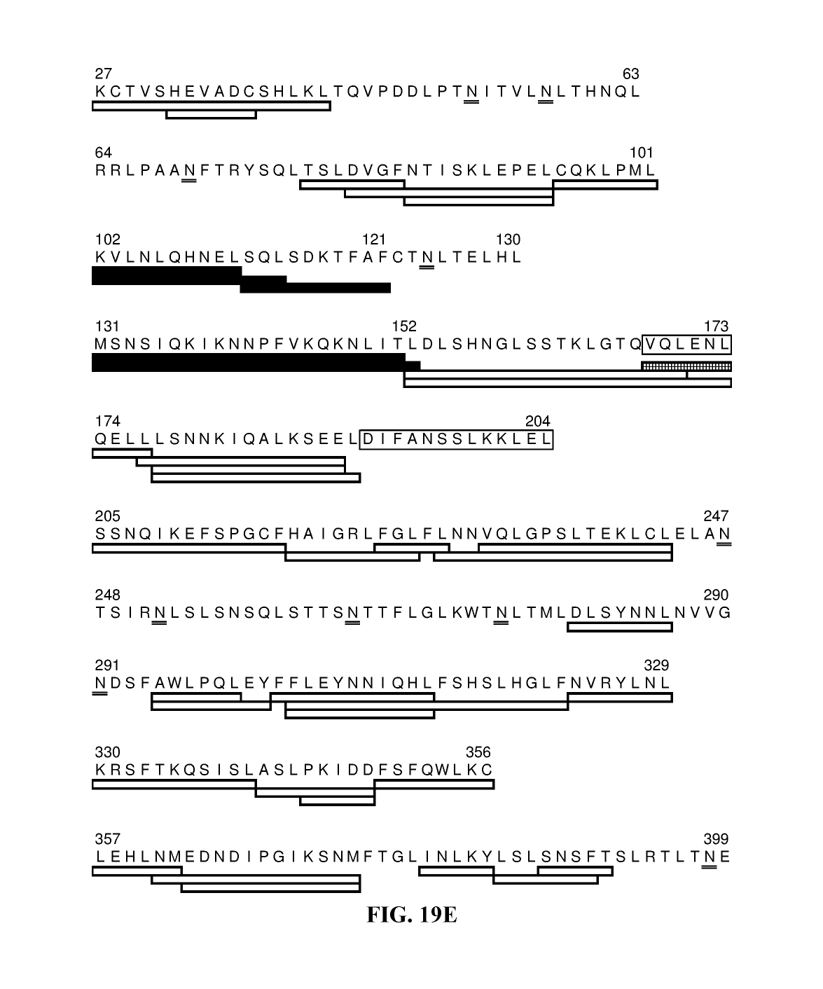

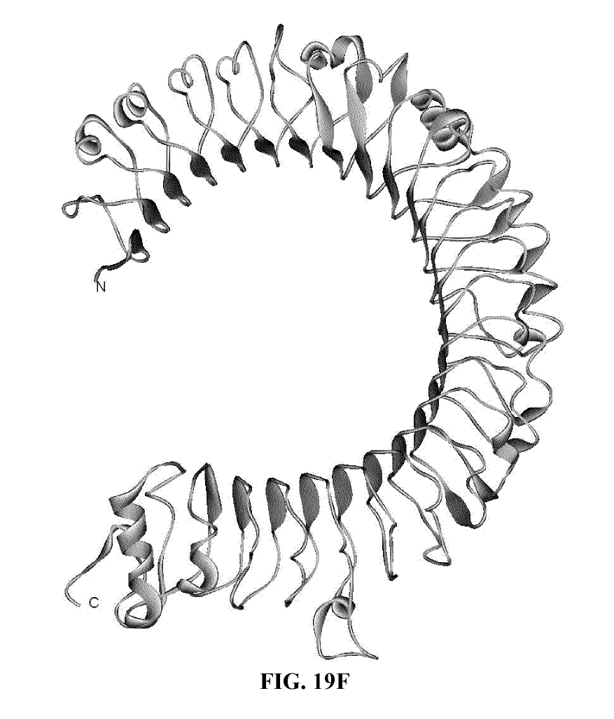

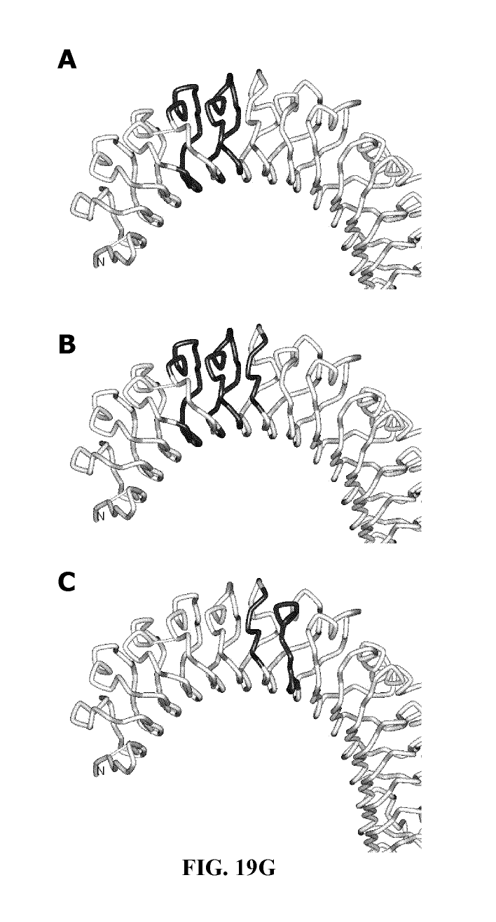

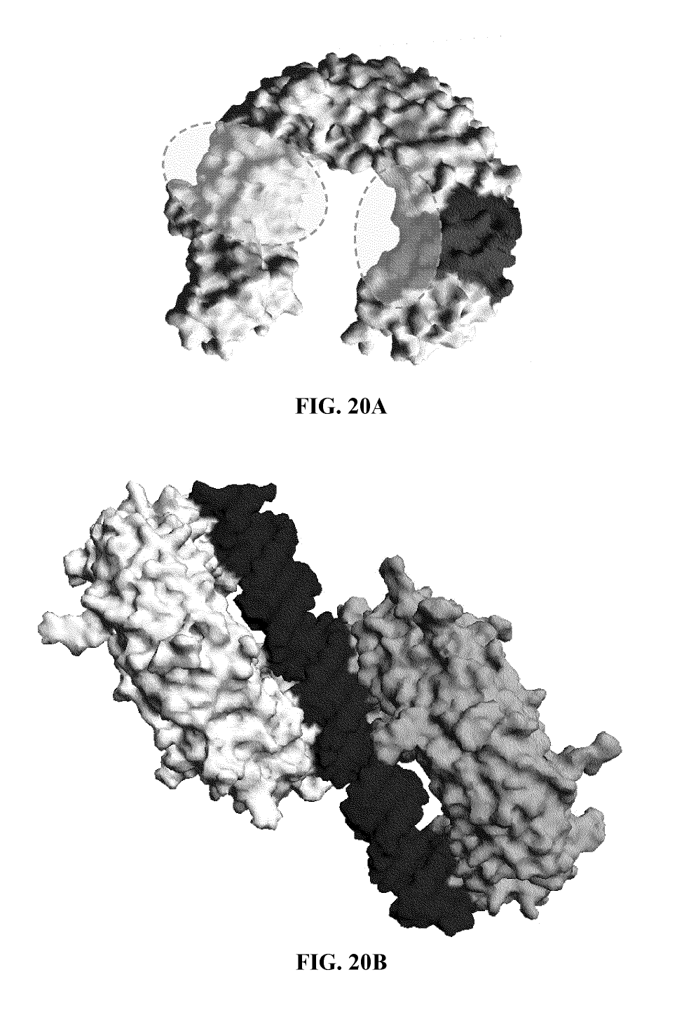

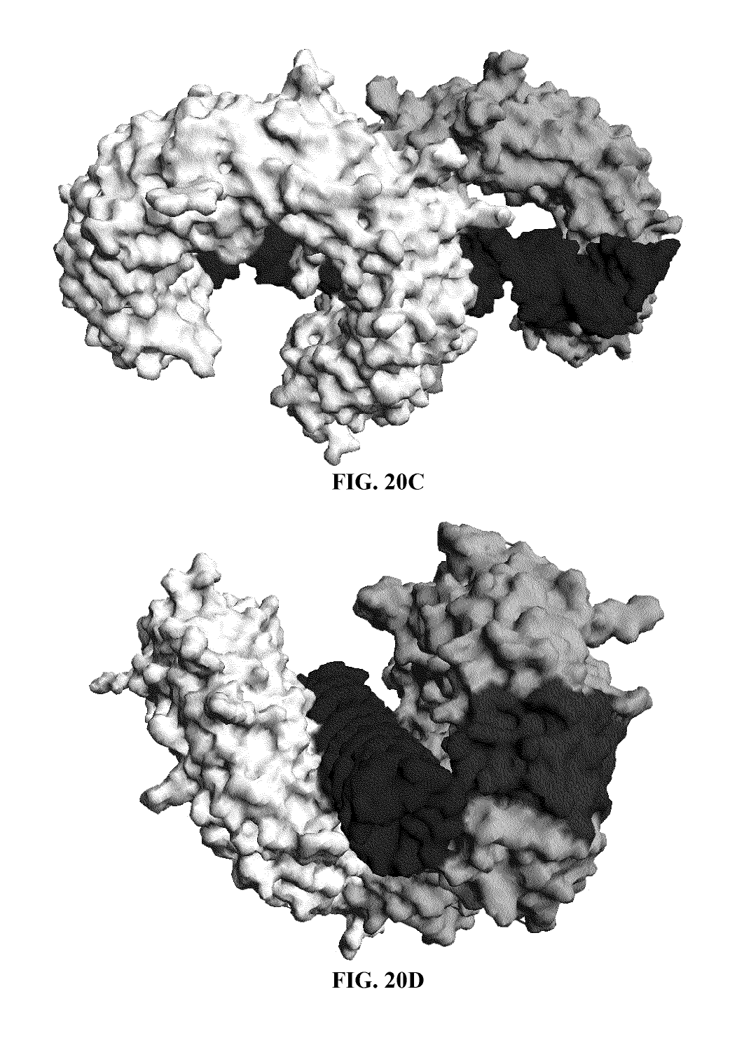

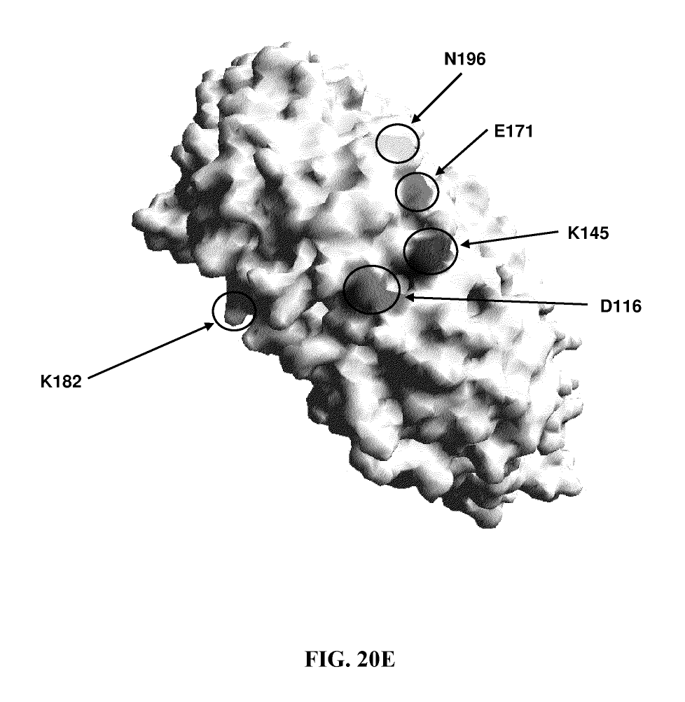

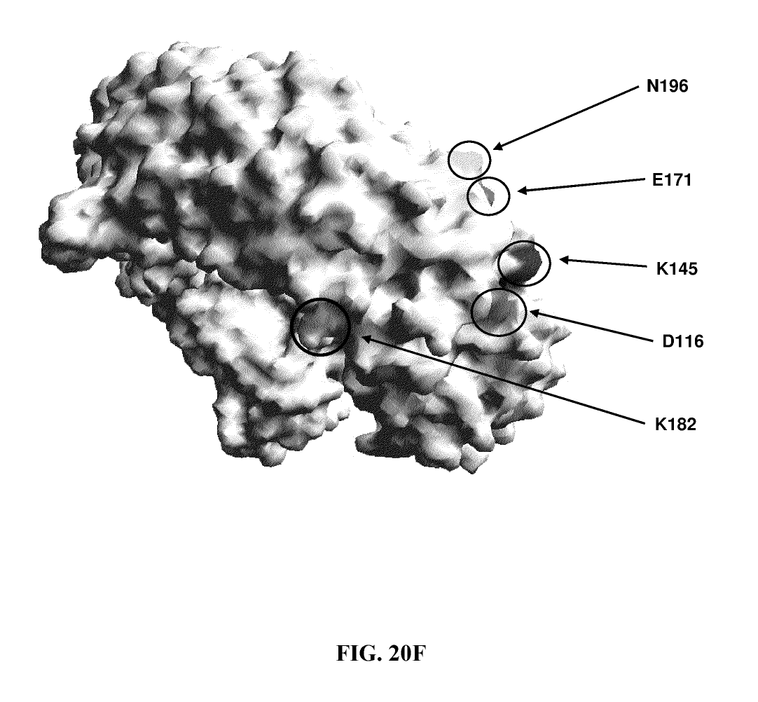

The present invention is also based, at least in part, on the discovery of high affinity monoclonal antibodies that specifically and efficiently inhibit the TLR3 signaling pathway. The inventors have identified epitopes present on human TLR3, including the epitope recognized by antibody 31C3, 29H3, 23C8, 28F11 or 34A3, which are particularly efficient in inhibiting TLR3 signaling, and inhibiting cytokine release in response to stimulation with a TLR3 ligand. The epitopes are shown in FIGS. 19G and 20A and 20D.

The antibodies of the present invention that bind TLR3 under acidic conditions will generally bind both cell surface TLR3 and endosomic TLR3 at high affinity, such that the antibodies will be useful in any situation (e.g. treatment or prevention of disease) where targeting (e.g. modulating) TLR3 is useful. TLR3 has been found in some cases of inflammation the surface of macrophages and blocking TLR3 upon chloroquine neutralization of endosomal acification nevertheless exhibited some anti-inflammatory activity (Cavassani et al. 2008, supra). However, the antibodies of the invention will have the greatest advantage over other antibodies in the treatment or prevention of diseases where the modulating (e.g. inhibiting) the signaling by TLR3 in the cytosolic (e.g. endosomic) compartments is useful or required, and the relative importance of modulating signaling of such compartments TLR3 may depend on the disease. One example of such as disease is rheumatoid arthritis; endosomic compartment-expressed TLR3 is believed to play an important role in rheumatoid arthritis, since treatment with chloroquine, an inhibitor of endosomal acidification, inhibits TLR3 signaling and inhibits production of inflammatory cytokines from synovial cultures from patients having rheumatoid arthritis (Sacre et al. (2008) J. Immunol. 181:8002-8009). Endosomic compartment-expressed TLR3 is believed to play an important role in a number of other diseases where DC (e.g. myeloid DC) are involved in exacerbating disease, as mDC have a well documented capacity to take up antigens from apoptotic or necrotic cells including during tissue necrosis during acute inflammation.

Since the present antibodies are specific for TLR3, they can also be used for other purposes, including purifying TLR3 or TLR3-expressing cells, modulating (e.g. activating or inhibiting) TLR3 receptors in vitro, ex vivo, or in vivo, targeting TLR3-expressing cells for destruction in vivo, or specifically labeling/binding TLR3 in vivo, ex vivo, or in vitro, including for methods such as immunoblotting, 1HC analysis, i.e. on frozen biopsies, FACS analysis, and immunoprecipitation.

### DEFINITIONS

As used herein, “TLR3 ligand” refer to any compound that can specifically bind to and alter the activity of TLR3 in vitro, ex vivo, or in vivo. The compound can be a naturally occurring ligand, e.g., generally dsRNA or viral dsRNA, or a synthetic ligand such as polyIC or polyAU. The compound can be any type of molecule, including inorganic or organic compounds or elements, including proteins (such as antibodies), nucleic acids, carbohydrates, lipids, or any other molecular entity. Further, such compounds can modulate TLR3 receptors in any way, including activating or inhibiting, and by any mechanism, including by binding to the receptor and triggering or shutting off activity in a manner similar to a naturally occurring ligand, or by binding to the receptor and blocking access to other ligands. Preferably, the ligand activates the receptor, and as such can be used to induce the production of cytokines by TLR3-expressing cells.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (VL) and variable heavy chain (VH) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation and because they are most easily made in a laboratory setting. Preferably the antibody of this invention is a monoclonal antibody. Particularly preferred are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g. TLR3, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody (e.g. 31C3, 29H3, 23C8, 28F11 or 34A3), it means that the antibody competes with the monoclonal antibody in a binding assay using either recombinant TLR3 molecules or surface expressed TLR3 molecules. For example, if a test antibody reduces the binding of 31C3, 29H3, 23C8, 28F11 or 34A3 to a TLR3 polypeptide or TLR3-expressing cell in a binding assay, the antibody is said to “compete” respectively with 31C3, 29H3, 23C8, 28F11 or 34A3.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

Within the context of this invention a “determinant” designates a site of interaction or binding on a polypeptide.

The term “epitope” is defined as an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

By “immunogenic fragment,” it is herein meant any polypeptidic or peptidic fragment that is capable of eliciting an immune response such as (i) the generation of antibodies binding said fragment and/or binding any form of the molecule comprising said fragment, including the membrane-bound receptor and mutants derived therefrom, (ii) the stimulation of a T-cell response involving T-cells reacting to the bi-molecular complex comprising any MHC molecule and a peptide derived from said fragment, (iii) the binding of transfected vehicles such as bacteriophages or bacteria expressing genes encoding mammalian immunoglobulins. Alternatively, an immunogenic fragment also refers to any construction capable of eliciting an immune response as defined above, such as a peptidic fragment conjugated to a carrier protein by covalent coupling, a chimeric recombinant polypeptide construct comprising said peptidic fragment in its amino acid sequence, and specifically includes cells transfected with a cDNA of which sequence comprises a portion encoding said fragment.

A “human-suitable” antibody refers to any antibody, derivatized antibody, or antibody fragment that can be safely used in humans for, e.g. the therapeutic methods described herein. Human-suitable antibodies include all types of humanized, chimeric, or fully human antibodies, or any antibodies in which at least a portion of the antibodies is derived from humans or otherwise modified so as to avoid the immune response that is generally provoked when native non-human antibodies are used.

For the purposes of the present invention, a “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5^{th} ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context of this invention, the term antibody that “binds” a common determinant designates an antibody that binds said determinant with specificity and/or affinity.

### Treatment of Disease

The present invention provides methods for the treatment of an individual having an autoimmune or inflammatory disease, comprising administering to the individual an anti-TLR3 antibody. In one embodiment, the individual has an autoimmune or inflammatory disease that has been declared for an extended period of time (e.g. more than one year), has signs of ongoing or active inflammation, has physical signs of disease (e.g. joint swelling, lesions, neurological symptoms, etc.), has chronic disease, has severe disease (as assessed by applicable criteria, e.g. DAS or ACR criteria in rheumatoid arthritis) or has progressing disease.

In one embodiment, the present invention provides methods for the treatment of an individual having an established autoimmune or inflammatory disease, comprising administering to the individual an anti-TLR3 antibody. In one embodiment, the present invention provides methods for the treatment of acute phases, or of an attack, crisis, exacerbation or flare, of autoimmune or inflammatory diseases using a TLR3 antibody (or related compositions), preferably wherein the antibody is administered to an individual during an acute phase or during an attack, crisis, exacerbation or flare of an autoimmune or inflammatory disease. In one embodiment, the disease is selected from the group consisting of rheumatoid arthritis, Juvenile idiopathic arthritis, multiple sclerosis, Crohn disease or rectocolitis, Lupus erythematosus, hepatitis, chronic obstructive pulmonary disease (COPD) or asthma, ankylosing spondylitis and related diseases. In one embodiment, the disease is characterized by the presence of a TLR3 ligand (e.g. extracellular dsRNA). In one embodiment, the disease is characterized by the presence of detectable levels of a proteolytic enzyme, an inflammatory mediator, a marker of ongoing inflammation or a proinflammatory cytokine (e.g. TNF-α and/or interleukin-1 (IL-1)). Preferably the antibody inhibits signaling by the TLR3 polypeptide, optionally further in acid conditions and with high binding affinity, optionally in a human dendritic cell.

Treatment generally involves the delivery of an effective amount of a composition comprising an anti-TLR3 antibody with the purpose of preventing any symptoms or disease state to develop or worsen, or with the purpose of preventing (e.g. preventing or postponing progression), easing, ameliorating, or eradicating (curing) such symptoms or disease states already developed. Disease diagnosis, evolution and rating (or staging) can be defined by standard medical criteria for the particular type of disease in order to determine whether an individual has disease that is established, is in an acute phase, is progressing, is chronic, has physical symptoms, or is of a certain level of severity. Likewise, attack, crisis, exacerbation or flares can be identified by any suitable medical criteria.

In one embodiment, the invention will comprises a step of conducting an evaluation or testing step to assess the presence, stage, evolution or rating of disease. Thus, in one aspect, the invention provides a method for the treatment of an autoimmune or inflammatory disease in a patient, comprising: (a) conducting an evaluation of disease in the patient; and (b) if said patient has a disease suitable for treatment with an anti-TLR3 antibody of the invention, administering to said patient an effective dose of anti-TLR3 antibody. Optionally such evaluation step may involve obtaining a biological sample from a patient suspected of having an autoimmune or inflammatory disease. Methods for evaluating disease (e.g. diagnosing, staging, etc.) can be achieved by any suitable technique known in the art, for example by performing a laboratory-based test. Examples of suitable techniques include conducting a PCR or RT-PCR based assay (e.g., to detect disease associated nucleic acids or genes, often referred to as “markers” or “biomarkers”), biopsy, endoscopy, stool studies, any noninvasive laboratory tests (e.g. anemia and infection, liver function tests to screen for liver and bile duct problems, tests for bacterial, viral and parasitic infections), ultrasound, CT, MRE, MRI and other imaging techniques, chromosomal analysis, immunoassay/immunocytochemical detection techniques (e.g. presence of autoantibodies), histological and/or histopathologic assays, serum protein electrophoresis, flow cytometry (e.g. detection of immune cells, T cells, etc.), arterial blood gas (ABG) analysis (in asthma or COPD), and physical examination techniques (e.g., for physical symptoms, numbers of joints with synovitis, etc.). In one embodiment, the methods comprise detecting the presence of auto-antibodies, for example detecting rheumatoid factor (RhF), anti-cyclic citrullinated peptide antibodies, anti-ssRNA, anti-dsRNA, anti-Smith, anti-phospholipid, anti-nuclear and/or anti-actin antibodies. In one embodiment, the methods comprise assessing levels of a proteolytic enzyme, an inflammatory mediator, a marker of ongoing inflammation or a proinflammatory cytokine. In one embodiment, the methods comprise determining c-reactive protein (CRP) level and/or erythrocyte sedimentation rate. A determination that an individual has abnormal results (indicative of disease, exacerbation, ongoing inflammation, etc.), for example abnormal levels of ABG, autoantibodies, CRP, any proteolytic enzyme, inflammatory mediator or marker of ongoing inflammation indicates the individual is suitable for treatment with an anti-TLR3 antibody.

Delivering anti-TLR3 antibodies to a subject (either by direct administration or expression from a nucleic acid therein, such as from a pox viral gene transfer vector comprising anti-TLR3 antibody-encoding nucleic acid sequence(s)) and practicing the other methods of the invention can be used to reduce, treat, prevent, or otherwise ameliorate any suitable aspect of disease or disease progression. The methods of the invention can be particularly useful in the reduction and/or amelioration of inflammation and/or tissue damage, and any parameter or symptom associated therewith (e.g. the presence of a marker of inflammation, number of pro-inflammatory cells in circulation or in a particular tissue).

Anti-TLR3 antibodies can advantageously be used to treat established disease. “Established disease” refers to an autoimmune or inflammatory disease which has been declared for an extended period of time, e.g. more than one year. Depending on the specific disease, established disease also means a disease which is not controlled e.g. which is still progressing or for which the patient does not experience remission, in the presence or in the absence of a treatment. In one aspect, the invention provides a method for the treatment of an autoimmune or inflammatory disease in a patient, comprising: (a) determining whether said patient has an established disease; and (b) if said patient has an established diseases, administering to said patient an effective dose of anti-TLR3 antibody.

Anti-TLR3 antibodies can also advantageously be used to treat chronic disease. “Chronic disease” refers to a disease that persists for an extended period of time. For instance, a chronic disease can be a disease lasting 3 months or more, as defined by the U.S. National Center for Health Statistics. In one aspect, the invention provides a method for the treatment of an autoimmune or inflammatory disease in a patient, comprising: (a) determining whether said patient has chronic disease; and (b) if said patient has chronic diseases, administering to said patient an effective dose of anti-TLR3 antibody.

Anti-TLR3 antibodies can also advantageously be used to treat individuals having an attack, crisis, exacerbation or flare. The terms “attack”, “crisis”, “exacerbation” and “flare”, designate a more rapid evolution of new symptoms or worsening of old symptoms related to an inflammatory or an autoimmune disease. Such phases last over a period of hours or days, as opposed to a slow progression of the disease that occurs over months and years. During such attacks, the patient experiences fever, pain, inflammatory syndrome (flu-like syndrome). In RA, the joints of the patient are swollen and painful. The patient can experience flu-like syndromes. A crisis can last from a few hours to many weeks. In Multiple Sclerosis, flare-ups can feature a new symptom or the worsening of an existing symptom but must last at least 24 hours to be considered a true exacerbation, a flare up denotes new lesions forming in the brain or spinal cord that disrupt neural transmission. Most flare-ups last a few days or weeks but can last for several months. Effects can for instance be: movement difficulties or spasms, balance and coordination problems; vision problems, uncoordinated eye movements, blurred vision or double vision, partial blindness during a flare-up; bladder and bowel symptoms; sexual problems, changes in mental function: memory loss, inattention and poor judgment or depression. In COPD, an exacerbation can be defined as “an event in the natural course of the disease characterized by a change in the patient's baseline dyspnea, cough, and/or sputum that is beyond normal day-to-day variations, is acute in onset and may warrant a change in medication in a patient with underlying COPD”. The patient experiencing an exacerbation has one of the following symptoms: increased cough and sputum production, change in the color and/or thickness of the sputum, wheezing, chest tightness, fever. In Crohn's disease or rectocolitis, a flare up is mainly the exacerbation of usual Crohn's disease symptoms: diarrhea, crampy abdominal pain, fever, loss of appetite. In one aspect, the invention provides a method for the treatment an autoimmune or inflammatory disease in a patient comprising: (a) determining whether said patient is experiencing an attack, crisis, exacerbation or flare; (b) if said patient experiences an attack, crisis, exacerbation or flare, administering to said patient an effective dose of anti-TLR3 antibody.

Anti-TLR3 antibodies can also advantageously be used to treat individuals having a relapse. The term “relapse” refers to improvement or stabilization in a patient's symptoms. A disease is relapsing when the health or condition of the patient improves. In one aspect, the invention provides a method for the treatment an autoimmune or inflammatory disease in a patient comprising: (a) determining whether said patient is experiencing a relapse, crisis, exacerbation or flare; (b) if said patient experiences a relapse, administering to said patient an effective dose of anti-TLR3 antibody.

Optionally, an assessment step can be carried out, comprising assessing the expression of a TLR3 polypeptide on cells (e.g. pro-inflammatory cells, dendritic cells, T cells, etc.) from a patient prior to treatment with an anti-TLR3 antibody. Generally, in this step, a sample of cells is taken from a patient, typically as a biopsy, and tested, e.g., using immunoassays, to determine the expression and optionally relative prominence of the TLR3 polypeptide on the cells. In one aspect, a determination that a patient has cells that prominently express the TLR3 polypeptide indicates that the anti-TLR3 antibody (and optionally any further therapeutic agent) is suitable for said patient. In a further step, the patient can then be treated with the anti-TLR3 antibody.

Optionally, in one embodiment, a TLR3 ligand detection step can be carried out, comprising detecting the presence of a TLR3 ligand in a patient, prior to treatment with an anti-TLR3 antibody. Generally, in this step, biological sample is taken from a patient, for example a sample of synovial fluid, e.g. in a patient having rheumatoid arthritis. The biological sample is assessed for the presence of a TLR3 ligand, such as the presence of extracellular dsRNA. If the biological sample is positive for the presence of a TLR3 ligand, the patient can then advantageously be treated with the anti-TLR3 antibody, preferably with an antibody that inhibits TLR3 signalling in a TLR3-expressing cell in the presence of a dsRNA TLR3 ligand.

The anti-TLR3 antibody administered to an individual having a disease can be any monoclonal antibody that specifically binds a TLR3 polypeptide, preferably any antibody inhibits signaling by the TLR3 polypeptide, as described herein. For example, the anti-TLR3 antibody is an antibody that specifically binds TLR3, wherein the antibody has a K_{D} for binding to a human TLR3 polypeptide of less than 10⁻⁹M under acid conditions, and optionally further also a K_{D} of less than 10⁻⁹M under neutral conditions.

In one embodiment, the anti-TLR3 antibody is an antibody that specifically binds TLR3, wherein the antibody has one or more (including any combination thereof, or all of) of the following properties:
a. the antibody has a subnanomolar binding affinity for a TLR3 polypeptide at an acidic pH, e.g. a pH less than about 6.5, or between about 4.5 to 6.5 or about pH 5.6;
b. the antibody inhibits TLR3 signaling in the presence of a TLR3 ligand;
c. the antibody inhibits TLR3 signaling in an inflammatory background, e.g. in the presence of inflammatory cytokines such as IFNα;
d. the antibody competes for binding to a TLR3 polypeptide with 31C3, 29H3, 28F11, 23C8 or 34A3;
e. the antibody does not compete with dsRNA for binding to a TLR3 polypeptide, preferably wherein the antibody does not compete with dsRNA for binding to C-terminal portion the TLR3 polypeptide;
f. the antibody inhibits IP-10 secretion in DC (e.g. in human myeloid DC);
g. the antibody binds one, two or more amino acids from the group consisting of amino acid residue positions 102, 103, 105, 107, 108, 109, 110, 112, 113, 114, 115, 116, 117, 118, 120, 121, 123, 124, 126, 127, 129, 131, 132, 133, 134, 136, 137, 139, 140, 141, 144, 145, 147, 148, 150, 151, 153, 155, 156, 157, 158, 160, 161, 163, 166, 167, 168, 171, 172 and 182 on the TLR3 polypeptide of SEQ ID NO:1; and/or
h. the antibody does not bind one, two or more amino acids sequences selected from the group consisting of amino acid residue positions 27 to 42, 116, 145, 171 and/or 196, 177 to 191, 224 to 243, 280 to 286, 295 to 374, 379 to 391, 428 to 459, 461 to 487, 524 to 529, 533 to 542, 546 to 569, 575 to 581, 583 to 605, 607 to 623, 641 to 657 and 670 to 705 on the TLR3 polypeptide of SEQ ID NO:1.

In one embodiment, the anti-TLR3 antibody is used as monotherapy (the sole therapeutic agent).

According to another embodiment, the treatment methods this invention may further comprise treatment an individual with an anti-TLR3 antibody and a second therapeutic agent, including agents normally utilized for the particular therapeutic purpose for which the antibody is being administered. The anti-TLR3 antibody and second therapeutic agent can be administered separately, together or sequentially, or in a cocktail. The second therapeutic agent will normally be administered in amounts typically used for that agent in a monotherapy for the particular disease or condition being treated. In one embodiment, the second therapeutic agent is administered in a dose less than the generally accepted efficacious dose; for example, in various embodiments, the composition comprises a dosage that is less than about 10% to 75% of the generally accepted efficacious dose is administered. Preferably, the second therapeutic agent is an agent that reduces proteolytic enzymes, an inflammatory mediator, or a proinflammatory cytokine such as TNF-α and/or interleukin-1 (IL-1). Preferably, the second therapeutic agent is DMARD or a DMD, optionally further wherein the second therapeutic agent is methotrexate (Rheumatrex™, Trexall™), dexamethasone, hydroxychloroquine (Plaquenil™), sulfasalazine (Azulfidine®), leflunomide (Arava™), a tumor necrosis factor inhibitor (e.g. etanercept (Enbrel®, adalimumab (Humira™), and infliximab (Remicade™)), a T-cell costimulatory blocking agent (e.g. abatacept (Orencia™)), a B cell depleting agent (e.g. rituximab (Rituxan™)), an interleukin-1 (IL-1) receptor antagonist therapy (anakinra (Kineret™)), an anti-BlyS antibody (Benlysta™), intramuscular gold, or another immunomodulatory or cytotoxic agent (e.g. azathioprine (Imuran™), cyclophosphamide, or cyclosporine A (Neoral™, Sandimmune™)).

In some embodiments, the anti-TLR3 antibody is administered prior to the administration of the second therapeutic agent. For example, an anti-TLR3 antibody can be administered approximately 0 to 30 days prior to the administration of the second therapeutic agent. In some embodiments, an anti-TLR3 antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, the anti-TLR3 antibody is administered concurrently with the administration of the therapeutic agents. In some embodiments, the anti-TLR3 antibody is administered after the administration of the second therapeutic agent. For example, an anti-TLR3 antibody can be administered approximately 0 to 30 days after the administration of the second therapeutic agent. In some embodiments, an anti-TLR3 antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

### Rheumatoid Arthritis

Rheumatoid arthritis (RA) is a chronic and typically progressive inflammatory disease in which the synovial membrane is the primary site of inflammation. Bone destruction occurs with the progression of inflammation, resulting in deformation or damage of bones and cartilages. Rheumatoid arthritis sometimes develops into a wasting disease accompanying not only inflammation of synovial membranes or osteoarticular tissues, but also systemic inflammation, causing disorders in various organs and tissues, and may even lead to severe symptoms affecting life prognosis. Rheumatoid arthritis (RA) affects up to 1% of the adult population worldwide (Gabriel, Rheum Dis CHn North Am 27:269-81, 2001). Since rheumatoid arthritis develops in people in their thirties and forties and gradually becomes advanced and aggravated during the middle to old age, it significantly affects daily life. The long-term prognosis of RA is poor, with as much as 50% of patients experiencing significant functional disability within 10 years from the time of diagnosis. (Keystone, Rheumatology, 44 (Suppl. 2): ii8-ii12 (2005)). Life expectancy is reduced by an average of 3-10 years. (Alamanos and Drosos). Patients with a high titer of rheumatoid factor (RF) (approximately 80% of patients) have more aggressive disease (Bukhari et al., Arthritis Rheum., 46: 906-912 (2002)), with a worse long-term outcome and increased mortality over those who are RF negative. (Heliovaara et al., Ann. Rheum. Dis., 54: 811-814 (1995)). Therefore, vigorous research and development of anti-rheumatic agents have been carried out.

The pathogenesis of chronic inflammatory bone diseases, such as RA, is not fully elucidated. TNF-α, IL-1β, and IL-1Ra gene polymorphisms are associated with increased RA susceptibility risk and disease severity. (Paradowska and Lacki, Centr Eur J Immunol., 31(3-4): 117-122 (2006)). IL-1 and TNF-α gene polymorphisms are associated with levels of anti-cytokine, including anti-TNF, clinical responses. Such diseases are accompanied by bone loss around affected joints due to increased osteoclastic resorption. This process is mediated largely by increased local production of pro-inflammatory cytokines. These cytokines can act directly on cells in the osteoclast lineage or indirectly by affecting the production of the essential osteoclast differentiation factor, receptor activator of NFKB ligand (RANKL), and/or its soluble decoy receptor, osteoprotegerin (OPG), by osteoblast/stromal cells. Tumor necrosis factor-alpha (TNF-α) is a major mediator of inflammation. Its importance in the pathogenesis of various forms of bone loss is supported by several lines of experimental and clinical evidence. However, TNF-α is not essential for osteoclastogenesis, erosive arthritis, or osteolysis, as these can occur in the absence of TNF-α.

In RA specifically, an immune response is thought to be initiated/perpetuated by one or several antigens presenting in the synovial compartment, producing an influx of acute inflammatory cells and lymphocytes into the joint. Successive waves of inflammation, also referred to as attacks, lead to the formation of an invasive and erosive tissue called pannus. This contains proliferating fibroblast-like synoviocytes and macrophages that produce proinflammatory cytokines such as TNF-α and interleukin-1 (IL-1). Local release of proteolytic enzymes, various inflammatory mediators, and osteoclast activation contributes to much of the tissue damage. There is loss of articular cartilage and the formation of bone erosions. Surrounding tendons and bursa may become affected by the inflammatory process. Ultimately, the integrity of the joint structure is compromised, producing disability.

Structural damage to joints is an important consequence of chronic synovial inflammation. Between 60% and 95% of patients with RA develop at least one radiographic erosion within 3-8 years of disease onset. (Paulus et al., J. Rheumatol., 23: 801-805 (1996); Hulsmans et al., Arthritis Rheum., 43: 1927-1940 (2000)). In early RA, the correlation between radiographic damage scores and functional capacity is weak, but after 8 years of disease, correlation coefficients can reach as high as 0.68. (Scott et al., Rheumatology, 39:122-132 (2000)).

### Disease Evolution and Rating

Rheumatoid arthritis (RA) progresses in stages. The first stage is the swelling of the synovial lining, causing pain, warmth, stiffness, redness and swelling around the joint. Second is the rapid division and growth of cells, or pannus, which causes the synovium to thicken. In the third stage, the inflamed cells release enzymes that may digest bone and cartilage, often causing the involved joint to lose its shape and alignment, more pain, and loss of movement. A patient affected with the disease can experience a period of remission, without pain, and then a rheumatoid arthritis crisis, also named flare or attack, where the pain will increase. The methods according to the invention propose to treat such patient experiencing a crisis to help them to deal with the pain.

The level of RA disease can be evaluated using different criteria. The most known criteria have been set up by the ACR (American College of Rheumatology). ACR criteria are indicated as ACR 20, ACR 50, and ACR 70. ACR criteria measure improvement in tender or swollen joint counts and improvement in three of the following five parameters: acute phase reactant (such as sedimentation rate), patient assessment, physician assessment, pain scale and disability/functional questionnaire.

The severity of the disease can also be measured by a score known as DAS (Disease Activity Score). DAS is a composite index of RA activity drawn up by EULAR (European League Against Rheumatism) initially developed for 44 joints for the numbers of joints with synovitis and the 53 Ritchie index sites. DAS is calculated according to the following formula:

DAS=[0.553938√Richie's index]+[0.06465√(number of joints with synovitis)]+[0.330 Ln (erythrocyte sedimentation rate)]+0.024

Ritchie's index covers 53 joints: temporomandibular, acromioclavicular, sternocostoclavicular, shoulder, elbow, wrist, metacarpophalangeal (MCP), proximal interphalangeal (PIP) in the fingers, hip, knee, ankle, subtalar, transverse tarsal, and metatarsophalangeal (MTP).

Three activity levels have been defined according to the value of DAS: RA with low activity level DAS≦2.4, moderate active RA 2.4<DAS≦3.7, active RA>3.7. Remission threshold value defined for DAS is <1.6.

The primary objective of the methods of treatment according to the invention is to control the activity of the disease and, also, to achieve remission, reduce pain, prevent and control joint destruction, prevent loss of function in everyday activities and at work, and optimise the patient's quality of life.

### Current Treatment Options

Current recommendations for treatment of RA include early treatment with disease modifying anti-rheumatic drugs (DMARDs) after the diagnosis has been established. Non-steroidal anti-inflammatory drugs (NSAIDs), and until recently, COX-2 inhibitors have been widely used while waiting to confirm the diagnosis or later in the course of the disease in conjunction with DMARDs. Methotrexate is the most widely used DMARD, but other agents, including hydroxychloroquine, sulfasalazine, gold, minocycline, and leflunomide, are also prescribed. Corticosteroids may be used in combination with DMARDs, but in general, only low doses are used to minimize adverse events (O'Dell, New Engl. J. Med. 350:2591-2603, 2004). In recent years, anti-cytokine therapies targeting inflammatory cytokines have been receiving attention, and novel biopharmaceuticals having effective anti-rheumatic actions, such as infliximab, etanercept, anakinra, and atlizumab, have been developed. However, there is currently no totally effective treatment and there remains a need for an efficient treatment of the disease, and improvement of the patient's comfort and pain relief and alternative therapies are needed to improve patient's daily life. Some of the main treatments are reviewed hereunder.

Non-steroidal anti-inflammatory agents (NSAIDs). These drugs inhibit the generation of prostaglandins by blocking cyclooxygenase enzymes, COX-1 and COX-2. Prostaglandins are mediators of inflammation and pain but also have important roles in maintenance of normal body functions including protection from stomach acid, maintenance of kidney blood flow, and contributing to platelet stickiness and vascular function. COX-2 selective inhibitors selectively block prostaglandins generated via COX-2 which have prominent roles in inflammation. Many different NSAIDS are available, some over the counter including aspirin, ibuprofen (Advil®, Motrin®, Nuprin ®) and naproxen (Alleve®) and many others are available by prescription including meloxicam (Mobic®), etodolac (Lodine®), nabumetone (Relafen®), sulindac tolementin (Tolectin®), choline magnesium salicylate (Trilasate®), diclofenac (Cataflam®, Voltaren®, Arthrotec®), Diflusinal (Dolobid®), indomethicin (Indocin®), Ketoprofen (Orudis®, Oruvail®), Oxaprozin (Daypro®), and piroxicam (Feldene®). Longer acting NSAIDs that allow daily or twice daily dosing may improve compliance. The NSAID class also includes drugs known as COX-2 inhibitors that are also effective in controlling inflammation. Only one of these agents is currently available in the United States (celecoxib, Celebrex®) while additional compounds are available in other countries (etoricoxib, Arcoxia®; lumiracoxib, Prexige®). These drugs were designed to decrease the gastrointestinal risk of NSAIDS, but concerns of possible increases in cardiovascular risk with these agents has led to the withdrawal of two of these drugs from the market (rofecoxib, Vioxx®; valdecoxib, Bextra®). While in some cases, lower doses of NSAIDS are effective, in rheumatoid arthritis and other forms of inflammatory arthritis a higher dose is often required to decrease inflammation. A lower dosage can initially be used if inflammation is mild, if mechanical pain is the major problem, if the patient is elderly or if the patient suffers from conditions that increase the risk for toxicity (see below). If a particular preparation is ineffective after a 4-week trial or is not tolerated, then another NSAID can be initiated. No one NSAID has been demonstrated to be better than another for the treatment of rheumatoid arthritis nor have the COX-2 agents been shown to be superior to traditional NSAIDS in terms of effectiveness.

Corticosteroids (prednisone; dexamethasone, methylprenisolone, Medrol®) have both anti-inflammatory and immunoregulatory activity. They can be given orally, intravenously, intramuscularly or can be injected directly into the joint. Corticosteroids are useful in early disease as temporary adjunctive therapy while waiting for DMARDs to exert their anti-inflammatory effects. Corticosteroids are also useful as chronic adjunctive therapy in patients with severe disease that is not well controlled on NSAIDs and DMARDs. The usual dose of predinisone is 5 to 10 mg daily. Although prednisone can be started at higher doses (15 to 20 mg daily), attempts should be made to taper the dose over a few weeks to less than 10 mg daily. Once started, corticosteroid therapy may be very difficult to discontinue and even at low doses. Some patients are very sensitive to the tapering of prednisone which is generally done slowly over a few weeks.

Weight gain and a cushingoid appearance (increased fat deposition around the face, redness of the cheeks, development of a “buffalo hump” over the neck) is a frequent problem and source of patient complaints. Other side effects of prednisone include weight gain, increased blood pressure, increased blood sugar, increased risk of cataracts, and avascular necrosis of bones. Steroid medications are also associated with accelerated osteoporosis even with relatively low dose prednisone at doses of 10 mg daily. Patients with and without osteoporosis risk factors on low dose prednisone should undergo bone densitometry (DEXA Scan) to assess fracture risk. Bisphosphonates such as alendronate (Fosamax®), risedronate (Actonel®), ibandronate (Boniva®) are recommended to prevent and/or treat osteoporosis in addition to adequate calcium and vitamin D supplementation. Recent studies suggest that low dose prednisone may have effects as a “disease modifying” agent in RA, especially when used in combination with other DMARD medications. Higher doses of prednisone are rarely necessary unless there is a life-threatening complication of RA and, if used for prolonged periods, may lead to serious steroid toxicity. Although a few patients can tolerate every other day dosing of corticosteroids which may reduce side effects, most require corticosteroids daily to avoid symptoms. Once a day dosing of prednisone is associated with fewer side effects than the equivalent dose given twice or three times daily. Generally steroids are given in the morning upon wakening to mimic the body's own steroid surge. Repetitive short courses of high-dose corticosteroids, intermittent intramuscular injections, adrenocorticotropic hormone injections, and the use of corticosteroids as the sole therapeutic agent are all to be avoided. Intra-articular corticosteroids (e.g., triamcinolone or methylprednisolone and others) are effective for controlling a local flare in a joint without changing the overall drug regimen.

Disease Modifying Anti-rheumatic Drugs (DMARDS): Although both NSAIDs and DMARD agents improve symptoms of active rheumatoid arthritis, only DMARD agents have been shown to alter the disease course and improve radiographic outcomes. DMARDs have an effect upon rheumatoid arthritis that is different and may be more delayed in onset than either NSAIDs or corticosteroids. In most cases, when the diagnosis of rheumatoid arthritis is confirmed, DMARD agents should be started. The presence of erosions or joint space narrowing on x-rays of the involved joints is a clear indication for DMARD therapy, however one should not wait for x-ray changes to occur. The currently available drugs include: Methotrexate (Rheumatrex®, Trexall®), Hydroxychloroquine (Plaquenil®), Sulfasalazine (Azulfidine®), Leflunomide (Arava®), Tumor Necrosis Factor Inhibitors—etanercept (Enbrel®, adalimumab (Humira®), and infliximab (Remicade®), T-cell Costimulatory Blocking Agents—abatacept (Orencia®), B cell Depleting Agents—rituximab (Rituxan®), Interleukin-1 (IL-1) Receptor Antagonist Therapy—anakinra (Kineret®), Intramuscular Gold, Other Immunomodulatory and Cytotoxic agents—azathioprine (Imuran®), cyclophosphamide, and cyclosporine A (Neoral®, Sandimmune®).

Methotrexate is now considered the first-line DMARD agent for most patients with RA. It has a relatively rapid onset of action at therapeutic doses (6-8 weeks), good efficacy, favorable toxicity profile, ease of administration, and relatively low cost. Methotrexate is effective in reducing the signs and symptoms of RA, as well as slowing or halting radiographic damage. Methotrexate is also effective in many other forms of inflammatory arthritis including psoriatic arthritis and other spondyloarthopathies, and is used in many other autoimmune diseases. Dosage: In a study comparing methotrexate to etanercept in early RA, methotrexate was started at a dose of 10 mg per week, and increased to 20 mg per week by week 8. This dosing regimen or regimens that start at even higher doses (up to 15 mg per week) with a dose escalation to 20 mg within the first three months is now fairly well accepted in clinical practice. Maximal dose is usually 25 mg per week but is sometimes increased further. Methotrexate can be given orally or by subcutaneous injection. The latter route of administration can be advantageous for patients who have methotrexate-associated nausea. Patients starting methotrexate should be carefully evaluated for renal insufficiency, acute or chronic liver disease, significant alcohol intake or alcohol abuse, leukopenia (low white blood cell counts), thrombocytopenia (low platelet counts), or untreated folate deficiency. Obesity, diabetes and history of hepatitis B or C are factors that have been suggested but not confirmed to increase methotrexate hepatotoxicity (liver injury). Salicylates (and other NSAIDs) and the antibiotic trimethoprim (Bactrim®, Septra®) block the renal excretion of methotrexate and increase serum levels with an increased risk of toxicity. If alternatives exist, concomitant use of methotrexate and trimethoprim is to be avoided. The coadministration of NSAIDS with methotrexate is routine in patients with rheumatoid arthritis and is considered safe by rheumatologists as long as liver function tests are closely monitored. Methotrexate can be combined safely with nearly every other FDA approved DMARDs for RA, including sulfasalazine, hydroxychloroquine, TNF inhibitors, abatacept, rituximab, anakinra, and leflunomide. In all clinical trials combining methotrexate with one of these DMARDs, no unexpected toxicities or synergistic toxicities were observed with the exception of higher liver toxicity with leflunomide which is also metabolized by the liver.

Hydroxychloroquine and chloroquine are antimalarial drugs which are relatively safe and well-tolerated agent for the treatment of rheumatoid arthritis. Because these drugs have limited ability to prevent joint damage on their own, their use should probably be limited to patients with very mild and nonerosive disease. Hydroxychloroquine is sometimes combined with methotrexate for additive benefits for signs and symptoms or as part of a regimen of “triple therapy” with methotrexate and sulfasalazine.

Sulfasalazine (Azulfidine®) is an effective DMARD for the treatment of RA. It is given in conjunction with methotrexate and hydroxychloroquine as part of a regimen of “triple therapy” which has been shown to provide benefits to patients who have had inadequate responses to methotrexate alone. Sulfasalazine is also used in the treatment of inflammatory bowel disease and spondyloarthropathies. Its mechanism of action in RA is unknown. Some of its effects may be due to folate depletion. Dosage: The usual dose is 2-3 grams per day in a twice daily dosing regimen. The dose may be initiated at 1 gram per day and increased as tolerated.

Leflunomide (Arava®) is also an effective DMARD. Its efficacy is similar to methotrexate in terms of signs and symptoms, and is a viable alternative to patients who have failed or are intolerant to methotrexate. Leflunomide has been demonstrated to slow radiographic progression. Studies have demonstrated that it can also be carefully combined with methotrexate in patients with no preexisting liver disease, as long as the liver function tests are carefully monitored. Leflunomide has also been studied in psoriatic arthritis with some efficacy demonstrated. Dosage: The half-life of the active metabolite of leflunomide is very long. Leflunomide and its metabolites are extensively protein bound and undergo further metabolism before excretion. When initially approved, the medication was given using a loading dose of 100 mg daily for three days then followed by 20 mg daily. Due to a significant incidence of GI side effects and diarrhea, most practitioners now use a shorter loading period with lower doses or initiate treatment at 10-20 mg/day with no loading dose. The dose may be reduced to 10 mg daily if not tolerated at the 20 mg dose.

Tumor necrosis factor (TNF) inhibitors. TNF is found in large quantities in the rheumatoid joint and is produced locally in the joint by synovial macrophages and lymphocytes infiltrating the joint synovium. TNF is one of the critical cytokines that mediate joint damage and destruction due to its activities on many cells in the joint as well as effects on other organs and body systems. TNF antagonists were the first of the biological DMARDS to be approved for the treatment of RA and have also been referred to as biological response modifiers or “biologics” to differentiate them from other DMARDS such as methotrexate, leflunomide, or sulfasalazine. Three TNF antagonists are approved for the treatment of RA and additional agents are under investigation. These drugs are similar in their efficacy at decreasing signs and symptoms of RA, slowing or halting radiographic damage, and improving function and quality of life. These agents are also now approved for the treatment of other forms of inflammatory arthritis including psoriatic arthritis, juvenile idiopathic arthritis and ankylosing spondylitis. There are currently three TNF inhibitors FDA approved for the treatment of RA (listed in order of their approval for RA); etanercept (Enbrel®), infliximab (Remicade®), and adalimumab (Humira®).

Etanercept (Enbrel®) is effective in reducing the signs and symptoms of RA, as well as in slowing or halting radiographic damage, when used either as monotherapy or in combination with methotrexate. Etanercept is also approved for the treatment of psoriatic arthritis and for ankylosing spondylitis as well as psoriasis. Etanercept is a fusion protein that combines two extracellular binding domains of the p75 form of the TNF receptor with the Fc portion of a human IgG1 antibody molecule. The components of the protein are entirely human, and anti-etanercept antibodies are relatively uncommon. Dosage: The most common dose currently used is 50 mg self-administered once per week by subcutaneous injection. Both prefilled syringes and an autoinjection system (SureClick®) are available. Etanercept is also available in a 25 mg dose which is administered twice per week at this dose. Intermittent or occasional dosing has not been studied. There is limited information on the safety or efficacy at doses beyond 50 mg per week. Etanercept has a half-life of 70 hours after a 25 mg dose.

Infliximab (Remicade®): in combination with methotrexate, is approved for the treatment of RA, and for the treatment of psoriatic arthritis, and ankylosing spondylitis, as well as psoriasis and Crohn's disease. Infliximab is a chimeric monoclonal antibody that binds TNF with high affinity and specificity. The antibody binding site for TNF is of mouse origin, with the remaining 75% of the infliximab antibody derived from a human IgG1 antibody sequence. Infliximab is effective as monotherapy in reducing the signs and symptoms of RA but anti-infliximab antibodies can develop which can, in turn, reduce the durability of the response. Co-treatment with methotrexate reduces the frequency of these antibodies and is therefore recommended along with infliximab. The combination of infliximab and methotrexate is very effective in reducing clinical manifestations of disease, as well as in slowing or halting radiographic progression of disease in RA. Dosage: Infliximab is administered via the intravenous route. Infusions typically take between 2-3 hours. The recommended starting dose of infliximab is 3 mg/kg for RA given as an intravenous infusion followed by additional dosing at 2 and 6 weeks, then every 8 weeks thereafter. Infliximab should be given in combination with methotrexate. If the clinical response is inadequate at a starting dose, infliximab can be increased incrementally to a maximum dose of 10 mg/kg and the frequency of infusion increased to every 4-6 weeks.

Adalimumab (Humira®) is a fully human anti-TNF monoclonal antibody with high specificity for TNF. Like the other TNF antagonists, it is effective as monotherapy and in combination with methotrexate, at reducing signs and symptoms of RA and in slowing or halting radiographic progression of disease. It is administered by subcutaneous injection every two weeks but can be increased to weekly, if needed. Adalimumab is effective in RA, Psoriatic arthritis, and ankylosing spondylitis, and Crohn's disease. Dosage: Adalimumab is currently available in a 40 mg dose and is given by self-administered subcutaneous (SC) injection every other week. Both prefilled syringes as well as an autoinjector system (Huimira Pent) are available. If response to this dose is inadequate, the frequency of injections can be increased to weekly. Adalimumab has a half-life of approximately 2 weeks (ranging from 10-20 days) after a standard 40 mg dose.

T-cell Costimulatory blockade: Abatacept (Orencia®): Abatacept is the first of a class of agents known as T-cell costimulatory blockers. These agent interfere with the interactions between antigen-presenting cells and T lymphocytes and affect early stages in the pathogenic cascade of events in rheumatoid arthritis. T lymphocytes become activated due to an unknown stimulus but likely involving the interaction between antigen presented in the context of the Class II Major Histocompatability Complex molecule on the surface of antigen presenting cells. T cells recognize antigens as foreign and if they receive a second stimulus, will become active, proliferate, traffic to inflamed sites, and secrete proinflammatory cytokines including TNF. One of the important second signals for T cell activation is mediated by the molecules CD80 and CD86 found on antigen presenting cells and the CD28 molecule on the T cell surface. Dosage: Abatacept is administered via intravenous infusion once per month after initial doses at baseline, 2 weeks, and 4 weeks. The dose is based on body weight, with patients <60 kg receiving 500 mg, 60-100 kg receiving 750 mg, and >100 kg receiving 1000 mg. The medication is administered over a period of approximately 30 minutes to one hour.

B-Cell Depletion: Rituximab (Rituxan®): B cells are an important inflammatory cell with multiple functions in the immune response. They serve as antigen presenting cells, can secrete cytokines, and differentiate into antibody-forming plasma cells. The depletion of B cells has been shown to be effective in reducing signs and symptoms of RA and in slowing radiographic progression. One B cell depleting agent, Rituximab, is currently available for the treatment of rheumatoid arthritis. Rituximab (Rituxan®) was originally developed to treat non-Hodgkin's lymphoma and has been used to treat this malignant condition of lymphocytes and lymph nodes for several years. Early studies in patients with rheumatoid arthritis showed rituximab caused a rapid and sustained depletion of circulating B cells in the circulation with clinical improvements in many patients as well. Further clinical studies have now demonstrated that rituximab is effective in decreasing signs and symptoms and in slowing radiographic progression in RA patients who have failed other DMARD therapies. The agent is currently approved in the US, however, only in patients who have failed TNF antagonists. Dosage: The currently approved dose is 1000 mg administered intravenously over 3-4 hours with two doses given 2 weeks apart. Patients typically receive intravenous corticosteroids with each infusion and premedication with diphenhydramine and acetaminophen. The optimal time for readministarion is not yet clear. Some have advocated a fixed dosing regimen of every 6 months, while others have advocated waiting until a patient begins to flare before retreating. Studies are ongoing to evaluate redosing schedules. The extent and duration of B cell depletion has not been clearly correlated with efficacy. Nor has the reconstitution of normal levels of B cells been well correlated with loss of efficacy.

Interleukin-1 (IL-1) is another proinflammatory cytokine implicated in the pathogenesis of RA. IL-1 receptor antagonist (IL1ra) is an endogenous blocker of the cytokine. Evidence supporting an anti-inflammatory role of IL-1ra in vivo is demonstrated by the observation that IL-1ra deficient mice spontaneously develop autoimmune diseases similar to rheumatoid arthritis as well as vasculitis. IL1 has effects on cartilage degradation leading to damage as well as inhibiting repair, and is a potent stimulus to osteoclasts leading to bone erosion. One IL1 antagonist, anakinra (Kineret®), is currently approved for the treatment of RA. Other agents have been studied as well in RA.

Anakinra (Kineret™), a human recombinant IL-1 receptor antagonist (hu rIL-1ra) is approved for the treatment of RA Anakinra can be used alone or in combination with DMARDs other than TNF blocking agents (Etanercept, Infliximab, Adalimumab) Anakinra is not recommended for use in combination with TNF inhibitors because studies have shown increased infections without additive clinical benefit. Dosage: The recommended dose of anakinra is 100 mg/day administered daily by subcutaneous injection. The dose should be administered at approximately the same time each day. An autoinjection system is available for the medication.

Intramuscular Gold is effective in the treatment of rheumatoid arthritis. Intramuscular gold salts were, until the 1990's, the most often used DMARD agents but have been replaced by Methotrexate and other DMARDS as the preferred agents to treat RA. Two injectable compounds are available, (Myochrysine® and Solganal®). Gold compounds are rarely used now due to their numerous side effects and monitoring requirements, their limited efficacy, and very slow onset of action. An oral gold compound (Auranofin®) is also available but its efficacy is even more limited than injectable compounds. Dosage: Myochrysine or Solganal therapy is started at 10 mg intramuscularly, 25 mg is then given the second week, then 50 mg is given weekly until a response has occurred or until a total of 1 g has been given. If there is a favorable response, therapy is tapered to 50 mg every 2 weeks for 3 months, then every 3 weeks for 3 months and then finally to a maintenance monthly dose. No response after a total of 1 g should be considered a treatment failure. Monthly gold should be continued indefinitely.

Other Immunomodulatory and Cytotoxic Agents: The most commonly used cytotoxic drugs are azathioprine (Imuran®), cyclosporin A (Sandimmune®, Neoral®), cyclophosphamide (Cytoxan®) and d-Penicillamine. Because the potential of high toxicity, these agents are typically utilized for life-threatening extra-articular manifestations of RA such as systemic vasculitis or with severe articular disease that is refractory to other therapy.

Azathioprine (Imuran®) has some activity in rheumatoid arthritis but may take 8-12 weeks to see an effect. It is a purine analog that can cause bone marrow suppression and lowering of blood cell counts (white blood cells, red blood cells, and platelets) particularly in patients with renal insufficiency or when used concomitantly with allopurinol or ACE inhibitors. Increased risk of secondary malignancy due to azathioprine is controversial. Screening for levels of the enzyme thiopurine methyltransferase (TPMT) is recommended before initiating therapy with azathioprine. Certain individuals have deficiencies in this enzyme that metabolizes azathioprine with a concomitantly increased risk of toxicity for the medication. Side effects include nausea, and alopecia. Blood tests to monitor blood counts and liver function tests are necessary for patients on azathioprine.

Cyclosporine (Sandimmune®, Neoral®) has some activity as a disease modifying therapy in rheumatoid arthritis. Studies have demonstrated that cyclosporine can be combined with methotrexate in RA patients to capture clinical responses. It is an immunosuppressive agent approved for use in preventing renal and liver transplant rejection and also has activity in psoriasis and other autoimmune diseases. Cyclosporine inhibits T cell function by inhibiting transcription of interleukin-2. Main toxicities include infection and renal insufficiency. Increase in blood pressure is common and may require treatment. Careful monitoring of renal function and blood pressure is needed for the entire time a patient is taking cyclosporine. Numerous medication interactions may affect blood levels of cyclosporine and lead to more toxicity. The package insert contains important information concerning these medication interactions. Cyclosporine increases risks of infection and may also increase the risk of malignancies including lymphoma.

Cyclophosphamide (Cytoxan®) is a potent immunosuppressive agent that is reserved for severe cases of refractory rheumatoid arthritis and those with manifestations such as vasculitis. It is used in the treatment of other autoimmune conditions including lupus and vasculitis. Cyclophosphamide is an alkylating agent with serious toxicities including bone marrow suppression, hemorrhagic cystitis, premature ovarian failure, infection and secondary malignancy particularly an increased risk of bladder cancer. Blood counts must be carefully monitored with this medication.

d-Penicillamine (Cuprimine®, Depen®) historically has some activity as a treatment for rheumatoid arthritis. It is prescribed primarily for patients with persistent aggressive disease who have failed other available DMARDS. Like gold it is a relatively toxic drug that has limited utility due to issues of tolerability and efficacy that is not as robust as other currently available agents. Major side effects include severe rash and effects on renal function. Careful monitoring of kidney function is required with this drug. Patients may develop a lupus like illness or other autoimmune diseases when taking d-Penicillamine.

Other DMARD compounds currently in development are also suitable for a combination in the treatment methods according to the invention, such as VX-702, ocrelizumab, compounds targeting SYK kinase such as fostimatinib (R788) and JAK1, JAK2 inhibitors such as INCB28050, tanezumab or tasocitinib^{M} (CP-690,550).

Treatment with an Anti-TLR3 Antibody

A patient having RA can be evaluated to assess the presence, stage, evolution or rating of disease. Optionally a biological sample (e.g. synovial fluid) is obtained and assessed for the presence of proinflammatory mediators or other markers of active inflammation, and/or dsRNA. In one embodiment, the presence of auto-antibodies is detected, for example detecting rheumatoid factor (RhF), anti-cyclic citrullinated peptide antibodies, anti-ssRNA, anti-dsRNA, anti-Smith, anti-phospholipid, anti-nuclear and/or anti-actin antibodies. In one embodiment, the methods comprise assessing levels of a proteolytic enzyme, an inflammatory mediator, a marker of ongoing inflammation or a proinflammatory cytokine. In one embodiment, the methods comprise determining c-reactive protein (CRP) level and/or erythrocyte sedimentation rate. A determination that a patient has RA, or that pro-inflammatory mediators or other markers of active inflammation, and/or dsRNA are present (e.g. in the inflamed tissue), that disease is acute, chronic, experiencing a flare or progressing indicates that the patient can be treated with an anti-TLR3 antibody.

In one aspect, a patient having RA, and optionally having active inflammation and/or established or chronic RA, and/or experiencing a flare is treated with an anti-TLR3 antibody. Preferably, established RA may be characterized as RA which has been progressing for over a year, or which has been progressing for less than a year but is unresponsive to a first disease modifying anti-rheumatic drug (DMARD). Established RA can also be assessed using the DAS or the CAS criteria. “RA and related diseases” refers to diseases that can cause or derive from the onset or evolution of rheumatoid arthritis such as e.g. episcleritis, pneumothorax, embolism and ischemic skin ulcer.

In one embodiment, the antibodies according to the invention are administered in combination with another RA treatment, such as those listed above.

The anti-TLR3 antibody can be injected or infused via subcutaneous, intravenous, intramuscular, intra-articular, intra-synovial, intrasternal, intrathecal, intrahepatic, intralesional and intracranial routes. In an embodiment, a TLR3 antibody is administered intra-articularly, preferably at the site of the inflammation.

### Multiple Sclerosis

Multiple sclerosis (MS) is a chronic inflammatory disease of the central nervous system (CNS) in which the fatty myelin sheaths around the axons of the brain and spinal cord are damaged, leading to demyelination and scarring as well as a broad spectrum of signs and symptoms. Pathophysiological cause remains unknown although different theories incriminate genetics or infections. Different environmental risk factors have also been proposed. Clinical manifestations are associated with the infiltration of the central nervous system by immune-competent cells. Specific T cell populations directed towards neuroantigens, such as myelin basic protein, can be demonstrated in the periphery. Disease onset usually occurs in young adults, and it is more common in females. It has a prevalence that ranges between 2 and 150 per 100,000.

Almost any neurological symptom can appear with the disease, and often progresses to physical and cognitive disability. MS progresses in two forms: new symptoms occurring either in discrete attacks (relapsing forms) or slowly accumulating over time (progressive forms). Between attacks, symptoms may go away completely, but permanent neurological problems often occur, especially as the disease advances.

### Disease Evaluation and Rating

Several subtypes, or patterns of progression, have been described. Subtypes use the past course of the disease in an attempt to predict the future course. They are important not only for prognosis but also for therapeutic decisions. In 1996 the United States National Multiple Sclerosis Society standardized four subtype definitions: relapsing remitting, secondary progressive, primary progressive, and progressive relapsing.

The relapsing-remitting subtype is characterized by unpredictable relapses followed by periods of months to years of remission with no new signs of disease activity. This describes the initial course of 80% of individuals with MS. Secondary progressive MS describes around 65% of those with an initial relapsing-remitting MS, who then begin to have progressive neurologic decline between acute attacks without any definite periods of remission. Occasional relapses and minor remissions may appear. The median time between disease onset and conversion from relapsing-remitting to secondary progressive MS is 19 years. The primary progressive subtype describes the approximately 10-15% of individuals who never have remission after their initial MS symptoms. It is characterized by progression of disability from onset, with no, or only occasional and minor, remissions and improvements. The age of onset for the primary progressive subtype is later than for the relapsing-remitting, but similar to mean age of progression between the relapsing-remitting and the secondary progressive. In both cases it is around 40 years of age. Progressive relapsing MS describes those individuals who, from onset, have a steady neurologic decline but also suffer clear superimposed attacks. This is the least common of all subtypes. Multiple sclerosis evolves either by progressive neurologic decline or by acute attacks, or by a combination of both depending on the MS type. Symptoms of MS include: fatigue, visual problems such as blurred or double vision, tingling, numbing, or burning sensations, muscle weakness, stiffness, tremor, and spasms, walking and gait problems, bladder and bowel dysfunction, sexual dysfunction, cognitive and memory problems, swallowing and speech problem, pain or depression. Those symptoms are exacerbated during an attack whereas the general condition of the patient declines. Typical variants of MS with non-standard behavior have been described; these include Devic's disease, Balo concentric sclerosis, Schilder's diffuse sclerosis and Marburg multiple sclerosis.

The multiple sclerosis diagnostic is established using different criteria. Historically, the Schumacher and Poser criteria were widely used. Currently, the McDonald criteria, established by the National Multiple Sclerosis Society (NMSS) of America, using IMR imaging, tend to replace the older criteria. (McDonald W I, Compston A, Edan G, et al. (2001), Ann. Neurol. 50 (1): 121-7).

### Current Treatment Options

There are many issues for the patient and physician to consider in treating multiple sclerosis. Goals may include: improving the speed of recovery from attacks (e.g. treatment with steroid drugs); reducing the number of attacks or the number of MRI lesions; attempting to slow progression of the disease (treatment with disease modifying drugs or DMDs), one additional goal is relief from complications due to the loss of function of affected organs.

Most neurologists will consider treatment with DMDs once the diagnosis of relapsing remitting multiple sclerosis is established. Many will begin treatment at the time of the first multiple sclerosis attack, since clinical trials have suggested that patients in whom treatment is delayed may not benefit as much as patients who are treated early.

It is important for patients to talk to their doctor before deciding to go on therapy since DMDs differ in their uses (for example, one DMD may be used for slowing progressing disability but not for treatment of the first attack of MS; another DMD may be used for reducing relapses but not for slowing progressing disability). Finally, utilizing support groups or counselling may be helpful for patients and their families whose lives may be affected directly by multiple sclerosis. Patients receive immunosuppressive therapy including azathioprine and corticosteroids in order to limit the extent of the inflammatory process. Immunosuppressive therapy of multiple sclerosis, however, is only partially effective, and in most cases only offers a delay in disease progression despite anti-inflammatory and immunosuppressive treatment. Current disease-modifying treatments for MS are, inter alia: IFNβ-1a (Avonex™, CinnoVex™, ReciGen™, Rebif™), INFβ-1b (Betaseron™, Betaferon™), glatiramer acetate (Copaxone™) which is a non-interferon, non-steroidal immunomodulator, mitoxantrone, an immunosuppressant, natalizumab (Tysabri™), fingolimod (Gilenia™). A number of treatments are under investigation. Emerging agents for RRMS that have shown promise in phase 2 trials include alemtuzumab (Campath™), daclizumab (Zenapax™) rituximab, dirucotide, BHT-3009, cladribine, dimethyl fumarate, estriol, fingolimod, laquinimod, minocycline, statins, temsirolimus and teriflunomide.

Treatment with a Anti-TLR3 Antibody

Optionally, in a first step a patient's disease can be evaluated. The patient is then treated with an anti-TLR3 antibody in an appropriate manner. A patient having MS can be evaluated to assess the presence, stage, evolution or rating of disease. In one advantageous aspect, a patient determined to have active inflammation, and/or established or chronic MS, and/or experiencing a flare is treated with an anti-TLR3 antibody. Preferably, established MS may be characterized as MS which has progressive neurologic decline, such as secondary progressive, primary progressive or progressive relapsing type disease alternatively, an “established MS” refers to a MS which has been progressing for over a year, or which has been progressing for less than a year but is unresponsive to a first line of treatment. Preferably a flare is defined as an exacerbation of the symptoms related to multiple sclerosis, optionally; such flare leads to a decline in the patient's general condition. Another aspect of the invention is to provide a composition which is able to treat an established MS, or to reduce or abort an MS attack, thereby leading to an improvement of the patient's health and comfort.

In one embodiment, the antibodies according to the invention are administered in combination with another MS treatment, such as those listed above.

The anti-TLR3 antibody can be injected or infused via subcutaneous, intravenous, intramuscular, intra-articular, intra-synovial, intrasternal, intrathecal, intrahepatic, intralesional and intracranial routes.

### Chronic Inflammatory Diseases of the Intestine (CIDI)—Crohn's Disease—Rectocolitis

Chronic inflammatory diseases of the intestine are a series of diseases affecting the gastrointestinal tractus. Most common CIDI are ulcerative colitis, Crohn's disease, inflammatory bowel disease, regional enteritis, rectocolitis and Granulomatous ileocolitis. Crohn's disease is an inflammatory disease of the intestines that may affect any part of the gastrointestinal tract from mouth to anus, causing a wide variety of symptoms. It primarily causes abdominal pain, diarrhea, vomiting, or weight loss, but may also cause complications outside of the gastrointestinal tract such as skin rashes, arthritis, inflammation of the eye, tiredness and augments the likelihood of a colon cancer within a few years.

Crohn's disease affects between 400,000 and 600,000 people in North America, with no prevalence according to gender. Prevalence estimates for Northern Europe have ranged from 27-48 per 100,000. Smokers are two times more likely to develop Crohn's disease. Crohn's disease onsets usually in young adults, although the disease can occur at any age.

### Disease Evaluation and Rating

Historically, Crohn's disease has been difficult to diagnose. In part, this is because its symptoms are similar to those of other bowel disorders, including ulcerative colitis and irritable bowel syndrome. A larger problem is that the small intestine has been difficult to examine using traditional methods.

Diagnosis tests include: noninvasive laboratory tests (anemia and infection, liver function tests to screen for liver and bile duct problems, and stool studies to rule out bacterial, viral and parasitic infections), endoscopy, endoscopic ultrasound (EUS), capsule endoscopy, radiology such as Multiphase CT enterography, MR enterography (MRE).

Chronic inflammatory diseases of the intestine are rather hard to score. In rectocolitis, a coloscopy can provide a quite complete overview of the wounds in the colon, but in Crohn's disease, as wound can appear anywhere from oesophagus to rectum, patient evaluation is much more difficult to obtain. A scoring system has been set up to evaluate Crohn's disease: the Crohn's disease activity index (CDAI, see for review Sandborn W J et al. Gastroenterology 2002; 112: 512). Score ranges from 0 to 600. Below 150 points, patients are scored as “very well”. Between 150 and 219, the disease is mildly active, between 220 and 449, the disease is moderately active. Above 450 points the disease is rated as very severe. However, such scoring may be patient dependant and more recently, another scoring system, the Crohn Disease Digestive Damage Score (CD₃S) or Lemann score has been established to score patients disease through more precise scientific values, such as a score established by tomodensitometry. (Pariente B. et al. Development of the Crohn's Disease Digestive Damage score, the Lemann score Innflamm Bowel Dis 2010; Nov. 28).

Crohn's disease evolves with crisis, also known as flare-ups. Flare-ups can be mild or severe, brief or prolonged. Such flare-ups or attacks can be associated with a CDAI score of more than 150, more than 219, more than 449 points. Severe flare-ups can lead to intense pain, dehydration, and blood loss. Recurrent inflammation tends to appear in the same area of the intestine, but it may spread to adjacent areas after a diseased segment has been removed surgically. When Crohn's disease causes a flare-up of gastrointestinal symptoms, the person may also experience inflammation of the joints (arthritis), inflammation of the whites of the eyes (episcleritis), mouth sores (aphthous stomatitis), inflamed skin nodules on the arms and legs (erythema nodosum), and blue-red skin sores containing pus (pyoderma gangrenosum). Even when Crohn's disease is not causing a flare-up of gastrointestinal symptoms, the person still may experience pyoderma gangrenosum, while inflammation of the spine (ankylosing spondylitis), inflammation of the pelvic joints (sacroiliitis), inflammation inside the eye (uveitis), or inflammation of the bile ducts (primary sclerosing cholangitis) are liable to occur entirely without relation to the clinical activity of the bowel disease.

### Current Treatment Options

Current treatment options are restricted to controlling symptoms, maintaining remission, and preventing relapse. Treatment of Crohn's disease involves first treating the acute symptoms of the disease, then maintaining remission. Treatment initially involves the use of medications to eliminate infections, generally antibiotics, and reduce inflammation, generally aminosalicylate anti-inflammatory drugs and corticosteroids. Surgery may be required for complications such as obstructions or abscesses, or if the disease does not respond to drugs within a reasonable time.

Aminosalicylate anti-inflammatory drugs: Mesalazine or mesalamine (Lialda™, Asacol™, Pentasa™, Salofalk™, Dipentum™ and Rowasa™), Sulfasalazine, which is converted to 5-ASA and sulfapyridine by intestinal bacteria. The sulfapyridine may have some therapeutic effect in rheumatoid arthritis. However, the sulfapyridine component is often the limiting factor in treatment of Crohn's disease because of high side-effect profile. 5-ASA compounds have been shown to be useful in the treatment of mild-to-moderate Crohn's disease. They are usually considered to be first line therapy for disease in the ileum and right side of the colon particularly due to their lower side effect profile compared to corticosteroids.

Corticosteroid anti-inflammatory drugs: Steroid enemas can be used for treatment of rectal disease symptoms. Corticosteroids are a class of anti-inflammatory drug that are used primarily for treatment of moderate to severe flares or attacks of Crohn's disease. They are used more sparingly due to the availability of effective treatments with less side-effects. The most commonly prescribed oral steroid is prednisone, which is typically dosed at 0.5 mg/kg for induction of remission. Intravenous steroids are used for cases refractory to oral steroids, or where oral steroids cannot be taken. Because corticosteroids reduce the ability to fight infection, care must be used to ensure that there is no active infection, particularly an intra-abdominal abscess before the initiation of steroids. Budesonide is an oral corticosteroid with limited absorption and high level of first-pass metabolism, meaning that less quantities of steroid enter into the bloodstream. It has been shown to be useful in the treatment of mild-to-moderate Crohn's disease and for maintenance of remission in Crohn's disease. Formulated as Entocort™, budesonide is released in the ileum and right colon, and is therefore has a topical effect against disease in that area. Budesonide is also useful when used in combination with antibiotics for active Crohn's disease.

Mercaptopurine immunosuppressing drugs: Azathioprine, shown here in tablet form, is a first line steroid-sparing immunosuppressant. Azathioprine and 6-mercaptopurine (6-MP) are the most used immunosuppressants for maintenance therapy of Crohn's disease. They are purine anti-metabolites, meaning that they interfere with the synthesis of purines required for inflammatory cells. They have a duration of action of months, making it unwieldy to use them for induction of remission. Both drugs are dosed at 1.5 to 2.5 mg/kg, with literature supporting the use of higher doses. Azathioprine and 6-MP have been found to be useful for the following indications: maintenance therapy for people who are dependent on steroids, fistulizing disease, induction of remission in steroid refractory disease, maintenance of remission after surgery for Crohn's disease. Azathioprine is however a particularly dangerous drug, with great potential for inviting a host of potentially fatal infections, and is also listed by the FDA as a human carcinogen.

Infliximab, marketed as Remicade™, is a mouse-human chimeric antibody that targets tumour necrosis factor, a cytokine in the inflammatory response. It is a monoclonal antibody that inhibits the pro-inflammatory cytokine tumour necrosis factor alpha. It is administered intravenously and dosed per weight starting at 5 mg/kg and increasing according to character of disease. Infliximab has found utility as follows: maintenance of remission for people with Crohn's disease, induction of remission for people with Crohn's disease, maintenance for fistulizing Crohn's disease, side effects of infliximab, like other immunosuppressants of the TNF class, can be serious and potentially fatal, and infliximab carries an FDA black-box warning on the label. Listed side effects include hypersensitivity and allergic reactions, risk of re-activation of tuberculosis, serum sickness, and risk of multiple sclerosis.

Adalimumab, marketed as Humira™, like infliximab is an antibody that targets tumour necrosis factor. Adalimumab has been shown to reduce the signs and symptoms of, and is approved for treatment of, moderate to severe Crohn's disease (CD) in adults who have not responded well to conventional treatments and who have lost response to, or are unable to tolerate infliximab.

Natalizumab, marketed as Tysabri™, is an anti-integrin monoclonal antibody that has shown utility as induction and maintenance treatment for moderate to severe Crohn's disease. Natalizumab may be appropriate in patients who do not respond to medications that block tumor necrosis factor-alpha such as infliximab.

Treatment with an Anti-TLR3 Antibody

In the present invention “established Crohn's disease” refers to a Crohn's disease which has been declared for more than one year. One aspect of the invention is to provide a composition which is able to treat an established Crohn's disease.

Another aspect of the invention is to provide a treatment method to reduce or abort a Crohn's disease attack, thereby leading to an improvement of the patient's health and comfort. A disease attack can refer to a patient who has a CDAI score of more than 150, more than 219, more than 449 points. Thus, the present invention also provides a method for treating a patient having a chronic inflammatory disease of the intestine comprising the step of assessing whether said patient is experiencing a flare-up or an attack, and if said patient is experiencing an attack, treating said patient with an effective amount of an anti-TLR3 antibody.

Still another aspect of the invention is to provide a method for the prophylactic treatment of a patient suffering from a Crohn's disease, thereby avoiding a flare up.

In an embodiment, the antibodies according to the invention are administered in combination with another Crohn's disease treatment, such as those listed above.

### COPD

Chronic obstructive pulmonary disease (COPD), also known as chronic obstructive lung disease (COLD), chronic obstructive airway disease (COAD), chronic airflow limitation (CAL) and chronic obstructive respiratory disease (CORD), refers to chronic bronchitis and emphysema, a pair of commonly co-existing diseases of the lungs in which the airways become narrowed.

In clinical practice, COPD is defined by its characteristically low airflow on lung function tests. Worldwide, COPD ranked as the sixth leading cause of death in 1990. It is projected to be the fourth leading cause of death worldwide by 2030 due to an increase in smoking rates and demographic changes in many countries. COPD is the 4th leading cause of death in the U.S., and the economic burden of COPD in the U.S. in 2007 was $42.6 billion in health care costs and lost productivity.

COPD is caused by noxious particles or gas, most commonly from tobacco smoking, which triggers an abnormal inflammatory response in the lung. The natural course of COPD is characterized by occasional sudden worsening of symptoms called acute exacerbations, most of which are caused by infections or air pollution.

### Disease Evaluation and Rating

A chronic obstructive pulmonary disease (COPD) diagnosis is confirmed by spirometry. Diagnosis of COPD should be considered in any patient who has symptoms of a chronic cough, sputum production, dyspnea and a history of exposure to risk factors for the disease. Where spirometry is unavailable, clinical symptoms and signs, such as abnormal shortness of breath and increased forced expiratory time, can be used to help with the diagnosis. A low peak flow is consistent with COPD, but may not be specific to COPD because it can be caused by other lung diseases and by poor performance during testing. Chronic cough and sputum production often precede the development of airflow limitation by many years, although not all individuals with cough and sputum production go on to develop COPD. Because COPD develops slowly, it is most frequently diagnosed in people aged 40 years or over.

### Current Treatment Options

Various treatments are available: Short-acting bronchodilators, both beta agonists and anticholinergics, are the mainstay of therapy for COPD, long-acting bronchodilators are indicated for moderate to severe COPD. Currently two beta agonists are available. Another treatment option is inhaled corticosteroids (e.g. dexamethasone, for example dexamethasone nasal spray such as Dexacort™) which are recommended for patients with moderate to severe COPD with frequent exacerbations (incidents which worsen symptoms). Systemic corticosteroids (IV or pills) are beneficial for treatment of severe exacerbations. Antibiotics may be beneficial for treatment of exacerbations. Theophylline in low doses may reduce frequency of exacerbations in patients who tolerate it, despite of the strong reported side effects. Other compounds currently in development are also suitable for a combination in the treatment methods, including NVA23 and QVA149, a combination of indacaterol and NVA237, an inhaled muscarinic receptor antagonist (Novartis AG), indacaterol (QAB 149), an adrenergic receptor beta 2 agonist (Novartis), and Relovair fluticasone furoate/vilanterol (GW685698/GW64244) (GlaxoSmith-Kline plc), a fixed dose combination of an inhaled corticosteroid and a long-acting adrenergic receptor beta 2 agonist (LABA).

Treatment with an Anti-TLR3 Antibody

A patient having COPD can be evaluated to assess the presence, stage, evolution or rating of disease. An individual suspected of having COPD, disease can be evaluated using spirometry, postbronchodilator spirometry, lung volumes, and diffusion capacity. In one embodiment, blood gases, preferably arterial blood gases are measured; in one embodiment, an individual having a percent saturation of oxygen in arterial blood that is diminished compared to a healthy individual is determined to have COPD (or an exacerbation of COPD). Optionally a biological sample (e.g. synovial fluid) is obtained and assessed for the presence of proinflammatory mediators or other markers of active inflammation, and/or dsRNA.

A determination that a patient has COPD, or that pro-inflammatory mediators or other markers of active inflammation, and/or dsRNA are present (e.g. in the inflamed tissue), indicates that the patient can be treated with an anti-TLR3 antibody.

In one advantageous aspect, a patient determined to have COPD, and optionally having active inflammation and/or established or chronic COPD, and/or experiencing an exacerbation is treated with an anti-TLR3 antibody.

One object of the present invention is to treat a patient during an acute exacerbation. (Barnes et al, Am J Respir Cell Mol Biol Vol 41. pp 631-638, 2009).

One aspect of the invention is to provide a composition which is able to treat the symptoms of COPD, in particular during an exacerbation. In one embodiment, the TLR3 antibody is administered during an exacerbation.

In another embodiment, the patient is administered an anti-TLR3 antibody to avoid an exacerbation, i.e. prophylactically.

In an embodiment, the antibodies according to the invention are administered in combination with another COPD disease treatment, such as those listed above.

The TLR3 antagonist can be injected or infused via subcutaneous, intravenous, intramuscular, intra-articular, intra-synovial, intrasternal, intrathecal, intrahepatic, intralesional and intracranial routes. In one embodiment, the anti-TLR3 antibody is administered by nasal or inhalation administration.

### Hepatitis

Hepatitis is an inflammation of the liver characterized by the presence of inflammatory cells in the tissue of the organ. Hepatitis may occur with limited or no symptoms, but often leads to jaundice, anorexia and malaise. Hepatitis is acute when it lasts less than six months and chronic, or established when it persists longer. Hepatitis is established when it has been diagnosed for more than 6 months. A group of viruses known as the hepatitis viruses cause most cases of hepatitis worldwide, but it can also be due to toxins (notably alcohol, certain medications and plants), other infections and autoimmune diseases. Yin et al, Gastroenterology Research and Practice, Volume 2010 have reviewed the role of TLR3 in hepatitis.

Immunohistochemistry analyses showed that the expression of TLR3 was markedly increased in biliary epithelial cells at sites of ductular reaction in primary biliary cirrhosis and autoimmune hepatitis. A strong positive correlation between the mRNA levels of TLR3 and type I IFN in the liver was found in the patients with primary biliary cirrhosis, suggesting TLR3 signaling is involved in the pathogenesis of primary biliary cirrhosis. (M. Nakamura, K. Funami, A. Komori et al, Hepatology International, vol. 2, no. 2, pp. 222-230, 2008 and Y. Takii, M. Nakamura, M. Ito et al., Laboratory Investigation, vol. 85, no. 7, pp. 908-920, 2005).

Hepatitis can be of various origin, for instance, hepatitis can be diagnosed following an autoimmune dysregulation, hepatic dysfunction due to an overdose of alcohol, or be a side effect of a immunosuppressive treatment.

### Autoimmune Hepatitis

Autoimmune hepatitis is an inflammation of the liver without a specific cause. The condition is chronic and progressive. Although the disease is chronic, many patients with autoimmune hepatitis present acutely ill with jaundice, fever and sometimes symptoms of severe hepatic dysfunction, a picture that resembles acute hepatitis. Patients usually present with evidence of moderate to severe hepatitis with elevated serum ALT and AST activities in the setting of normal to marginally elevated alkaline phosphatase and gamma-glutamyltranspeptidase activities. Blood tests identify ANA or smooth muscle antibodies (SMA) in the majority of patients (60%). More than 80% of affected individuals have increased gamma globulin in the blood. Autoimmune hepatitis usually occurs in women (70%) between the ages of 15 and 40 and an estimated 70% will be forced to continue drug therapy for the rest of their lives or in some cases be candidates for a liver transplant. An estimated 10% to 30% will be able to discontinue all drug therapy after a remission of four years.

### Disease Evaluation and Rating

Serum protein electrophoresis and testing for autoantibodies are of central importance in the diagnosis of autoimmune hepatitis. Patients with one subtype of autoimmune hepatitis have serum gamma-globulin concentrations more than twice normal and sometimes antinuclear antibodies and/or anti-smooth muscle (anti-actin) antibodies. Patients with another subtype may have normal or only slightly elevated serum gamma-globulin concentrations but will have antibodies against a particular cytochrome p450 isoenzyme that are called anti-LKM (liver kidney microsome).

The most common symptoms of autoimmune hepatitis are fatigue, abdominal discomfort, aching joints, itching, jaundice, enlarged liver, and spider angiomas (tumors) on the skin. Patients may also have complications of more advanced chronic hepatitis with cirrhosis, such as ascites (abdominal fluid) or mental confusion called encephalopathy. A liver biopsy is important to confirm the diagnosis and provide a prognosis. Liver biopsy may show mild chronic active hepatitis, more advanced chronic active hepatitis with scarring (fibrosis), or a fully developed cirrhosis.

### Current Treatment Options

The 10-year survival rate in untreated patients is approximately 10%. Patients in whom a diagnosis of autoimmune hepatitis is suspected should have a liver biopsy. If the biopsy is consistent, treatment with steroids (prednisone or pednisolone) and azathioprine (Imuran) is begun immediately. These are tapered over the next 6 to 24 months depending upon the patient's course. This medical therapy has been shown to decrease symptoms, improve liver tests, and prolong survival in the majority of patients. Therapy is usually begun with prednisone 30 to 40 mg per day and then this dosage is reduced after a response is achieved. The standard dosage used in the majority of patients is prednisone 10-15 mg per day, either alone or with azathioprine 50 mg per day. Higher doses of prednisone given long-term are associated with an increase in serious side effects, including: hypertension, diabetes, peptic ulcer, bone thinning, and cataracts. Lower doses of prednisone may be used when combined with azathioprine. If immediate liver biopsy is contraindicated because of a prolonged prothrombin time or thrombocytopenia, steroids and azathioprine should be started prior to biopsy if the diagnosis of autoimmune hepatitis is likely based on clinical criteria (e.g. a young woman with severe hepatitis, elevated serum gamma-globulin concentration, negative risk factors and serologies for viral hepatitis). The patient will often rapidly improve and biopsy should be performed to confirm the diagnosis as soon as the prothrombin time decreases and platelet count increases to within safe ranges. Azathioprine at a relatively high dose (2 mg per kilogram of body weight) induces a risk of development of a cancer.

The goal of treatment of autoimmune hepatitis is to cure or control the disease. About two thirds to three quarters of patients with autoimmune hepatitis respond to treatment based on the return of serum ALT and AST activities to normal and an improved biopsy after several months. Long-term follow-up studies show that autoimmune hepatitis appears more often to be a controllable rather than a curable disease, because the majority of patients relapse within six months after therapy is ended. Therefore, most patients need long-term maintenance therapy. Some patients relapse as steroids and azathioprine doses are tapered or stopped and need chronic maintenance medications. Not all patients with autoimmune hepatitis respond to prednisone treatment. Approximately 15-20% of patients with severe disease continue to deteriorate despite initiation of appropriate therapy. Such patients are unlikely to respond to further medical therapy, and liver transplantation should be considered. Over the long term, many patients develop cirrhosis despite having a response to treatment, and patients who do not respond to treatment will almost always progress to cirrhosis. If end-stage liver disease develops, orthotopic liver transplantation is an effective procedure.

Treatment with an Anti-TLR3 Antibody

One object of the present invention is to provide a method for the treatment of hepatitis, autoimmune hepatitis or alcoholic hepatitis. One aspect of the invention is to provide a composition which is able to treat the symptoms of hepatitis, in particular during an exacerbation. The method can also be used for the treatment of an established hepatitis, preferably of autoimmune origin, comprising administering to a subject in need thereof, an effective amount of an anti-TLR3 antibody.

The TLR3 antagonist can be injected or infused via subcutaneous, intravenous, intramuscular, intra-articular, intra-synovial, intrasternal, intrathecal, intrahepatic, intralesional and intracranial routes.

In an embodiment, the antibodies according to the invention are administered in combination with another hepatitis disease treatment, such as those listed above.

### Lupus Erythematosus

Four main types of lupus exist—systemic lupus erythematosus, discoid lupus erythematosus, drug-induced lupus erythematosus, and neonatal lupus erythematosus. Of these, systemic lupus erythematosus is the most common and serious form of lupus.

Discoid lupus erythematosus (DLE) is a chronic skin condition of sores with inflammation and scarring favoring the face, ears, and scalp and at times on other body areas. These lesions develop as a red, inflamed patch with a scaling and crusty appearance. The center areas may appear lighter in color with a rim darker than the normal skin.

Drug-induced lupus erythematosus (DIL or DILE) is an autoimmune disorder caused by chronic use of certain drugs. These drugs cause an autoimmune response producing symptoms similar to those of SLE. There are 38 known medications to cause DIL but there are three that report the highest number of cases: hydralazine, procainamide, and isoniazid. While the criteria for diagnosing DIL has not been thoroughly established, symptoms of DIL typically present as myalgia and arthralgia. Generally, the symptoms recede after discontinuing use of the drugs.

Neonatal lupus erythematosus presents in infants, most often girls, born to mothers who carry the Ro/SSA antibody. The infants have no skin lesions at birth, but develop them during the first weeks of life.

Systemic lupus erythematosus is a chronic systemic autoimmune disease that can affect any part of the body. As occurs in other autoimmune diseases, the immune system attacks the body's cells and tissue, resulting in inflammation and tissue damage. SLE most often harms the heart, joints, skin, lungs, blood vessels, liver, kidneys, and nervous system. The course of the disease is unpredictable, with periods of illness (flares) alternating with remissions. The disease occurs nine times more often in women than in men, especially in women in child-bearing years ages 15 to 35, and is more common in those also of non-European descent. SLE is treatable through addressing its symptoms, mainly with cyclophosphamide, corticosteroids and immunosuppressants; there is currently no cure. SLE can be fatal, although with recent medical advances, fatalities are becoming increasingly rare. SLE is considered incurable, but highly treatable. In the 1950s, most people diagnosed with SLE lived fewer than five years. Advances in diagnosis and treatment have improved survival to the point where over 90% now survive for more than ten years, and many can live relatively asymptomatically.

### Disease Evaluation and Rating

Steroids should be used at the lowest dose for the shortest possible period, to reduce potential for cardiovascular issues, and other drugs that can reduce symptoms should be used whenever possible. High serum creatinine, hypertension, nephrotic syndrome, anemia and hypoalbuminemia are poor prognostic factors. The ANA is the most sensitive screening test for evaluation, whereas anti-Sm (anti-Smith) is the most specific. The dsDNA antibody is also fairly specific and often fluctuates with disease activity; as such, the dsDNA titer is sometimes useful to monitor disease flares or response to treatment.

Some physicians make a diagnosis on the basis of the American College of Rheumatology (ACR) classification criteria. The criteria, however, were established mainly for use in scientific research including use in randomized controlled trials which require higher confidence levels, so some people with SLE may not pass the full criteria.

The American College of Rheumatology established eleven criteria in 1982, revised in 1997 as a classificatory instrument to operationalise the definition of SLE in clinical trials. For the purpose of identifying patients for clinical studies, a person has SLE if any 4 out of 11 symptoms are present simultaneously or serially on two separate occasions: Serositis: Pleuritis or pericarditis, Oral ulcers, Arthritis: nonerosive arthritis of two or more peripheral joints, with tenderness, swelling, or effusion, photosensitivity, blood (hematologic disorder, hemolytic anemia (low red blood cell count) or leukopenia (white blood cell count<4000/μl), lymphopenia (<1500/μl) or thrombocytopenia (<100000/μl) in the absence of offending drug; renal disorder; antinuclear antibody test positive; immunologic disorder: Positive anti-Smith, anti-ds DNA, antiphospholipid antibody, and/or false positive serological test for syphilis; presence of anti-ss DNA in 70% of cases, neurologic disorder: Seizures or psychosis, Malar rash, Discoid rash.

### Current Treatment Options

The treatment of SLE involves preventing flares and reducing their severity and duration when they occur. Treatment can include corticosteroids and anti-malarial drugs. Certain types of lupus nephritis such as diffuse proliferative glomerulonephritis require bouts of cytotoxic drugs. These drugs include cyclophosphamide and mycophenolate.

Disease-modifying antirheumatic drugs (DMARDs) are used preventively to reduce the incidence of flares, the process of the disease, and lower the need for steroid use; when flares occur, they are treated with corticosteroids. DMARDs commonly in use are antimalarials such as plaquenil and immunosuppressants (e.g. methotrexate and azathioprine). Hydroxychloroquine (HCQ) was the last medication approved by the FDA for lupus in 1955. Anti-BlyS antibodies (Benlysta™, Human Genomce Science, Inc.) are pending approval and can be used as a DMARD. Hydroxychloroquine is an antimalarial used for constitutional, cutaneous, and articular manifestations. Hydroxychloroquine has relatively few side effects, and there is evidence that it improves survival among people who have SLE. Cyclophosphamide is used for severe glomerulonephritis or other organ-damaging complications. Some drugs approved for other diseases are used for SLE ‘off-label’; Immunosuppressive drugs are also used to control the disease and prevent recurrence of symptoms (known as flares). Depending on the dosage, people who require steroids may develop Cushing's syndrome, side-effects of which may include obesity, puffy round face, diabetes mellitus, large appetite, difficulty sleeping and osteoporosis. Those side-effects can subside if and when the large initial dosage is reduced, but long-term use of even low doses can cause elevated blood pressure and cataracts. Numerous new immunosuppressive drugs are being actively tested for SLE. Rather than suppressing the immune system nonspecifically, as corticosteroids do, they target the responses of individual immune cells; analgesics, such as indomethacin and diclofenac, may be used if over-the-counter drugs (mainly nonsteroidal anti-inflammatory drugs) do not provide effective relief. Moderate pain is typically treated with mild prescription opiates such as dextropropoxyphene and co-codamol. Moderate to severe chronic pain is treated with stronger opioids, such as hydrocodone or longer-acting continuous-release opioids, such as oxycodone, MS Contin, or Methadone. The Fentanyl duragesic transdermal patch is also a widely-used treatment option for the chronic pain caused by complications because of its long-acting timed release and ease of use. When opioids are used for prolonged periods, drug tolerance, chemical dependency, and addiction may occur. Opiate addiction is not typically a concern, since the condition is not likely to ever completely disappear. Thus, lifelong treatment with opioids is fairly common for chronic pain symptoms, accompanied by periodic titration that is typical of any long-term opioid regimen; at last, intravenous immunoglobulins may be used to control SLE with organ involvement, or vasculitis. Due to the variety of symptoms and organ system involvement with SLE, its severity in an individual must be assessed in order to successfully treat SLE. Mild or remittant disease can sometimes be safely left untreated.

Treatment with an Anti-TLR3 Antibody

In the present invention “established lupus” refers to a lupus disease which has been progressing for over a year or which has been declared for more than one year. Another aspect of the invention is to provide a composition which is able to treat an established lupus, or to reduce or abort a lupus flare, thereby leading to an improvement of the patient's health and comfort.

In an embodiment, the antibodies according to the invention are administered in combination with another lupus treatment, such as those listed above.

### Sepsis

Sepsis (systemic inflammatory response syndrome or SIRS) is a serious medical condition that is characterized by a whole-body inflammatory state.

### Disease Evaluation and Rating

In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. SIRS is characterized by hemodynamic compromise and resultant metabolic derangement. Outward physical symptoms of this response frequently include a high heart rate, high respiratory rate, elevated WBC count and elevated or lowered body temperature. Sepsis is differentiated from SIRS by the presence of a known pathogen. For example SIRS and a positive blood culture for a pathogen indicate the presence of sepsis. Without a known infection, it's not possible to classify the above symptoms as sepsis, only SIRS. This immunological response causes widespread activation of acute-phase proteins, affecting the complement system and the coagulation pathways, which then cause damage to the vasculature as well as to the organs. Various neuroendocrine counter-regulatory systems are then activated as well, often compounding the problem. Even with immediate and aggressive treatment, this may progress to multiple organ dysfunction syndrome and eventually death.

The American College of Chest Physicians and the Society of Critical Care Medicine has established different levels of sepsis:

Systemic inflammatory response syndrome (SIRS). Defined by the presence of two or more of the following findings: Body temperature <36° C. (97° F.) or >38° C. (100° F.) (hypothermia or fever), Heart rate >90 beats per minute, Respiratory rate >20 breaths per minute or, on blood gas, a PaCO2 less than 32 mm Hg (4.3 kPa) (tachypnea or hypocapnia due to hyperventilation), White blood cell count <4,000 cells/mm3 or >12,000 cells/mm3 (<4×109 or >12×109 cells/L), or greater than 10% band forms (immature white blood cells). (leukopenia, leukocytosis, or bandemia).

Sepsis. Defined as SIRS in response to a confirmed infectious process. Infection can be suspected or proven, or a clinical syndrome pathognomonic for infection.

Severe sepsis. Defined as sepsis with organ dysfunction, hypoperfusion, or hypotension.

Septic shock. Defined as sepsis with refractory arterial hypotension or hypoperfusion abnormalities in spite of adequate fluid resuscitation.

### Current Treatment Options

Sepsis is usually treated in the intensive care unit with intravenous fluids and antibiotics. If fluid replacement is insufficient to maintain blood pressure, specific vasopressor medications can be used. Mechanical ventilation and dialysis may be needed to support the function of the lungs and kidneys, respectively. To guide therapy, a central venous catheter and an arterial catheter may be placed. Sepsis patients require preventive measures for deep vein thrombosis, stress ulcers and pressure ulcers, unless other conditions prevent this. Some patients might benefit from tight control of blood sugar levels with insulin (targeting stress hyperglycemia), low-dose corticosteroids or activated drotrecogin alfa (recombinant protein C).

Treatment with an Anti-TLR3 Antibody

One aspect of the invention is to provide a composition which is able to treat a SIRS, a sepsis, a severe sepsis or a septic shock.

Another aspect of the invention is to provide a method for the prophylactic treatment of patients who is at risk of developing a sepsis. For instance, patients that have had their spleen surgically removed, patients with an impaired immune system (i.e. chemotherapy treatment, immunodepression) but also other causes such as long term steroids medication, diabetes, AIDS, or cirrhosis, large burns or severe injuries, infections such as pneumonia, meningitis, peritonitis, appendicitis, cellulitis, urinary tract infection or infections occurring after a major surgical act.

In an embodiment, the antibodies according to the invention are administered in combination with another sepsis treatment, such as those listed above.

### Other Autoimmune and Inflammatory Disorders

The anti-TLR3 antibodies can be used to treat any other suitable autoimmune and inflammatory disorders, including for example diseases involving a deregulation of the immune system in a patient. Autoimmune diseases may, but are not limited to: Achlorhydra Autoimmune Active Chronic Hepatitis, Acute Disseminated Encephalomyelitis, Acute hemorrhagic leukoencephalitis, Addison's Disease, Agammaglobulinemia, Alopecia greata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Unknown or Multiple Autoimmune progesterone dermatitis, Autoimmune thrombocytopenic purpura, Autoimmune uveitis, Balo disease/Balo concentric sclerosis, Bechets Syndrome, Berger's disease, Bickerstaffs encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (one of two types of idiopathic inflammatory bowel disease “IBD”), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressive, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barré syndrome (GBS), Hashimoto's encephalitis, Hashimoto's thyroiditis, Haemolytic anaemia, Henoch-Schonlein purpura, Herpes gestationis, Hidradenitis suppurative, Hughes syndrome, Hypogammaglobulinemia, Idiopathic Inflammatory Demyelinating Diseases, Idiopathic pulmonary fibrosis, Idiopathic thrombocytopenic purpura, IgA nephropathy, Inclusion body myositis, Inflammatory demyelinating polyneuopathy, Interstitial cystitis, Irritable Bowel Syndrome (IBS), Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease, Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica, Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis (SBE), Susac's syndrome, Sweet syndrome, Sydenham Chorea, Sympathetic ophthalmia, Takayasu's arteritis, Temporal arteritis, Tolosa-Hunt syndrome, Transverse Myelitis, Ulcerative Colitis, Undifferentiated connective tissue disease, Undifferentiated spondyloarthropathy, Vasculitis, Vitiligo, Wegener's granulomatosis, Wilson's syndrome and Wiskott-Aldrich syndrome.

In a further embodiment, an anti-TLR3 antibody that inhibitors signalling by a TLR3 polypeptide is used for the treatment or prevention of graft-versus-host disease (GvHD), e.g. in transplantation or transfusions. Particularly, after bone marrow transplantation, T cells present in the graft, either as contaminants or intentionally introduced into the host, attack the tissues of the transplant recipient after perceiving host tissues as antigenically foreign. The T cells produce an excess of cytokines, including TNF-α and interferon-gamma (IFNγ). Anti-TLR3 antibodies can be administered before, during or following a transplantation or transfusion, e.g. allogeneic bone marrow transplantation, particularly in the treatment of cancer, for example leukemias. The antibody may be any anti-TLR3 antibody that inhibits signalling of a TLR3 polypeptide. Since GvHD is believed to be largely driven by antigen presenting cells, anti-TLR3 antibodies that inhibit TLR3 signalling in DC are believed to be particularly useful.

### Dosing Regimens

Based on efficacy data collected during mice experiment, the inventors have established that a dose as low as 100 μg/mouse produces a therapeutic effect. Such dosage is equivalent to 4 mg/kg in the mouse and therefore 0.5 mg/kg in a human subject. Therefore, in the methods of the invention, the anti TLR3-antibody can be administered at a dosage comprised between 0.05 and 20 mg/kg in human, preferably 0.1 and 10 mg/kg, further preferably between 0.5 and 5 mg/kg (for example a unit dose of between about 25 mg and 500 mg).

An exemplary treatment regime entails administration twice per week, once per week, once every two weeks, once every three weeks, once every four weeks, once a month, once every 2 to 3 months, or once every 3 to 6 months. Exemplary dosage regimens for an anti-TLR3 antibody include between 0.05 and 20 mg/kg (preferably 0.1 and 10 mg/kg, further preferably between 0.5 and 5 mg/kg) body weight body weight via intravenous administration or subcutaneous injection, with the antibody being given using one of the following dosing schedules: (i) loading doses about every 1, 2, 3 of 4 weeks (e.g., for 2-4 dosages), then every one to three months; (ii) once per month or once per two month period; (iii) every one to two weeks, or any other optimal dosing.

The anti-TLR3 antibody is optionally administered at a dose that is suitable to induce substantially full TLR3 receptor saturation (90%, optionally 95% receptor saturation), e.g. saturation of TLR3 polypeptide expressed in targeted cells. As the TLR3 receptor is though to dimerize before signaling, an inhibition of less than fully saturation by at least 20%, 30%, 40%, 50% receptor saturation may be useful in the treatment of a disease. In one embodiment, a dose of anti-TLR3 antibody resulting in at least about 20%, 30%, 40%, 50%, 90% or 95% receptor saturation is administered from about 2 times per week to about once per month, or from about once per month to about once per 2 months. The dose can be, e.g., administered at least 3 times, at least 6 times, or more. For example, the method may comprise administering an anti-TLR3 antibody at a dose and a dosing frequency achieving at least about 20%, 30%, 40%, 50%, 90% or 95% TLR3 receptor saturation on targeted cells for at least about two weeks, one month, 6 months, 9 months or 12 months. In one preferred embodiment, a regimen results in sustained substantially full receptor saturation. A dose of anti-TLR3 antibody resulting in substantially full receptor saturation for a period of at least about 1 week, 2 weeks or 1 month is administered.

Receptor occupancy can be evaluated on human samples where target cells are present (e.g. whole blood, any tissue which is the site of an inflammation, synovial fluid). Saturation percentage of the TLR3 receptor can be measured by FACS analysis using methods known in the art, via intracellular staining since the TLR3 receptor is present in the cells. Alternatively, saturation percentage can be determined using a test of cytokine inhibition secretion profile in response to a TLR3 ligand such as a dsRNA (i.e. polyAU) in mononuclear cells (preferable PBMCs) obtained from a patient. An efficient cytokine inhibition is correlated with an efficient therapeutic effect and the dosage can then be adapted for each patient. Cytokines that can be measured in this assay are for instance IP-10 or IL-6. In another embodiment, receptor saturation is assessed as receptor occupancy, for example by conducting free site and bound site assays. Briefly, free and bound TLR3 receptor levels are assessed on target cells from a biological sample obtained from an individual treated with the anti-TLR3 antibody, where a free site assay assesses unbound TLR3 by staining with PE-conjugated form of the anti-TLR3 antibody administered to an individual. A bound site assay assesses TLR3 polypeptides occupied by anti-TLR3 antibody by staining with a PE-conjugated mouse anti-human IgG4 monoclonal antibody (when the anti-TLR3 antibody is of human IgG4 isotype) that recognizes the anti-TLR3 antibody bound to the TLR3 polypeptides. In one embodiment, the invention further provides a method for treating an individual comprising: (a) administering an anti-TLR3 antibody to an individual and (b) determining TLR3 receptor saturation in the individual, optionally further determining a dosage of anti-TLR3 antibody to be administered to the individual.

### Antibodies

The antibodies suitable for the present invention are antibodies binding TLR3. In an embodiment, the antibodies are antagonistic TLR3 antibodies. In another embodiment, the antibodies are antibodies blocking the signalling induced through TLR3.

In another embodiment, the antibodies have an affinity at an acidic pH, i.e. a pH of about 5.6, of less than 10⁻⁹ M, preferably less than 10⁻¹⁰M. In another embodiment, the antibodies have an affinity at a neutral pH, i.e. a pH of about 7.2, of less than 10⁻⁹ M, preferably less than 10⁻¹⁰M. In another embodiment, the antibodies have an affinity at an acidic pH, i.e. a pH of about 5.6, and at a neutral pH, i.e. a pH of about 7.2, of less than 10⁻⁹ M, preferably less than 10⁻¹⁰M.

In another embodiment, the antibodies are able to inhibit TLR3 signaling in the presence of a TLR3 ligand, i.e. dsRNA (polyAU, polyIC). In another embodiment, the antibodies are able to inhibit TLR3 signaling when administered after a TLR3 ligand. In another embodiment, the antibodies are able to inhibit TLR3 signaling when administered before a TLR3 ligand. In another embodiment, the antibodies are able to inhibit TLR3 signaling when administered simultaneously with a TLR3 ligand.

In another embodiment, the antibodies do not compete for binding with dsRNA on the TLR3 polypeptide, particularly on the C-terminal dsRNA binding site of the TLR3 polypeptide. Such property can be assessed as described in example 7 for instance.

### Antibody Epitopes

In another embodiment, the antibodies bind substantially the same epitope as antibodies 31C3, 29H3, 34A3 and 28F11. In another embodiment, the antibodies at least partially overlaps, or includes at least one residue in the segment corresponding to residues 102 to 204, residues 102 to 151 (or 102 to 152), residues 102 to 173, residues 152 to 204 (or 153 to 204) or residues 152 to 173 (or 153 to 173) of the mature TLR3 polypeptide of SEQ ID NO: 1. In one embodiment, all key residues of the epitope is in a segment corresponding to residues 102 to 204, residues 102 to 151 (or 102 to 152), residues 102 to 173, residues 152 to 204 (or 153 to 204), residues 152 to 173 (or 153 to 173), or residues 174 to 191 of the mature TLR3 polypeptide of SEQ ID NO: 1. In one embodiment, the antibodies bind an epitope comprising 1, 2, 3, 4, 5, 6, 7 or more residues in the segment corresponding to residues 102 to 204, residues 102 to 151 (or 102 to 152), residues 102 to 173, residues 152 to 204 (or 153 to 204) or residues 152 to 173 (or 153 to 173) of the mature TLR3 polypeptide of SEQ ID NO: 1. In another embodiment, the antibodies bind an epitope comprising 1, 2, 3, 4, 5, 6, 7 or more residues in the segment corresponding to residues 102 to 168, residues 102 to 121, residues 131 to 152, residues 153 to 173, residues 168 to 173, residues 174 to 191, residues 192 to 204 or residues 173 to 204 of the mature TLR3 polypeptide of SEQ ID NO: 1.

Optionally, in any embodiment, the antibodies can optionally further be characterized by not substantially binding to one, two, three, or more residues in the segment corresponding to residues 174 to 191, residues 153 to 173, residues 168 to 173 or residues 465-619, or to residues 116 and/or 145, of the mature TLR3 polypeptide of SEQ ID NO: 1.

In another embodiment, the antibodies bind one or more amino acids present on the surface of the TLR3 polypeptide within the epitopes bound by the anti-TLR3 antibodies of the invention. In one such embodiment, the antibodies bind 1, 2, 3, 4, 5, 6, 7 or more residues selected from the group consisting of 102, 103, 105, 107, 108, 109, 110, 112, 113, 114, 115, 116, 117, 118, 120, 121, 123, 124, 126, 127, 129, 131, 132, 133, 134, 136, 137, 139, 140, 141, 144, 145, 147, 148, 150 and 151 of the mature TLR3 polypeptide of SEQ ID NO: 1. In one such embodiment, the antibodies bind 1, 2, 3, 4, 5, 6, 7 or more residues selected from the group consisting of 153, 155, 156, 157, 158, 160, 161, 163, 166, 167, 168, 171 and 172 of the mature TLR3 polypeptide of SEQ ID NO: 1. In one such embodiment, the antibodies bind 1, 2, 3, 4, 5, 6, 7 or more residues selected from the group consisting of 102, 103, 105, 107, 108, 109, 110, 112, 113, 114, 115, 116, 117, 118, 120, 121, 123, 124, 126, 127, 129, 131, 132, 133, 134, 136, 137, 139, 140, 141, 144, 145, 147, 148, 150, 151, 153, 155, 156, 157, 158, 160, 161, 163, 166, 167, 168, 171, 172 and 182 of the mature TLR3 polypeptide of SEQ ID NO: 1. Optionally, the antibody does not bind residues 116, 145, 171 and/or 196.

In one such embodiment, the antibodies bind 1, 2, 3, 4, 5, 6, 7 or more residues selected from the group consisting of 102, 103, 105, 107, 108, 110, 113, 115, 116, 117, 121, 123, 124, 126, 127, 129, 131, 132, 134, 136, 137, 139, 140, 141, 144, 145, 147, 148, 150 and 151 of the mature TLR3 polypeptide of SEQ ID NO: 1. In one such embodiment, the antibodies bind 1, 2, 3, 4, 5, 6, 7 or more residues selected from the group consisting of 156, 158, 160, 163, 166, 167, 168, 171 and 172 of the mature TLR3 polypeptide of SEQ ID NO: 1. In one such embodiment, the antibodies bind 1, 2, 3, 4, 5, 6, 7 or more residues selected from the group consisting of 102, 103, 105, 107, 108, 110, 113, 115, 116, 117, 121, 123, 124, 126, 127, 129, 131, 132, 134, 136, 137, 139, 140, 141, 144, 145, 147, 148, 150, 151, 156, 158, 160, 163, 166, 167, 168, 171 and 172 of the mature TLR3 polypeptide of SEQ ID NO: 1. Alternatively, the antibody does not bind residue 116, 145, 171 and/or 196.

In another embodiment, the antibodies bind to, or optionally do not bind to, an epitope comprising one or more residues in the segment corresponding to residues 27 to 42, 177 to 191, 224 to 243, 280 to 286, 295 to 374, 379 to 391, 428 to 459, 461 to 487, 524 to 529, 533 to 542, 546 to 569, 575 to 581, 583 to 605, 607 to 623, 641 to 657 and/or 670 to 705 of the mature TLR3 polypeptide of SEQ ID NO: 1.

In one embodiment, the antibodies bind an epitope comprising one or more residues in the segment corresponding to residues 174 to 191 of SEQ ID NO: 1, wherein said antibody does not bind residue 116 and/or residue 145 of the TLR3 polypeptide of SEQ ID NO: 1, optionally further wherein said antibody does not have a significant reduction in binding to a TLR3 polypeptide having a mutation at residues 116, 145, 171 and/or residue 196 of the TLR3 polypeptide of SEQ ID NO: 1, relative to binding between the antibody and a wild-type TLR3 polypeptide of SEQ ID NO: 1. Optionally, said antibody has a significant reduction in binding to a TLR3 polypeptide having a mutation at residue 182 of the TLR3 polypeptide of SEQ ID NO: 1, relative to binding between the antibody and a wild-type TLR3 polypeptide of SEQ ID NO: 1. Optionally, the antibody binds to an epitope comprising at residue 182 of the TLR3 polypeptide of SEQ ID NO: 1. Optionally, the antibody binds to an epitope which does not comprise residues 116, 145, 171 and/or residue 196 of the TLR3 polypeptide of SEQ ID NO: 1.

Example 17 describes the construction of a series of mutant human TLR3 polypeptides having mutations in residues K145, D116, K182, N196 and E171, with each mutant having a single mutation. Binding of anti-TLR3 antibody to cells transfected with the TLR3 mutants was measured and compared to the ability of anti-TLR3 antibody to bind wild-type TLR3 polypeptide (SEQ ID NO:1). A reduction in binding between an anti-TLR3 antibody and a mutant TLR3 polypeptide as used herein means that there is a reduction in binding affinity (e.g., as measured by known methods such FACS testing of cells expressing a particular mutant, or by Biacore testing of binding to mutant polypeptides) and/or a reduction in the total binding capacity of the anti-TLR3 antibody (e.g., as evidenced by a decrease in Bmax in a plot of anti-TLR3 antibody concentration versus polypeptide concentration). A significant reduction in binding indicates that the mutated residue is directly involved in binding to the anti-TLR3 antibody or is in close proximity to the binding protein when the anti-TLR3 antibody is bound to TLR3.

In some embodiments, a significant reduction in binding means that the binding affinity and/or capacity between an anti-TLR3 antibody and a mutant TLR3 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type TLR3 polypeptide (e.g., the polypeptide shown in SEQ ID NO:1). In certain embodiments, binding is reduced below detectable limits. In some embodiments, a significant reduction in binding is evidenced when binding of an anti-TLR3 antibody to a mutant TLR3 polypeptide is less than 50% (e.g., less than 45%, 40%, 35%, 30%, 25%, 20%, 15% or 10%) of the binding observed between the anti-TLR3 antibody and a wild-type TLR3 polypeptide (e.g., the extracellular domain shown in SEQ ID NO:1). Such binding measurements can be made using a variety of binding assays known in the art. A specific example of one such assay is described in Example 17.

In some embodiments, anti-TLR3 antibodies are provided that exhibit significantly lower binding for a mutant TLR3 polypeptide in which a residue in a wild-type TLR3 polypeptide (e.g., SEQ ED NO:1) is substituted with arginine, alanine or glutamic acid. In one such embodiment, binding of an anti-TLR3 antibody is significantly reduced for a mutant TLR3 polypeptide having mutation K 182E as compared to a wild-type TLR3 (e.g., SEQ ID NO:1). In the shorthand notation used here, the format is: Wild type residue: Position in polypeptide: Mutant residue, with the numbering of the residues as indicated in SEQ ID NO: 1. In some embodiments, binding of an anti-TLR3 antibody is not significantly reduced for a mutant TLR3 polypeptide having any one or more (e.g., 1, 2, 3 or 4) of the following mutations: K145E, D116R, N196A and E171A, as compared to a wild-type TLR3 (e.g., SEQ ID NO:1).

Although the mutant forms just listed are referenced with respect to the wild-type extracellular domain sequence shown in SEQ ID NO: 1, it will be appreciated that in an allelic variant of TLR3 the amino acid at the indicated position could differ. Anti-TLR3 antibodies showing significantly lower binding for such allelic forms of TLR3 are also contemplated. Accordingly, in one embodiment, an anti-TLR3 antibody does not have significantly reduced binding for an allelic TLR3 polypeptide as compared to a wild-type TLR3 (e.g., SEQ ID NO:1) where one or more of the following residues (e.g., 1, 2, 3 or 4) of the allelic polypeptide are replaced with arginine, alanine or glutamic acid as indicated: K145, D116, N196 and E171 (Position in polypeptide: Mutant residue, with the numbering of the residues as indicated in SEQ ID NO:1). In some embodiments, an anti-TLR3 antibody exhibits significantly reduced binding for an allelic TLR3 polypeptide in which residue 182 is replaced with arginine, alanine or glutamic acid (e.g. K182E).

In some embodiments, binding of an anti-TLR3 antibody is or is not significantly reduced for a mutant TLR3 polypeptide in which the residue at a selected position in the wild-type TLR3 polypeptide is mutated to any other residue.

In another embodiment, the antibodies are able of inhibiting cytokine (e.g. IP-10) secretion in myeloid dendritic cells (MdDC). Such property can be assessed as described in example 5 for instance.

In another embodiment, the antibodies are able of internalizing into a TLR3-expressing cell rapidly and efficiently. In another embodiment, the antibodies are able of internalizing without inducing or requiring hTLR3 down-modulation.

### Producing Anti-TLR3 Antibodies

The antibodies suitable for the method of the invention specifically bind TLR3. Antibodies of the invention furthermore bind TLR3 under acidic conditions corresponding to that encountered in an acidified endosomic compartment. Antibodies of the invention are furthermore capable of inhibiting the TLR3 signaling pathway. The ability of the inhibitory antibodies to specifically inhibit TLR3 signaling pathway makes them useful for numerous applications, in particular for treating or preventing diseases wherein the inhibition of TLR3 signaling pathway is desirable, i.e. avoid further cytokine and chemokine secretion as well as cellular activation, as described herein. Antibodies of the invention are furthermore capable of binding to TLR3 protein without blocking dsRNA binding.

In one embodiment, the invention provides methods using an antibody that binds human TLR3, and competes for binding to human TLR3 with monoclonal antibody 31C3, 29H3, 23C8, 28F11 or 34A3. Antibody 31C3 is produced by the cell deposited as 31C3.1 with the Collection Nationale de Culture de Microorganismes (CNCM), Institut Pasteur, 25 rue de Docteur Roux, F-75724 Paris on 3 Jul. 2009, under the number CNCM 1-4186. Antibody 29H3 is produced by the cell deposited as 29H3.7 with the Collection Nationale de Culture de Microorganismes (CNCM), Institut Pasteur, 25 rue de Docteur Roux, F-75724 Paris on 3 Jul. 2009, under the number CNCM 1-4187.

“TLR3”, “TLR3 polypeptide” and “TLR3 receptor”, used interchangeably, are used herein to refer to Toll-Like Receptor 3, a member of the Toll-like receptor (TLRs) family. The amino acid sequence of human TLR3 is shown in SEQ ID NO: 1 (NCBI accession number NP_{—}003256, the disclosure of which is incorporated herein by reference). The human TLR3 mRNA sequence is described in NCBI accession number NM_{—}003265. Human TLR3 sequences are also described in PCT patent publication no. WO 98/50547, the disclosure of which is incorporated herein by reference.

In one aspect, the invention provides an antibody that competes with monoclonal antibody 31C3, 29H3, 23C8, 28F11 or 34A3 and recognizes, binds to, or has immunospecificity for substantially or essentially the same, or the same, epitope or “epitopic site” on a TLR3 molecule as monoclonal antibody 31C3, 29H3, 23C8, 28F11 or 34A3. In other embodiments, the monoclonal antibody consists of, or is a derivative or fragment of, antibody 31C3, 29H3, 23C8, 28F11 or 34A3.

It will be appreciated that, while preferred antibodies bind to the same epitope as antibody 31C3, 29H3, 23C8, 28F11 or 34A3, the present antibodies can recognize and be raised against any part of the TLR3 polypeptide. For example, any fragment of TLR3, preferably but not exclusively human TLR3, or any combination of TLR3 fragments, can be used as immunogens to raise antibodies, and the antibodies of the invention can recognize epitopes at any location within the TLR3 polypeptide, so long as they can do so on TLR3 expressing cells such as MdDC or MoDC as described herein. In an embodiment, the recognized epitopes are present on the cell surface, i.e. they are accessible to antibodies present outside of the cell. Most preferably, the epitope is the epitope specifically recognized by antibody 31C3, 29H3, 23C8, 28F11 or 34A3. Further, antibodies recognizing distinct epitopes within TLR3 can be used in combination, e.g. to bind to TLR3 polypeptides with maximum efficacy and breadth among different individuals.

The antibodies of this invention may be produced by a variety of techniques known in the art. Typically, they are produced by immunization of a non-human animal, preferably a mouse, with an immunogen comprising a TLR3 polypeptide, preferably a human TLR3 polypeptide. The TLR3 polypeptide may comprise the full length sequence of a human TLR3 polypeptide, or a fragment or derivative thereof, typically an immunogenic fragment, i.e., a portion of the polypeptide comprising an epitope exposed on the surface of cells expressing a TLR3 polypeptide, preferably the epitope recognized by the 31C3, 29H3, 23C8, 28F11 or 34A3 antibody. Such fragments typically contain at least about 7 consecutive amino acids of the mature polypeptide sequence, even more preferably at least about 10 consecutive amino acids thereof. Fragments typically are essentially derived from the extra-cellular domain of the receptor. In a preferred embodiment, the immunogen comprises a wild-type human TLR3 polypeptide in a lipid membrane, typically at the surface of a cell. In a specific embodiment, the immunogen comprises intact cells, particularly intact human cells, optionally treated or lysed. In another preferred embodiment, the polypeptide is a recombinant TLR3 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way on the present invention. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

For polyclonal antibody preparation, serum is obtained from an immunized non-human animal and the antibodies present therein isolated by well-known techniques. The serum may be affinity purified using any of the immunogens set forth above linked to a solid support so as to obtain antibodies that react with TLR3 polypeptides.

In an alternate embodiment, lymphocytes from a non-immunized non-human mammal are isolated, grown in vitro, and then exposed to the immunogen in cell culture. The lymphocytes are then harvested and the fusion step described below is carried out.

For preferred monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages are preferably from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to TLR3 polypeptide gene products, optionally the epitope specifically recognized by antibody 31C3, 29H3, 23C8, 28F11 or 34A3. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to TLR3 polypeptides, e.g., TLR3-expressing cells, in paraffin-embedded tissue sections, as described below.

Hybridomas that are confirmed to produce a monoclonal antibody of this invention can be grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that bind(s) to TLR3, particularly substantially or essentially the same epitope as monoclonal antibody 31C3, 29H3, 23C8, 28F11 or 34A3, can be readily determined using any one of a variety of immunological screening assays in which antibody competition can be assessed. Many such assays are routinely practiced and are well known in the art (see, e.g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same epitope as the monoclonal antibody described herein.

For example, where the test antibodies to be examined are obtained from different source animals, or are even of a different Ig isotype, a simple competition assay may be employed in which the control (31C3, 29H3, 23C8, 28F11 or 34A3, for example) and test antibodies are admixed (or pre-adsorbed) and applied to a sample containing TLR3 polypeptides. Protocols based upon western blotting and the use of BIACORE analysis are suitable for use in such competition studies.

In certain embodiments, one pre-mixes the control antibodies (31C3, 29H3, 23C8, 28F11 or 34A3, for example) with varying amounts of the test antibodies (e.g., about 1:10 or about 1:100) for a period of time prior to applying to the TLR3 antigen sample. In other embodiments, the control and varying amounts of test antibodies can simply be admixed during exposure to the TLR3 antigen sample. As long as one can distinguish bound from free antibodies (e.g., by using separation or washing techniques to eliminate unbound antibodies) and 31C3, 29H3, 23C8, 28F11 or 34A3 from the test antibodies (e.g., by using species-specific or isotype-specific secondary antibodies or by specifically labeling 31C3, 29H3, 23C8, 28F11 or 34A3 with a detectable label) one can determine if the test antibodies reduce the binding of 31C3, 29H3, 23C8, 28F11 or 34A3 to the antigens, indicating that the test antibody recognizes substantially the same epitope as 31C3, 29H3, 23C8, 28F11 or 34A3. The binding of the (labeled) control antibodies in the absence of a completely irrelevant antibody can serve as the control high value. The control low value can be obtained by incubating the labeled (31C3, 29H3, 23C8, 28F11 or 34A3) antibodies with unlabelled antibodies of exactly the same type (31C3, 29H3, 23C8, 28F11 or 34A3), where competition would occur and reduce binding of the labeled antibodies. In a test assay, a significant reduction in labeled antibody reactivity in the presence of a test antibody is indicative of a test antibody that recognizes substantially the same epitope, i.e., one that “cross-reacts” or competes with the labeled (31C3, 29H3, 23C8, 28F11 or 34A3) antibody. Any test antibody that reduces the binding of 31C3, 29H3, 23C8, 28F11 or 34A3 to TLR3 antigens by at least about 50%, such as at least about 60%, or more preferably at least about 80% or 90% (e.g., about 65-100%), at any ratio of 31C3, 29H3, 23C8, 28F11 or 34A3:test antibody between about 1:10 and about 1:100 is considered to be an antibody that binds to substantially the same epitope or determinant as 31C3, 29H3, 23C8, 28F11 or 34A3. Preferably, such test antibody will reduce the binding of 31C3, 29H3, 23C8, 28F11 or 34A3 to the TLR3 antigen by at least about 90% (e.g., about 95%).

Competition can also be assessed by, for example, a flow cytometry test. In such a test, cells bearing a given TLR3 polypeptide can be incubated first with 31C3, 29H3, 23C8, 28F11 or 34A3, for example, and then with the test antibody labeled with a fluorochrome or biotin. The antibody is said to compete with 31C3, 29H3, 23C8, 28F11 or 34A3 if the binding obtained upon preincubation with a saturating amount of 31C3, 29H3, 23C8, 28F11 or 34A3 is about 80%, preferably about 50%, about 40% or less (e.g., about 30%, 20% or 10%) of the binding (as measured by mean of fluorescence) obtained by the antibody without preincubation with 31C3, 29H3, 23C8, 28F11 or 34A3. Alternatively, an antibody is said to compete with 31C3, 29H3, 23C8, 28F11 or 34A3 if the binding obtained with a labeled 31C3, 29H3, 23C8, 28F11 or 34A3 antibody (by a fluorochrome or biotin) on cells preincubated with a saturating amount of test antibody is about 80%, preferably about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a TLR3 antigen is immobilized may also be employed. The surface in the simple competition assay is preferably a BIACORE chip (or other media suitable for surface plasmon resonance analysis). The control antibody (e.g., 31C3, 29H3, 23C8, 28F11 or 34A3) is then brought into contact with the surface at a TLR3-saturating concentration and the TLR3 and surface binding of the control antibody is measured. This binding of the control antibody is compared with the binding of the control antibody to the TLR3-containing surface in the absence of test antibody. In a test assay, a significant reduction in binding of the TLR3-containing surface by the control antibody in the presence of a test antibody indicates that the test antibody recognizes substantially the same epitope as the control antibody such that the test antibody “cross-reacts” with the control antibody. Any test antibody that reduces the binding of control (such as 31C3, 29H3, 23C8, 28F11 or 34A3) antibody to a TLR3 antigen by at least about 30% or more, preferably about 40%, can be considered to be an antibody that binds to substantially the same epitope or determinant as a control (e.g., 31C3, 29H3, 23C8, 28F11 or 34A3). Preferably, such a test antibody will reduce the binding of the control antibody (e.g., 31C3, 29H3, 23C8, 28F11 or 34A3) to the TLR3 antigen by at least about 50% (e.g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the TLR3 antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein by reference.

Determination of whether an antibody binds within an epitope region can be carried out in ways known to the person skilled in the art. As one example of such mapping/characterization methods, an epitope region for an anti-TLR3 antibody may be determined by epitope “foot-printing” using chemical modification of the exposed amines/carboxyls in the TLR3 protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et al., Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downward, J Mass Spectrom. 2000 April; 35 (4): 493-503 and Kiselar and Downard, Anal Chem. 1999 May 1; 71 (9): 1792-801. Protease digestion techniques also can be useful in the context of epitope mapping and identification. Antigenic determinant-relevant regions/sequences can be determined by protease digestion, e.g. by using trypsin in a ratio of about 1:50 to TLR3 or o/n digestion at and pH 7-8, followed by mass spectrometry (MS) analysis for peptide identification. The peptides protected from trypsin cleavage by the anti-TLR3 binder can subsequently be identified by comparison of samples subjected to trypsin digestion and samples incubated with antibody and then subjected to digestion by e.g. trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the TLR3 polypeptide that is not surface exposed and, accordingly, most likely not relevant in terms of immunogenicity/antigenicity. See, e.g., Mama, Ann Ist Super Sanita. 1991; 27: 15-9 for a discussion of similar techniques.

Site-directed mutagenesis is another technique useful for elucidation of a binding epitope. For example, in “alanine-scanning”, each residue within a protein segment is re-placed with an alanine residue, and the consequences for binding affinity measured. If the mutation leads to a significant resuction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron micros-copy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, BIACORE) and reflectometric interference spectroscopy (RifS). See, e.g., Fagerstam et al., Journal Of Molecular Recognition 1990; 3:208-14; Nice et al., J. Chroma-togr. 1993; 646:159-168; Leipert et al., Angew. Chem. Int. Ed. 1998; 37:3308-3311; Kröger et al., Biosensors and Bioelectronics 2002; 17:937-944.

It should also be noted that an antibody binding the same or substantially the same epitope as an antibody of the invention can be identified in one or more of the exemplary competition assays described herein.

Once antibodies are identified that are capable of binding TLR3 and/or having other desired properties, they will also typically be assessed, using standard methods including those described herein, for their ability to bind to other polypeptides, including unrelated polypeptides and other TLR family members (e.g., human TLR1, 2, or 4-10). Ideally, the antibodies only bind with substantial affinity to TLR3, e.g., human TLR3, and do not bind at a significant level to unrelated polypeptides or to other TLR family members (e.g., TLR2 or TLR4; the amino acid sequence of human precursor TLR4 including a signal peptide at amino acid residues 1-23 is found in NCBI accession number NP 612564, the disclosure of which is incorporated herein by reference). However, it will be appreciated that, as long as the affinity for TLR3 is substantially greater (e.g., 5×, 10×, 50×, 100×, 500×, 1000×, 10,000×, or more) than it is for other TLR family members (or other, unrelated polypeptides), then the antibodies are suitable for use in the present methods.

The binding of the antibodies to TLR3-expressing cells can also be assessed in non-human primates, e.g. cynomolgus monkeys, or other mammals such as mice. The invention therefore provides an antibody, as well as fragments and derivatives thereof, wherein said antibody, fragment or derivative specifically binds TLR3, and which furthermore bind TLR3 from non-human primates, e.g., cynomolgus monkeys. Optionally, cellular uptake or localization, optionally localization in a subcellular compartment such as the endocytic pathway, is assessed in order to select an antibody that is readily taken up into the cell and/or into the cellular compartment where it TLR3 is expressed. Cellular uptake or localization will generally be measured in the cells in which the antibody is sought or believed to exert its activity, such as in DC. Cellular uptake or localization can be assessed by standard methods, such as by confocal staining using an antibody marked with a detectable moiety (e.g. a fluorescent moiety).

Upon immunization and production of antibodies in a vertebrate or cell, particular selection steps may be performed to isolate antibodies as claimed. In this regard, in a specific embodiment, the invention also relates to methods of producing such antibodies, comprising: (a) immunizing a non-human mammal with an immunogen comprising a TLR3 polypeptide; and (b) preparing antibodies from said immunized animal; and (c) selecting antibodies from step (b) that are capable of binding TLR3. The antibodies can be tested for binding to TLR3 under acidic conditions corresponding to those in cytosolic compartments (e.g. the endosomic compartments), such as at a pH of between about 5.5 to 6.5.

The antibodies' bivalent binding affinity for human TLR3 under acidic conditions can determined. Antibodies can be characterized for example by a mean KD of no more than about (i.e. better affinity than) 100, 60, 10, 5, or 1 nanomolar, preferably sub-nanomolar or optionally no more than about 300, 200, 100 or 10 picomolar. KD can be determined for example for example by immobilizing recombinantly produced human TLR3 proteins on a chip surface, followed by application of the antibody to be tested in solution, e.g. as shown in the present Examples. To select antibodies that retain binding similar binding under acidic and neutral conditions, one can seek to minimize the difference observed between binding at neutral pH (e.g. 7.2) and acidic pH (e.g. a pH in the range of 4.5-6-5), for example where binding affinity at acidic pH is not substantially lower, e.g. where the KD for binding to TLR3 decreases by no more than 0.2-, 0.3-, 0.5-, 1.0-, or 1.5-log 10, than that observed at non-acid pH. In one embodiment, the method further comprises a step (d), selecting antibodies from (b) that are capable of competing for binding to TLR3 with antibody 31C3, 29H3, 23C8, 28F11 or 34A3.

In one aspect of any of the embodiments, the antibodies prepared according to the present methods are monoclonal antibodies. In another aspect, the non-human animal used to produce antibodies according to the methods of the invention is a mammal, such as a rodent, bovine, porcine, fowl, horse, rabbit, goat, or sheep. The antibodies of the present invention encompass 31C3, 29H3, 23C8, 28F11 or 34A3. However, it will be appreciated that other antibodies can be obtained using the methods described herein, and thus antibodies of the invention can be antibodies other than 31C3, 29H3, 23C8, 28F11 or 34A3. Additionally, antibodies of the invention can optionally be specified to be antibodies other than any of antibodies TLR3.7 (eBioScience Inc., San Diego), antibody C1068 of WO 06/060513, antibody C1130 of WO 2007/051164, any of the antibodies disclosed WO2010/051470, e.g., antibodies 1-19 and F17-F19, and their variants such as 15EVQ and 12QVQ/QSV, antibody 40C1285 (Abcam), or antibodies 619F7, 713E4, 716G10, IMG-5631, IMG-315 or IMG-5348 (all from Imgenex. Corp.) or derivatives of the foregoing, e.g. that comprise the antigen binding region in whole or in part.

According to an alternate embodiment, the DNA encoding an antibody that binds an epitope present on TLR3 polypeptides is isolated from the hybridoma of this invention and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the invention, e.g., antibody 31C3, 29H3, 23C8, 28F11 or 34A3, can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody.

Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

### Assessing the Ability of Antibodies to Modulate TLR3 Signaling

In certain embodiments, the antibodies of this invention are able to modulate, e.g., inhibit signaling by, TLR3 polypeptides, and consequently to modulate the activity or behavior of TLR3-expressing cells. For example, antibodies may inhibit the activation of TLR3-expressing cells, e.g. they can inhibit the TLR3 signaling pathway, optionally without blocking the binding to TLR3 of natural or endogenous ligands such as dsRNA; optionally they may block the ability of TLR3 protein to form homodimers in the presence of a TLR3 ligand, thus blocking the initiation a signaling cascade. These antibodies are thus referred to as “neutralizing” or “inhibitory” or “blocking” antibodies. Such antibodies are useful, inter alia, for decreasing the activity of TLR3-expressing immune cells, e.g. for the treatment or prevention of conditions involving excess TLR3-expressing cell activity or number, or where decreased TLR3-expressing cell activity can ameliorate, prevent, eliminate, or in any way improve the condition or any symptom thereof.

A range of cellular assays can be used to assess the ability of the antibodies to modulate TLR3 signaling. Any of a large number of assays, including molecular, cell-based, and animal-based models can be used to assess the ability of anti-TLR3 antibodies to modulate TLR3-expressing cell activity. For example, cell-based assays can be used in which cells expressing TLR3 are exposed to dsRNA, viral dsRNA, polyIC, or poly AU, or another TLR3 ligand and the ability of the antibody to disrupt the binding of the ligand or the stimulation of the receptor (as determined, e.g., by examining any of the TLR3 cell activities addressed herein, such as interferon expression, NFkB activity, NK cell activation, etc.) is assessed. The TLR3 ligand used in the assays may be in any suitable form, including but not limited to as a purified ligand composition, in a mixture with non-TLR3 ligands, in a naturally occurring composition, in a cell or on the surface of a cell, or secreted by a cell (e.g. a cell that produces ligand is used in the assay), in solution or on a solid support.

The activity of TLR3-expressing cells can also be assessed in the absence of a ligand, by exposing the cells to the antibody itself and assessing its effect on any aspect of the cells' activity or behavior. In such assays, a baseline level of activity (e.g., cytokine production, proliferation, see below) of the TLR3-expressing cells is obtained in the absence of a ligand, and the ability of the antibody or compound to alter the baseline activity level is detected. In one such embodiment, a high-throughput screening approach is used to identify compounds capable of affecting the activation of the receptor.

Any suitable physiological change that reflects TLR3 activity can be used to evaluate test antibodies or antibody derivatives. For example, one can measure a variety of effects, such as changes in gene expression (e.g., NFkB-responding genes), protein secretion (e.g., interferon), cell growth, cell proliferation, pH, intracellular second messengers, e.g., Ca²⁺, IP3, cGMP, or cAMP, or activity such as ability to activate NK cells. In one embodiment, the activity of the receptor is assessed by detecting production of cytokines, e.g. TLR3-responsive cytokines, proinflammatory cytokines.

TLR3 modulation can be assessed using any of a number of possible readout systems, most based upon a TLR/IL-1R signal transduction pathway, involving, e.g., the MyD88-independent/TRIF dependent signal transduction pathway, involving, e.g., IRF3, IRF7, IKKε and/or TBK1 (Akira and Takeda (2004) Nature Review Immunol. 4:499-511). These pathways activate kinases including KB kinase complex. TLR3 activation can be assessed by examining any aspect of TLR signaling. For example, activation of TLR signaling triggers alterations in protein-protein associations (e.g., TRIF with TBK and/or IKKε), in intracellular localization of proteins (such as movement of NK-kB into the nucleus), and in gene expression (e.g., in expression of NK-kB sensitive genes), and cytokine production (e.g., production and secretion of IFN-gamma, IL-6, IP10, MCP-1). Any such alteration can be detected and used to detect TLR3 activation. In one embodiment, TLR3 stimulation is detected by collecting supernatants after 18-20 hr of culture and measuring levels of IFN-gamma, IL-6, IP-10 and/or MCP-1 by sandwich ELISA. In another embodiment, TLR3 stimulation is detected by collecting supernatants after 18-20 hr of culture and measuring levels of IFN-gamma, IL-6, IP-10 and/or MCP-1 by sandwich ELISA.

In one embodiment, cells that naturally express TLR3 are used, such as DC (e.g. myeloid DC or monocyte derived DC. In another embodiment, cells are used that contain a reporter construct that causes the expression of a detectable gene product upon TLR3 stimulation and consequent activation of the signal transduction pathway. Reporter genes and reporter gene constructs particularly useful for the assays include, e.g., a reporter gene operatively linked to a promoter sensitive to NF-kB or to signaling mediated by, particularly TRIF, IRF3, IRF7, IKKε, TBK1. Examples of such promoters include, without limitation, those for IL-1alpha, IL-6, IL-8, IL-12 p40, IP-10, CD80, CD86, and TNF-alpha. The reporter gene operatively linked to the TLR-sensitive promoter can include, without limitation, an enzyme (e.g., luciferase, alkaline phosphatase, beta-galactosidase, chloramphenicol acetyltransferase (CAT), etc.), a bioluminescence marker (e.g., green-fluorescent protein (GFP, e.g., U.S. Pat. No. 5,491,084), blue fluorescent protein (BFP, e.g., U.S. Pat. No. 6,486,382), etc.), a surface-expressed molecule (e.g., CD25, CD80, CD86), and a secreted molecule (e.g., IL-1, IL-6, IL-8, IL-12 p40, TNF-alpha). See, e.g., Hcker H et al. (1999) EMBO J. 18:6973-82; Murphy T L et al. (1995) Mol Cell Biol 15:5258-67, the disclosures of which are herein incorporated by reference. Reporter plasmids suitable for use are commercially available (InvivoGen, San Diego, Calif.). In one embodiment, the assay includes determining, in a host cell made to express a human TLR3 polypeptide, whether a test composition induces luciferase expression (or other reporter) under the control of a promoter responsive to TLR3 signaling (e.g. ISRE, IFN-stimulated response element).

In assays relying on enzyme activity readout, substrate can be supplied as part of the assay, and detection can involve measurement of chemoluminescence, fluorescence, color development, incorporation of radioactive label, drug resistance, optical density, or other marker of enzyme activity. For assays relying on surface expression of a molecule, detection can be accomplished using flow cytometry (FACS) analysis or functional assays. Secreted molecules can be assayed using enzyme-linked immunosorbent assay (ELISA) or bioassays. Many of these and other suitable readout systems are well known in the art and are commercially available. Preferably, the reporter system, whichever used, is quantifiable.

In another embodiment, the effect of the antibodies on TLR3-expressing cells is assessed in non-human primates in vivo. For example, a pharmaceutical composition comprising an anti-TLR3 antibody of the present invention is administered to a non-human primate that is either healthy or affected by a condition, e.g. an autoimmune disease or inflammation and the effect of the administration on, e.g., the number or activity of TLR3-expressing cells in the primate, the presence and/or levels of cytokines, or on the progression of the condition is assessed. Any antibody or antibody derivative or fragment that effects a detectable change in any of these TLR3-related parameters is a candidate for use in the herein-described methods.

In any of the herein-described assays, an increase or decrease of 5%, 10%, 20%, preferably 30%, 40%, 50%, most preferably 60%, 70%, 80%, 90%, 95%, or greater in any detectable measure of TLR3-stimulated activity in the cells indicates that the test antibody is suitable for use in the present methods.

When assessing inhibitory anti-TLR3 antibodies, the antibodies can be advantageously selected to modify any parameter associated with inflammation or autoimmunity. For example, antibodies can be selected to reduce activation, particularly production of pro-inflammatory cytokines, in cells. The cells may be, for example, cells obtained from an individual suffering from an inflammatory or autoimmune disorder.

### Antibody CDR Sequences

In one aspect of any of the embodiments of the invention, an antibody may comprise a heavy and/or light chain having CDR1, 2 and/or 3 sequences according to the respective formula selected from Formulas (I) to (XXV). In any embodiment herein, a particular LCDR1 or -2 or HCDR-1 or 2 may be specified as having a sequence of Formulas (I) to (VII) and (XI to XXI). In any embodiment herein, a particular HCDR1-3 or LCDR-1-3 may be specified as having a sequence of Formulas (I) to (XXV). In one preferred embodiment, the antibody comprises a light chain comprising the three LCDRs and a heavy chain comprising the three HCDRs. Optionally, provided is an antibody where any of the light and/or heavy chain variable regions are fused to an immunoglobulin constant region of the IgG type, optionally a human constant region, optionally an IgG1 or IgG4 isotype.

In one embodiment, LCDR1 is of Formula (I):

| | |
|---|---|
| R-A-S-E-N-I-Y-S-Xaa₁-L-A (I), | (SEQ ID NO: 61) |

wherein Xaa₁ may be a conservative or non conservative substitution or a deletion or insertion, preferably, wherein Xaa₁ may be Ser, Tyr or Asn.

In one embodiment, LCDR2 is of Formula (II):

| | |
|---|---|
| Xaa₂-A-K-T-L-A-E (II), | (SEQ ID NO: 62) |

wherein Xaa₂ may be a conservative or non conservative substitution or a deletion or insertion, preferably, wherein Xaa₂ may be Asn or Tyr.

In one embodiment, LCDR3 is of Formula (III):

| | |
|---|---|
| Q-H-H-Y-G-T-P-Xaa₃-T (III), | (SEQ ID NO: 63) |

wherein Xaa₃ may be a conservative or non conservative substitution or a deletion or insertion, preferably, wherein Xaa₃ may be Tyr, Phe, Pro.

In one embodiment, LCDR1 is of Formula (IV):

| | |
|---|---|
| Xaa₄-A-S-Xaa₅-Xaa₆-Xaa₇-Xaa₈-Xaa₉- | (SEQ ID NO: 64) |
| Xaa₁₀-Xaa₁₁-Xaa₁₂ (IV), | |

wherein Xaa₄ to Xaa₁₂ may be a conservative or non conservative substitution or a deletion or insertion, preferably, wherein Xaa₄ may be Arg, Ser or Lys, and/or Xaa₅ may be Glu, Ser or Gln, and/or Xaa₆ may be Asn or Ser, and/or Xaa₇ may be Ile or Val, and/or Xaa₈ may be a deletion, Tyr or Arg, and/or Xaa₉ may be Ser or Thr, and/or Xaa₁₀ may be Tyr, Asn or Ser, and/or Xaa₁₁ may be Leu, Met or Val, and/or Xaa₁₂ may be Ala or Phe.

In one embodiment, LCDR1 is of Formula (V):

| | |
|---|---|
| Xaa₁₃-Xaa₁₄-Xaa₁₅-Xaa₁₆-L-A-Xaa₁₇ (V), | (SEQ ID NO: 65) |

wherein Xaa₁₃ to Xaa₁₇ may be a conservative or non conservative substitution or a deletion or insertion, preferably, wherein Xaa₁₃ may be Leu, Asn or Tyr, and/or Xaa₁₄ may be Ala or Thr, and/or Xaa₁₅ may be Lys or Ser, and/or Xaa₁₆ may be Asn or Thr, and/or Xaa₁₇ may be Glu or Ser. Optionally, Xaa₁₇ is a deletion (absent).

In one embodiment, LCDR2 is of Formula (VI):

| | |
|---|---|
| Xaa₁₈-A-Xaa₁₉-Xaa₂₀-Xaa₂₁-Xaa₂₂- | (SEQ ID NO: 66) |
| Xaa₂₃ (VI), | |

wherein Xaa₁₉ to Xaa₂₃ may be a conservative or non conservative substitution or a deletion or insertion, preferably, wherein Xaa₁₈ may be Tyr, Asn or Leu, and/or Xaa₁₉ may be Ser or Lys, and/or Xaa₂₀ may be Asn or Thr, and/or Xaa₂₁ may be Leu or Arg, and/or Xaa₂₂ may be Ala or His, and/or Xaa₂₃ may be Thr or Glu.

In one embodiment, LCDR2 is of Formula (VII):

| | |
|---|---|
| L-Xaa₂₄-S-N-Xaa₂₅-Xaa₂₆-Xaa₂₇ (VII), | (SEQ ID NO: 67) |

wherein Xaa₂₄ to Xaa₂₇ may be a conservative or non conservative substitution or a deletion or insertion, preferably, wherein Xaa₂₄ may be Thr or Ala, and/or Xaa₂₅ may be Leu or Arg, and/or Xaa₂₆ may be Ala or His, and/or Xaa₂₇ may be Ser or Thr.

In one embodiment, LCDR3 is of Formula (VIII):

| | |
|---|---|
| Q-Xaa₂₈-Xaa₂₉-Xaa₃₀-G-Xaa₃₁-P- | (SEQ ID NO: 68) |
| Xaa₃₂-T (VIII), | |

wherein Xaa₂₈ to Xaa₃₂ may be a conservative or non conservative substitution or a deletion or insertion, preferably, wherein Xaa₂₈ may be His or Gln, and/or Xaa₂₉ may be His or Trp, and/or Xaa₃₀ may be Tyr or Thr, and/or Xaa₃₁ may be Thr or Asn, and/or Xaa₃₂ may be Tyr, Phe or Pro.

In one embodiment, LCDR3 is of Formula (IX):

| | |
|---|---|
| Q-Xaa₃₃-H-Xaa₃₄-Xaa₃₅-Xaa₃₆- | (SEQ ID NO: 69) |
| P-Xaa₃₇-Xaa₃₈ (IX), | |

wherein Xaa₃₃ to Xaa₃₈ may be a conservative or non conservative substitution or a deletion or insertion, preferably, wherein Xaa₃₃ may be His or Gln, and/or Xaa₃₄ may be Trp or Tyr, and/or Xaa₃₅ may be Asn or Gly, and/or Xaa₃₆ may be Tyr or Thr, and/or Xaa₃₇ may be Tyr, Phe or Pro and/or Xaa₃₈ may be Thr, Met, or any other amino acid other than Thr.

In one embodiment, LCDR3 is of Formula (X):

| | |
|---|---|
| Xaa₃₉-Q-Xaa₄₀-Xaa₄₁-Xaa₄₂-Xaa₄₃-P- | (SEQ ID NO: 70) |
| Xaa₄₄-T (X), | |

wherein Xaa₄₀ to Xaa₄₄ may be a conservative or non conservative substitution or a deletion or insertion, preferably, wherein Xaa₃₉ may be Gln or Leu, and/or Xaa₄₀ may be Trp or His, and/or Xaa₄₁ may be Thr or Trp, and/or Xaa₄₂ may be Gly or Asn, and/or Xaa₄₃ may be Asn or Tyr, and/or Xaa₄₄ may be Pro or Tyr.

In one embodiment, HCDR1 is of Formula (XI):

| | |
|---|---|
| G-Y-S-F-T-G-Y-Xaa₄₅-Xaa₄₆-H (XI), | (SEQ ID NO: 71) |

wherein Xaa₄₅ to Xaa₄₆ may be a conservative or non conservative substitution or a deletion or insertion, preferably, wherein Xaa₄₅ may be Phe or Tyr, and/or Xaa₄₆ may be Met or Ile.

In one embodiment, HCDR1 is of Formula (XII):

| | |
|---|---|
| G-Y-S-F-T-Xaa₄₇-Y-Xaa₄₈-M-H (XII), | (SEQ ID NO: 72) |

wherein Xaa₄₇ to Xaa₄₈ may be a conservative or non conservative substitution or a deletion or insertion, preferably, wherein Xaa₄₇ may be Gly or Ala, and/or Xaa₄₈ may be Phe or Tyr.

In one embodiment, HCDR1 is of Formula (XIII):

| | |
|---|---|
| G-Y-S-F-T-Xaa₄₉-Y-Y-Xaa₅₀-H (XIII), | (SEQ ID NO: 73) |

wherein Xaa₄₉ to Xaa₅₀ may be a conservative or non conservative substitution or a deletion or insertion, preferably, wherein Xaa₄₉ may be Gly or Ala, and/or Xaa₅₀ may be Ile or Met.

In one embodiment, HCDR1 is of Formula (XIV):

(SEQ ID NO: 74)
G-Y-Xaa₅₁-F-T-Xaa₅₂-Y-Xaa₅₃-Xaa₅₄-Xaa₅₅ (XIV),

wherein Xaa₅₁ to Xaa₅₅ may be a conservative or non conservative substitution or a deletion or insertion, preferably, wherein Xaa₅₁ may be Val or Ser, and/or Xaa₅₂ may be Thr, Gly or Ala, and/or Xaa₅₃ may be Ser, Tyr or Phe, and/or Xaa₅₄ may be Ile or Met, and/or Xaa₅₅ may be Tyr or His.

In one embodiment, HCDR1 is of Formula (XV):

(SEQ ID NO: 75)
G-Y-S-Xaa₅₆-T-Xaa₅₇-G-Y-Xaa₅₈-Xaa₅₉-H (XV),

wherein Xaa₅₆ to Xaa₅₉ may be a conservative or non conservative substitution or a deletion or insertion, preferably, wherein Xaa₅₆ may be Ile or Phe, and/or Xaa₅₇ may be a deletion or Ser, and/or Xaa₅₈ may be Ser, Tyr or Phe, and/or Xaa₅₉ may be Trp, Ile or Met.

In one embodiment, HCDR1 is of Formula (XVI):

(SEQ ID NO: 76)
G-Y-Xaa₆₀-Xaa₆₁-T-Xaa₆₂-Xaa₆₃-Y-S-Xaa₆₄-Xaa₆₅ (XVI),

wherein Xaa₆₀ to Xaa₆₅ may be a conservative or non conservative substitution or a deletion or insertion, preferably, wherein Xaa₆₀ may be Val or Ser and/or Xaa₆₁ may be Phe or Ile and/or Xaa₆₂ may be Thr or Ser and/or Xaa₆₃ may be deletion or Gly and/or Xaa₆₄ may be Ile or Trp and/or Xaa₆₅ may be Tyr or His.

In one embodiment, HCDR2 is of Formula (XVII):

(SEQ ID NO: 77)
R-I-N-P-Y-Xaa₆₆-G-A-T-S-Xaa₆₇-N-Xaa₆₈-N-F-K-D (XVII),

wherein Xaa₆₆ to Xaa₆₈ may be a conservative or non conservative substitution or a deletion or insertion, preferably, wherein Xaa₆₆ may be Asn or Tyr and/or Xaa₆₇ may be deletion or Tyr and/or Xaa₆₈ may be Arg or Gln.

In one embodiment, HCDR2 is of Formula (XVIII):

(SEQ ID NO: 78)
R-I-N-P-Y-Xaa₆₆-G-A-T-S-(XVIII),

wherein Xaa₆₆ may be a conservative or non conservative substitution or a deletion or insertion, preferably, wherein Xaa₆₆ may be Asn or Tyr,

In one embodiment, HCDR2 is of Formula (XIX):

(SEQ ID NO: 79)
R-I-N-P-Y-N-G-A-T-S-Y-N-Xaa₆₈-N-F-K-D (XIX),

wherein Xaa₆₈ may be a conservative or non conservative substitution or a deletion or insertion, preferably, wherein Xaa₆₈ may be Arg or Gln.

In one embodiment, HCDR2 is of Formula (XX):

(SEQ ID NO: 80)
Y-I-Xaa₆₉-Xaa₇₀-Y-Xaa₇₁-G-Xaa₇₂-T-Xaa₇₃-Y-N-Xaa₇₄-
Xaa₇₅-Xaa₇₆-Xaa₇₇-Xaa₇₈ (XX),

wherein Xaa₆₉ to Xaa₇₈ may be a conservative or non conservative substitution or a deletion or insertion, preferably, wherein Xaa₆₉ may be Asp or His and/or Xaa₇₀ may be a deletion or Pro and/or Xaa₇₁ may be Ser or Asn and/or Xaa₇₂ may be Ile or Asp and/or Xaa₇₃ may be Ser or Asn and/or Xaa₇₄ may be Gln or Pro and/or Xaa₇₅ may be Lys or Ser and/or Xaa₇₆ may be Phe or Leu and/or Xaa₇₇ may be Lys or Arg and/or Xaa₇₈ may be Gly or Ser.

In one embodiment, HCDR2 is of Formula (XXI):

(SEQ ID NO: 81)
Xaa₇₉-I-Xaa₈₀-Xaa₈₁-Y-Xaa₈₂-G-Xaa₈₃-T-Xaa₈₄-Xaa₈₅-
N-Xaa₈₆-Xaa₈₇-Xaa₈₈-Xaa₈₉-Xaa₉₀ (XXI),

wherein Xaa₇₉ to Xaa₉₀ may be a conservative or non conservative substitution or a deletion or insertion, preferably, wherein Xaa₇₉ may be Arg or Tyr and/or Xaa₈₀ may be Asn, Asp or His and/or Xaa₈₁ may be a deletion or Pro and/or Xaa₈₂ may be Tyr, Asn or Ser and/or Xaa₈₃ may be Ile, Asp or Ala and/or Xaa₈₄ may be Ser or Asn and/or Xaa₈₅ may be a deletion or Tyr and/or Xaa₈₆ may be Pro, Arg or Gln and/or Xaa₈₇ may be Ser, Lys or Asn and/or Xaa₈₈ may be Phe or Leu and/or Xaa₈₉ may be Lys or Arg and/or Xaa₉₀ may be Asp or Gly. Optionally Xaa₈₅, Xaa₈₆, Xaa₈₇, Xaa₈₈, Xaa₈₉ and/or Xaa₉₀ are a deletion (i.e. are absent).

In one embodiment, HCDR3 is of Formula (XXII):

(SEQ ID NO: 82)
Xaa₉₁-Xaa₉₂-G-Xaa₉₃-Xaa₉₄-Y-Xaa₉₅-F-D-Y (XXII),

wherein Xaa₉₁ to Xaa₉₅ may be a conservative or non conservative substitution or a deletion or insertion, preferably, wherein Xaa₉₁ may be Asp or Ser, and/or Xaa₉₂ may be Asp or Gly, and/or Xaa₉₃ may be Gly or Asn, and/or Xaa₉₄ may be Asn or Thr, and/or Xaa₉₅ may be Pro or a deletion.

In one embodiment, HCDR3 is of Formula (XXIII):

(SEQ ID NO: 83)
Xaa₉₆-Xaa₉₇-Xaa₉₈-Xaa₉₉-Xaa₁₀₀-Y-Xaa₁₀₁-Xaa₁₀₂-D-Y
(XXIII),

wherein Xaa₉₆ to Xaa₁₀₂ may be a conservative or non conservative substitution or a deletion or insertion, preferably, wherein Xaa₉₆ may be S or D, and/or Xaa₉₇ may be G, T D, and/or Xaa₉₈ may be G, K or a deletion, and/or Xaa₉₉ may be G, L, N or a deletion, and/or Xaa₁₀₀ may be Y, T, G, N, and/or Xaa₁₀₁ may be P, G or a deletion, and/or Xaa₁₀₂ may be M, F or L.

In one embodiment, HCDR3 is of Formula (XXIV):

(SEQ ID NO: 84)
Xaa₁₀₃-Xaa₁₀₄-Xaa₁₀₅-Xaa₁₀₆-Xaa₁₀₇-Xaa₁₀₈-F-D-
Xaa₁₀₉ (XXIV),

wherein Xaa₁₀₃ to Xaa₁₀₉ may be a conservative or non conservative substitution or a deletion or insertion, preferably, wherein Xaa₁₀₃ may be Asp, Ser, Glu, and/or Xaa₁₀₄ may be Asp or Gly, and/or Xaa₁₀₅ may be Gly or Asn, and/or Xaa₁₀₆ may be Asn, Tyr or Gly, and/or Xaa₁₀₇ may be Asn, Thr or Tyr, and/or Xaa₁₀₈ may be Tyr or Gly, and/or Xaa₁₀₉ may be Tyr, Val, or any amino acid other than Tyr. Optionally, the formula further comprises an amino acid insertion between Xaa₁₀₈ and F, wherein the amino acid inserted is a Tyr or a Pro.

In one embodiment, HCDR3 is of Formula (XXV):

(SEQ ID NO: 85)
Xaa₁₁₀-G-Xaa₁₁₁-Xaa₁₁₂-Y-Xaa₁₁₃-Xaa₁₁₄-Xaa₁₁₅-D-Y
(XXV),

wherein Xaa₁₁₀ to Xaa₁₁₅ may be a conservative or non conservative substitution or a deletion or insertion, preferably, wherein Xaa₁₁₀ may be Glu or Asp, and/or Xaa₁₁₁ may be Asn or a deletion, and/or Xaa₁₁₂ may be Tyr or a deletion, and/or Xaa₁₁₃ may be Tyr, or Gly, and/or Xaa₁₁₄ may be Tyr or Gly, and/or Xaa₁₁₅ may be Met or Phe.

In one embodiment, an antibody of the invention may comprise a light chain comprising:
a a light chain CDR1 (LCDR1) amino acid sequence selected from SEQ ID NOS: 61, 64 and 65; and/or
b a light chain CDR2 (LCDR2) amino acid sequence selected from SEQ ID NOS: 62, 66 and 67; and/or
c a light chain CDR3 (LCDR3) amino acid sequence selected from SEQ ID NOS: 63, 68, 69 and 70.

In one embodiment, an antibody of the invention may comprise a heavy chain comprising:
d a heavy chain CDR1 (HCDR1) amino acid sequence selected from SEQ ID NOS: 71 to 76; and/or
e a heavy chain CDR2 (HCDR2) amino acid sequence selected from SEQ ID NOS: 77 to 81; and/or
f a heavy chain CDR3 (HCDR3) amino acid sequence selected from SEQ ID NOS: 82 to 85.

### Antibody 29H3

Cells producing antibody 29H3 have been deposited at the CNCM under accession number 1-4187; the antibody 29H3 has also been sequenced. The amino acid sequence of the heavy chain variable region is listed as SEQ ID NO:10, the amino acid sequence of the light chain variable region is listed as SEQ ID NO:11. The nucleic acid sequence encoding the heavy and light chain variable regions are listed in SEQ ID NOS: 53 and 54, respectively. In one embodiment, the invention provides an antibody that binds essentially the same epitope or determinant as monoclonal antibodies 29H3; optionally the antibody comprises an antigen binding region of antibody 29H3. In any of the embodiments herein, antibody 29H3 can be characterized by its amino acid sequence and/or nucleic acid sequence encoding it. In one preferred embodiment, the monoclonal antibody comprises the Fab or F(ab′)₂ portion of 29H3. Also provided is a monoclonal antibody that comprises the heavy chain variable region of 29H3. According to one embodiment, the monoclonal antibody comprises the three CDRs of the heavy chain variable region of 29H3. Also provided is a monoclonal antibody that further comprises the variable light chain variable region of 29H3 or one, two or three of the CDRs of the light chain variable region of 29H3. Optionally any one or more of said light or heavy chain CDRs may contain one, two, three, four or five amino acid modifications (e.g. substitutions, insertions or deletions). Optionally, provided is an antibody where any of the light and/or heavy chain variable regions comprising part or all of an antigen binding region of antibody 29H3 are fused to an immunoglobulin constant region of the IgG type, optionally a human constant region, optionally an IgG1 or IgG4 isotype. In another preferred embodiment the antibody is 29H3.

In another aspect, the invention provides a purified polypeptide which encodes an antibody, wherein the antibody comprises: a VHCDR1 region comprising an amino acid sequence as set forth in SEQ ID NO:12, wherein one or more of these amino acids may be substituted by a different amino acid; a VHCDR2 region comprising an amino acid sequence as set forth in SEQ ID NO:13, wherein one or more of these amino acids may be substituted by a different amino acid; a VHCDR3 region comprising an amino acid sequence as set forth in SEQ ID NO:14, wherein one or more of these amino acids may be substituted by a different amino acid; a VLCDR1 region comprising an amino acid sequence as set forth in SEQ ID NO:15, wherein one or more of these amino acids may be substituted by a different amino acid; a VLCDR2 region comprising an amino acid sequence as set forth in SEQ ID NO:16, wherein one or more of these amino acids may be substituted by a different amino acid; and/or a VLCDR3 region comprising an amino acid sequence as set forth in SEQ ID NO:17, wherein one or more of these amino acids may be substituted by a different amino acid.

In still another aspect, the invention provides an antibody, which comprises a heavy chain and/or a light chain each having at least three CDRs, wherein one, two or three of at least three CDRs has the sequence of SEQ ID NO:12 to 14 and 15 to 17 for the respective the heavy and light chains, and which antibody specifically binds to TLR3 in acidic conditions.

In another aspect, the invention provides an antibody that binds human TLR3, comprising:
a. the heavy chain variable region of SEQ ID NO:10, wherein one, two, three or more of these amino acids may be substituted by a different amino acid; or
b. the light chain variable region of SEQ ID NO:11, wherein one, two, three or more of these amino acids may be substituted by a different amino acid; or
c. the heavy chain variable region of SEQ ID NO:10, wherein one, two, three or more of these amino acids may be substituted by a different amino acid; and the light chain variable region of SEQ ID NO:11, wherein one or more of these amino acids may be substituted by a different amino acid; or
d. the heavy chain CDR 1, 2 and 3 (HCDR1, HCDR2, HCDR3) amino acid sequences as shown in SEQ ID NO:12, 13 and 14, wherein one, two, three or more of these amino acids may be substituted by a different amino acid; or
e. the light chain CDR 1, 2 and 3 (LCDR1, LCDR2, LCDR3) e amino acid sequences as shown in SEQ ID NO:15, 16 and 17, respectively, wherein one, two, three or more of these amino acids may be substituted by a different amino acid; or
f. the heavy chain CDR 1, 2 and 3 (HCDR1, HCDR2, HCDR3) amino acid sequences as shown in SEQ ID NO: 12, 13 and 14, wherein one, two, three or more of these amino acids may be substituted by a different amino acid; and the light chain CDR 1, 2 and 3 (LCDR1, LCDR2, LCDR3) amino acid sequences as shown in SEQ ID NO: 15, 16 and 17, wherein one, two, three or more of these amino acids may be substituted by a different amino acid; or
g. the heavy chain variable region which is at least 60%, 70%, 80%, 85%, 90% or 95% identical to the variable region having an amino acid sequence of SEQ ID NO:10, wherein one, two, three or more of these amino acids may be substituted by a different amino acid; or
h. the light chain variable region which is at least 60%, 70%, 80%, 85%, 90% or 95% identical to the variable region having an amino acid sequence of SEQ ID NO:11, wherein one, two, three or more of these amino acids may be substituted by a different amino acid.

In another aspect of any of the embodiments herein, any of the CDRs 1, 2 and 3 of the heavy and light chains may be characterized as having an amino acid sequence that shares at least 50%, 60%, 70%, 80%, 85%, 90% or 95% sequence identity with the particular CDR or set of CDRs listed in the corresponding SEQ ID NO.

In another aspect, the invention provides an antibody that competes for TLR3 binding with a monoclonal antibody of (a) to (h), above.

### Antibody 31C3

Cells producing antibody 31C3 have been deposited at the CNCM under accession number 1-4186, also the antibody 31C3 has also been sequenced. The amino acid sequence of the heavy chain variable region is listed as SEQ ID NO: 2, the amino acid sequence of the light chain variable region is listed as SEQ ID NO: 3. The nucleic acid sequence encoding the heavy and light chain variable regions are listed in SEQ ID NOS: 51 and 52, respectively. In a specific embodiment, the invention provides an antibody that binds essentially the same epitope or determinant as monoclonal antibodies 31C3; optionally the antibody comprises an antigen binding region of antibody 31C3. In any of the embodiments herein, antibody 31C3 can be characterized by its amino acid sequence and/or nucleic acid sequence encoding it. In one preferred embodiment, the monoclonal antibody comprises the Fab or F(ab′)₂ portion of 31C3. Also provided is a monoclonal antibody that comprises the heavy chain variable region of 31C3. According to one embodiment, the monoclonal antibody comprises the three CDRs of the heavy chain variable region of 31C3 Also provided is a monoclonal antibody that further comprises the variable light chain variable region of 31C3 or one, two or three of the CDRs of the light chain variable region of 31C3. Optionally any one or more of said light or heavy chain CDRs may contain one, two, three, four or five amino acid modifications (e.g. substitutions, insertions or deletions). Optionally, provided is an antibody where any of the light and/or heavy chain variable regions comprising part or all of an antigen binding region of antibody 31C3 are fused to an immunoglobulin constant region of the IgG type, optionally a human constant region, optionally a human IgG1 or IgG4 isotype. In another preferred embodiment the antibody is 31C3.

In another aspect, the invention provides a purified polypeptide which encodes a antibody, wherein the antibody comprises: a VHCDR1 region comprising an amino acid sequence as set forth in SEQ ID NO:4, wherein one or more of these amino acids may be substituted by a different amino acid; a VHCDR2 region comprising an amino acid sequence as set forth in SEQ ID NO:5, wherein one or more of these amino acids may be substituted by a different amino acid; a VHCDR3 region comprising an amino acid sequence as set forth in SEQ ID NO:6, wherein one or more of these amino acids may be substituted by a different amino acid; a VLCDR1 region comprising an amino acid sequence as set forth in SEQ ID NO:7, wherein one or more of these amino acids may be substituted by a different amino acid; a VLCDR2 region comprising an amino acid sequence as set forth in SEQ ID NO:8, wherein one or more of these amino acids may be substituted by a different amino acid; a VLCDR3 region comprising an amino acid sequence as set forth in SEQ ID NO:9, wherein one or more of these amino acids may be substituted by a different amino acid.

In still another aspect, the invention provides an antibody, which comprises a heavy chain and/or a light chain each having at least three CDRs, wherein one, two or three of the at least three CDRs has the sequence of SEQ ID NO:4 to 6 and 7 to 9 for the respective the heavy and light chains, and which antibody specifically binds to TLR3 in acidic conditions.

In another aspect, the invention provides an antibody that binds human TLR3, comprising:
a. the heavy chain variable region of SEQ ID NO:2, wherein one, two, three or more of these amino acids may be substituted by a different amino acid; or
b. the light chain variable region of SEQ ID NO: 3, wherein one, two, three or more of these amino acids may be substituted by a different amino acid; or
c. the heavy chain variable region of SEQ ID NO: 2, wherein one or more of these amino acids may be substituted by a different amino acid; and the light chain variable region of SEQ ID NO: 3, wherein one, two, three or more of these amino acids may be substituted by a different amino acid; or
d. the heavy chain CDR 1 and 2 (HCDR1, HCDR2) amino acid sequences as shown in SEQ ID NO:4 and 5, wherein one, two, three or more of these amino acids may be substituted by a different amino acid, optionally wherein the heavy chain comprises CDR 1, 2 and 3 (HCDR1, HCDR2, HCDR3) amino acid sequences as shown in SEQ ID NO:4, 5 and 6, wherein one, two, three or more of these amino acids may be substituted by a different amino acid; or
e. the light chain CDR 1, 2 and 3 (LCDR1, LCDR2, LCDR3) amino acid sequences as shown in SEQ ID NO: 7, 8 and 9, wherein one, two, three or more of these amino acids may be substituted by a different amino acid; or
f. the heavy chain CDR 1, 2 and 3 (HCDR1, HCDR2, HCDR3) amino acid sequences as shown in SEQ ID NO: 4, 5 and 6, wherein one or more of these amino acids may be substituted by a different amino acid; and the light chain CDRs 1, 2 and 3 (LCDR1, LCDR2, LCDR3) amino acid sequences as shown in SEQ ID NO: 7, 8 and 9, wherein one, two, three or more of these amino acids may be substituted by a different amino acid; or
g. the heavy chain variable region which is at least 60%, 70%, 80%, 85%, 90% or 95% identical to the variable region having an amino acid sequence of SEQ ID NO: 2, wherein one, two, three or more of these amino acids may be substituted by a different amino acid; or
h. the light chain variable region which is at least 60%, 70%, 80%, 85%, 90% or 95% identical to the variable region having an amino acid sequence of SEQ ID NO: 3, wherein one, two, three or more of these amino acids may be substituted by a different amino acid.

In another aspect of any of the embodiments herein, any of the CDRs 1, 2 and 3 of the heavy and light chains may be characterized as having an amino acid sequence that shares at least 50%, 60%, 70%, 80%, 85%, 90% or 95% sequence identity with the particular CDR or set of CDRs listed in the corresponding SEQ ID NO.

In another aspect, the invention provides an antibody that competes for TLR3 binding with a monoclonal antibody of (a) to (h), above.

### Antibody 23C8

The antibody 23C8 has been sequenced. The amino acid sequence of the heavy chain variable region is listed as SEQ ID NO:26, the amino acid sequence of the light chain variable region is listed as SEQ ID NO:27. The nucleic acid sequence encoding the heavy and light chain variable regions are listed in SEQ ID NOS: 57 and 58, respectively. In a specific embodiment, the invention provides an antibody that binds essentially the same epitope or determinant as monoclonal antibodies 23C8; optionally the antibody comprises an antigen binding region of antibody 23C8. In any of the embodiments herein, antibody 23C8 can be characterized by its amino acid sequence and/or nucleic acid sequence encoding it. In one preferred embodiment, the monoclonal antibody comprises the Fab or F(ab′)2 portion of 23C8. Also provided is a monoclonal antibody that comprises the heavy chain variable region of 23C8. According to one embodiment, the monoclonal antibody comprises the three CDRs of the heavy chain variable region of 23C8. Also provided is a monoclonal antibody that further comprises the variable light chain variable region of 23C8 or one, two or three of the CDRs of the light chain variable region of 23C8. Optionally any one or more of said light or heavy chain CDRs may contain one, two, three, four or five amino acid modifications (e.g. substitutions, insertions or deletions). Optionally, provided is an antibody where any of the light and/or heavy chain variable regions comprising part or all of an antigen binding region of antibody 23C8 are fused to an immunoglobulin constant region of the IgG type, optionally a human constant region, optionally a human IgG1 or IgG4 isotype. In another preferred embodiment the antibody is 23C8.

In another aspect, the invention provides a purified polypeptide which encodes an antibody, wherein the antibody comprises: a VHCDR1 region comprising an amino acid sequence as set forth in SEQ ID NO:28, wherein one or more of these amino acids may be substituted by a different amino acid; a VHCDR2 region comprising an amino acid sequence as set forth in SEQ ID NO:29, wherein one or more of these amino acids may be substituted by a different amino acid; a VHCDR3 region comprising an amino acid sequence as set forth in SEQ ID NO:30, wherein one or more of these amino acids may be substituted by a different amino acid; a VLCDR1 region comprising an amino acid sequence as set forth in SEQ ID NO:31, wherein one or more of these amino acids may be substituted by a different amino acid; a VLCDR2 region comprising an amino acid sequence as set forth in SEQ ID NO:32, wherein one or more of these amino acids may be substituted by a different amino acid; a VLCDR3 region comprising an amino acid sequence as set forth in SEQ ID NO:33, wherein one or more of these amino acids may be substituted by a different amino acid.

In still another aspect, the invention provides an antibody, which comprises a heavy chain and/or a light chain each having at least three CDRs, wherein one, two or three of at least three CDRs has the sequence of SEQ ID NO:28 to 30 and 31 to 33 for the respective the heavy and light chains, and which antibody specifically binds to TLR3 in acidic conditions.

In another aspect, the invention provides an antibody that binds human TLR3, comprising:
a. the heavy chain variable region of SEQ ID NO: 26, wherein one, two, three or more of these amino acids may be substituted by a different amino acid; or
b. the light chain variable region of SEQ ID NO: 27, wherein one, two, three or more of these amino acids may be substituted by a different amino acid; or
c. the heavy chain variable region of SEQ ID NO: 26, wherein one, two, three or more of these amino acids may be substituted by a different amino acid; and the light chain variable region of SEQ ID NO: 27, wherein one, two, three or more of these amino acids may be substituted by a different amino acid; or
d. the heavy chain CDR 1 and 2 (HCDR1, HCDR2) amino acid sequences as shown in SEQ ID NO: 28 and 29, wherein one, two, three or more of these amino acids may be substituted by a different amino acid; optionally wherein the heavy chain comprises the CDR 1, 2 and 3 (HCDR1, HCDR2, HCDR3) amino acid sequences as shown in SEQ ID NO: 28, 29 and 30, wherein one, two, three or more of these amino acids may be substituted by a different amino acid; or
e. the light chain CDR 1, 2 and 3 (LCDR1, LCDR2, LCDR3) amino acid sequences as shown in SEQ ID NO: 31, 32 and 33, wherein one, two, three or more of these amino acids may be substituted by a different amino acid; or
f. the heavy chain CDR 1, 2 and 3 (HCDR1, HCDR2, HCDR3) amino acid sequences as shown in SEQ ID NO: 28, 29 and 30, wherein one, two, three or more of these amino acids may be substituted by a different amino acid; and the light chain CDR 1, 2 and 3 (LCDR1, LCDR2, LCDR3) amino acid sequences as shown in SEQ ID NO: 31, 32 and 33, wherein one, two, three or more of these amino acids may be substituted by a different amino acid; or
g. the heavy chain variable region which is at least 60%, 70%, 80%, 85%, 90% or 95% identical to the variable region having an amino acid sequence of SEQ ID NO: 26, wherein one, two, three or more of these amino acids may be substituted by a different amino acid; or
h. the light chain variable region which is at least 60%, 70%, 80%, 85%, 90% or 95% identical to the variable region having an amino acid sequence of SEQ ID NO: 27, wherein one, two, three or more of these amino acids may be substituted by a different amino acid.

In another aspect of any of the embodiments herein, any of the CDRs 1, 2 and 3 of the heavy and light chains may be characterized as having an amino acid sequence that shares at least 50% 60%, 70%, 80%, 85%, 90% or 95% sequence identity with the particular CDR or set of CDRs listed in the corresponding SEQ ID NO.

In another aspect, the invention provides an antibody that competes for TLR3 binding with a monoclonal antibody of (a) to (h), above.

### Antibody 28F11

The antibody 28F11 has been sequenced. The amino acid sequence of the heavy chain variable region is listed as SEQ ID NO:18, the amino acid sequence of the light chain variable region is listed as SEQ ID NO:19. The nucleic acid sequence encoding the heavy and light chain variable regions are listed in SEQ ID NOS: 55 and 56, respectively. In a specific embodiment, the invention provides an antibody that binds essentially the same epitope or determinant as monoclonal antibodies 28F11; optionally the antibody comprises an antigen binding region of antibody 28F 11. In any of the embodiments herein, antibody 28F11 can be characterized by its amino acid sequence and/or nucleic acid sequence encoding it. In one preferred embodiment, the monoclonal antibody comprises the Fab or F(ab′)2 portion of 28F11. Also provided is a monoclonal antibody that comprises the heavy chain variable region of 28F11. According to one embodiment, the monoclonal antibody comprises the three CDRs of the heavy chain variable region of 28F 11. Also provided is a monoclonal antibody that further comprises the variable light chain variable region of 28F11 or one, two or three of the CDRs of the light chain variable region of 28F 11. Optionally any one or more of said light or heavy chain CDRs may contain one, two, three, four or five amino acid modifications (e.g. substitutions, insertions or deletions). Optionally, provided is an antibody where any of the light and/or heavy chain variable regions comprising part or all of an antigen binding region of antibody 28F 11 are fused to an immunoglobulin constant region of the IgG type, optionally a human constant region, optionally a human IgG1 or IgG4 isotype. In another preferred embodiment the antibody is 28F11.

In another aspect, the invention provides a purified polypeptide which encodes a antibody, wherein the antibody comprises: a VHCDR1 region comprising an amino acid sequence as set forth in SEQ ID NO:20, wherein one or more of these amino acids may be substituted by a different amino acid; a VHCDR2 region comprising an amino acid sequence as set forth in SEQ ID NO:21, wherein one or more of these amino acids may be substituted by a different amino acid; a VHCDR3 region comprising an amino acid sequence as set forth in SEQ ID NO:22, wherein one or more of these amino acids may be substituted by a different amino acid; a VLCDR1 region comprising an amino acid sequence as set forth in SEQ ID NO:23, wherein one or more of these amino acids may be substituted by a different amino acid; a VLCDR2 region comprising an amino acid sequence as set forth in SEQ ID NO:24, wherein one or more of these amino acids may be substituted by a different amino acid; a VLCDR3 region comprising an amino acid sequence as set forth in SEQ ID NO:25, wherein one or more of these amino acids may be substituted by a different amino acid.

In still another aspect, the invention provides an antibody, which comprises a heavy chain and/or a light chain each having at least three CDRs, wherein one, two or three of at least three CDRs has the sequence of SEQ ID NO: 20 to 22 and 23 to 25 for the respective heavy and light chains, and which antibody specifically binds to TLR3 in acidic conditions.

In another aspect, the invention provides an antibody that binds human TLR3, comprising:
a. the heavy chain variable region of SEQ ID NO: 18, wherein one, two, three or more of these amino acids may be substituted by a different amino acid; or
b. the light chain variable region of SEQ ID NO: 19, wherein one, two, three or more of these amino acids may be substituted by a different amino acid; or
c. the heavy chain variable region of SEQ ID NO: 18, wherein one, two, three or more of these amino acids may be substituted by a different amino acid; and the light chain variable region of SEQ ID NO: 19, wherein one, two, three or more of these amino acids may be substituted by a different amino acid; or
d. the heavy chain CDR 1 and 2 (HCDR1, HCDR2) amino acid sequences as shown in SEQ ID NO: 20 and 21, wherein one, two, three or more of these amino acids may be substituted by a different amino acid; optionally wherein heavy chain comprises CDR 1, 2 and 3 (HCDR1, HCDR2, HCDR3) amino acid sequences as shown in SEQ ID NO: 20, 21 and 22, wherein one, two, three or more of these amino acids may be substituted by a different amino acid; or
e. the light chain CDR 1, 2 and 3 (LCDR1, LCDR2, LCDR3) amino acid sequences as shown in SEQ ID NO: 23, 24 and 25, wherein one, two, three or more of these amino acids may be substituted by a different amino acid; or
f. the heavy chain CDR 1, 2 and 3 (HCDR1, HCDR2, HCDR3) amino acid sequences as shown in SEQ ID NO: 20, 21 and 22, wherein one, two, three or more of these amino acids may be substituted by a different amino acid; and the light chain CDR 1, 2 and 3 (LCDR1, LCDR2, LCDR3) amino acid sequences as shown in SEQ ID NO: 23, 24 and 25, wherein one, two, three or more of these amino acids may be substituted by a different amino acid; or
g. the heavy chain variable region which is at least 60%, 70%, 80%, 85%, 90% or 95% identical to the variable region having an amino acid sequence of SEQ ID NO: 18, wherein one, two, three or more of these amino acids may be substituted by a different amino acid; or
h. the light chain variable region which is at least 60%, 70%, 80%, 85%, 90% or 95% identical to the variable region having an amino acid sequence of SEQ ID NO: 19, wherein one, two, three or more of these amino acids may be substituted by a different amino acid.

In another aspect of any of the embodiments herein, any of the CDRs 1, 2 and 3 of the heavy and light chains may be characterized as having an amino acid sequence that shares at least 50%, 60%, 70%, 80%, 85%, 90% or 95% sequence identity with the particular CDR or set of CDRs listed in the corresponding SEQ ID NO.

In another aspect, the invention provides an antibody that competes for TLR3 binding with a monoclonal antibody of (a) to (h), above.

### Antibody 34A3

The antibody 34A3 has been sequenced. The amino acid sequence of the heavy chain variable region is listed as SEQ ID NO:34, the amino acid sequence of the light chain variable region is listed as SEQ ID NO:35. The nucleic acid sequence encoding the heavy and light chain variable regions are listed in SEQ ID NOS: 59 and 60, respectively. In a specific embodiment, the invention provides an antibody that binds essentially the same epitope or determinant as monoclonal antibodies 34A3; optionally the antibody comprises an antigen binding region of antibody 34A3. In any of the embodiments herein, antibody 34A3 can be characterized by its amino acid sequence and/or nucleic acid sequence encoding it. In one preferred embodiment, the monoclonal antibody comprises the Fab or F(ab′)2 portion of 34A3. Also provided is a monoclonal antibody that comprises the heavy chain variable region of 34A3. According to one embodiment, the monoclonal antibody comprises the three CDRs of the heavy chain variable region of 34A3. Also provided is a monoclonal antibody that further comprises the variable light chain variable region of 34A3 or one, two or three of the CDRs of the light chain variable region of 34A3. Optionally any one or more of said light or heavy chain CDRs may contain one, two, three, four or five amino acid modifications (e.g. substitutions, insertions or deletions). Optionally, provided is an antibody where any of the light and/or heavy chain variable regions comprising part or all of an antigen binding region of antibody 34A3 are fused to an immunoglobulin constant region of the IgG type, optionally a human constant region, optionally a human IgG1 or IgG4 isotype. In another preferred embodiment the antibody is 34A3.

In another aspect, the invention provides a purified polypeptide which encodes a antibody, wherein the antibody comprises: a VHCDR1 region comprising an amino acid sequence as set forth in SEQ ID NO:36, wherein one or more of these amino acids may be substituted by a different amino acid; a VHCDR2 region comprising an amino acid sequence as set forth in SEQ ID NO:37, wherein one or more of these amino acids may be substituted by a different amino acid; a VHCDR3 region comprising an amino acid sequence as set forth in SEQ ID NO:38, wherein one or more of these amino acids may be substituted by a different amino acid; a VLCDR1 region comprising an amino acid sequence as set forth in SEQ ID NO:39, wherein one or more of these amino acids may be substituted by a different amino acid; a VLCDR2 region comprising an amino acid sequence as set forth in SEQ ID NO:40, wherein one or more of these amino acids may be substituted by a different amino acid; a VLCDR3 region comprising an amino acid sequence as set forth in SEQ ID NO:41, wherein one or more of these amino acids may be substituted by a different amino acid.

In still another aspect, the invention provides an antibody, which comprises a heavy chain and/or a light chain each having at least three CDRs, wherein one, two or three of at least three CDRs has the sequence of SEQ ID NO:36 to 38 and 29 to 41 for the respective heavy and light chains, and which antibody specifically binds to TLR3 in acidic conditions.

In another aspect, the invention provides an antibody that binds human TLR3, comprising:
a. the heavy chain variable region of SEQ ID NO: 34, wherein one, two, three or more of these amino acids may be substituted by a different amino acid; or
b. the light chain variable region of SEQ ID NO: 35, wherein one, two, three or more of these amino acids may be substituted by a different amino acid; or
c. the heavy chain variable region of SEQ ID NO: 34, wherein one, two, three or more of these amino acids may be substituted by a different amino acid; and the light chain variable region of SEQ ID NO: 35, wherein one, two, three or more of these amino acids may be substituted by a different amino acid; or
d. the heavy chain CDRs 1, 2 and 3 (HCDR1, HCDR2, HCDR3) amino acid sequences as shown in SEQ ID NO: 36, 37 and 38, wherein one, two, three or more of these amino acids may be substituted by a different amino acid; or
e. the light chain CDRs 1, 2 and 3 (LCDR1, LCDR2, LCDR3) amino acid sequences as shown in SEQ ID NO: 39, 40 and 41, wherein one or more of these amino acids may be substituted by a different amino acid; or
f. the heavy chain CDRs 1, 2 and 3 (HCDR1, HCDR2, HCDR3) amino acid sequences as shown in SEQ ID NO: 36, 37 and 38, wherein one, two, three or more of these amino acids may be substituted by a different amino acid; and the light chain CDRs 1, 2 and 3 (LCDR1, LCDR2, LCDR3) amino acid sequences as shown in SEQ ID NO: 39, 40 and 41, wherein one, two, three or more of these amino acids may be substituted by a different amino acid; or
g. the heavy chain variable region which is at least 60%, 70%, 80%, 85%, 90% or 95% identical to the variable region having an amino acid sequence of SEQ ID NO: 34, wherein one, two, three or more of these amino acids may be substituted by a different amino acid; or
h. the light chain variable region which is at least 60%, 70%, 80%, 85%, 90% or 95% identical to the variable region having an amino acid sequence of SEQ ID NO: 35, wherein one, two, three or more of these amino acids may be substituted by a different amino acid.

In another aspect of any of the embodiments herein, any of the CDRs 1, 2 and 3 of the heavy and light chains may be characterized as having an amino acid sequence that shares at least 50%, 60%, 70%, 80%, 85%, 90% or 95% sequence identity with the particular CDR or set of CDRs listed in the corresponding SEQ ID NO.

In another aspect, the invention provides an antibody that competes for TLR3 binding with a monoclonal antibody of (a) to (h), above.

In any of the antibodies of the invention, e.g., 31C3, 29H3, 23C8, 28F11 or 34A3, the specified variable region and CDR sequences may comprise conservative sequence modifications. Conservative sequence modifications refers to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the properties set forth herein) using the assays described herein.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

The sequences of the CDRs of the antibodies according to the invention, according to AbM (Oxford Molecular's AbM antibody modelling software definition), Kabat and Chothia definitions systems, have been summarized in Table A below. The amino acids sequences described herein are numbered according to Abm, Kabat and Chothia numbering systems. While any suitable numbering system may be used to designated CDR regions, in the absence of any other indication, the numbering used herein is Abm. Such numbering has been established using the following indications: CDR-L1: Start: approx residue 24, residue before: always a Cys, residue after: always a Trp (typically Trp-Tyr-Gln, but also, Trp-Leu-Gln, Trp-Phe-Gln, Trp-Tyr-Leu), length: 10 to 17 residues; CDR-L2: Start: always 16 residues after the end of L1, Residues before: generally Ile-Tyr (but also, Val-Tyr, Ile-Lys, Ile-Phe), Length: always 7 residues; CDR-L3, Start: always 33 residues after end of L2, Residue before: always Cys, Residues after: always Phe-Gly-Xaa-Gly, Length: 7 to 11 residues; CDR-H1, Start: approx residue 26 (always 4 after a Cys) (Chothia/AbM definition, the Kabat definition starts 5 residues later), Residues before: always Cys-Xaa-Xaa-Xaa, Residues after: always a Trp (typically Trp-Val, but also, Trp-Ile, Trp-Ala), Length: 10 to 12 residues (AbM definition, Chothia definition excludes the last 4 residues); CDR-H2, Start: always 15 residues after the end of Kabat/AbM definition of CDR-H1, Residues before: typically Leu-Glu-Trp-11e-Gly (but a number of variations, Residues after Lys/Arg-Leu/Ile/Val/Phe/Thr/Ala-Thr/Ser/Ile/Ala), Length: Kabat definition 16 to 19 residues; AbM (and Chothia) definition ends 7 residues earlier; CDR-H3, Start: always 33 residues after end of CDR-H2 (always 2 after a Cys), Residues before: always Cys-Xaa-Xaa (typically Cys-Ala-Arg), Residues after: always Trp-Gly-Xaa-Gly, Length: 3 to 25 residues.

The sequences of the variable chains of the antibodies according to the invention are listed in Table B below, signal peptide sequence is represented in italics, and the CDRs are provided in bold. The term x/x indicates that any of the two indicated amino acids can be present at the particular amino acid residue, for instance, the term F/S means that at the given position, the amino acid can be either phenylalanine or serine. In any embodiment herein, a VL or VH sequence can be specified or numbered so as to contain or lack the signal peptide or any part thereof.

In an embodiment, the antibodies of the invention are of the human or mouse IgG1 isotype. In another embodiment, the antibodies of the invention are of the human IgG4 isotype In an embodiment, the antibodies of the invention are antibody fragments that retain their binding and/or functional properties.

TABLE A

| | CDR | HCDR1 | | HCDR2 | | HCDR3 | |
|---|---|---|---|---|---|---|---|
| | defini- | SEQ | | SEQ | | SEQ | |
| mAb | tion | ID | Sequence | ID | Sequence | ID | Sequence |
| 23C8 | Abm | 28 | GYSFTGYFMH | 29 | RINPYNGATS | 30 | DDGGNYPFDY |
| | Chotia | | GYSFTG | | RINPYNGATS | | DDGGNYPFDY |
| | Kabat | | GYFMH | | RINPYNGATSYNQNF | | DDGGNYPFDY |
| | | | | | KD | | |
| 29H3 | Abm | 12 | GYSITSGYSWH | 13 | YIHYSGITN | 14 | DGYYGMDY |
| | Chotia | | GYSITSG | | YIHYSGITN | | DGYYGMDY |
| | Kabat | | SGYSWH | | YIHYSGITNYNPSLRS | | DGYYGMDY |
| 28F11 | Abm | 20 | GYSFTGYYIH | 21 | RINPYYGAT | 22 | STKLGYLDY |
| | Chotia | | GYSFTG | | RINPYYGAT | | STKLGYLDY |
| | Kabat | | GYYIH | | RINPYYGATSNQNFK | | STKLGYLDY |
| | | | | | D | | |
| 31C3 | Abm | 4 | GYSFTAYYMH | 5 | RINPYNGATS | 6 | SGGNTYFDY |
| | Chotia | | GYSFTA | | RINPYNGATS | | SGGNTYFDY |
| | Kabat | | AYYMH | | RINPYNGATSYNRNF | | SGGNTYFDY |
| | | | | | KD | | |
| 34A3 | Abm | 36 | GYVFTTYSIY | 37 | YIDPYNGDTS | 38 | EGNYYGYFDY |
| | Chotia | | GYVFTT | | YIDPYNGDTS | | EGNYYGYFDY |
| | Kabat | | TYSIY | | YIDPYNGDTSYNQKF | | EGNYYGYFDY |
| | | | | | KG | | |

TABLE B

| | SEQ | |
|---|---|---|
| Antibody | ID | |
| portion | NO | |
| 23C8 VL | 27 | MSVPTQVLGL LLLWLTGARC DIQMTQSPAS LSASVGETVT ITCRASENIY |
| | | SYLAWYQQKQ GKSPQLLVYY AKTLAEGVPS RFSGSGTGTQ FSLKINSLQP |
| | | EDFGSYYCQH HYGTPYTFGG GTKLEIK |
| 23C8 VH | 26 | MGWSWIFLFL LSGTAGVLSE VQLQQSGPEL VKPGASVKIS CKASGYSFTG |
| | | YFMHWVKQSH VKSLEWIGRI NPYNGATSYN QNFKDKASLT VDKSSSTSYM |
| | | ELHSLTSEDS AVYYCVRDDG GNYPFDYWGQ GTTLTVS |
| 28F11VL | 19 | MSVPTQVLGL LLLWLTGARC DIQMTQSPAS LSASVGETVT ITCRASENIY |
| | | SNLAWYQQKQ GKSPQLLIYN AKTLAEGVPS RFSGSGSGTQ YFLKINSLQP |
| | | EDFGSYYCQH HYGTPFTFGG GTKLEIK |
| 28F11 VH | 18 | MGWSWIFLFL LSGTAGVLSE VQLQQSGPEL VKPGASVKIS CKASGYSFTG |
| | | YYIHWVKQSH VKSLEWIGRI NPYYGATSNQ NFKDKANLTV DKSSSTAYME |
| | | LHSLTSDDSA VYYCARSTKL GYLDYWGQGT TLTVS |
| 29H3 VL | 11 | MDFQTQVFVF VLLWLSGVDG DIVMTQSQKF MSTSVGDRVS ITCKASQNVR |
| | | TSVAWYQQKP GQSPKALIYL ASNRHTGVPD RFTGSGSGTD FTLTVSNIQS |
| | | EDLADYFCLQ HWNYPYTFGG GTKLEIK |
| 29H3 VH | 10 | MRVLILLCLF TAFPGILSDV QLQESGPDLV KPSQSLSLTC TVTGYSITSG |
| | | YSWHWIRQFL GNKLEWMGYI HYSGITNYNP SLRSRISFTR DTSKNQFFLQ |
| | | LNSVTTEDTA TYYCARDGYY GMDYWGQGTS VTVS |
| 31C3 VL | 3 | MSVPTQVLGL LLLWLTGARC DIQMTQSPAS LSASVGETVT ITCRASENIY |
| | | SSLAWYQQKQ GKSPQLLVYN AKTLAEGVPS RFSGSGSGTQ |
| | | F/SSLKINSLQP EDFGTYYCQH HYGTPPTFGG GTKLEIK |
| 31C3 VH | 2 | MGWSWIFLFL LSGTAGVLSE VQLQQSGPEL VKPGASVKIS CKPSGYSFTA |
| | | YYMHWVKQSH VKSLEWIGRI NPYNGATSYN RNFKDKASLT VDKSSSTAYM |
| | | ELHSLTSEDS AVYYCARSGG NTYFDYWGQG TTLTVS |
| 34A3 VL | 35 | MDFQVQIFSF LLMSASVIMS RGQIVLTQSP ALMSASPGEK |
| | | VTMTCSASSSV SYMFWYQQKP RSSPKPWIYL TSNLASGVPA |
| | | RFSGSGSGTS YSLTISSMEA EDAATYYCQQ WTGNPPTFGG GTKLEIK |
| 34A3 VH | 34 | MEWRWIFLFL LSGTTGVHSE IQLQQSGPEL VKPGASVKVS CKASGYVFTT |
| | | YSIYWVKQSH GKSLEWIGYI DPYNGDTSYN QKFKGKATLT VDKSSSTAYM |
| | | HLNSLTSEDS TVYYCAREGN YYGYFDYWGQ GTTLTVS |

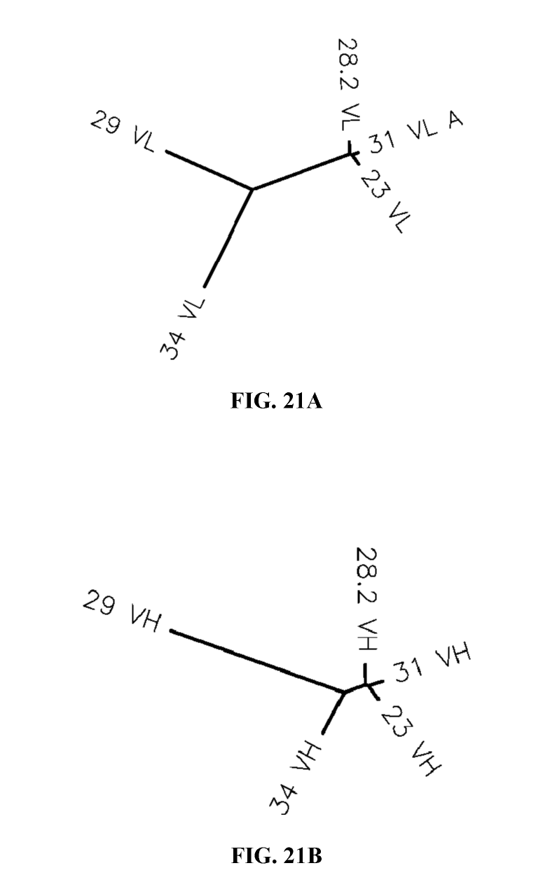

The sequencing of light and heavy chains of the anti-TLR3 antibodies according to the invention led to the identification of the genes rearrangement involved in the generation of such antibodies, as summarized in Table C below. (The gene sequences indicated are can be retrieved at http://www.ncbi.nlm.nih.igov/igblast/showGermline.cgi). FIGS. 21A and 21B represent the phylogenetic trees (generated by Phylip's Drawtree) of the light and heavy chains, indicating a high degree of homology between antibodies 28F11, 31C3 and 23C8.

TABLE C

| | Light Chain | | Heavy Chain | |
|---|---|---|---|---|
| | V gene | J gene | V gene | J gene |
| 29H3 | VK 19-14 | JK2 | VH3 VH36-60.a1.85 | JH4 |
| | SEQ ID NO: 42 | SEQ ID NO: 45 | SEQ ID NO: 46 | SEQ ID NO: 50 |
| 34A3 | VK aq4 (VK) | JK2 | VH1 VH J558.1 | JH2 |
| | SEQ ID NO: 43 | SEQ ID NO: 45 | SEQ ID NO: 47 | SEQ ID NO: 49 |
| 23C8 | VK 12-41 or 12-44 | JK2 | VH1 VH J558.2 | JH2 |
| | SEQ ID NO: 44 | SEQ ID NO: 45 | SEQ ID NO: 48 | SEQ ID NO: 49 |
| 31C3 | VK 12-41 or 12-44 | JK2 | VH1 VH J558.2 | JH2 |
| | SEQ ID NO: 44 | SEQ ID NO: 45 | SEQ ID NO: 48 | SEQ ID NO: 49 |
| 28F11 | VK 12-41 or 12-44 | JK2 | VH1 VH J558.2 | JH2 |
| | SEQ ID NO: 44 | SEQ ID NO: 45 | SEQ ID NO: 48 | SEQ ID NO: 49 |

Table D below provides the percentage sequence identity between the different CDRs for each antibody, in amino acids.

TABLE D

| | | | | |
|---|---|---|---|---|
| 31C3 | 29H3 | 23C8 | 28F11 | 34A3 |

CDR VL

| | | | | | |
|---|---|---|---|---|---|
| 31C3 | 100.00 | 33.33 | 88.89 | 92.59 | 37.04 |
| 29H3 | | 100.00 | 33.33 | 29.63 | 29.63 |
| 23C8 | | | 100.00 | 88.89 | 33.33 |
| 28F11 | | | | 100.00 | 33.33 |
| 34A3 | | | | | 100.00 |

CDR1 VL

| | | | | | |
|---|---|---|---|---|---|
| 31C3 | 100.00 | 45.45 | 90.91 | 90.91 | 27.27 |
| 29H3 | | 100.00 | 45.45 | 27.27 | 27.27 |
| 23C8 | | | 100.00 | 90.91 | 27.27 |
| 28F11 | | | | 100.00 | 36.36 |
| 34A3 | | | | | 100.00 |

CDR2 VL

| | | | | | |
|---|---|---|---|---|---|
| 31C3 | 100.00 | 14.29 | 85.71 | 100.00 | 28.57 |
| 29H3 | | 100.00 | 14.29 | 14.29 | 42.86 |
| 23C8 | | | 100.00 | 85.71 | 28.57 |
| 28F11 | | | | 100.00 | 28.57 |
| 34A3 | | | | | 100.00 |

CDR3 VL

| | | | | | |
|---|---|---|---|---|---|
| 31C3 | 100.00 | 33.33 | 88.89 | 85.71 | 33.33 |
| 29H3 | | 100.00 | 44.44 | 27.27 | 22.22 |
| 23C8 | | | 100.00 | 88.89 | 44.44 |
| 28F11 | | | | 100.00 | 44.44 |
| 34A3 | | | | | 100.00 |

CDR VH

| | | | | | |
|---|---|---|---|---|---|
| 31C3 | 100.00 | 39.47 | 78.38 | 70.27 | 50.00 |
| 29H3 | | 100.00 | 44.74 | 39.47 | 39.47 |
| 23C8 | | | 100.00 | 70.27 | 52.63 |
| 28F11 | | | | 100.00 | 47.37 |
| 34A3 | | | | | 100.00 |

CDR1 VH

| | | | | | |
|---|---|---|---|---|---|
| 31C3 | 100.00 | 54.55 | 80.00 | 80.00 | 45.45 |
| 29H3 | | 100.00 | 63.64 | 63.64 | 45.45 |
| 23C8 | | | 100.00 | 80.00 | 45.45 |
| 28F11 | | | | 100.00 | 54.55 |
| 34A3 | | | | | 100.00 |

CDR2 VH

| | | | | | |
|---|---|---|---|---|---|
| 31C3 | 100.00 | 35.29 | 94.12 | 82.35 | 64.71 |
| 29H3 | | 100.00 | 35.29 | 29.41 | 35.29 |
| 23C8 | | | 100.00 | 88.24 | 70.59 |
| 28F11 | | | | 100.00 | 58.82 |
| 34A3 | | | | | 100.00 |

CDR3 VH

| | | | | | |
|---|---|---|---|---|---|
| 31C3 | 100.00 | 40.00 | 50.00 | 44.44 | 40.00 |
| 29H3 | | 100.00 | 40.00 | 30.00 | 40.00 |
| 23C8 | | | 100.00 | 30.00 | 30.00 |
| 28F11 | | | | 100.00 | 20.00 |
| 34A3 | | | | | 100.00 |

Table E provides the identity percentage has been calculated between the different VL and VH (italics) nucleotide sequences for each antibody using LALIGN software.

TABLE E

| | VH | | | | |
|---|---|---|---|---|---|
| VL | 23C8 | 28F11 | 29H3 | 31C3 | 34A3 |
| 23C8 | 100 | 93.5 | 60.6 | 94.9 | 87.3 |
| 28F11 | 97.6 | 100 | 60.4 | 94.4 | 87.8 |
| 29H3 | 65.8 | 65.2 | 100 | 61.0 | 60.6 |
| 31C3 | 96.1 | 95.5 | 63.9 | 100 | 89.1 |
| 34A3 | 64.6 | 65.3 | 67.2 | 64.0 | 100 |

Table F provides the identity percentage has been calculated between the different VL and VH (italics) amino acid sequences using LALIGN software.

TABLE F

| | VH | | | | |
|---|---|---|---|---|---|
| VL | 23C8 | 28F11 | 29H3 | 31C3 | 34A3 |
| 23C8 | 100 | 89.8 | 48.9 | 92.7 | 79.1 |
| 28F11 | 94.5 | 100 | 47.0 | 91.2 | 79.6 |
| 29H3 | 54.3 | 54.3 | 100 | 47.8 | 48.1 |
| 31C3 | 96.1 | 95.3 | 55.1 | 100 | 79.6 |
| 34A3 | 52.7 | 53.5 | 55.4 | 54.3 | 100 |

### Fragments and Derivatives of the Present Monoclonal Antibodies

Fragments and derivatives of antibodies of this invention (which are encompassed by the term “antibody” or “antibodies” as used in this application, unless otherwise stated or clearly contradicted by context), preferably a 31C3, 29H3, 23C8, 28F11 or 34A3-like antibody, can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Included, inter alia, are a nanobody, domain antibody, single domain antibody or a “dAb”.

Fragments of the present antibodies can be obtained using standard methods. For instance, Fab or F (ab′) 2 fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al, 16 (3): 106-119 (2001) and Delgado et al, Br. J. Cancer 73 (2): 175-182 (1996), the disclosures of which are incorporated herein by reference.

Alternatively, the DNA of a hybridoma producing an antibody of the invention, preferably a 31C3, 29H3, 23C8, 28F11 or 34A3-like antibody, may be modified so as to encode a fragment of the invention. The modified DNA is then inserted into an expression vector and used to transform or transfect an appropriate cell, which then expresses the desired fragment.

In certain embodiments, the DNA of a hybridoma producing an antibody of this invention, preferably a 31C3, 29H3, 23C8, 28F11 or 34A3-like antibody, can be modified prior to insertion into an expression vector, for example, by substituting the coding sequence for human heavy- and light-chain constant domains in place of the homologous non-human sequences (e.g., Morrison et al., PNAS pp. 6851 (1984)), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention.

Thus, according to another embodiment, the antibody of this invention, preferably a 31C3, 29H3, 23C8, 28F11 or 34A3-like antibody, is humanized. “Humanized” forms of antibodies according to this invention are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies of this invention are well known in the art.

The choice of human variable domains, both light and heavy, to be used in making the humanized antibodies is very important to reduce antigenicity. According to the so-called “best-fit” method, the sequence of the variable domain of an antibody of this invention is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for TLR3 receptors and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, Calif.) as the mouse used for immunization. A XenoMouse is a murine host according to this invention that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et Nature 362 (1993) 255), or by selection of antibody repertoires using phage display methods. Such techniques are known to the skilled person and can be implemented starting from monoclonal antibodies as disclosed in the present application.

The antibodies of the present invention, preferably a 31C3, 29H3, 23C8, 28F11 or 34A3-like antibody, may also be derivatized to “chimeric” antibodies (immunoglobulins) in which a portion of the heavy/light chain(s) is identical with or homologous to corresponding sequences in the original antibody, while the remainder of the chain (s) is identical with or homologous to corresponding sequences in antibodies derived from another species or belonging to another antibody class or subclass, as well as fragments of such antibodies, so long as they exhibit the desired biological activity and binding specificity (Cabilly et al., supra; Morrison et al., Proc. Natl. Acad. Sci. U.S.A., pp. 6851 (1984)).

### Dosage Forms

Therapeutic formulations of the antagonists used in accordance with the present invention are prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al. (eds.), The Pharmacological Bases of Therapeutics, 8^{th} Ed. (Pergamon Press, 1990); Gennaro (ed.), Remington's Pharmaceutical Sciences, 18^{th} Edition (Mack Publishing Co., Easton, Pa., 1990); Avis et al. (eds.), Pharmaceutical Dosage Forms: Parenteral Medications (Dekker, New York, 1993); Lieberman et al. (eds.), Pharmaceutical Dosage Forms: Tablets (Dekker, New York, 1990); Lieberman et al. (eds.) Pharmaceutical Dosage Forms: Disperse Systems (Dekker, New York, 1990); and Walters (ed.), Dermatological and Transdermal Formulations (Drugs and the Pharmaceutical Sciences), Vol 119 (Dekker, New York, 2002).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as ethylenediaminetetraacetic acid (EDTA); sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™, or PEG.

Exemplary antibody formulations are described for instance in WO 1998/56418, which describes a liquid multidose formulation for an anti-CD20 antibody, comprising 40 mg/mL rituximab, 25 mM acetate, 150 mM trehalose, 0.9% benzyl alcohol, and 0.02% polysorbate20™ at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate80™, and Sterile Water for Injection, pH 6.5.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antagonist are also contemplated. See, for example, US 2002/0136719A1 (Shenoy et al.).

The formulation herein may also contain more than one active compound (a second medicament as noted above), preferably those with complementary activities that do not adversely affect each other. The type and effective amounts of such medicaments depend, for example, on the amount and type of B-cell antagonist present in the formulation, and clinical parameters of the subjects. The preferred such second medicaments are noted above.

The active ingredients may also be entrapped in microcapsules prepared, e.g., by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra, for example.

Sustained-release formulations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes. Pharmaceutically acceptable carriers that may be used in these compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The antibodies of this invention may be employed in a method of modulating, e.g. inhibiting, the activity of TLR3-expressing cells in a patient. This method comprises the step of contacting said composition with said patient. Such method will be useful for both prophylaxis and therapeutic purposes.

Formulations may be adapted to nasal or inhalation routes. A formulation may comprise a pharmaceutically acceptable nasal carrier. For nasal delivery, any well known delivery methods such as drops, a nasal spray, a nasal liquid or powder aerosol, a capsule or a nasal insert can be used. For aerosol delivery, any well known delivery methods such as a nebulizer, inhaler, atomizer, aerosolizer, mister, dry powder inhaler, metered dose inhaler, metered dose sprayer, metered dose mister, metered dose atomizer, or other suitable delivery device can be used.

Further aspects and advantages of this invention will be disclosed in the following experimental section, which should be regarded as illustrative and not limiting the scope of this application.

### EXAMPLES

### Materials and Methods

Interferon-alpha (IntronATM) was purchased from Schering Plough Corp. Tumor Cell lines: A375 malignant melanoma tumor cell lines (CRL-1619) and 293T Human Embryonic Kidney cells (#CRL-1573) are purchased from ATCC. Antibodies (antigen, supplier, reference): Anti-TLR3 antibody pAb, R&D Systems, ref. AF1487, anti-TLR3 antibody mAb clone TLR3.7 from eBioscience, anti-TLR3 mAb from Imgenex clone 40C1285. Instrumentation: FACSCalibur™ flow cytometer (BD Biosciences). PolyAU, also referred to as IPH3102, is an at least partially double stranded molecule made of polyadenylic acid(s) and polyuridylic acid(s), prepared as described in WO2009/130616 (Innate Pharma), the disclosure of which is incorporated herein by reference. PolyAU was a high molecular weight polyAU having an Mₙ (also referred to as “number average molecular weight” or “mean molecular weight”) above 2000 kD, a PI of 1.4-1.6, and thermal stability: 62.3-63.2° C., hyperchromicity of 53-60%.

### Surface Plasmon Resonance (SPR)

(a) General Biacore T100 methods. SPR measurements were performed on a Biacore T100 apparatus (Biacore GE Healthcare) at 25° C. In all Biacore experiments HBS-EP+ buffer (Biacore GE Healthcare) or 10 mM sodium acetate pH 5.6, 150 mM NaCl, 0.05% P20 served as running buffer and sensorgrams were analyzed with Biaevaluation 4.1 and Biacore T100 Evaluation software. Recombinant human and mouse TLR3 were purchased from R&D Systems.
(b) Protein immobilization. Recombinant TLR3 protein was immobilized covalently to carboxyl groups in the dextran layer of a Biacore Series 5 Sensor Chip CM5 (chip). The chip surface was activated with EDC/NHS (0.2M N-ethyl-N′-(3-dimethylaminopropyl) carbodiimidehydrochloride, 0.05M N-hydroxysuccinimide (Biacore GE Healthcare)). Proteins were diluted to 10 μg/ml in coupling buffer (10 mM sodium acetate, pH 5.6) and injected until the appropriate immobilization level was reached (i.e. approximately 2000 RU for binding experiments and 600 RU for affinity experiments). Deactivation of the remaining activated groups was performed using 100 mM ethanolamine pH 8 (Biacore GE Healthcare).
(c) Antibody binding analysis was run using HBS-EP+ (neutral pH). Antibodies at a concentration of 10 μg/ml were injected for 2 min at a constant flow rate of 10 μl/min over the immobilized proteins and allowed to dissociate for 3 min before regeneration by a ten second injection of 10 mM NaOH, 500 mM NaCl regeneration buffer. Blank correction was performed on line by co-injecting the soluble antibodies onto the reference dextran flow cell.
(d) Competition assay in acidic buffer (pH 5.6). Flow rate was set to 10 μl/min, the first antibody at a concentration of 50 μg/ml (or polyAU at 100 μg/ml) was injected for 2 min, 3 times successively in order to saturate the rhTLR3 surface. The second antibody (or polyAU at 100 μg/ml when the polyAU was added after first antibody) was then injected for 2 min also at 50 μg/ml and allowed to dissociate for 3 min before regeneration by a 15 second injection of 10 mM NaOH, 500 mM NaCl regeneration buffer. Blank correction was also performed on line and the curve using the saturating antibody (or neucleic acid) followed by an injection of buffer subtracted to remove the signal due to the dissociation of the first complex. The resulting signal was compared to that obtained by the injection of the second antibody directly onto the rhTLR3 surface.

### Luciferase Reporter Assay.

A reporter gene assay using as promoter ISRE (IFN-stimulated response element) and as reporter gene and protein luciferase was set up. A 293T cell line (ATCC, #CRL-1573) was stably transfected with pISRE-luc plasmid (#219089—Stratagene), further selected by cloning as inducing optimal response to IFN-alpha stimulation and referred to as control 293T-ISRE. This cell line was further stably transfected with pUNO-humanTLR3 plasmid (#puno-htlr3—InVivogen) or pUNO-mouseTLR3 plasmid (#puno-mtlr3—InVivogen) and referred to as 293T-TLR3-ISRE and 293T-mTLR3-ISRE respectively. On day 0, cells are seeded at 4×105 cells/mL in complete culture medium in 96-well culture plate (100 μl/well). Cells are first incubated at 37° C. for 20 hours, then 50 μL of medium are discarded and cells are activated with 100 μL/well final of increasing amounts of polyAU together with various concentrations of anti-TLR3 antibodies. Cells incubated with fresh medium will be used as background luciferase activity. Cells are incubated at 37° C. for 6 hours. 100 μL of freshly thawed Steady Glo (Promega) are added to each well, plates were incubated 10 min at RT in the dark and the light emitted in each well is quantified as Count Per Second (CPS) on a gamma-counter (TopCount) apparatus.

### MdDC Secretion and Expression Inhibition In Vitro Tests.

Myeloid DC (MdDC) were obtained from PBMC by isolating PBMC from normal healthy human donors. Monocytes were purified from PBMC using positive selection with human CD14 microbeads (Miltenyi Biotech) following general instructions. Monocytes were further derived into DC (MdDC) by 5-6 days incubation in human GM-CSF (Leucomax, SP) and human IL-4 (R&D Systems) at respectively 200 ng/ml and 20 ng/ml.

The resulting MdDC were then seeded at 10⁶ cells/ml, in duplicate, in flat bottom 96-well plates. Cells were activated for 20 hours in a final volume of 200 μl, together with the anti-TLR3 antibodies at the indicated concentrations. Increasing amounts of polyAU were added to the wells to obtain a dose effect read-out at the dose and timepoint as indicated.

Supernatants were collected after 20 h of stimulation, frozen at −20° C., and further assayed for IL-6 and IP-10 using Enzyme-linked immunosorbent assay. Cells were then harvested, stained for activation markers CD86 (with a.huCD86 mouse, IgG1, FITC, BD Biosciences, ref 555657), with detection using FACSCanto™ flow cytometer (BD Biosciences).

### Generation of Mutant TLR3 Constructs

The generation of the TLR3 mutants K145E, D116R, K182E, N196A and E171A was performed using the Stratagene's QuikChange® Site-Directed Mutagenesis Kit according to the manufacturer instructions. The oligonucleotides used are listed in Table A. Mutagenesis was performed on wild type human TLR3 inserted into a pcDNA3.1 vector. After sequencing, the vectors containing the mutated sequences were prepared as Maxiprep using the Promega PureYield™ Plasmid Maxiprep System. Vectors were then used for HEK-293T cell transfection using Invitrogen's Lipofectamine 2000 according to the manufacturer instructions.

TABLE A

| Oligonucleotide name | Sequence |
|---|---|
| K145E forward | 5′ GAA AAT TAA AAA TAA TCC CTT TGT CGA GCA |
| | GAA GAA TTT AAT CAC ATT AG 3′ (SEQ ID NO 86) |
| K145E reverse | 5′ CT AAT GTG ATT AAA TTC TTC TGC TCG ACA |
| | AAG GGA TTA TTT TTA ATT TTC 3′ (SEQ ID NO 87) |
| D116R forward | 5′ CAA TGA GCT ATC TCA ACT TTC TCG TAA AAC |
| | CTT TGC CTT CTG CAC 3′ (SEQ ID NO 88) |
| D116R reverse | 5′ GTG CAG AAG GCA AAG GTT TTA CGA GAA AGT |
| | TGA GAT AGC TCA TTG 3′ (SEQ ID NO 89) |
| K182E forward | 5′ CAA GAG CTT CTA TTA TCA AAC AAT GAG ATT |
| | CAA GCG CTA AAA AGT GAA G 3′ (SEQ ID NO 90) |
| K182E reverse | 5′ C TTC ACT TTT TAG CGC TTG AAT CTC ATT GTT |
| | TGA TAA TAG AAG CTC TTG 3′ (SEQ ID NO 91) |
| N196A forward | 5′ GAA GAA CTG GAT ATC TTT GCC GCT TCA TCT |
| | TTA AAA AAA TTA GAG TTG 3′ (SEQ ID NO 92) |
| N196A reverse | 5′ CAA CTC TAA TTT TTT TAA AGA TGA AGC GGC |
| | AAA GAT ATC CAG TTC TTC 3′ (SEQ ID NO 93) |
| E171A forward | 5′ GGA ACT CAG GTT CAG CTG GCC AAT CTC CAA |
| | GAG CTT CTA TTA TCA 3′ (SEQ ID NO 94) |
| E171A reverse | 5′ TGA TAA TAG AAG CTC TTG GAG ATT GGC CAG |
| | CTG AAC CTG AGT TCC 3′ (SEQ ID NO 95) |

### Example 1

### Generation of TLR3-Specific Monoclonal Human Antibodies

### Immunization Series #1

Primary screen. To obtain anti-human TLR3 antibodies, Balb/c mice were immunized with a recombinant human His-tagged TLR3 extracellular domain recombinant protein (R&D systems, #1487-TR-050). Mice received one primo-immunisation with an emulsion of 50 μg TLR3 protein and Complete Freund Adjuvant, intraperitoneally, a 2^{nd} immunization with an emulsion of 50 μg TLR3 protein and Incomplete Freund Adjuvant, intraperitoneally, and three boosts with 10 μg TLR3 protein, intravenously. Immune spleen cells were fused with X63.Ag8.653 immortalized B cells, and cultured in the presence of irradiated spleen cells. 40 culture plates were obtained and evaluated in a first screen for TLR3 binding using an ELISA developed for detection of binding to TLR3. Briefly, His-tagged recombinant TLR3 protein (R&D systems, #1487-TR-050) was coated on Ni-NTA 96-wells plates (Qiagen). Supernatant (SN) from hybridoma culture plates and incubated in TLR3-plates, and the presence of TLR3 binding Ig was revealed with goat anti-mouse F(ab) IgG-HRP. Positive supernatants were selected and tested for lack of binding to TLR4. Briefly, His-tagged rec TLR4 protein (R&D systems, 43146-TR-050) were coated on Ni-NTA 96-wells plates (Qiagen). SN from hybridoma culture plates were incubated in TLR4-plates, and the presence of TLR4 binding Ig was revealed with goat anti-mouse F(ab) IgG-HRP. TLR4 was chosen as a 2^{nd} screen in order to discriminate among wells selected in the 1^{st} screen, where anti-His specific antibody from TLR3 specific antibody were used. Secondly, given the homology between TLR3 and other members of TLR family, the initial assessment demonstrated that at least one commercially available monoclonal antibody (mAb) indicated on its packaging as specific for TLR3 protein nevertheless recognized paraffin-embedded 293T cells stably transfected with TLR4.

Secondary screen; selection of hybridomas of interest. 168 supernatants were retained and tested in a further screen in a Biacore assay using rec TLR3 chips, followed by various assays formats based on binding to human TLR3-expressing 293T cells. A 293T cell line (ATCC, #CRL-1573), stably transfected with pISRE-luc plasmid (#219089—Stratagene), was further selected as inducing optimal response to IFN-alpha stimulation and referred to as control 293T cells. This cell line was further stably transfected with pUNO-hTLR3 plasmid (#puno-htlr3—InVivogen), or pUNO-hTLR4 plasmid (#puno-tlr4—InVivogen) and referred to respectively as 293T-TLR3 and 293T-TLR4. Supernatants were screened in a FACS based screen for binding to 293T-TLR3 cells with no binding to 293T control cells, and in parallel in an IHC screen for binding to 293T-TLR3 cells as a frozen cell pellet, with no binding to 293T-TLR4 cells. Briefly, for FACS screening, the presence of reacting antibodies in supernanants were revealed by Goat anti-mouse polycolonal antibody (pAb) labeled with biotin, streptavidin labeled with PE. For IHC screening, presence of reacting antibodies (Abs) in supernanants were revealed by donkey anti-mouse pAb labeled with biotin (#715-065-150, Jackson Immunoresearch Laboratories), streptavidin labeled with peroxydase (#E2886, SIGMA) and revealed with DAB (#SK-4105, Vector Laboratories).

Cloning of hybridomas of potential interest. 42 potentially interesting hybridomas selected from the initial screening were cloned by limiting dilution techniques in 96-wells plates, and 304 subclones were tested in a series of screens as follows. The 304 subclones were first evaluated in a screen for TLR3 binding using the same ELISA developed for detection of binding to TLR3, and positive supernatants were selected and tested for lack of binding to TLR4 in ELISA assay, yielding 228 clones which were selective for TLR3. All supernatants yielding a ratio above 10 for DO obtained in TLR3 ELISA to DO obtained in TLR4 ELISA were selected as specific for TLR3.

Among the 304 clones, 63 clones, selected as issuing from preclones tested positive in frozen IHC, were also tested in a frozen IHC screen for binding to 293T-TLR3 cells as a frozen cell pellet, with no binding to 293T-TLR4 cells, yielding 31 positive clones in frozen IHC.

Among 71 clones positive in FACS staining and the 31 clones positives in frozen IHC, 41 clones were selected for cryopreservation from the 304 initial clones. Those 41 clones were tested in a further screen in an inhibition test on IP-10, IL-6 and IL12p40 secretion, in response to a TLR3 specific ligand, poly(A:U) dsRNA. Clones having the highest inhibitory effect were selected. Among them were supernatants from well C3 of plate 31 (31C3), well H3 of plate 29 (29H3), well C8 of plate 23 (23C8) and well F11 of plate 28 (28F11). 31C3, 29H3, 23C8 and 28F11 are of IgG1 isotype. We further selected as a negative control in functional inhibitory assay well E7 of plate 23 (23E7).

### Immunization Series #2

Primary screen. A further series of immunization were carried out in order to generate different antibodies. Using an experimental setup similar to that of the first immunization series, Balb/c mice were immunized, immune spleen cells were fused and cultured in the presence of irradiated spleen cells. Culture plates were obtained and evaluated in a first screen for TLR3 binding using an ELISA developed for detection of binding to TLR3. 263 clones out of 2840 were selected for the secondary screen.

Secondary screen; selection of hybridomas of interest. 263 supernatants were retained and tested in a further screen in an inhibition test on 293T-TLR3 cells. Wells from supernatants having an inhibitory effect superior to 95% were selected for further cloning.

Cloning of hybridomas of potential interest. 4 potentially interesting hybridomas selected from the initial screening were cloned by limiting dilution techniques in 96-wells plates, and 17 subclones were tested in a series of screens as follows. The 304 subclones were first evaluated in a screen for TLR3 binding using an ELISA developed for detection of binding to TLR3. The 7 positive clones were tested in a further screen in an inhibition test on 293T-TLR3 cells as above. Among them was supernatant from well A3 of plate 34 (34A3).

### Immunization #3

A further series of immunization were carried out in order to generate different antibodies that bind to K182 on human TLR3. The primary and secondary screens are as follows.

Primary screen. Using an experimental setup similar to that of the first immunization series, rats were immunized, immune spleen cells were fused and cultured in the presence of irradiated spleen cells. Culture plates were obtained and evaluated in a first screen for TLR3 binding using an ELISA developed for detection of binding to TLR3. 279 clones out of 2000 were selected for the secondary screen.

Secondary screen; selection of hybridomas of interest. 279 supernatants were retained and tested in a further screen by FACS staining using a D116R transiently expressing 293T cell line to identify antibodies that do not lose binding to the mutant TLR3 at position 116. 144 clones on the 279 did not lose binding to D116R. Clones were also tested in a TLR3 inhibition test on 293T-TLR3 cells for TLR3 antagonist activity. Wells from supernatants having an inhibitory effect superior to 85% were selected for further cloning. 59 clones had antagonist activity and 43 clones had both antagonist activity and D116R binding.

Cloning of hybridomas of potential interest. Potentially interesting hybridomas from the initial screening were cloned by limiting dilution techniques in 96-wells plates, andsubclones were tested in the same series of secondary screens as for hybridomas. Clones from 14 hybridomas had both antagonist activity and D116R binding.

Clones were further assessed for epitope binding to mutant TLR3 K145E and K182E (for methods, see Example 17). 12 of 14 antibodies did not show any loss of binding to the K145E variant of TLR3 but showed loss in binding to the mutant K182E. 2 of the 14 antibodies did not show any loss of binding to either of the K145E and K182 variant of TLR3.

### Example 2

### Generation of TLR3-Specific Monoclonal Rat Anti-Mouse Antibodies

Primary screen. To obtain anti-TLR3 antibodies, LOU/c rats were immunized with a recombinant His-tagged mouse TLR3, carrier free extracellular domain recombinant protein (R&D systems, #3005-TR) and recombinant His-tagged human TLR3, carrier free extracellular domain recombinant protein (R&D systems, #1487-TR). Rats received, on day 0, one primo-immunisation with an emulsion of 50 μg of mouse TLR3+50 μg of human TLR3 diluted in PBS and Complete Freund Adjuvant, intraperitoneally, a 2^{nd} immunization on day 14 with an emulsion of 50 μg of mouse TLR3+50 μg of human TLR3 diluted in PBS and Incomplete Freund Adjuvant, intraperitoneally, and one boost with 25 μg of mouse TLR3+25 μg of human TLR3 diluted in PBS, intravenously. Immune spleen cells were fused with X63.Ag8.653 immortalized B cells, and cultured in the presence of irradiated spleen cells.

40 culture plates were obtained and evaluated in a first screen for mouse TLR3 binding using an ELISA developed for detection of binding to TLR3. Briefly, His-tagged recombinant mouse TLR3 protein (R&D systems, #1487-TR-050) was coated on Ni-NTA 96-wells plates (Qiagen). Supernatant (SN) from hybridoma culture plates and incubated in TLR3-plates, and the presence of TLR3 binding Ig was revealed with goat anti-mouse F(ab) IgG-HRP.

Secondary screen: selection of hybridomas of interest. 181 supernatants were retained and tested in a further screen in an inhibition test on 293T-mTLR3 cells. Wells from supernatants having an inhibitory effect superior to 95% were selected for further cloning by limiting dilution.

Cloning of hybridomas of potential interest. 27 potentially interesting hybridomas selected from the initial screening were cloned by limiting dilution techniques in 96-wells plates, and 370 subclones were evaluated in a screen for mouse TLR3 binding using an ELISA as above. The 178 positive clones were tested in a further screen in an inhibition test on 293T-TLR3 cells as above. Among them was supernatant from well G7 from plate 28 (28G7), D1 from plate 13 (13D1), D4 from plate 32 (32D4), well H10 from plate 4 (4H10). Well D5 from plate 5 (5D5) was selected as binding mouse TLR3 but having no functional property in 293T-mTLR3 luciferase assay.

### Example 3

### Reporter Assay

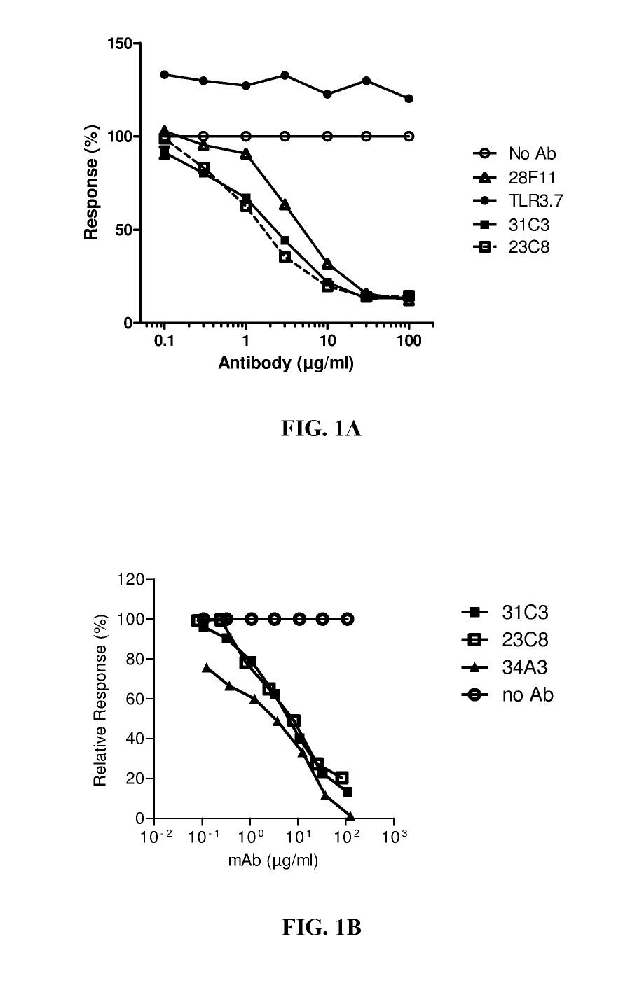

Antibodies were tested for inhibition of TLR3 signaling in a luciferase based reporter gene activity (293T-TLR3-ISRE). Engagement of TLR3 receptor using TLR3-agonists such as poly (I:C) has been reported to activate the type-IFN pathway including the promoter ISRE (Wietek et al. J. Biol. Chem., 278(51), p50923, 2003). Briefly, dsRNA TLR3 agonists were used to induce TLR3 signaling in the reporter assay in the presence of anti-TLR3 antibodies, and TLR3 signaling was assessed. The results, shown in FIG. 1, show that anti-TLR3 antibody 31C3, 23C8, 28F11 and 34A3 strongly inhibited TLR3 signaling in a dose dependent fashion, compared to a control anti-TLR3 antibody (TLR3.7) previously determined to have no effect on TLR3 signaling.

In a further set of experiments, 293-huTLR3 cells were incubated with different concentrations of the anti-TLR3 mAbs for 1 to 2 hours (see FIG. 1A; mAb concentration is represented in axis in μg/ml on a logarithmic scale) followed by the addition to the medium of the TLR3 agonist polyAU at a concentration of 300 μg/ml. Luciferase expression was measured after 6 h. The results are represented in FIG. 1A, and the IC50 are represented in the Table 1 below indicating that the antibodies according to the invention have excellent inhibition properties.

TABLE 1

| Antibody | IC50 (μg/ml) |
|---|---|
| 31C3 | 2.25 |
| 23C8 | 1.83 |
| 28F11 | 5.56 |

In another set of experiments, the same inhibition test was carried out with 100 μg/ml of polyAU and test antibodies 31C3, 23E8 and 34A3. IC50 values were calculated and all of the antibodies had an IC50 below 5 μg/ml. Furthermore, 34A3 exhibited an enhanced inhibition effect at low concentrations (FIG. 1B). FIGS. 1A and 1B show the inhibition properties of increasing doses of the antibodies according to the invention, the inhibition of the TLR3 signalling is dose dependant. This assay confirms the excellent inhibition properties of the antibodies according to the invention.

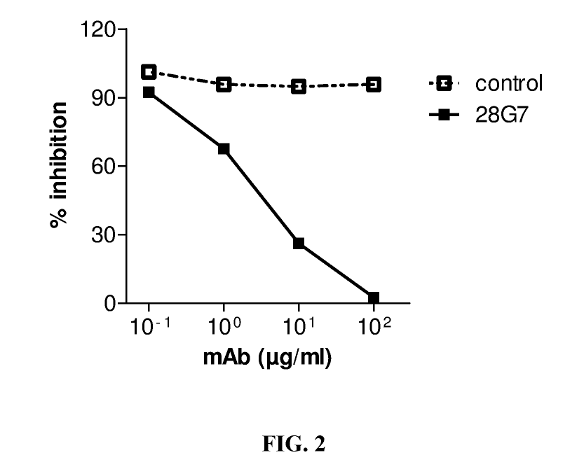

Similarly, rat anti-mouse TLR3 antibodies have been assessed similarly for their ability to inhibit TLR3 signaling in a luciferase based reporter gene activity (293T-mTLR3-ISRE). FIG. 2 show the inhibition properties of increasing doses of the antibody 28G7 (black squares, full line), in comparison with a non relevant control antibody (control, open squares, dashed line) according to the invention, the inhibition of the TLR3 signalling is dose dependant. This assay confirms the excellent inhibition properties of the antibodies according to the invention.

TABLE 2

| Antibody | IC50 (μg/ml) |
|---|---|
| 28G7 | 2.6 |

### Example 4

### Kinetic Studies

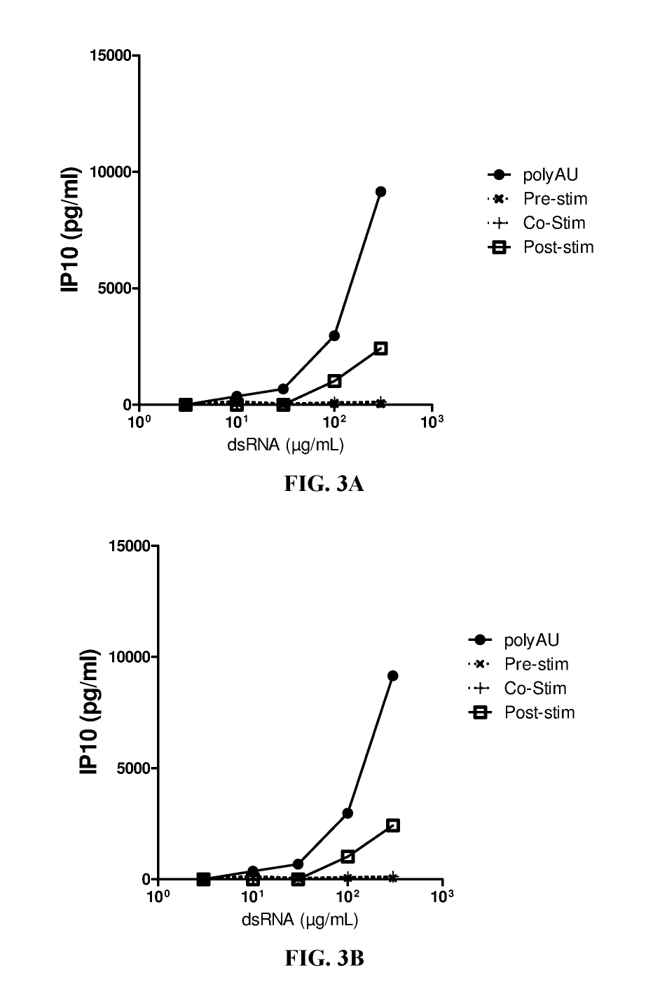

Another set of assays were performed to determine the kinetics of inhibition of the antibodies according to the invention. Briefly, MdDC were incubated with an anti TLR3 antibody (31C3 or 23C8, at a concentration of 50 μg/ml) and a dose range of polyAU in various time settings. The cells were then incubated for 24 hours and IP-10 secretion in culture supernatants was measured by ELISA. FIG. 3A represents the IP-10 secretion in ng/ml (depending on the polyAU doses) for the 31C3 antibody (control, with no antibody is represented by dots, full line); FIG. 3B represents the results for the 23C8 antibody. Experiments were carried out as follows:
Pre-stimulation: The anti-TLR3 antibody was incubated 1 h30 prior to dsRNA addition (black crosses, dotted line).
Co-stimulation: The anti-TLR3 antibody and the dsRNA were added simultaneously. (plus sign “+”, dotted line)
Post-stimulation: The dsRNA was incubated 1 h30 prior to anti-TLR3 antibody addition. (open squares, full line)

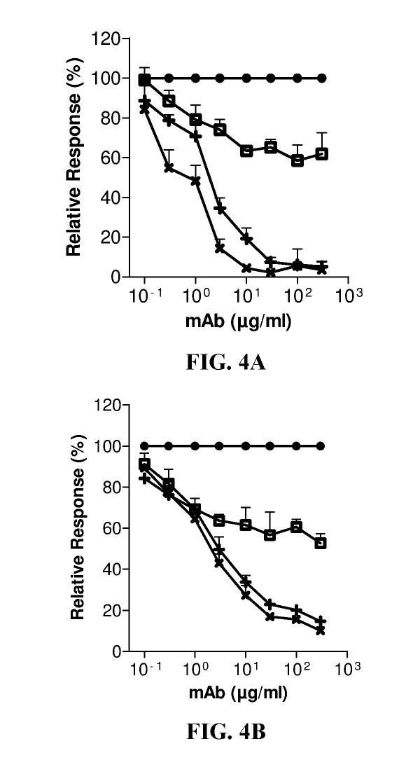

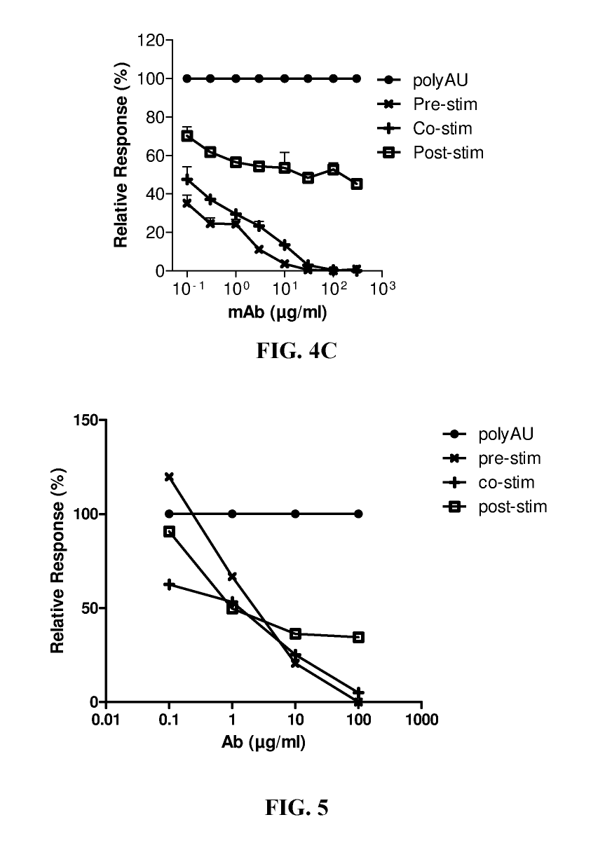

Similarly the kinetics of inhibition of the antibodies was evaluated in 293T-TLR3 luciferase assay. Briefly, 293T-TLR3-ISRE were incubated with a dose range of anti TLR3 antibody (31C3, 23C8, 34A3) and a fixed dose of polyAU (100-300 μg/ml) in various time settings. Luciferase expression was measured at 6 h post poly(A:U) addition. FIG. 4 represents the Ab dose-dependant inhibition of maximal TLR3 response, as obtained with poly(A:U) in the absence of anti-TLR3 antibodies 31C3 (FIG. 4A), 23C8 (FIG. 4B) and 32A4 (FIG. 4C). Experiments were carried out as follows:
Pre-stimulation: The anti-TLR3 antibody was incubated 1 h prior to dsRNA addition.
Co-stimulation: The anti-TLR3 antibody and the dsRNA were added simultaneously.
Post-stimulation: The dsRNA was incubated 1 h prior to anti-TLR3 antibody addition.

These results underline that the antibodies according to the invention are efficient for TLR3 inhibition irrespective of the binding state of the dsRNA to the TLR3 protein. The antibodies do not compete with the binding site of dsRNA but still are able to inhibit TLR3 signaling, even when dsRNA is already bound to the TLR3 protein.

Similarly, the same experiment has been conducted with the mouse surrogate antibody 28G7, in 293T-mTLR3 luciferase assay. Briefly, 293T-mTLR3-ISRE were incubated with a dose range of anti mTLR3 antibodies and a fixed dose of polyAU (100-300 μg/10 in various time settings. Luciferase expression was measured at 6 h post poly(A:U) addition. FIG. 5 represents the Ab dose-dependant inhibition of maximal TLR3 response, as obtained with poly(A:U) in the absence of anti-mouseTLR3 antibodies. Experiments were carried out as follows:
Pre-stimulation: The anti-mTLR3 antibody was incubated 30 min prior to dsRNA addition.
Co-stimulation: The anti-TLR3 antibody and the dsRNA were added simultaneously.
Post-stimulation: The dsRNA was incubated 30 min prior to anti-mTLR3 antibody addition.

Those results show that the surrogate antibody exhibits the same properties in term of inhibition of the TLR3 response, in conditions of pre-, co- and post-incubation of dsRNA than the human anti-TLR3 antibodies.

### Example 5

### TLR3 Modulation

Myeloid DC (MdDC) secretion inhibition test is performed according to the section titled Materials and Methods.

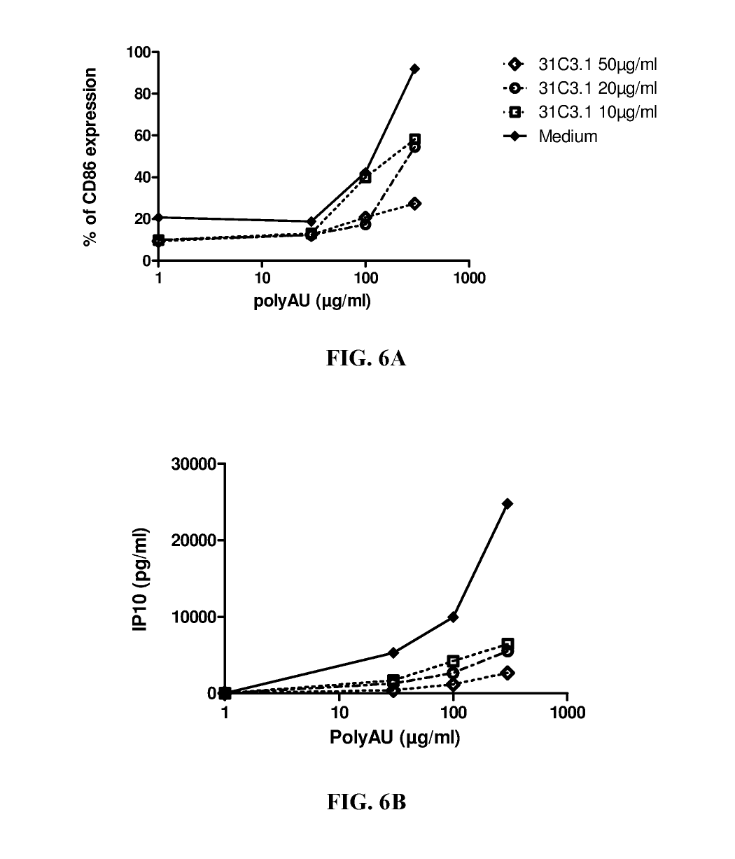

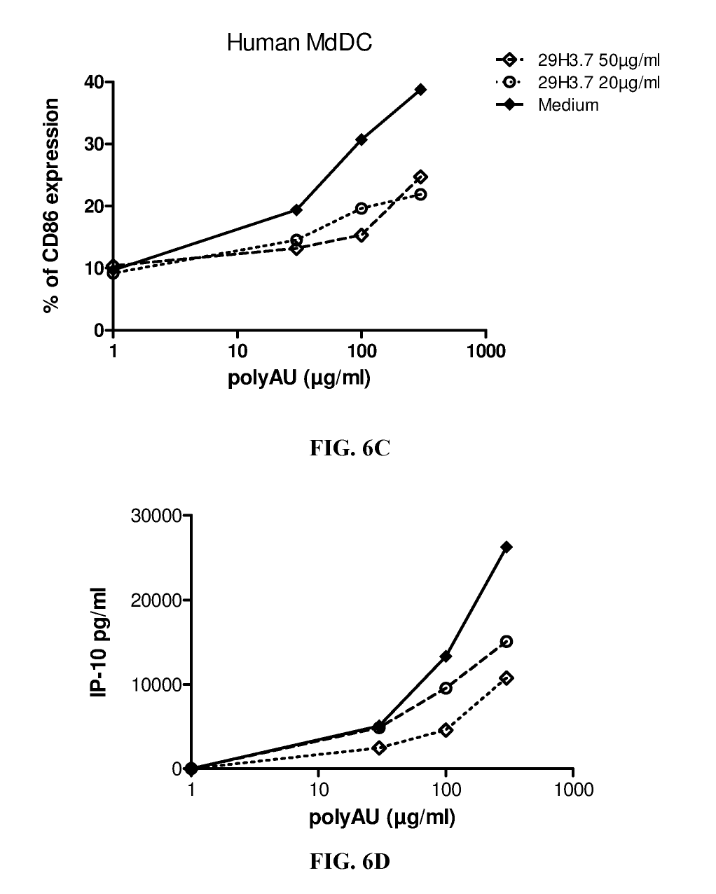

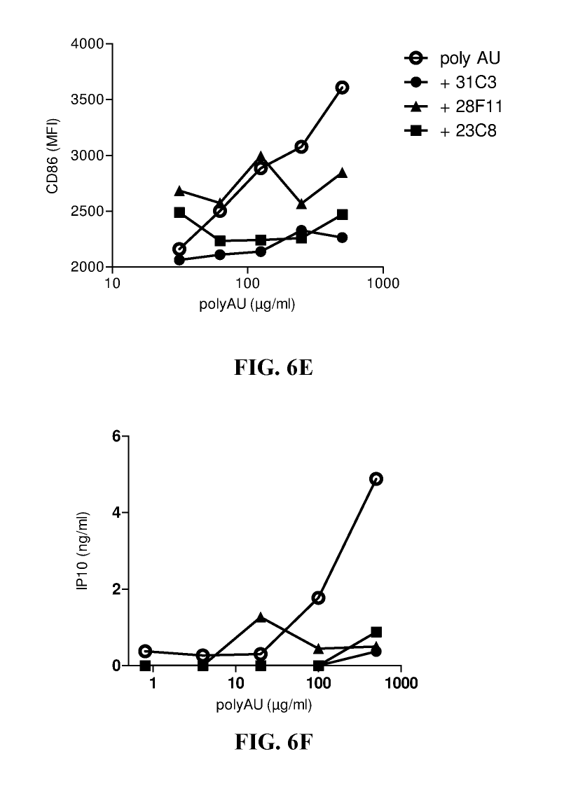

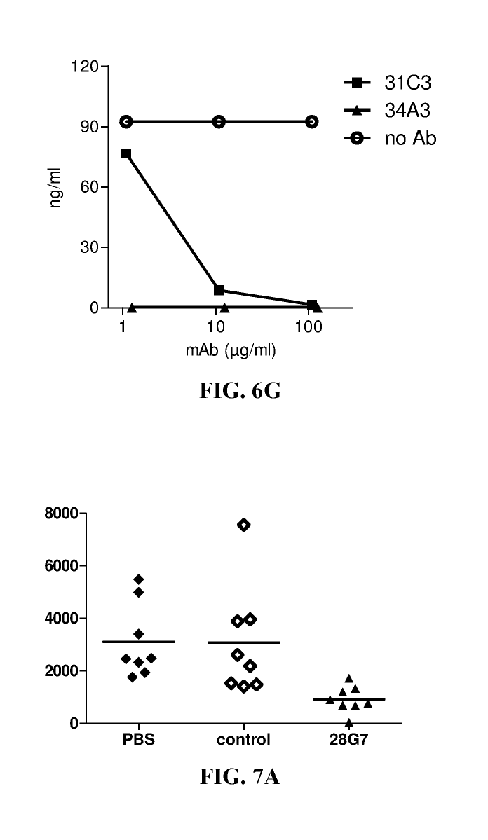

FIGS. 6A to 6G illustrate the inhibitory properties of antibodies 31C3 and 29H3 (both are of IgG1 isotype) in terms of CD86 cellular activation (FIGS. 6A and 6C) and IP-10 secretion (FIGS. 6B and 6D). The anti-TLR3 antibodies 31C3.1 and 29H3.7 antagonize, in vitro, TLR3-mediated myeloid DC activation, moreover, these antibodies effectively downregulated TLR3-mediated CD83/CD86 expression and abrogated TLR3-mediated cytokine/chemokine secretion, in particular IP-10 and IL-6.

F(ab)′2 fragments were generated from antibodies 31C3.1 and 29H3.7 by papain cleavage and purification by ion-exchange chromatography on MonoQ 5/50 GL, analyzed by SDS-PAGE, and tested for inhibition in terms of CD86 cellular activation and cytokine secretion. F(ab)′2 fragments of both antibodies 31C3.1 and 29H3.7 effectively downregulated TLR3-mediated CD83/CD86 expression and abrogated TLR3-mediated cytokine/chemokine secretion, to a similar extent as full length antibodies. F(ab)′2 fragments of antibody 31C3 and whole 31C3 IgG abrogate TLR3-mediated CD86 expression and IP-10 secretion to a similar extent; the same was observed for F(ab)′2 fragments of antibody 29H3.

In a similar experiment, the ability of 28F11 and 23C8 antibodies to inhibit TLR3 signaling were assessed, compared to antibody 31C3. The antibodies were tested at a concentration of 50 μg/ml in the presence of an increasing dose of polyAU. The results are represented in terms of CD86 cellular activation (FIG. 6F) and IP-10 secretion (FIG. 6E), underlining the inhibitory properties of the antibodies according to the invention.

Antibody 34A3 has also been tested in a similar setting. This time, the cells (MdDC) are stimulated with a fixed dose of polyAU (300 μg/ml) and increasing doses of the antibody is added to the medium. FIG. 6G illustrates the results for antibody 34A3, in comparison with 31C3 in terms of IP-10 secretion.

The mouse antibodies have also been tested for their ability to inhibit TLR3 induced signalling in vivo. Briefly, groups of 5 mice (C57Bl/6J, 8-10 weeks old) are constituted. PBS or anti-TLR3 antibodies (100 μg per mice or 200 μg per mice) is injected IP three hours before polyAU administration IV (20 or 100 μg). Two hours later, blood is withdrawn, serum is prepared and a serum dosage of IL-6 is performed (BD optEIA™ set mIL-6). Results are reported in FIGS. 7A and 7B. In FIG. 7A, is shown the inhibition of 100 μg of anti-mouse TLR3 antibodies 28G7, 4H10, 13D1, and the non functional anti-mouse TLR35D5 mAb, on IL-6 secretion induced by 20 μg IPH3102. In FIG. 7B, is shown the inhibition of 200 μg of anti-mouse TLR3 antibodies 28G7, 32D4, the non functional anti-mouse TLR3 control mAb and a control irrelevant rat IgG pAb, on IL-6 secretion induced by 100 μg IPH3102.

The figures underline the fact that the anti-TLR3 mouse antibodies are able to inhibit a TLR3 ligand (here polyAU) induced signalling.

### Example 6

### Bivalent Affinity

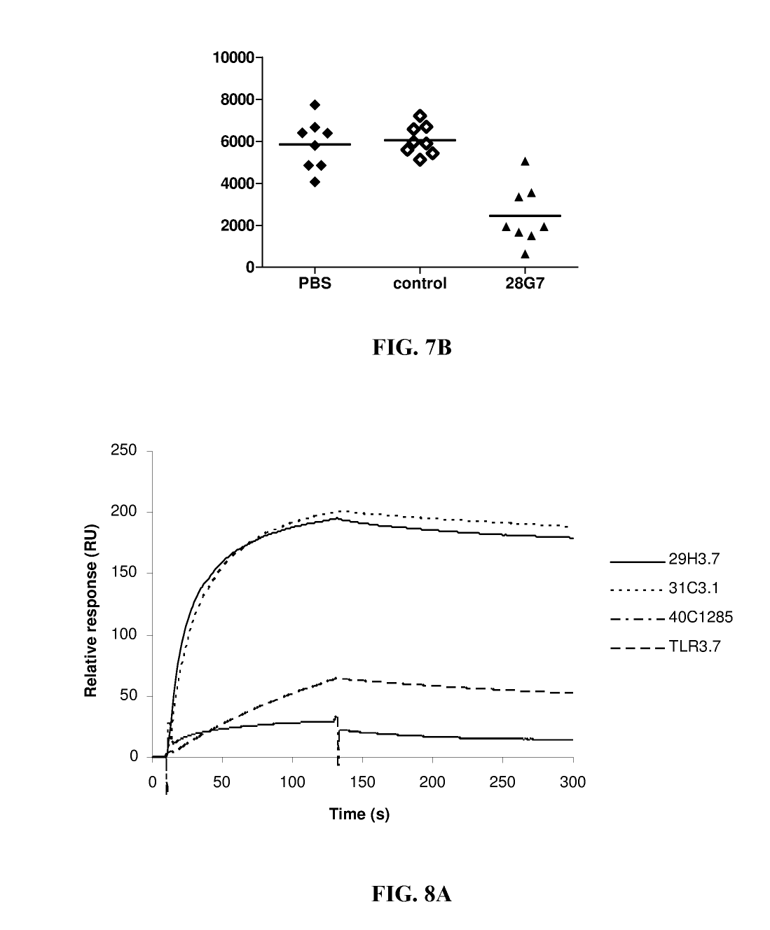

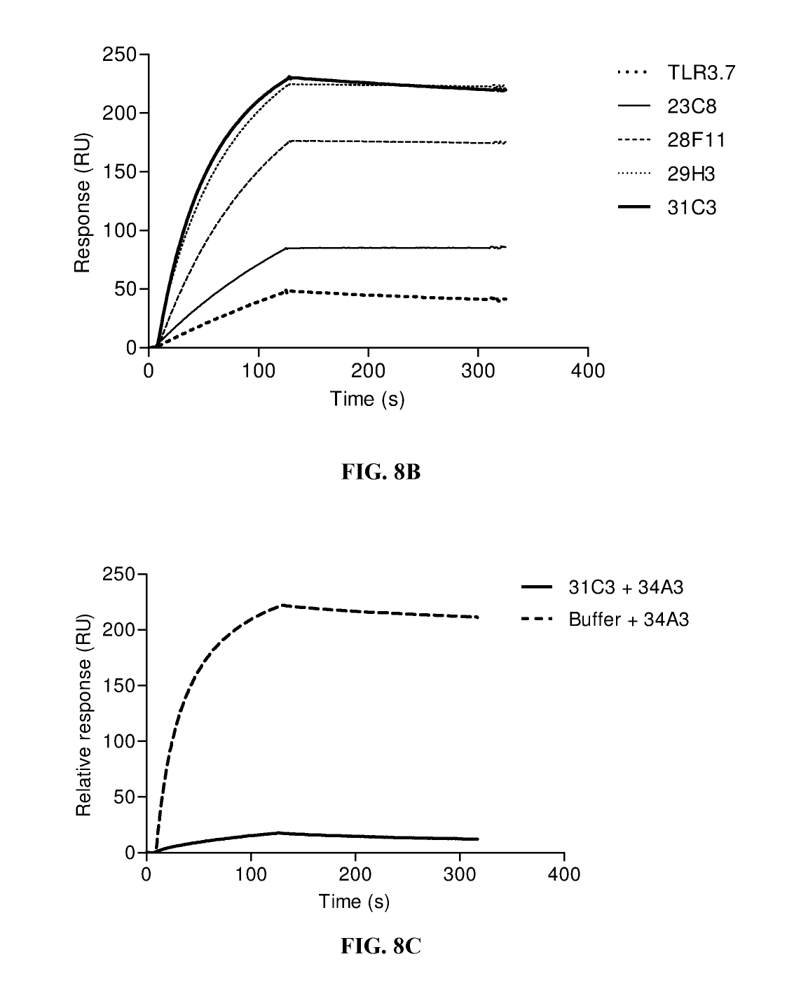

Binding properties of the antibodies 29H3, 23E7, 31C3 and commercially available antibodies TLR3.7 (eBiosciences) and 40C1285 (Abcam) were compared using the methods described for SPR, item c). FIG. 8A show that the binding affinity for TLR3 is significantly better in the case of 29H3.7 and 31C31 than in the case of commercially available antibodies.

Binding to TLR3 was determined at neutral (pH 7.2) and acid (pH 5.6) conditions, and K_{D} values were calculated. The results (mean of 2 or 3 experiments) are shown in Table 1. At neutral pH, 23E7, 29H3 and 31C3, 23C8, 28F11 and 34A3 all showed strong and similar bivalent affinity (K_{D}) for recombinant human TLR3 better than 500 picomolar (around 50 picomolar or lower for 23E7, 29H3, 31C3 and 34A3). Antibody TLR3.7 (eBiosciences) in comparison showed a significantly lower binding affinity. At acid pH, however, 23E7 lost considerable binding affinity and its K_{D} was at about 4 nanomolar. The affinity of 31C3, 23C8, 28F11 and 34A3 were measured in the same conditions in separate assays, the results are represented in Table 3 and in FIGS. 8A, 8B and 8C. These results indicate that the antibodies according to the invention have a high affinity (lower than 500 μM), especially at acidic pH.

TABLE 3

| | Mean | Mean |
|---|---|---|
| Antibody | K_{D} (M) at pH 7.2 | K_{D} (M) at pH 5.6 |
| 23E7.3 | 3.51 * 10⁻¹¹ | 4.32 * 10⁻⁹ |
| 29H3.7 | 4.74 * 10⁻¹¹ | 1.10 * 10⁻¹¹ |
| 31C3.1 | 5.05 * 10⁻¹¹ | 6.14 * 10⁻¹¹ |
| TLR3.7 | 4.5 * 10⁻⁸ | 9 * 10⁻⁹ |
| 23C8 | 1.38 * 10⁻¹⁰ | 1.03 * 10⁻¹¹ |
| 28F11 | 6.50 * 10⁻¹⁰ | 3.35 * 10⁻¹⁰ |
| 34A3 | 3.01 * 10⁻¹² | 5.17 * 10⁻¹¹ |

Similarly, binding to mouse TLR3 was determined at neutral (pH 7.2) and acid (pH 5.6) conditions, and K_{D} values were calculated. The results (mean of 2 or 3 experiments) are shown in Table 4. At neutral and acid pH, mAb 32D4, 28G7 and 13D1 all showed strong and similar bivalent affinity (K_{D}) for recombinant mouse TLR3 better than 500 picomolar. The affinity of mAb 32D4, 28G7 and 13D1 were measured in the same conditions in a separate assay, the results are represented in Table 4. These results indicate that the antibodies according to the invention have a high affinity, especially at acidic pH.

TABLE 4

| | Mean | Mean |
|---|---|---|
| Antibody | K_{D} (M) at pH 7.2 | K_{D} (M) at pH 5.6 |
| 28G7 | 7.05 * 10⁻¹³ | 1.26 * 10⁻¹³ |

### Example 7

### Epitope Mapping

Epitope Mapping at Acid pH.

Competition assays were conducted according to the methods described for SPR, item d) at pH 5.6.

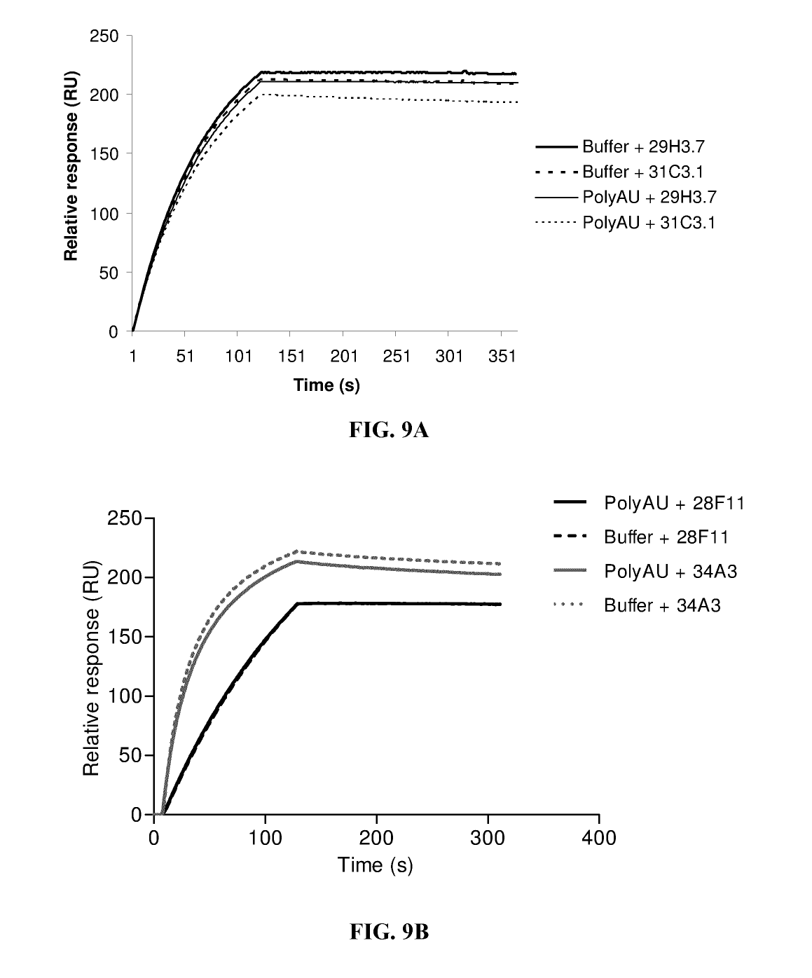

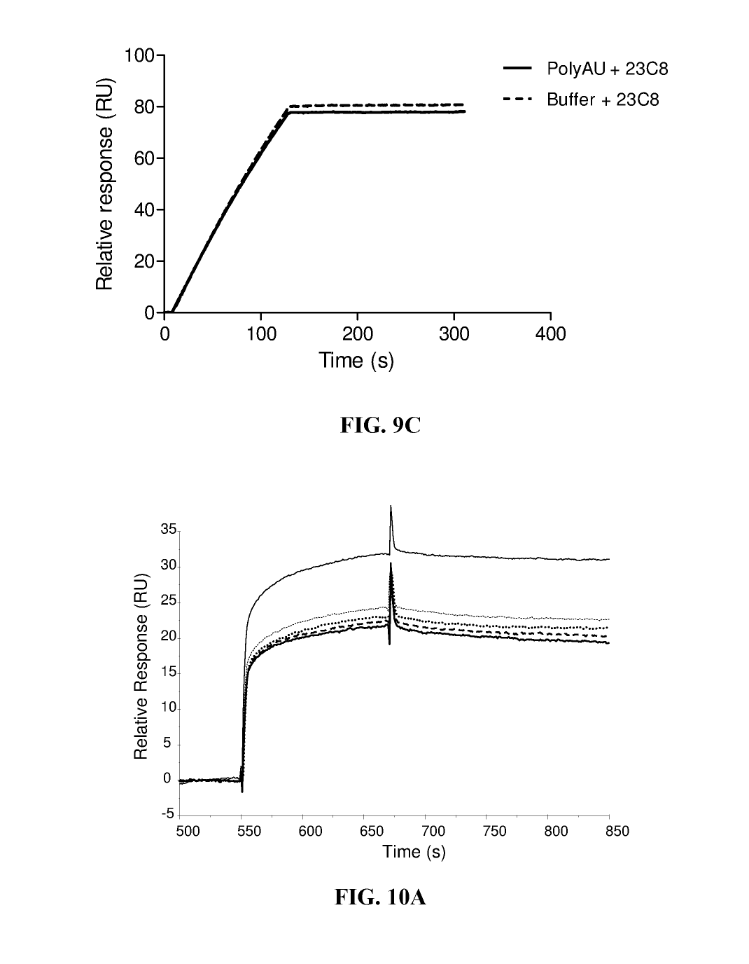

FIG. 9A illustrates that antibodies 29H3.7 and 31C3.1 were able to bind TLR3 even in the presence of polyAU, similarly, FIGS. 9B and 9C illustrate that antibodies 28F11, 34A3 and 23C8 were able to bind TLR3 even in the presence of polyAU. 29H3.7, 31C3.1, 28F11, 34A3 and 23C8 are thus able to inhibit dsRNA signaling even in the presence of a dsRNA bound to the TLR3 protein.

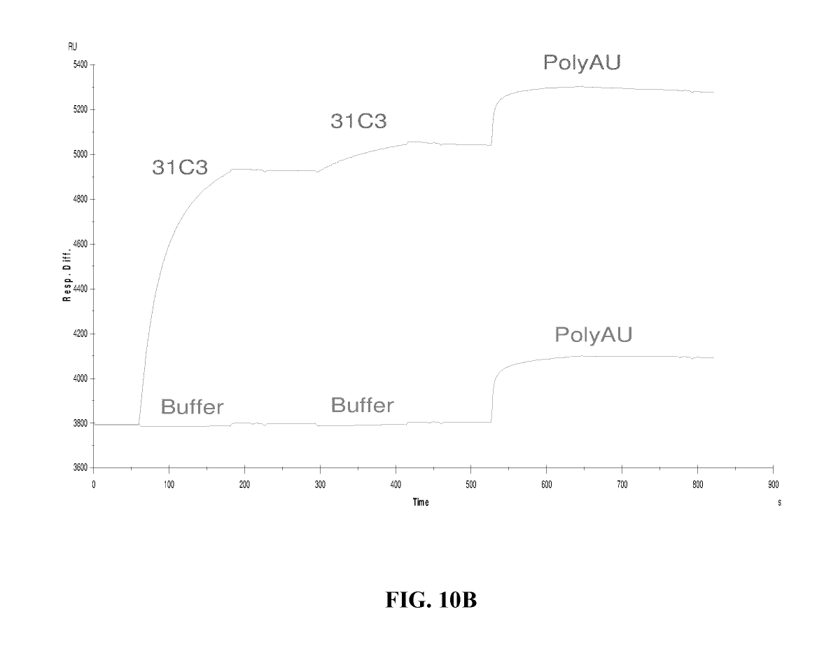

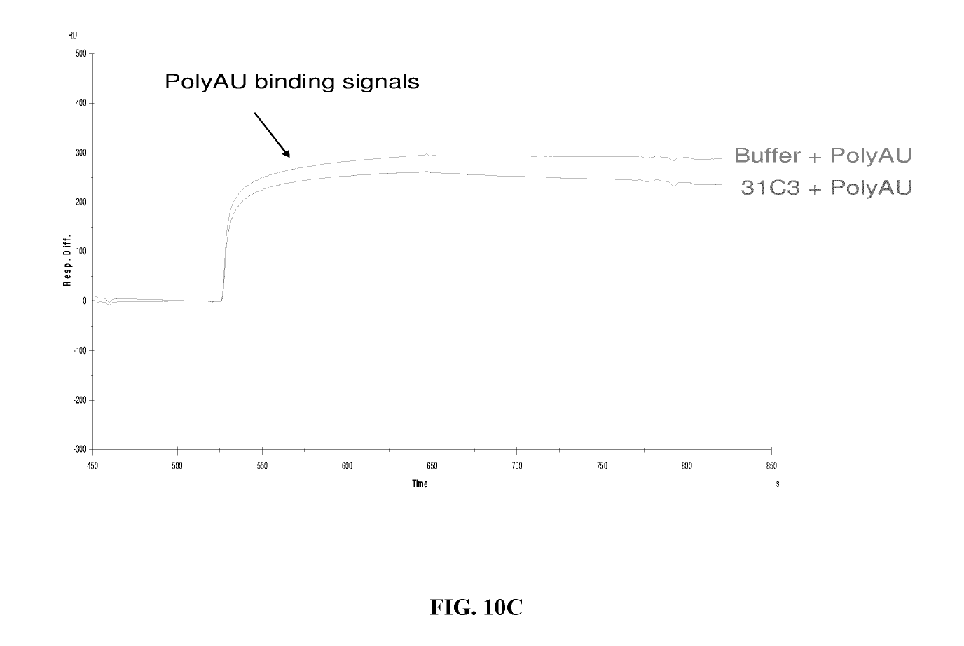

FIG. 10A shows the binding of the antibodies according to the invention on a human TLR3 chip, when the chip has been previously incubated with polyAU, a TLR3 ligand. Buffer is represented in a thin full line, 31C3 is a bold full line, 28F11 in a thin dashed line, 23C3 in a bold dashed line and 29H3 in a dotted line. FIG. 10A underlines that in the presence of dsRNA, the antibodies are able to be bound efficiently.

FIG. 10B shows the binding of the polyAU on a human TLR3 chip when the chip was previously incubated with antibodies of the invention. Buffer (followed by polyAU) is represented in the lower line, while 31C3 followed by polyAU is in the upper line. Incubation of TLR3 with anti-TLR3 antibody therefore does not prevent dsRNA from binding to TLR3. FIG. 10C shows the polyAU binding signals on a human TLR3 chip when the chip was first incubated with antibody 31C3; the upper line shows buffer followed by polyAU while the lower line shows 31C3 followed by polyAU. The figures show that the anti-TLR3 antibodies do not prevent dsRNA from binding to TLR3 polypeptides.

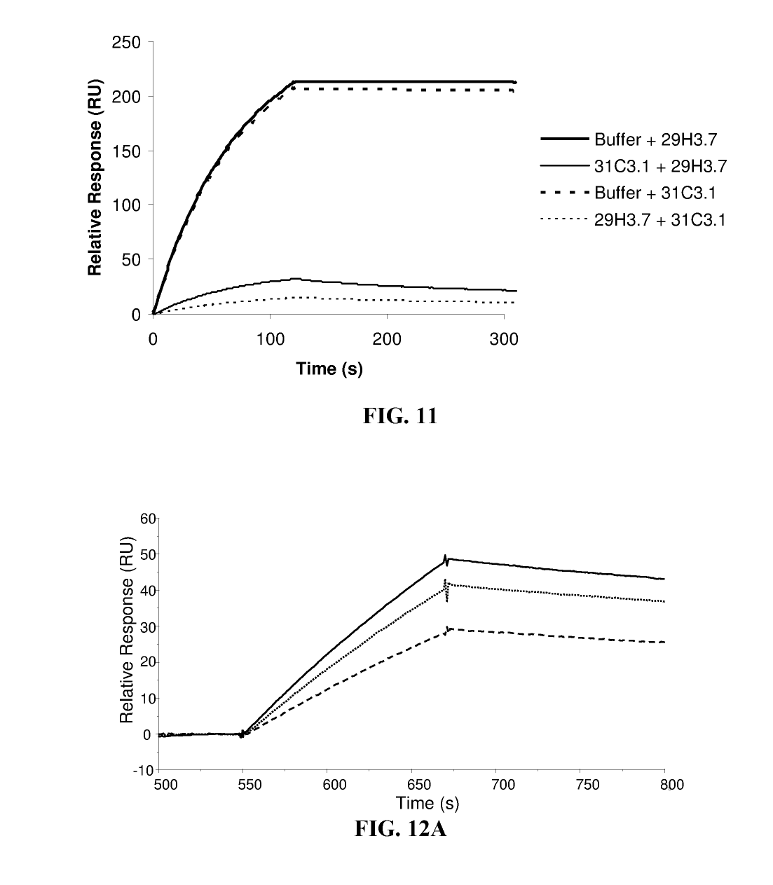

FIG. 11 reports binding for antibodies 29H3 (full bold line) and 31C3 (dashed bold line), when human TLR3 chip has been previously saturated with 31C3 and then incubated with 29H3 (thin full line) or when human TLR3 chip has been previously saturated with 29H3 and then incubated with 31C3 (thin dashed line).

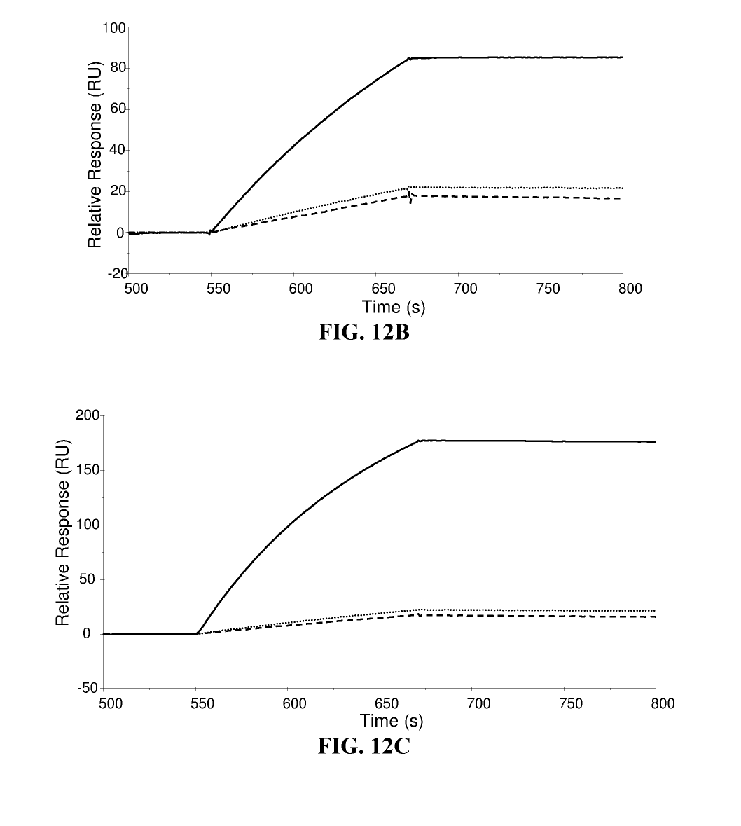

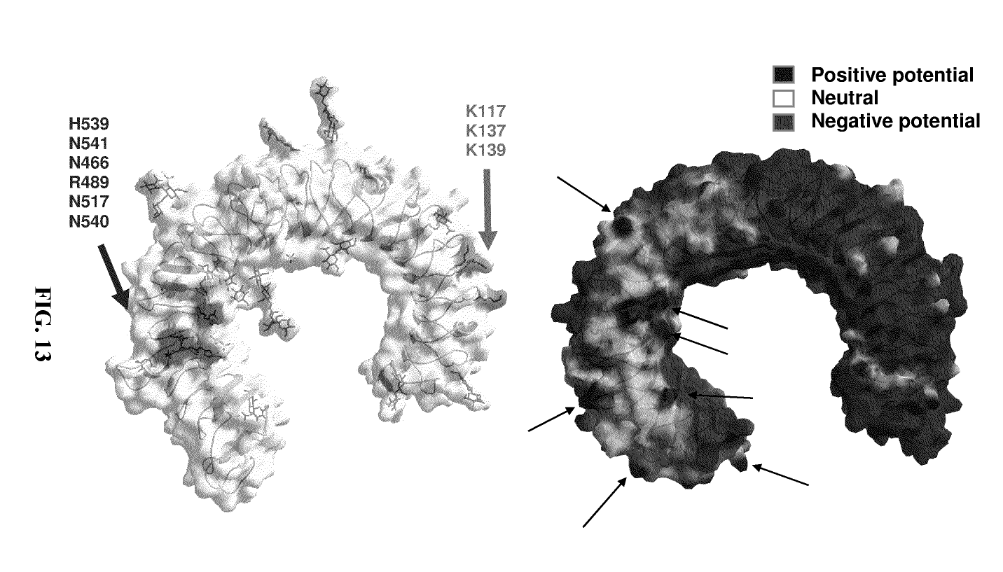

FIG. 12 represents the binding of antibodies either alone (full line), after saturation with the 31C3 antibody and then binding of the test antibody (dotted line), and saturation of 31C3 after saturation with 31C3, as a control (dashed line). Results are provided for antibodies TLR3.7 (FIG. 12A), 23C8 (FIG. 12B) and 28F11 (FIG. 12C). This comparison of binding levels underlines that the antibodies according to the invention have an impaired binding to hTLR3 when the chip has previously been saturated with the 31C3 antibody, on the contrary, the commercial TLR3.7 antibody retains the same binding level in the presence or in the absence of 31C3 antibody.

Epitope Mapping at Neutral pH.

Competition assays were conducted according to the methods described for SPR, at pH 7.2. Antibodies 29H3.7 and 31C3.1 competed with one another for binding to TLR3 since binding by one antibody impaired the binding to TLR3 of the other. Epitope mapping was explored also with commercial antibody TLR3.7 and antibody 23E7.3. Neither of these antibodies competed for binding to TLR3 with either of antibodies 29H3.7 and 31C3. However, antibody TLR3.7 did compete for binding to TLR3 with antibody 23E7.3, indicating that they have overlapping binding sites.

The 34A3 antibody binding affinity was measured either alone on a rhTLR3 chip or in conditions where the rhTLR3 chip was saturated with the 31C3 antibody prior to 34A3 addition. The results are shown in FIG. 7C, and provide evidence that the two antibodies compete for binding to hTLR3, thus sharing a common epitopic determinant in view of their overlapping binding sites.

FIG. 13 shows molecular surface maps of the extracellular domains of the human TLR3 protein, generated by computer modeling using SwissPdb Viewer 4.0 (Guex and Peitsch (2007) Electrophoresis 18: 2714-2723) based on data publicly available as data file 1ZIW from the Resource for Studying Biological Macromolecules database from the Protein Data Bank (PDB) project of the European Bioinformatics Institute (Hinxton U.K.). The C-terminal is on the left and the N-terminal on the right in this view, showing the face of TLR3 which is substantially glycosylation free, the other face being extensively masked by carbohydrates (see e.g. Choe et al. (2005) Science 309:581-585).

The surface map on the right hand side in FIG. 13 shows the electrostatic potential of the human TLR3 polypeptide at neutral pH, mapped to the molecular surface. The main areas of positive electrostatic potential are indicated with arrows, forming a general region of positive and neutral potential on the glycosylation free face of the C-terminal side. The glycosylation-free face is believed to be accessible for interaction with ligands accessory molecules or TLR3 monomers or other oligomer assemblies, and the region of positive electrostatic potential on the C-terminal side (in the darkest shading, the main positive areas indicated by arrows) is thought to correspond to the dsRNA binding region. These regions are also shown in Figure 5 of Choe et al. (2005). The map on the left hand side indicates amino acid residues determined by mutation studies to be involved in dsRNA binding, where residues dsRNA binding was inhibited (>80%) by mutation of residues H539, N541, N466, R489, N517 or N540, and where dsRNA binding was inhibited (>80%) by mutation of residues K117, K137 and K139. The model proposed in Choe et al. (2005) is that TLR3 forms a homodimer where the dimerization interface is proximal to the C-terminal region on the glycosylation-free face and that dsRNA binds to only the glycosylation-free face of TLR3, such that two TLR3 monomers could sandwich dsRNA in a complex of two TLR3 monomers and one dsRNA strand. A model involving an oligomeric complex of TLR3 formed by multiple dimers of TLR3 binding to longer dsRNA strands has also been proposed (see e.g. Bell et al. (2006) PNAS USA 103(23): 8792-8797). Ranjith-Kumar et al. (2007) J. Biol. Chem. 282(10): 7668-7677 propose that TLR3 can exist in an oligomerized stage in the absence of ligand, and that dsRNA binding can cause rearrangement in the dimer leading to lateral sliding of the monomers toward each other and adjust to accommodate the dsRNA, and where the resulting conformation change can stimulate the interaction of the TIR domain to induce signaling. One explanation could therefore be that the anti-TLR3 antibodies 31C3, 29H3, 23C8, 28F11 and 34A3 bind TLR3, e.g. on glycosylation-free face, and possibly in an epitope having a negative charge at neutral pH which is therefore not in the region of dsRNA binding to TLR3. The antibodies therefore would not prevent interaction of dsRNA with TLR3 at the C-terminal dsRNA binding site, but could inhibit signaling, e.g. by preventing a TLR3 dimer or oligomer that has bound dsRNA from rearranging or adopting the configuration required to bring about interaction of the TIR domains, or by interfering with the proper formation of TLR3 dimers and/or a consequential TLR3-dsRNA ternary complex.

FIG. 20 was generated by computer modeling using SwissPdb Viewer 4.0 (Guex and Peitsch (2007) Electrophoresis 18: 2714-2723) based on data publicly available as data file 1ZIW from the Resource for Studying Biological Macromolecules database from the Protein Data Bank (PDB) project of the European Bioinformatics Institute (Hinxton U.K.). FIG. 20A represents a single TLR3 protein with the amino acids 102 to 173 represented in dark grey, the N-terminal is on the right and the C-terminal is on the left. Contact zones to RNA are indicated by circles. FIGS. 20B and 20C represent two TLR3 protein dimerized in the presence of a dsRNA ligand (in black) from a top view and a side view. The C-terminals are dimerized, in the center of the construction whereas the N-terminals are on the extremities of the construction. FIG. 20D also represents two TLR3 protein dimerized in the presence of a dsRNA ligand (in black), the amino acids 102 to 173 are represented in dark grey. One can see from these figures that the regions on TLR3 identified by epitope mapping as binding the TLR3 antibodies are close to the second interaction zone of TLR3 with the dsRNA ligand. Example 16 shows that the antibodies bind to a region distant to the main dsRNA binding site on the C-terminal half of the polypeptide, while Example 4 shows that antibodies do not block dsRNA binding. It is thus possible that while the antibodies do not block the binding of dsRNA to the main C-terminal dsRNA binding site of TLR3, they may (or may not) to some extent block binding of dsRNA to the second interaction zone, however in each case inhibiting TLR3 signalling. High molecular weight dsRNA is believed to have high affinity for TLR3 polypeptides and is believed to induce multimerization of TLR3 polypeptides and consequent TLR3 signaling. Low molecular weight dsRNA however is not believed to induce such multimerization. It is therefore possible that anti-TLR3 antibodies may prevent multimerization of TLR3 polypeptides that have bound high molecular weight dsRNA such as IPH3102 and/or signalling by such TLR3 multimer complex.

### Example 8

### TLR3 Internalization Assays

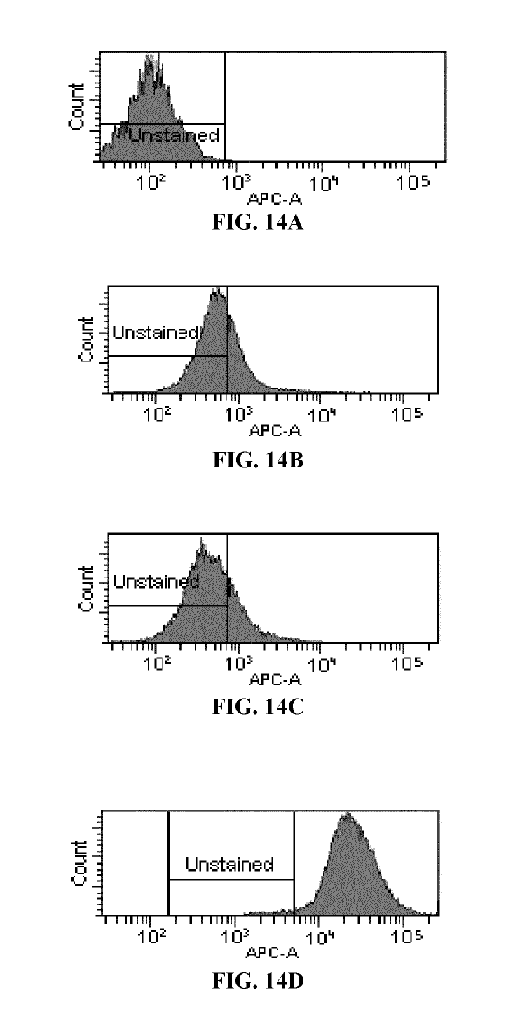

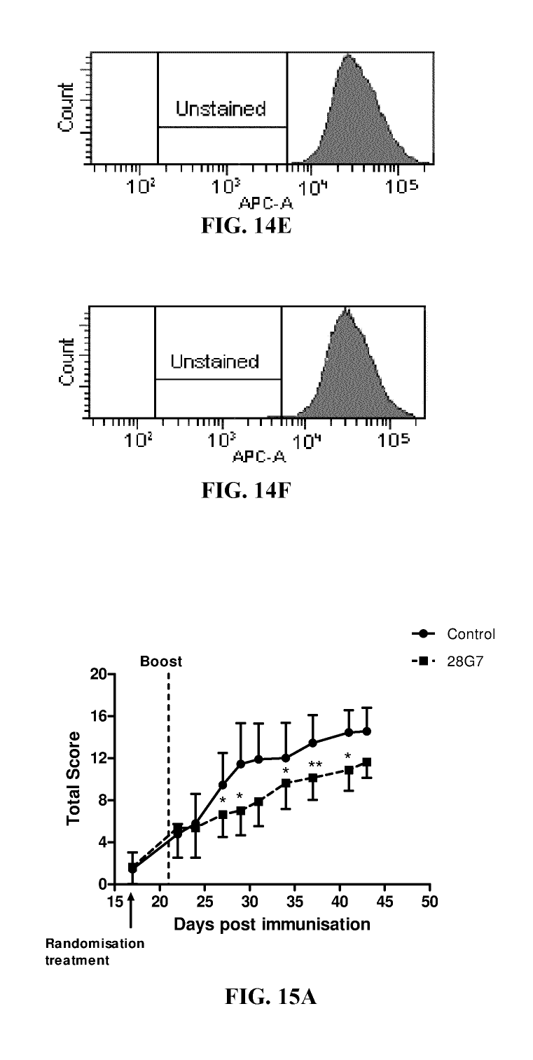

Briefly, either no antibody or 50 m/mL of the anti-TLR3 antibody 31C3 was incubated with live 293T-ISRE/TLR3 cell lines, for 2 h or 24 h at 37° C. Cells were then washed, fixed and permeabilized using IntraPrep permabilization reagent from Beckman Coulter. Presence of TLR3-bound anti-TLR3 31C3 Ab is revealed with a goat anti-mouse Ab, labelled with APC. Alternatively, permeabilized cells were incubated with either control isotype or with a TLR3 specific mAb efficient in FACS, noncompeting with 31C3 Ab for TLR3 binding, both labelled with biotine, and further revealed through cell incubation with fluorescent streptavidin derivative. The graphs are represented in FIG. 14. FIG. 14A represents the negative control, representing fluorescence intensity of the 293T-ISRE/TLR3 cells in the absence of an antibody linking TLR3 proteins. FIGS. 14B and C represent the fluorescence induced by the binding to TLR3 protein of internalized 31C3 antibody, after 24 h or 2 h incubation, respectively. FIG. 14D indicates the steady state level of TLR3 expression in 293T-ISRE/TLR3 cell lines, without pre-incubation with 31C3 antibody. FIGS. 14E and 14F, showing a similar fluorescence than FIG. 14D confirm that the binding of TLR3 by antibody 31C3 does not down-modulate the expression of TLR3 on 293T-ISRE/TLR3 cell lines.

Those results show that the antibodies according to the invention are rapidly and efficiently internalized, moreover without any hTLR3 down-modulation. These results underline the efficiency of the binding of the antibodies according to the invention. Furthermore, this rapid internalization provides evidence that the antibodies are promising candidates for therapy.

### Example 9

### In Vivo Efficacy Model for the Treatment of RA—Preventive Setting

Briefly 20 mice were immunized on day 0 with 100 μg of collagen emulsified in CFA complemented with Mycobater tuberculosis (2 mg/ml) and injected intradermally (ID) at the base of the tail. At day 17, animals were scored (clinical signs often appear prior to the boost), randomized into 2 groups of 8 or 9 mice according to the sum of the 4 limbs clinical score and treated. At days 21, the collagen immunization was boosted by ID administration of collagen alone (100 μg in 500).

The following groups were constituted:
group 1 (PBS, n=9): treated 2000 twice/week IP
group 2 (28G7, n=8): treated 500 m/mice twice/week IP

Scoring of the four limbs of the animal was evaluated thrice a week for 3 to 4 weeks. Scoring was evaluated according to table 5.

TABLE 5

| Score | Inflammation level |
|---|---|
| 0 | No evidence of erythema and swelling |
| 1 | Erythema and mild swelling confined to the tarsals or ankle joint |
| 2 | Erythema and mild swelling extending from the ankle to the tarsals |
| 3 | Erythema and moderate swelling extending from the ankle to |
| | metatarsal joints |
| 4 | Erythema and severe swelling encompass the ankle, foot and |
| | digits, or ankylosis of the limb |

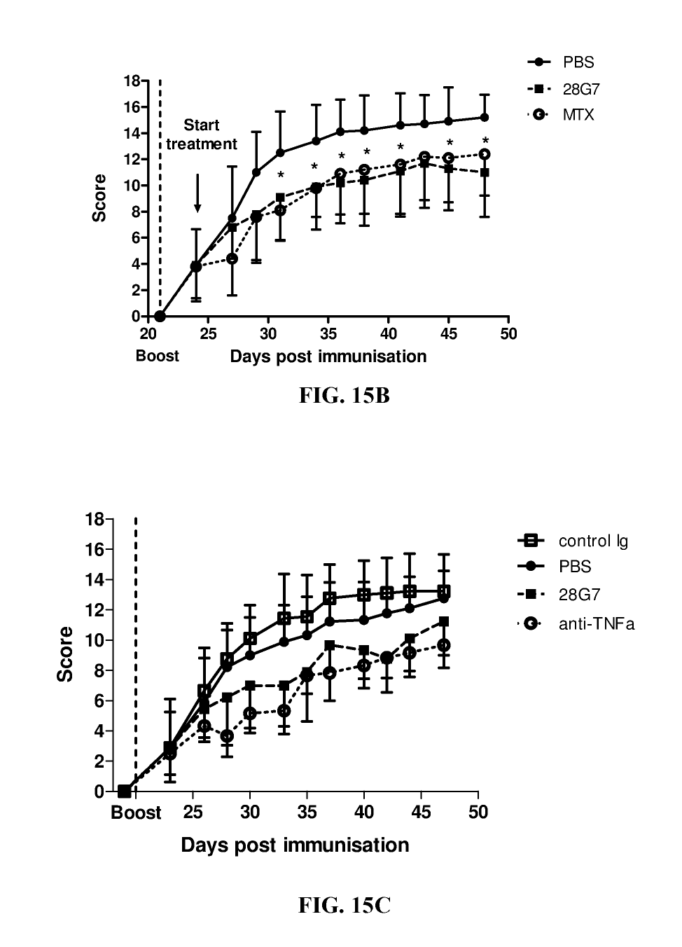

The results are reported in FIG. 15A. The present experiment underlines that the antibodies according to the invention are statistically effective (* p>0.05, ** p>0.005, in Dunnett's test) in the curative treatment of Rheumatoid Arthritis (RA) in comparison with PBS.

### Example 10

### In Vivo Efficacy Model for the Treatment of RA—Curative Setting

### Experiment #1: 28G7, PBS, MTX

Briefly 30 mice were immunized on day 0 with intradermal injection of 100 μg of collagen emulsified in CFA complemented with Mycobater tuberculosis (2 mg/ml) at the base of the tail. 21 days later, the collagen immunization is boosted by ID administration of collagen alone (100 μg in 500/mice). At day 24, animals were randomized into 3 groups of 10 mice according to the sum of the 4 limbs clinical score and treatment began.

Group 1 (PBS, n=10): treated 2000 twice/week IP
Group 2 (Methotrexate—MTX, n=10): treated 2.5 mg/kg twice/week IP
Group 3 (28G7, n=10): treated 500 m/mice twice/week IP

Scoring of the four limbs of the animal was evaluated thrice a week for 3 to 4 weeks. Scoring was evaluated according to table 5.

The results are reported in FIG. 15B. The present experiment underlines that the antibodies according to the invention are statistically effective in the treatment of an established Rhumatoid Arthritis (RA) in comparison with PBS and Methotrexate (* p>0.05, in Dunnett's test). MTS having a different mechanism of action than the anti-TLR3 antibody, a combination of the two drugs could be beneficial.

### Experiment #2: 28G7, PBS, Anti-TNFα Humira™

Briefly 35 mice were immunized on day 0 with intradermal injection of 100 μg of collagen emulsified in CFA complemented with Mycobater tuberculosis (2 mg/ml) at the base of the tail. 21 days later, the collagen immunization is boosted by ID administration of collagen alone (100 μg in 500/mice). At day 24, animals were randomized into 4 groups according to the sum of the 4 limbs clinical score and treatment began.

Group 1 (PBS, n=9): treated 2000 twice/week IP.
Group 2 (control Ig antibody, n=9): treated 5000 twice/week IP.
Group 3 (28G7, n=9): treated 500 m/mice twice/week IP.
Group 4 (Humira™, n=6): treated 1000 twice/week IP.

Scoring of the four limbs of the animal was evaluated thrice a week for 3 to 4 weeks. Scoring was evaluated according to table 5.

The results are reported in FIG. 15C. The present experiment underlines that the antibodies according to the invention are effective in the treatment of an established Rheumatoid Arthritis (RA) in comparison with PBS and anti-RNFα antibody Humira™. The anti-TNFα having a different mechanism of action than the anti-TLR3 antibody, a combination of the two drugs could be beneficial.

### Example 11

### In Vivo Efficacy Model for the Treatment of Colitis

Four groups of 10 male mice (Balb/c) were used for the model of colitis and one extra group of 8 mice without colitis were used as control (no dosage, intracolonic instillation of saline).

The treated groups were divided as follows:

Group 1: 10 mice received antibody 28G7 (ip, 500 m/mouse).
Group 2: 10 mice received a non TLR3-relevant antibody administration (ip, 500 m/mouse).
Group 3: 10 mice received the rat anti-mouse TNF antibody (ip, 15 mg/kg, Humira™)
Group 4: 10 mice received a PBS (ip, 200 m/mouse).

One hour after in injections, colitis was induced by intracolonic instillation of 2,4,6-trinitrobenzen-sulfonic acid (TNBS) (2 mg/mouse in 40% ethanol of TNBS) in male Balb/C mice (5 to 6 weeks-old). In groups 1, 2 and 3, another injection of either 28G7 or non TLR3-relevant antibody or the anti-TNF antibody was repeated 72 hours after the first antibody injection.

For all groups, several parameters of disease progression were assessed daily: body weight, presence of blood in the feces, presence and severity of diarrhea. All animals were sacrificed for tissue collection 7-days after the induction of colitis. Macroscopic damage score, wall thickness and myeloperoxydase activity (index of granulocyte infiltration), were measured in colonic tissues. Macroscopic damage score is evaluated by observing the parameters as detailed in table 6.

TABLE 6

| | Score |
|---|---|
| Faecal blood | |
| Diarrhea | |
| Haemorrhage | 0 |
| | 1 (blood in lumen) |
| Adhesion | 0 |
| | 1 (1 point) |
| | 2 (more than 1) |
| | 3 (very severe) |
| Mucus | 0 |
| | 1 |
| Erythema | 0 |
| | 1 (less than 1 cm) |
| | 2 (more than 1 cm) |
| Edema | 0 |
| | 1 (intermediate) |
| | 2 (severe) |
| Ulcer | 0 |
| | 1 (less than 1 cm) |
| | 2 (more than 1 cm) |
| Stricture | 0 |
| | 1 (1 stricture) |
| | 2 (2 stricture) |
| | 3 (more than 2) |
| Total | |

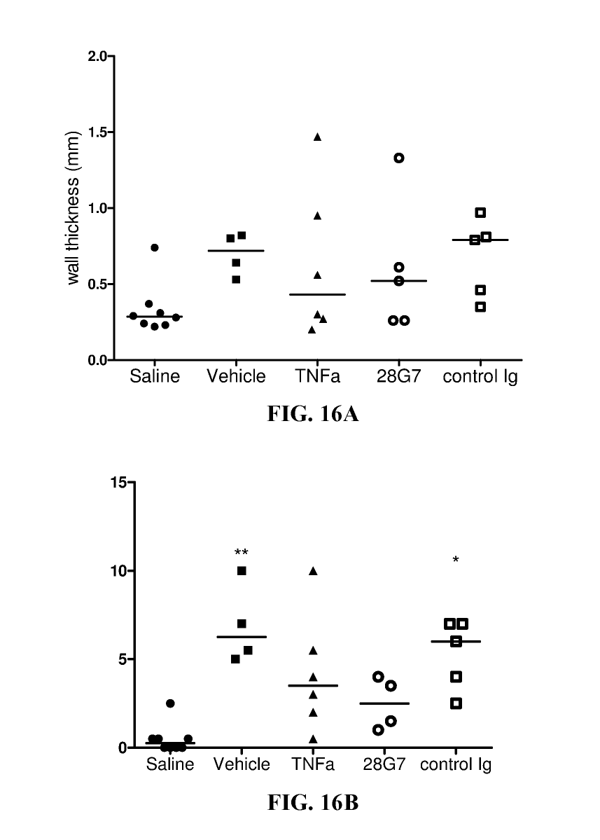

FIG. 16 shows the results of the experiment. FIG. 16A shows the wall thickness measurements for the mice treated with saline (black dots), with TNBS only (black squares) with an anti-TNFα antibody and TNBS (black triangles), with 28G7 and TNBS (open dots), and with a control Ab and TNBS (open squares). FIG. 16B shows the macroscopic damage score for the mice treated with saline (black dots), with TNBS only (black squares) with an anti-TNFα antibody and TNBS (black triangles), with 28G7 and TNBS (open dots), and with a control Ab and TNBS (open squares). These results underline an effect of the anti-TLR3 antibody according to the invention in the development of the disease, under stringent conditions.

### Example 12

### In Vivo Efficacy Model for the Treatment of Hepatitis

Preventive setting: 20 mice are administered the 28G7 antibody (ip, 500 μg) one hour prior to concanavaline A administration (10 or 20 mg/kg, iv). Plasmatic cytokines and ALAT (alanine amino transferase) are dosed and histology is performed after sacrifice.

### Example 13

### In Vivo Efficacy Model for the Treatment of COPD (Chronic Obstructive Pulmonary Disease)

### A. Single Agent Study

Three groups of 10 male mice were treated as follows:
1 group of 10 mice with a vehicle.
1 group of 10 mice with the anti-mouse TLR328G7 antibody administration (i.p., 500 μg/per mouse) on days 0, 3, 7, 10, 14, 17, 21 and 24.
1 group of 10 mice with the positive control roflumilast (approved as Daxas™; antagonist of PDE-4) (oral, 15 mg/kg, five times per week).

All mice were treated at days 0, 7, 14 and 21 with Elastin and LPS (i.n.), to induce COPD. At day 28 mice were sacrificed for analysis.

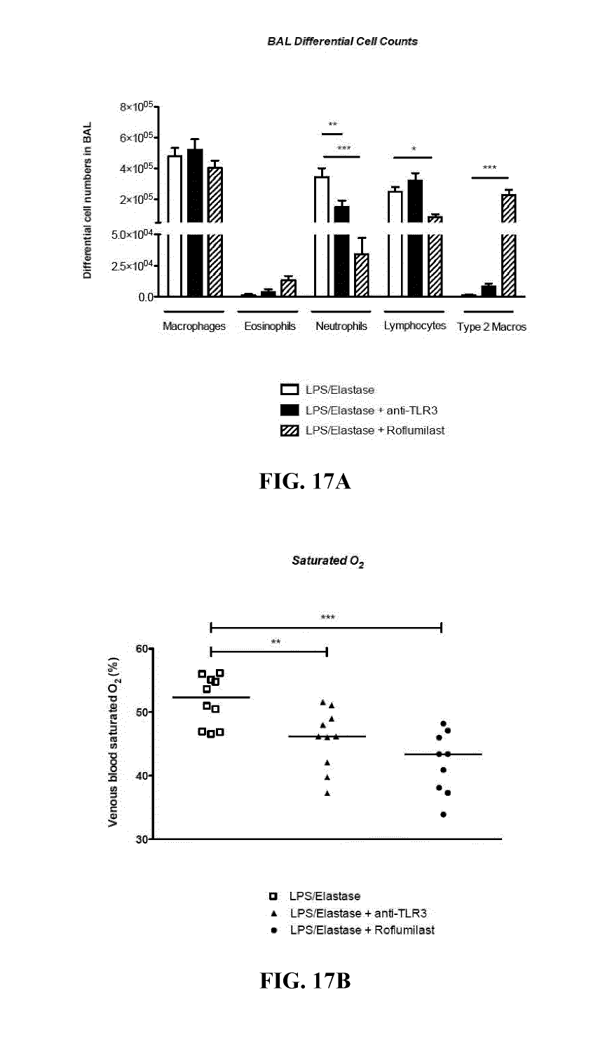

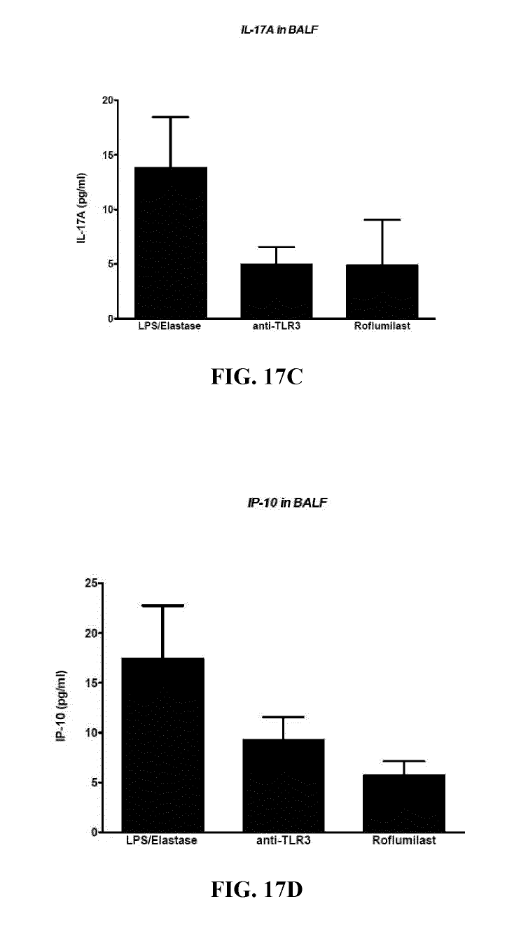

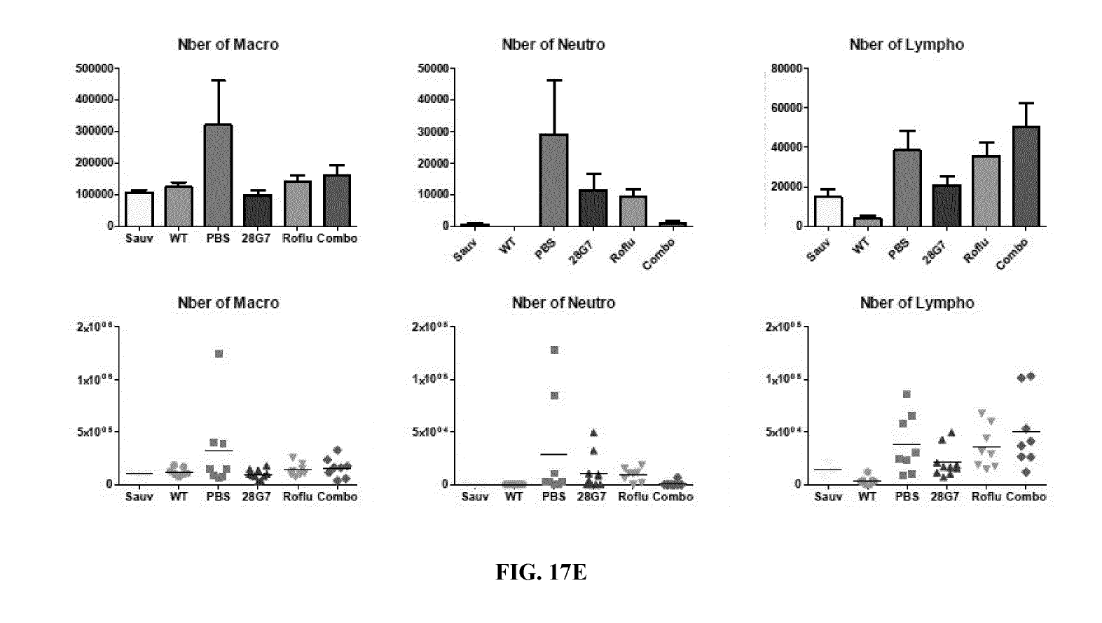

The cellular infiltrates into airways was measured by analysis of bronchoalveolar lavage (BAL) fluid by differential cell count on day 28. The oxygenation of venous blood was measured by gasometry on day 28. The level inflammatory mediators in BALF, through the analysis for protein levels of TNF-alpha, IL-6, IL-17A and IP-10 was performed by multiplex assay. Results are shown in FIG. 17. The figures show that the anti-TLR3 antibodies were highly effective in treating COPD, including in decreasing neutrophil airway infiltration, venous blood oxygen saturation and pro-inflammatory cytokines. Statistical analysis was performed on all data utilizing a Student's t test: ‘*’ p<0.05; ‘**’ p<0.005; ‘***’p<0.0005.

FIG. 17A shows BAL differential cell counts for macrophages, eosinophils, neutrophils and lymphocytes. The anti-TLR3 antibodies strongly decreased the infiltration of neutrophils into the airways, while not substantially affecting macrophages eosinophils or lymphocytes. COPD is primarily mediated by neutrophils rather than macrophages or eosinophils (nor lymphocytes, used here as a control).

FIG. 17B shows venous blood saturated oxygen (in percent) for each of LPS/elastase alone and LPS/elastase in combination with anti-TLR3 antibodies or roflumilast. The percent saturation of oxygen in arterial blood is diminished in COPD patients compared to healthy individuals, while the venous blood saturation in oxygen is increased in COPD patients compared to healthy individuals. A decrease in percent of O₂ in venous blood is therefore an important indicator of a favourable response in disease treatment (see O'Connor et al. Respiration 2011; 81:18-25). It can be seen that anti-TLR3 antibodies decreased percent of O₂ in venous blood substantially, almost as much as roflumilast. Anti-TLR3 antibodies are therefore effective in treating COPD. In medical practice, arterial blood gas (ABG) analysis is used both in the acute setting and for assessing patients' gas exchange status during periods of clinical stability. Blood gas analysis, including ABG analysis can therefore be a particularly useful tool to assess whether patients are suitable for treatment with anti-TLR3 antibodies and to assess or monitor whether treatment with anti-TLR3 antibodies is effective, during an acute phase or a phase of clinical stability.

FIG. 17C shows IL17A in BAL fluid (BALF). Anti-TLR3 antibodies decreased IL17A (pg/ml) substantially, and as much as roflumilast. FIG. 17D shows IP-10 in BALF. Anti-TLR3 antibodies decreased IP-10 (pg/ml) substantially.

B. Drug Combination Study

Four groups of mice were treated as follows:
1 group of mice with a vehicle (PBS, i.p., twice per week).
1 group of mice with the anti-mouse TLR328G7 antibody administration (i.p., 500 μg/per mouse, twice per week).
1 group of mice with the positive control roflumilast (approved as Daxas™; antagonist of PDE-4) (3 mg/kg, oral, five times per week).
1 group of mice with both roflumilast and 28G7 antibody, each agent according to the schedule used as single agent.

All mice were treated with Elastin and LPS (i.n.), to induce COPD and sacrified as in study A.

The cellular infiltrates into airways was measured by analysis of bronchoalveolar lavage (BAL) fluid by differential cell count as in Study A.

FIG. 17E shows BAL differential cell counts for macrophages, neutrophils and lymphocytes. The anti-TLR3 antibodies strongly decreased the infiltration of neutrophils into the airways, with decreases in macrophages and lymphocytes also observed. COPD is primarily mediated by neutrophils rather than macrophages or eosinophils (nor lymphocytes, used here as a control). Roflumilast also caused decreased infiltration of neutrophils into the airways, together with decreases in macrophages. Interestingly, the combination of anti-TLR3 antibodies and roflumilast caused decreased infiltration of neutrophils into the airways substantially to the level seen in wild-type mice, a decrease far beyond that observed with either agent alone.

### Example 14

### In Vivo Efficacy Model for the Treatment of Multiple Sclerosis

Briefly, 30 mice are administered pertussis toxin 200 mg, iv on day 0 and day 2 to allow blood brain barrier permeation.

On day 0, MBut-CFA and MTub 5 mg/ml+MOG 100 μg are administered s.c. 100 μl×2. Then, 10 mice are administered the antibody 28G7 (500 μg, ip, twice a week), whereas the 20 other mice are administered PBS (100 μl ip, twice a week). On day 14, which is the time of the peak of the disease, the PBS group are again be pooled into two groups: 10 mice receive the antibody 28G7 (500 μg, ip, twice a week), while the 10 other mice continue the PBS treatment.

Clinical observation is conducted every day and clinical score is assessed according to the following table (table 7).

TABLE 7

| score | Observations |
|---|---|
| 0 | No sign |
| 1 | Tail is soft but rear body section has a normal tonicity |
| 1.5 | Rear body section is lowered but a tail has normal tonicity |
| 2 | Rear body section is lowered and tail is soft |
| | Mouse moves normally on a grid, without hesitation |
| 2.5 | Rear body section is lowered and tail is soft |
| | Mouse cannot properly walk on a grid |
| | Loss in equilibriliun or trembling when mouse on a flat area |
| 3 | Partial paralysis of the back (the mouse can bend |
| | a leg, but not the other) |
| 4 | Total paralysis of the rear body section |
| 5 | Total paralysis of the rear body section |
| | Partial or total paralysis of the fore legs. |
| 6 | Death |

### Example 15

### In Vivo Efficacy Model for the Treatment of Sepsis—Canal Ligature and Puncture (CLP)—Curative Setting

Briefly 30 mice were operated: the surgery consists in cæcal ligature and puncture. By this way the content of the caecal lumen is draining of in the abdominal cavity leading to peritonitis and consequently a septic shock. The CLP is mid-grade, e.g. ligature is performed approximately in the middle of the cecum.

Mice were treated with 28G7 (100 μg/mouse, ip), a control antibody with no TLR3 specificity (“control”, 100 μg/mouse, ip) or the PBS (300 μl/mouse, ip) 6 hours and 24 hours after operation.

Survival was assessed at hours 24, 28, 32, 48, 52, 56, 72, 76, 80, 96, 100, 104, 120, 124, 128, 144, 148, 152, 168, 172, 176, 192, 196, 200, 216, 220, 224, 240, 244, 248, 264, 270, 274, 288, 292, 296, 312, 316, 320 and 336. After 336 hours, the mice which have survived have cleared the acute phase infection. The experiment was stopped and mice were sacrificed.

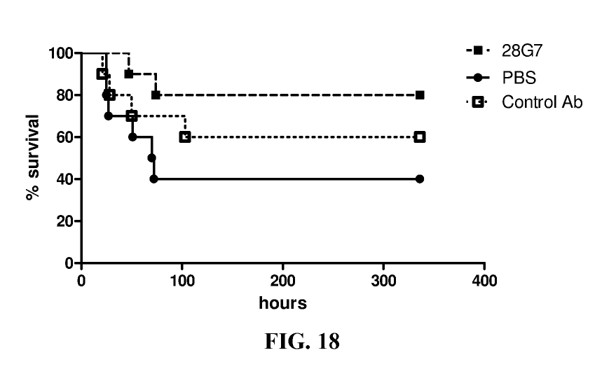

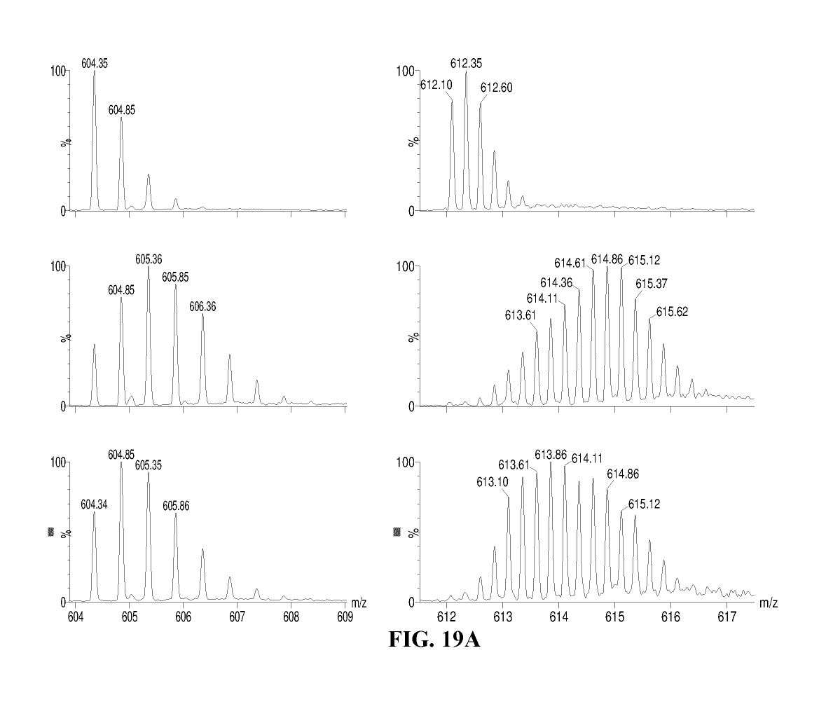

FIG. 18 shows the results of the experiment. The 28G7 treated group has a 80% survival whereas the non treated group experiences a 40% survival. These results demonstrate that TLR3 antibodies are efficient for the treatment of mice in a CLP mouse model. In this acute model, mice experience an acute infection, mimicking septic shock. The mice which survive have efficiently cleared the acute infection. Therefore, the antibodies according to the invention are suitable agents to treat a patient experiencing a severe acute infection such as a SIRS, a sepsis, a severe sepsis or a septic shock.

### Example 16

### Epitope Mapping by H/D Exchange

Introduction to HXMS: The FIX-MS technology exploits that hydrogen exchange (HX) of a protein can readily be followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction. The deuterium labelling information can be sub-localized to regions in the protein by pepsin digestion under quench conditions and following the mass increase of the resulting peptides.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent. Protein-protein complex formation may be detected by HX-MS simply by measuring the total amount of deuterium incorporated in either protein members in the presence and absence of the respective binding partner as a function of time. The FIX-MS technique uses the native components, i.e. protein and antibody or Fab fragment, and is performed in solution. Thus FIX-MS provides the possibility for mimicking the in vivo conditions (for a recent review on the FIX-MS technology, see Wales and Engen, Mass Spectrom. Rev. 25, 158 (2006)).

### Materials:

TLR3: TLR3 consisting of residues Lys27-Ser711. Purchased from R&D Systems and supplied as 0.713 mg/ml (9 μM) solution in PBS.

mAbs: 23C8, 31C3 and 34A3. All mAbs were buffer exchanged and concentrated to approximately 25 mg/ml in PBS pH 7.4. The mAbs were then inspected for fragmentation and aggregation by SDS-PAGE and SEC-MALS, respectively (see appendix 2 for details).

### Methods: HX-MS Experiments

Instrumentation and data recording: The FIX experiments were automated by a Leap robot (H/D-x PAL; Leap Technologies Inc.) operated by the LeapShell software (Leap Technologies Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and FIX reactions and maintained at 2° C. for storage of protein and quench solution, respectively. The Leap robot furthermore contained a cooled Trio VS unit (Leap Technologies Inc.) holding the pre- and analytical columns, and the LC tubing and switching valves at 1° C. The switching valves of the Trio VS unit have been upgraded from HPLC to Microbore UHPLC switch valves (Cheminert, VICI AG). For the inline pepsin digestion, 150 μL quenched sample containing 90 pmol TLR3 was loaded and passed over a Poroszyme® Immobilized Pepsin Cartridge (2.1×30 mm (Applied Biosystems)) placed at 20° C. using a isocratic flow rate of 125 μL/min (0.1% formic acid:CH3CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (2.1×100 mm (Waters Inc.)), and the peptides separated using a 9 min gradient of 10-40% B delivered at 200 μl/min from an AQUITY UPLC system (Waters Inc.). The mobile phases consisted of A: 0.1% formic acid and B: 0.1% formic acid in CH3CN. The ESI MS data, and the separate data dependent MS/MS acquisitions (CID) and elevated energy (MSE) experiments were acquired in positive ion mode using a Q-TOF Premier MS (Waters Inc.). Leucine-enkephalin was used as the lock mass ([M+H]+ ion at m/z 556.2771) and data was collected in continuum mode (For further description of the set-up, see Andersen and Faber, International Journal of Mass Spectrometry, In Press, Corrected Proof, DOI: 10.1016/j.ijms.2010.09.010).

### Data Analysis:

Peptic peptides were identified in separate experiments using standard CID MS/MS or MSE methods (Waters Inc.). MSE data were processed using BiopharmaLynx 1.2 (version 017). CID data-dependent MS/MS acquisition was analyzed using the MassLynx software and in-house MASCOT database.

HX-MS raw data files were subjected to continuous lock mass-correction. Data analysis, i.e., centroid determination of deuterated peptides and plotting of in-exchange curves, was performed using prototype custom software (HDX browser, Waters Inc.) and FIX-Express ((Version Beta); Weis et al., J. Am. Soc. Mass Spectrom. 17, 1700 (2006)). All data were also visually evaluated to ensure only resolved peptide isotopic envelopes were subjected to analysis.

### Epitope Mapping Experiments:

Amide hydrogen/deuterium exchange (HX) was initiated by a 10-fold dilution of TLR3 in the presence or absence of 23C8.4, 31C3.1 or 34A3.5 into the corresponding deuterated buffer (i.e. PBS prepared in D20, 96% D2O final, pH 7.4 (uncorrected value)). All FIX reactions were carried out at 20° C. and contained 0.9 μM TLR3 in the absence or presence of 0.9 μM mAb thus giving a 2 fold molar excess of mAb binding sites. At appropriate time intervals ranging from 17 sec to 3000 sec, 100 μl aliquots of the FIX reaction were quenched by 50 μl ice-cold quenching buffer (1.35M TCEP) resulting in a final pH of 2.6 (uncorrected value). Examples of raw data identifying the 34A3.5 epitope is shown in FIG. 19A.

### Results: Epitope Mapping of 34A3

The FIX time-course of 68 peptides, covering 70% of the primary sequence of TLR3, were monitored in the presence and absence 23C8, 31C3 or 34A3 for 17 sec to 3000 sec (FIGS. 19A, 19B, 19C).

The observed exchange pattern in the presence or absence of 34A3 can be divided into two different groups: One group of peptides display an exchange pattern that is unaffected by the binding of 34A3.5 to TLR3. In contrast, another group of peptides in TLR3 show protection from exchange upon 34A3.5 binding (FIG. 19B). For example at 100 sec exchange with D₂O, approximately 1.5 amides difference in exchange is observed in the region 131-151 upon 34A3.5 binding (FIG. 19B). The regions displaying protection upon mAb 34A3 binding encompass peptides covering residues 102-121 and 131-152 with similar exchange protection observed in each region. No information could be obtained for region 122-130.

Weak exchange protection was also observed in the 153-173 region upon mAb 34A3 binding (See next section for details).

### Results, Epitope Mapping of 24C8, 31C3 and 34A3

The epitopes for all three mAbs 24C8, 31C3 and 34A3 are presumably overlapping since the mAbs compete for TLR3 binding in a BiaCore assay (see example 7).

The tertiary structure of TLR3 has been solved from two independent crystal structures (pdb codes 1 Z1W and 2A0Z; FIG. 19F). Because of the structural organization of the TLR3 molecule, regions close in space will also be close in sequence (FIG. 19F).

The 34A3 epitope regions within residues 102-121 and 131-152 did not show sign of 24C8 or 31C3 binding (FIG. 19B). Neither did preceding regions in TLR3. However, the subsequent region containing residues 152-173 displayed weak exchange protection upon binding of either one of the three mAbs 24C8, 31C3 or 34A3 (FIG. 19D). The exchange protection is most evident in region 168-173.

Apart from residues 152-173, the epitopes for 23C8 and 31C3 were not detected in other regions of TLR3. However, the weak exchange protection observed most likely cannot account for a full epitope. Furthermore the region containing residues 192-204 where no HX-MS information could be obtained is very close in the structure (FIG. 19G-C) and thus could hold the remaining part of the epitope.

### Conclusion

The epitope of mAb 34A3 have been mapped on TLR3 using HX-MS technology. The region corresponding to amino acid residues 102-121 and 131-152 of the mature TLR3 polypeptide (SEQ ID NO: 1) are involved in binding mAb 34A3 (FIG. 19G-A), and the region corresponding to amino acid residues 153-173, particularly 168-173, can also be involved in binding mAb 34A3 (FIG. 19G-B).

Given that the mAbs 23C8, 31C3 and 34A3 have overlapping epitopes, the residue 153-173 region, and particularly the region of residues 168-173, is the sole region on TLR3 that could contain the overlapping parts of the epitopes. This region show weak exchange protection upon binding of all the three mAbs.

No HX-MS information could be obtained for residues 192-204. However, this region is structurally adjacent to 168-173 and can hold the remaining part of the epitopes for 23C8 and 31C3.

### Example 17

### Epitope Mapping by Binding to TLR3 Mutants

TLR3 mutant polypeptides having mutations K145E, D116R, K182E, N196A and E171A (reference to SEQ ID NO 1) were prepared as described herein in the Materials and Methods and anti-TLR3 antibody staining to cells expressing TLR3 mutant polypeptides was assessed by FACS. The binding profile for antibody 31C3 is summarized in Table 8 (“+” indicates staining by the antibody and “−” indicates no staining by the antibody. The antibody did not show any loss of binding to unmutated wild type (WT) TLR3 as well as each of K145E, D116R, N196A and E171A. The antibody showed a substantial loss in binding, however, to mutant K182E. The principal epitope of the antibody may therefore include residue 182 but not residues K145, D116, N196 or E171. FIG. 20D shows a view of the side of the N-terminal end of the TLR3 polypeptide, showing amino acid residues K145, D116, K182, N196 and E171. FIG. 20E shows a view of the non-glycosylated face of the TLR3 polypeptide, with the N-terminal end of the TLR3 polypeptide in the foreground, showing amino acid residues K145, D116, K182, N196 and E171.

TABLE 8

| TLR3 polypeptide | Staining |
|---|---|
| TLR3 WT | + |
| K145E | + |
| D116R | + |
| E171A | + |
| K182E | − |
| N196A | + |

### Example 18

### IP-10 Production by Donors in Response to dsRNA and Drug Combinations

IP-10 production was assessed in human donors in response to polyAU (IPH3102), believed to be a specific agonist of TLR3, or pIC, believed to be an agonist of TLR3 as well as other dsRNA receptors such as RIG-I and MDA-5. Fresh PBMC were isolated from whole blood of two independent donors. 1.5×10⁶ (donor #1) and 3×10⁶ (donor #2) PBMC per ml were incubated in flat-bottom 96W plates in the presence of 50 μg/ml 31C3 or 34A3 anti-human TLR3 mAbs and a dose range of methotrexate (300, 30 and 3 μg/ml), dexamethasone (200, 20, 2 μg/ml) or Humira® (100, 10, 1 μg/ml). Cells were incubated 1 hr at 37° C. prior addition of 300 μg/ml IPH3102 or 30 μg/ml poly(I:C). Cells were incubated for 24 additional hours at 37° C. Supernatant were then harvested to quantify IP10 production by ELISA. Cells were then recovered, stained with 7-AAD and analyzed by flow cytometry (FACS) to evaluate potential toxic effects of any drugs.

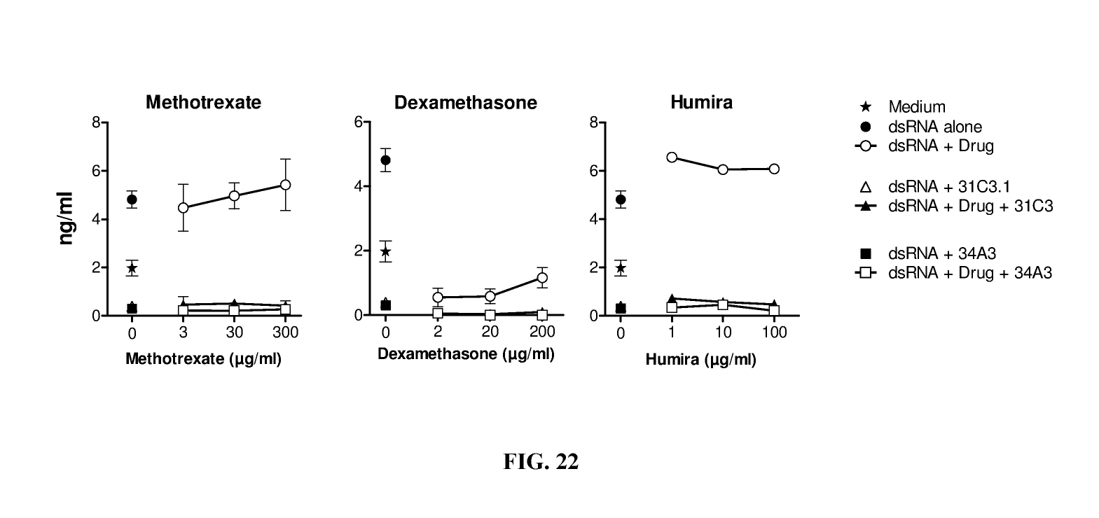

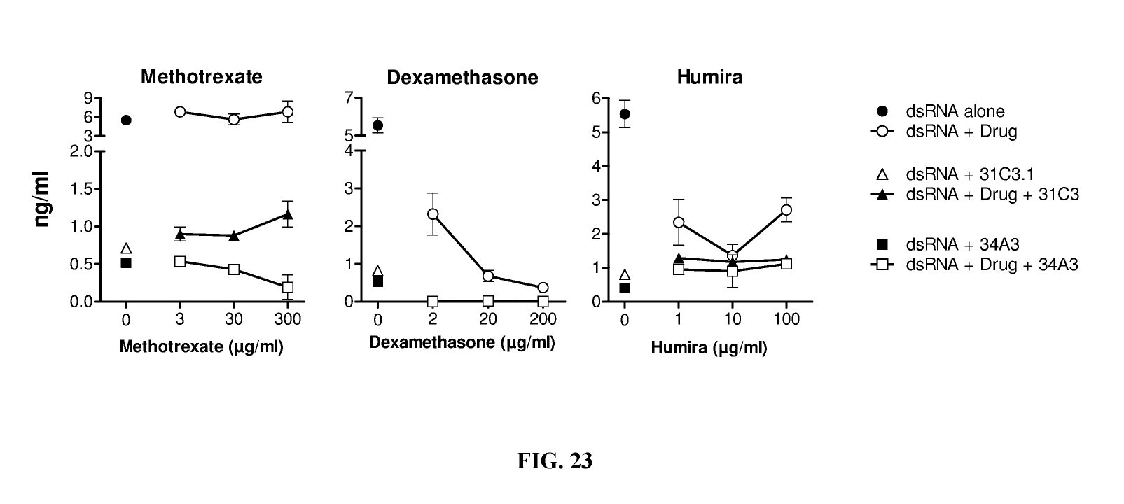

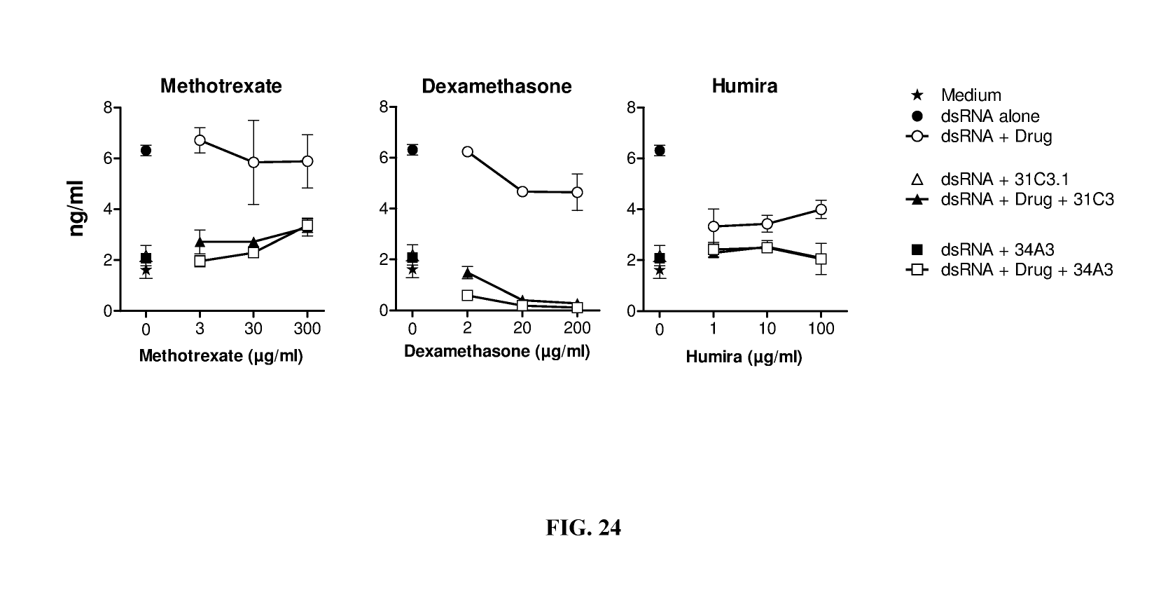

Result of drug combinations with anti-human TLR3 mAbs 31C3 or 34A3 in combination with methotrexate, dexamethasone or Humira® are shown in FIGS. 22, 23 and 24. As shown in FIG. 22 for human donor 2, antibodies 31C3 and 34A3 each substantially reduce IP-10 production in response to polyAU and that the antibodies reduce IP-10 further when combined with methotrexate, dexamethasone or Humira®. In FIG. 22, for each of methotrexate, dexamethasone or Humira®, the data points for antibodies 31C3 and 34A3 (alone or in combination with dsRNA and drug) are superposed. As shown in FIGS. 23 and 24 for human donors 1 and 2 respectively, antibodies 31C3 and 34A3 each substantially reduce IP-10 production in response to polyIC and that the antibodies reduce IP-10 further when combined with methotrexate, dexamethasone or Humira®. In FIG. 23, for dexamethasone the data points for antibodies 31C3 and 34A3 are superposed. In FIG. 24, for each of methotrexate, dexamethasone or Humira, the data points for antibodies 31C3 and 34A3 (alone or in combination with dsRNA and Drug) are superposed, unless indicated otherwise by diverging data points. For human donor 2, IP-10 production in the presence of medium is indicated and IP-10 is shown in intervals of 2 ng/ml because production in the absence of dsRNA was not null. finally, FACS analysis was performed on cells after activation to evaluate the toxic effect of the tested drug combinations. No toxicity was observed.

Anti-TLR3 antibodies can therefore provide an additional effect when used in combination with methotrexate, dexamethasone or Humira. In particular, in response to polyIC the antibodies potentiate the effects of dexamethasone, the treatment of reference in rheumatoid arthritis. Furthermore, the anti-TLR3 antibodies appeared to be more effective that Humira, suggesting that based on IP-10 production, the TNFα pathway is included in the TLR3 response to dsRNA. The anti-TLR3 antibodies can operate by modulating a signaling pathway that is complementary to those modulated by methotrexate, dexamethasone or Humira®, without antagonistic effects, and can therefore be used advantageously in combination with such drugs.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents, The description herein of any aspect or embodiment of the invention using terms such as reference to an element or elements is intended to provide support for a similar aspect or embodiment of the invention that “consists of”,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A monoclonal antibody that specifically binds a TLR3 polypeptide, wherein said antibody:
(i) inhibits signaling by the TLR3 polypeptide signaling without blocking binding of a dsRNA TLR3 ligand to the C-terminal dsRNA binding site of the TLR3 polypeptide,
(ii) is capable of being internalized by a TLR3-expressing cell, and
(iii) has a Kd of less than 10⁻⁹M for binding to a TLR3 polypeptide at acidic pH.

2. The antibody of claim 1, wherein said antibody binds to a TLR3 polypeptide within the segment corresponding to residues 102 to 204 and does not bind residue 116 and/or residue 145 of the TLR3 polypeptide of SEQ ID NO: 1.

3. The antibody of claim 1, wherein said antibody comprises a human IgG4 heavy chain comprising a serine to proline mutation in residue 241, corresponding to position 228 according to the EU-index.

4.-8. (canceled)

9. The antibody of claim 1, wherein said antibody does not have a significant reduction in binding to a TLR3 polypeptide having a mutation at residues 116, 145, 171 and/or residue 196 of the TLR3 polypeptide of SEQ ID NO: 1, relative to binding between the antibody and a wild-type TLR3 polypeptide of SEQ ID NO: 1.

10. The antibody of claim 1, wherein said antibody has a significant reduction in binding to a TLR3 polypeptide having a mutation at residue 182 of the TLR3 polypeptide of SEQ ID NO: 1, relative to binding between the antibody and a wild-type TLR3 polypeptide of SEQ ID NO: 1.

11. The antibody of claim 1, wherein said antibody binds to a TLR3 polypeptide within the segment corresponding to residues 174 to 191 and does not bind residue 116 and/or residue 145 of the TLR3 polypeptide of SEQ ID NO: 1.

12. A monoclonal antibody that specifically binds a TLR3 polypeptide, wherein said antibody inhibits signaling by the TLR3 polypeptide without blocking binding of a dsRNA TLR3 ligand to the C-terminal dsRNA binding site of the TLR3 polypeptide, wherein said antibody binds to at least one residue in the segment corresponding to residues 102 to 204 of the mature TLR3 polypeptide of SEQ ID NO: 1, wherein said antibody does not bind residue 116 and/or residue 145 of the TLR3 polypeptide of SEQ ID NO: 1.

13-14. (canceled)

15. The antibody of claim 12, wherein said antibody binds to at least one residue in the segment corresponding to residues 174 to 191 of the mature TLR3 polypeptide of SEQ ID NO: 1.

16. The antibody of claim 12, wherein said antibody binds amino acid residue 182 of the TLR3 polypeptide of SEQ ID NO:1.

17.-19. (canceled)

20. The antibody of claim 12, wherein said antibody comprises an IgG4 heavy chain comprising a serine to proline mutation in residue 228 according to the EU-index.

21. The antibody of claim 1, wherein the antibody comprises:
(a) heavy chain CDR 1, 2 and 3 (HCDR1, HCDR2, HCDR3) amino acid sequences as shown in SEQ ID NO: 74, 78 and 84, respectively and/or
(b) light chain CDR 1, 2 and 3 (LCDR1, LCDR2, LCDR3) amino acid sequences as shown in SEQ ID NO: 64, 62 and 69, respectively;
wherein one or two of the amino acids in any of said sequences may optionally be substituted by a different amino acid.

22. (canceled)

23. The antibody of claim 12, wherein said antibody comprises an IgG4 heavy chain comprising a serine to proline mutation in residue 228 according to the EU-index.

24.-25. (canceled)

26. The antibody of claim 1, wherein said antibody is conjugated or covalently bound to a toxic moiety.

27.-29. (canceled)

30. A pharmaceutical composition comprising an antibody of claim 1 and a pharmaceutically acceptable carrier.

31. The composition of claim 30, wherein the antibody is present in an amount of between about 25 mg and 500 mg.

32. The composition of claim 30, wherein the composition is formulated for administration to nasal or pulmonary tissue.

33.-35. (canceled)

36. A hybridoma or recombinant host cell producing the antibody of claim 1.

37. A method of producing an antibody that specifically binds a TLR3 polypeptide in a mammalian subject, said method comprising the steps of: a) providing a plurality of antibodies, optionally; and b) selecting an antibody from said plurality of antibodies, optionally from said immunized animal, that inhibits TLR3 signaling without blocking binding of a TLR3 ligand to the C-terminal dsRNA binding site of the TLR3 polypeptide.

38. A method of treating an individual having an autoimmune or inflammatory disease, comprising administering to the individual an effective amount of an antibody according to claim 1.

39.-41. (canceled)

42. The method of claim 38, wherein the individual has an established or chronic autoimmune or inflammatory disease.

43. The method of claim 42, wherein the individual has an attack, crisis, flare or exacerbation.

44.-49. (canceled)

50. The method of claim 38, further comprising administering to the individual a DMARD.

51. (canceled)

52. The method of claim 50, wherein said DMARD is an anti-TNFα antibody and/or methotrexate.

53. A method for determining the suitability of treatment with an antibody that binds a TLR3 polypeptide of claim 1 for a patient, comprising determining whether said patient has an established autoimmune or inflammatory disease, whether said patient is experiencing an attack, crisis, exacerbation or flare, and/or whether said patient has a disease characterized by the presence of dsRNA.

54. (canceled)

55. A recombinant DNA encoding the antibody of claim 1.

56. The method of claim 38, wherein the effective amount is between about 0.05 and 20 mg/kg, and the antibody is administered to the individual at a frequency of from about once per week to about once every 2 months.


SEQUENCE LISTING (95 sequences)

SEQ ID NO: 1 - protein, 904 aa, homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 2 - protein, 136 aa, Mus musculus
  SIGNAL (1)..(19)
MGWSWIFLFLLSGTAGVLSEVQLQQSGPELVKPGASVKISCKPSGYSFTAYYMHWVKQSHVKSLEWIGRINPYNGATSYNRNFKDKASLTVDKSSSTAYMELHSLTSEDSAVYYCARSGGNTYFDYWGQGTTLTVS

SEQ ID NO: 3 - protein, 127 aa, Mus musculus
  SIGNAL (1)..(20)
  VARIANT (91)..(91): Xaa = Phe or Ser
MSVPTQVLGLLLLWLTGARCDIQMTQSPASLSASVGETVTITCRASENIYSSLAWYQQKQGKSPQLLVYNAKTLAEGVPSRFSGSGSGTQXSLKINSLQPEDFGTYYCQHHYGTPPTFGGGTKLEIK

SEQ ID NO: 4 - protein, 9 aa, Mus musculus
GYSFTAYYM

SEQ ID NO: 5 - protein, 10 aa, Mus musculus
RINPYNGATS

SEQ ID NO: 6 - protein, 9 aa, Mus musculus
SGGNTYFDY

SEQ ID NO: 7 - protein, 10 aa, Mus musculus
RASENIYSSL

SEQ ID NO: 8 - protein, 7 aa, Mus musculus
NAKTLAE

SEQ ID NO: 9 - protein, 9 aa, Mus musculus
QHHYGTPPT

SEQ ID NO: 10 - protein, 134 aa, Mus musculus
  SIGNAL (1)..(18)
MRVLILLCLFTAFPGILSDVQLQESGPDLVKPSQSLSLTCTVTGYSITSGYSWHWIRQFLGNKLEWMGYIHYSGITNYNPSLRSRISFTRDTSKNQFFLQLNSVTTEDTATYYCARDGYYGMDYWGQGTSVTVS

SEQ ID NO: 11 - protein, 127 aa, Mus musculus
  SIGNAL (1)..(20)
MDFQTQVFVFVLLWLSGVDGDIVMTQSQKFMSTSVGDRVSITCKASQNVRTSVAWYQQKPGQSPKALIYLASNRHTGVPDRFTGSGSGTDFTLTVSNIQSEDLADYFCLQHWNYPYTFGGGTKLEIK

SEQ ID NO: 12 - protein, 11 aa, Mus musculus
GYSITSGYSWH

SEQ ID NO: 13 - protein, 9 aa, Mus musculus
YIHYSGITN

SEQ ID NO: 14 - protein, 8 aa, Mus musculus
DGYYGMDY

SEQ ID NO: 15 - protein, 11 aa, Mus musculus
KASQNVRTSVA

SEQ ID NO: 16 - protein, 7 aa, Mus musculus
LASNRHT

SEQ ID NO: 17 - protein, 8 aa, Mus musculus
LQHWNYPY

SEQ ID NO: 18 - protein, 136 aa, Mus musculus
  SIGNAL (1)..(19)
MGWSWIFLFLLSGTAGVLSEVQLQQSGPELVKPGASVKISCKASGYSFTGYYIHWVKQSHVKSLEWIGRINPYYGATSYNQNFKDKANLTVDKSSSTAYMELHSLTSDDSAVYYCARSTKLGYLDYWGQGTTLTVS

SEQ ID NO: 19 - protein, 127 aa, Mus musculus
  SIGNAL (1)..(20)
MSVPTQVLGLLLLWLTGARCDIQMTQSPASLSASVGETVTITCRASENIYSNLAWYQQKQGKSPQLLIYNAKTLAEGVPSRFSGSGSGTQYFLKINSLQPEDFGSYYCQHHYGTPFTFGGGTKLEIK

SEQ ID NO: 20 - protein, 10 aa, Mus musculus
GYSFTGYYIH

SEQ ID NO: 21 - protein, 9 aa, Mus musculus
RINPYYGAT

SEQ ID NO: 22 - protein, 9 aa, Mus musculus
STKLGYLDY

SEQ ID NO: 23 - protein, 11 aa, Mus musculus
RASENIYSNLA

SEQ ID NO: 24 - protein, 7 aa, Mus musculus
NAKTLAE

SEQ ID NO: 25 - protein, 9 aa, Mus musculus
QHHYGTPFT

SEQ ID NO: 26 - protein, 137 aa, Mus musculus
  SIGNAL (1)..(19)
MGWSWIFLFLLSGTAGVLSEVQLQQSGPELVKPGASVKISCKASGYSFTGYFMHWVKQSHVKSLEWIGRINPYNGATSYNQNFKDKASLTVDKSSSTSYMELHSLTSEDSAVYYCVRDDGGNYPFDYWGQGTTLTVS

SEQ ID NO: 27 - protein, 127 aa, Mus musculus
  SIGNAL (1)..(20)
MSVPTQVLGLLLLWLTGARCDIQMTQSPASLSASVGETVTITCRASENIYSYLAWYQQKQGKSPQLLVYYAKTLAEGVPSRFSGSGTGTQFSLKINSLQPEDFGSYYCQHHYGTPYTFGGGTKLEIK

SEQ ID NO: 28 - protein, 10 aa, Mus musculus
GYSFTGYFMH

SEQ ID NO: 29 - protein, 10 aa, Mus musculus
RINPYNGATS

SEQ ID NO: 30 - protein, 10 aa, Mus musculus
DDGGNYPFDY

SEQ ID NO: 31 - protein, 11 aa, Mus musculus
RASENIYSYLA

SEQ ID NO: 32 - protein, 7 aa, Mus musculus
YAKTLAE

SEQ ID NO: 33 - protein, 9 aa, Mus musculus
QHHYGTPYT

SEQ ID NO: 34 - protein, 137 aa, Mus musculus
  SIGNAL (1)..(19)
MEWRWIFLFLLSGTTGVHSEIQLQQSGPELVKPGASVKVSCKASGYVFTTYSIYWVKQSHGKSLEWIGYIDPYNGDTSYNQKFKGKATLTVDKSSSTAYMHLNSLTSEDSTVYYCAREGNYYGYFDYWGQGTTLTVS

SEQ ID NO: 35 - protein, 128 aa, Mus musculus
  SIGNAL (1)..(22)
MDFQVQIFSFLLMSASVIMSRGQIVLTQSPALMSASPGEKVTMTCSASSSVSYMFWYQQKPRSSPKPWIYLTSNLASGVPARFSGSGSGTSYSLTISSMEAEDAATYYCQQWTGNPPTFGGGTKLEIK

SEQ ID NO: 36 - protein, 10 aa, Mus musculus
GYVFTTYSIY

SEQ ID NO: 37 - protein, 10 aa, Mus musculus
YIDPYNGDTS

SEQ ID NO: 38 - protein, 10 aa, Mus musculus
EGNYYGYFDY

SEQ ID NO: 39 - protein, 10 aa, Mus musculus
SASSSVSYMF

SEQ ID NO: 40 - protein, 7 aa, Mus musculus
LTSNLAS

SEQ ID NO: 41 - protein, 9 aa, Mus musculus
QQWTGNPPT

SEQ ID NO: 42 - DNA, 285 bp, Mus musculus
gacattgtgatgacccagtctcaaaaattcatgtccacatcagtaggagacagggtcagcatcacctgcaaggccagtcagaatgttcgtactgctgtagcctggtatcaacagaaaccagggcagtctcctaaagcactgatttacttggcatccaaccggcacactggagtccctgatcgcttcacaggcagtggatctgggacagatttcactctcaccattagcaatgtgcaatctgaagacctggcagattatttctgtctgcaacattggaattatcct

SEQ ID NO: 43 - DNA, 285 bp, Mus musculus
caaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccaccc

SEQ ID NO: 44 - DNA, 285 bp, Mus musculus
gacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtactcct

SEQ ID NO: 45 - DNA, 39 bp, Mus musculus
tacacgttcggaggggggaccaagctggaaataaaacgt

SEQ ID NO: 46 - DNA, 294 bp, Mus musculus
gatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttgaattctgtgactactgaggacacagccacatattactgtgcaaga

SEQ ID NO: 47 - DNA, 294 bp, Mus musculus
gaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaaga

SEQ ID NO: 48 - DNA, 294 bp, Mus musculus
gaggtccagctgcaacagtctggacctgagctggtgaagcctggggcttcagtgaagatatcctgcaaggcttctggttactcattcactggctactacatgcactgggtgaagcaaagccatgtaaagagccttgagtggattggacgtattaatccttacaatggtgctactagctacaaccagaatttcaaggacaaggccagcttgactgtagataagtcctccagcacagcctacatggagctccacagcctgacatctgaggactctgcagtctattactgtgcaagg

SEQ ID NO: 49 - DNA, 45 bp, Mus musculus
tactttgactactggggccaaggcaccactctcacagtctcctca

SEQ ID NO: 50 - DNA, 53 bp, Mus musculus
attactatgctatggactactggggtcaaggaacctcagtcaccgtctcctca

SEQ ID NO: 51 - DNA, 408 bp, Mus musculus
atgggatggagctggatctttctctttctcctgtcaggaactgcaggtgtcctctctgaggtccagctgcaacagtctggacctgagctggtgaagcctggggcttcagtgaagatatcctgcaagccttctggttactcattcactgcctactacatgcactgggtgaagcaaagccatgtaaagagccttgagtggattggacgtattaatccttacaatggtgctactagctacaaccggaatttcaaggacaaggccagcttgactgtagataagtcctccagcacagcctacatggagctccacagcctgacatctgaggactctgcagtctattactgtgcaagaagtgggggtaatacgtactttgactactggggccaaggcaccactctcacagtctcc

SEQ ID NO: 52 - DNA, 381 bp, Mus musculus
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatatttacagttctttagcatggtatcagcagaaacagggaaaatctcctcagctcctggtctataatgcaaaaaccttagcagaaggtgtgccatcgaggttcagtggcagtggatcaggcacacagttttctctgaagatcaacagcctgcagcctgaagatyttgggacttattactgtcaacatcattatggtactcctccgacgttcggtggaggcaccaagctagaaatcaaa

SEQ ID NO: 53 - DNA, 402 bp, Mus musculus
atgagagtgctgattcttttgtgcctgttcacagcctttcctggtatcctgtctgatgtgcagcttcaggagtcaggacctgacctggtgaaaccttctcagtcactttcactcacctgcactgtcactggctactccatcaccagtggttatagctggcactggatccggcagtttctaggaaacaaactggaatggatgggctacattcactacagtggtatcactaactacaacccatctctcagaagtcgaatctctttcactcgagacacatccaagaaccagttcttcctgcagttgaattctgtgactactgaggacacagccacatattactgtgcaagagatggttattatggtatggactactggggtcaaggaacctcagtcaccgtctcc

SEQ ID NO: 54 - DNA, 381 bp, Mus musculus
atggactttcagacccaggtctttgtattcgtgttgctctggttgtctggtgttgatggagacattgtgatgacccagtctcaaaaattcatgtccacatcagtaggagacagggtcagcatcacctgcaaggccagtcagaatgttcgtacttctgtagcctggtatcaacagaaaccagggcagtctcctaaagcactgatttacttggcatccaaccggcacactggagtccctgatcgcttcacaggcagtggatctgggacagatttcactctcaccgttagcaatattcaatctgaggacctggcagattatttctgtctgcaacattggaattatccgtacacgttcggaggggggaccaagctggaaataaaa

SEQ ID NO: 55 - DNA, 408 bp, Mus musculus
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctactacatacactgggtgaaacaaagccatgtaaagagccttgagtggattggacgtattaatccttactatggtgctactagctacaaccagaatttcaaggacaaggccaacttgactgtagataagtcctccagcacagcctacatggagctccacagtctgacatctgacgactctgcagtctattactgtgcaagatcgaccaaactggggtatcttgactactggggccaaggcaccactctcacagtctcc

SEQ ID NO: 56 - DNA, 381 bp, Mus musculus
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatatttacagtaatttagcatggtatcagcagaaacagggaaaatctcctcagctcctgatctataatgcaaaaaccttagcagaaggtgtgccatcaaggttcagtggcagtggatcaggcacacaatattttctgaagatcaacagcctgcagcctgaagattttgggagttattactgtcaacatcattatggtactccgttcacgttcggaggggggaccaagctggaaataaaa

SEQ ID NO: 57 - DNA, 411 bp, Mus musculus
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctacttcatgcactgggtgaagcaaagccatgtaaagagccttgagtggattggacgtattaatccttacaatggcgctactagctacaaccagaatttcaaggacaaggccagcttgactgtagataagtcctccagcacatcctacatggaactccacagcctgacatctgaggactctgcagtctattactgtgtaagagacgacggtggtaactacccttttgactactggggccagggcaccactctcacagtctcc

SEQ ID NO: 58 - DNA, 381 bp, Mus musculus
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatatttacagttatttagcatggtatcagcagaaacagggaaaatctcctcagctcctggtctattatgcaaaaaccttagcagaaggtgtgccatcaaggttcagtggcagtggaacaggcacacagttttctctgaagatcaacagcctgcagcctgaagattttgggagttactactgtcaacatcattatggtactccgtacacgttcggaggggggaccaagctggaaataaaa

SEQ ID NO: 59 - DNA, 411 bp, Mus musculus
atggaatggagatggatctttctcttcctcctgtcaggaactacaggtgtccactctgagatccagctgcagcagtctggacctgagttggtgaagcctggggcttcagtgaaggtatcctgcaaggcttctggttatgtattcactacctacagcatttactgggtgaagcagagccatggaaagagccttgagtggattggatatattgatccttacaatggtgatactagctacaaccagaagttcaagggcaaggccacattgactgttgacaagtcctccagcacagcctacatgcatctcaacagcctgacatctgaggactctacagtctattactgcgcaagagagggcaattactacggctactttgactactggggccaaggcaccactctcacagtctcc

SEQ ID NO: 60 - DNA, 384 bp, Mus musculus
atggattttcaagtgcagattttcagcttcctgctaatgagtgcctcagtcataatgtccaggggacaaattgttctcacccagtctccagcactcatgtctgcatctccaggggagagggtcaccatgacctgcagtgccagctcaagtgtaagttacatgttctggtaccagcagaagccaagatcctcacccaaaccctggatttatctcacatccaacctggcttctggagtccctgctcgcttcagtggcagtgggtctgggacctcttactctctcacaatcagcagcatggaggctgaagatgctgccacttattactgccagcagtggactggtaacccacccacgttcggaggggggaccaagctggaaataaaa

SEQ ID NO: 61 - protein, 11 aa, Mus musculus
  misc_feature (9)..(9): Xaa can be any naturally occurring amino acid
RASENIYSXLA

SEQ ID NO: 62 - protein, 7 aa, Mus musculus
  misc_feature (1)..(1): Xaa can be any naturally occurring amino acid
XAKTLAE

SEQ ID NO: 63 - protein, 9 aa, Mus musculus
  misc_feature (8)..(8): Xaa can be any naturally occurring amino acid
QHHYGTPXT

SEQ ID NO: 64 - protein, 11 aa, Mus musculus
  misc_feature (1)..(1): Xaa can be any naturally occurring amino acid
XASXXXXXXXX

SEQ ID NO: 65 - protein, 7 aa, Mus musculus
  misc_feature (1)..(4): Xaa can be any naturally occurring amino acid
XXXXLAX

SEQ ID NO: 66 - protein, 7 aa, Mus musculus
  misc_feature (1)..(1): Xaa can be any naturally occurring amino acid
XAXXXXX

SEQ ID NO: 67 - protein, 7 aa, Mus musculus
  misc_feature (2)..(2): Xaa can be any naturally occurring amino acid
LXSNXXX

SEQ ID NO: 68 - protein, 9 aa, Mus musculus
  misc_feature (2)..(4): Xaa can be any naturally occurring amino acid
QXXXGXPXT

SEQ ID NO: 69 - protein, 9 aa, Mus musculus
  Xaa (2)..(2): Xaa can be any naturally occurring amino acid
QXHXXXPXX

SEQ ID NO: 70 - protein, 9 aa, Mus musculus
  misc_feature (1)..(1): Xaa can be any naturally occurring amino acid
XQXXXXPXT

SEQ ID NO: 71 - protein, 10 aa, Mus musculus
  misc_feature (8)..(9): Xaa can be any naturally occurring amino acid
GYSFTGYXXH

SEQ ID NO: 72 - protein, 10 aa, Mus musculus
  misc_feature (6)..(6): Xaa can be any naturally occurring amino acid
GYSFTXYXMH

SEQ ID NO: 73 - protein, 10 aa, Mus musculus
  misc_feature (6)..(6): Xaa can be any naturally occurring amino acid
GYSFTXYYXH

SEQ ID NO: 74 - protein, 10 aa, Mus musculus
  misc_feature (3)..(3): Xaa can be any naturally occurring amino acid
GYXFTXYXXX

SEQ ID NO: 75 - protein, 11 aa, Mus musculus
  misc_feature (4)..(4): Xaa can be any naturally occurring amino acid
GYSXTXGYXXH

SEQ ID NO: 76 - protein, 11 aa, Mus musculus
  misc_feature (3)..(4): Xaa can be any naturally occurring amino acid
GYXXTXXYSXX

SEQ ID NO: 77 - protein, 17 aa, Mus musculus
  misc_feature (6)..(6): Xaa can be any naturally occurring amino acid
RINPYXGATSXNXNFKD

SEQ ID NO: 78 - protein, 10 aa, Mus musculus
  misc_feature (6)..(6): Xaa can be any naturally occurring amino acid
RINPYXGATS

SEQ ID NO: 79 - protein, 17 aa, Mus musculus
  misc_feature (13)..(13): Xaa can be any naturally occurring amino acid
RINPYNGATSYNXNFKD

SEQ ID NO: 80 - protein, 17 aa, Mus musculus
  misc_feature (3)..(4): Xaa can be any naturally occurring amino acid
YIXXYXGXTXYNXXXXX

SEQ ID NO: 81 - protein, 17 aa, Mus musculus
  misc_feature (1)..(1): Xaa can be any naturally occurring amino acid
XIXXYXGXTXXNXXXXX

SEQ ID NO: 82 - protein, 10 aa, Mus musculus
  misc_feature (1)..(2): Xaa can be any naturally occurring amino acid
XXGXXYXFDY

SEQ ID NO: 83 - protein, 10 aa, Mus musculus
  misc_feature (1)..(5): Xaa can be any naturally occurring amino acid
XXXXXYXXDY

SEQ ID NO: 84 - protein, 9 aa, Mus musculus
  Xaa (1)..(6): Xaa can be any naturally occurring amino acid
XXXXXXFDX

SEQ ID NO: 85 - protein, 10 aa, Mus musculus
  misc_feature (1)..(1): Xaa can be any naturally occurring amino acid
XGXXYXXXDY

SEQ ID NO: 86 - DNA, 50 bp, Artificial Sequence
  K145E forward primer
gaaaattaaaaataatccctttgtcgagcagaagaatttaatcacattag

SEQ ID NO: 87 - DNA, 50 bp, Artificial Sequence
  K145E reverse primer
ctaatgtgattaaattcttctgctcgacaaagggattatttttaattttc

SEQ ID NO: 88 - DNA, 45 bp, Artificial Sequence
  D116R forward primer
caatgagctatctcaactttctcgtaaaacctttgccttctgcac

SEQ ID NO: 89 - DNA, 45 bp, Artificial Sequence
  D116R reverse primer
gtgcagaaggcaaaggttttacgagaaagttgagatagctcattg

SEQ ID NO: 90 - DNA, 49 bp, Artificial Sequence
  K182E forward primer
caagagcttctattatcaaacaatgagattcaagcgctaaaaagtgaag

SEQ ID NO: 91 - DNA, 49 bp, Artificial Sequence
  K182E reverse primer
cttcactttttagcgcttgaatctcattgtttgataatagaagctcttg

SEQ ID NO: 92 - DNA, 48 bp, Artificial Sequence
  N196A forward primer
gaagaactggatatctttgccgcttcatctttaaaaaaattagagttg

SEQ ID NO: 93 - DNA, 48 bp, Artificial Sequence
  N196A reverse primer
caactctaatttttttaaagatgaagcggcaaagatatccagttcttc

SEQ ID NO: 94 - DNA, 45 bp, Artificial Sequence
  E171A forward primer
ggaactcaggttcagctggccaatctccaagagcttctattatca

SEQ ID NO: 95 - DNA, 45 bp, Artificial Sequence
  E171A reverse primer
tgataatagaagctcttggagattggccagctgaacctgagttcc